(19) United States
(12) Patent Application Publication
(10) Pub. No.: US 2017/0143712 A1
(43) Pub. Date: May 25, 2017

(54) Methods of Treating Cancers, Immune and Autoimmune Diseases, and Inflammatory Diseases Based on BTK Occupancy and BTK Resynthesis Rate

(71) Applicant: Acerta Pharma B.V., Oss (NL)

(72) Inventors: Brian Lannutti, Solana Beach, CA (US); Todd Covey, San Carlos, CA (US); Allard Kaptein, Zaltbommel (NL); Dave Johnson, Aptos, CA (US); Jay Stamatis, San Mateo, CA (US); Cecile M. Krejsa, Seattle, WA (US); John Gregory Slatter, Seattle, WA (US)

(21) Appl. No.: 15/426,774

(22) Filed: Feb. 7, 2017

Related U.S. Application Data
(63) Continuation of application No. PCT/IB2016/050590, filed on Feb. 4, 2016.
Application No. PCT/IB2016/050590 is a continuation-in-part of application No. PCT/IB2015/056038, filed on Aug. 7, 2015.
(60) Provisional application No. 62/034,762, filed on Aug. 7, 2014.

(51) Int. Cl. A61K 31/4985; A61K 9/00; A61K 31/454; A61K 31/519; A61K 31/522

(52) U.S. Cl. CPC A61K 31/4985; A61K 31/519; A61K 31/522; A61K 31/454; A61K 9/0053; A61K 9/0014

(57) ABSTRACT

In an embodiment, therapeutic methods and uses of Bruton's Tyrosine Kinase (BTK) inhibitors for treatment of cancer, inflammation, immune disorders, and autoimmune disorders, including dermatoses, and for transplantation prophylaxis, based on BTK occupancies and/or BTK resynthesis rates for B cells in various diseases, tissue compartments, including bone marrow and lymph nodes, are described. In an embodiment, dosing regimens for a BTK inhibitor for treatment of cancer, inflammation, immune disorders, and autoimmune disorders, including dermatoses, and for transplantation prophylaxis, based on BTK occupancies and/or BTK resynthesis rates for B cells in various diseases, tissue compartments, including bone marrow and lymph nodes, are described.

### CROSS-REFERENCE TO RELATED APPLICATION

This application claims the benefit of International Patent Application No. PCT/IB2015/056038 filed on Aug. 7, 2015, which is incorporated by reference herein in its entirety.

### FIELD OF THE INVENTION

Therapeutic compositions and uses of Bruton's tyrosine kinase (BTK) inhibitors to treat lymphomas, leukemias, solid tumors, immune and autoimmune diseases, and inflammatory diseases based on BTK occupancies and/or resynthesis rates in cellular and tissue compartments are disclosed herein.

### BACKGROUND OF THE INVENTION

Bruton's tyrosine kinase (BTK) is a Tec family non-receptor protein kinase expressed in B cells and myeloid cells. The function of BTK in signaling pathways activated by the engagement of the B cell receptor (BCR) and FCεR1 on mast cells is well established. BTK is a key enzyme in BCR activation and plays a critical role in the maturation of B cells in bone marrow and in lymphoid tissues where antigen encounters drive the selection of high-affinity clones, immunoglobulin class switch, and development of antibody-producing plasma cells. Functional mutations in BTK in humans result in a primary immunodeficiency disease (X-linked agammaglobulinemia, XLA) characterized by a defect in B cell development with a block between pro- and pre-B cell stages. The result is an almost complete absence of B lymphocytes, causing a pronounced reduction of serum immunoglobulin of all classes. Furthermore, engagement of the BCR induces signaling through BTK and its downstream substrate PLCγ2, which activates the NFκB, a transcription factor that is essential for the development of innate and adaptive immune responses. In B lymphocytes, NEκB up-regulates the expression of pro-survival factors that support proliferation and reduce the apoptosis of B cell clones. In BCR stimulated autoreactive or malignant B cell clones, signaling through BTK can result in the inappropriate growth or survival of disease-inducing B cells leading to auto-antibody production, inflammation, lymphadenopathy, and reactive cytopenias. In mice with spontaneous mutations in BTK, constitutive activation or inactivation of BTK signaling activity leads to severe immunodeficiency disease, suggesting that in B cells, tight developmental control over BTK expression and signaling is essential for properly tuned adaptive immune function. In addition to BCR signals, activation of BTK occurs in response to other signals that lead to the induction of auto-reactive B cells, such as TLR9, a receptor for nucleic acids, and in response to signals that initiate inflammatory processes causing structural damage in autoimmune disease, such as FcεR1 in mast cells and RANKL in osteoclasts. These findings support a key role for BTK in the regulation of the production of auto-antibodies and inflammation in autoimmune diseases.

Regulation of BTK expression levels during B cell development and activation is tightly controlled; in hematopoeisis and in pre- and pro-B cell stages, the BTK level is relatively high. In peripheral tissues, resting B lymphocytes have lower BTK than is observed in bone marrow. Stimulation of BCR leads to rapid induction of BTK expression, with increases of 10-fold protein levels within several hours of stimulation, as described in (Nisitani, et al., PNAS. 2000, 97, 2737-2742). In B cells, expression of BTK results from NFκB-mediated transcriptional activation in addition to a post-translational mechanism that occurs rapidly after BCR stimulation (Yu, et al., Blood, 2008, 111(9), 4617-4626). Since BTK signaling induces NFκB, there is a positive feedback loop in activated B cells. These findings suggest that inhibition of BTK affects both the downstream sequelae of BCR engagement, such as antibody production, as well as inhibiting the expression of BTK itself, which could further regulate the reactivity of autoimmune B cells.

Other diseases with an important role for dysfunctional B cells are B cell malignancies. The reported role of BTK in BCR signaling, and in the regulation of proliferation and apoptosis of B cells, indicates the potential for BTK inhibitors in the treatment of B cell lymphomas. BTK inhibitors have thus been developed as potential therapies, as described in: Cruz, et al., OncoTargets & Therapy 2013, 6, 161-176; Hendriks, et al., Nat. Rev. Cancer, 2014, 14, 219-232; Byrd, et al., N Engl. J. Med. 2013, 369, 32-42.

B cells are a key component of the adaptive immune system. In adults, B cells initially develop from hematopoietic stem cells in the bone marrow, and mature into progenitor B cells (pro-B cells), pre-B cells, immature B cells, and naïve B cells in the marrow, with their stage of development characterized by the expression of cell surface proteins, as described in Perez-Andres, et al., Cytometry B (Clinical Cytometry), 2013, 78B (Suppl. 1), S47-S60 and Allman, et al., Curr. Opin. Immunol. 2008, 20, 149-157. B cells that exit the bone marrow may migrate to the spleen and secondary lymphoid organs and undergo additional development following antigen stimulation, which also leads to the expression of cell surface proteins that characterize the activation and developmental stage of the B cell, and which depends on functional T cell help. The skewing of T cells in part depends on the context in which antigens are presented, by B cells or professional antigen producing cells (APCs) of myeloid origin, such as dendritic cell subsets (e.g., follicular dendritic cells, Langerhans cells) and activated monocytes and/or macrophases. In fact many myeloid derived cells also contain functional BTK. The quality of antigen presentation by these cells, with regard to promoting inflammation and T cell fate (as helper, inflammatory, or suppressor cells), depends on the activation and maturation status of the APC, which may be affected by stimulation through the BTK pathway. Therefore, multiple signals integrate to direct the development of B cells in peripheral compartments following migration from bone marrow. After antigen stimulation occurs in specialized peripheral tissue compartments, B cells may further differentiate into subsets and may recirculate into different tissues including mucosa and the BM, where long-living plasma cells produce antibodies, and to sites of inflammation such as synovial tissue in rheumatoid arthritis (RA) and osteoarthritis (OA), brain parenchyma in multiple sclerosis (MS), exocrine glands in Sjogren's syndrome (SS) and skin/connective tissue in bullous pemphigoid, psoriasis vulgaris, systemic lupus erythmatosis (SLE), and scleroderma/systemic sclerosis.

The present invention includes the unexpected discovery that the rate of BTK resynthesis per cell and the rate of regeneration of BTK expressing B cells following treatment of a human with a covalent inhibitor of BTK, differs between disease states and and healthy individuals, and can also differ between individuals that are otherwise affected by the same disease indication.

The present invention includes the unexpected discovery that the inhibition of BTK at therapeutically relevant sites within the body of a mammal can be achieved by treatment with low doses of an agent that covalently inactivates the BTK kinase, provided the low doses are delivered at intervals that match or exceed the rate of synthesis of new BTK positive target cells or the re-synthesis of BTK within existing and newly generated target cells.

Additionally, the present invention includes the novel finding that in humans treatment with an inhibitor of BTK that covalently inactivates the BTK kinase directly impacts the resynthesis rate of BTK, causing a decrease in BTK resynthesis rates once full inhibition has been attained and leading to reduced BTK content on a per-cell basis in target B cells of healthy volunteers and leukemic B cells of patients with chronic lymphocytic leukemia (CLL).

The compartments in which BCR signaling is most active, and the compartments in which immune cell proliferation is most rapid, will have higher BTK resynthesis rates. The novelty in this invention is due to the unexpected effect of the covalent BTK inhibitor on the BTK resynthesis rate, and the tight correlation between BTK resynthesis and BTK target occupancy. Because of the irreversible nature of the BTK kinase interaction with the covalent inhibitor, the pharmacokinetic/pharmacodynamic effects of BTK signaling inhibition are tied to the resynthesis rate of BTK.

Depending on the degree of BTK inhibition, impaired signaling through the BTK pathway can result in different effects on the BTK resynthesis rate. In humans, following treatment with a covalent inhibitor of BTK that results in lower levels of measured BTK target occupancy, an increased rate of BTK resynthesis is observed; whereas, after treatment with an appropriate dose and schedule to attain higher levels of BTK inhibition, a decreased of BTK resynthesis is observed. This novel result in humans demonstrates the importance of achieving the correct degree of BTK inhibition in the tissue compartment of interest.

The present invention includes the discovery that BTK target occupancy, as measured in peripheral blood of humans treated with an agent that covalently inactivates the BTK kinase, reflects BTK target occupancy in one or more tissue compartments outside of the peripheral blood. BTK target occupancy can also be accurately measured in the tissue compartments by a variety of methods. The rate of de novo BTK resynthesis in a human or a mammal treated with an agent that covalently inactivates the BTK kinase is directly proportional to the generation of unoccupied BTK at target sites as measured in a BTK target occupancy assay. The rate of BTK resynthesis can be predicted with computational models utilizing concentration-time profiles of the covalent BTK inhibitor and BTK target occupancy data from peripheral blood and tissue compartments. The prediction of BTK resynthesis in the compartment of interest may be used to identify target doses and/or dosing schedules that will provide sufficient exposure to the BTK inhibitor to fully inhibit BTK in the compartment of interest and to reduce the resynthesis rate of BTK during the dosing interval.

The present invention includes the unexpected discovery that dosing schedules can be adjusted to effect BTK inhibition of a desired magnitude, such that functional inhibition of B cell receptor (BCR) signaling is maintained in the disease tissue compartment of interest, without necessarily increasing the plasma Cmax following oral administration.

Additionally, different compartments within the body have different BTK resynthesis rates. In an embodiment, the method of use for treating specific diseases with a BTK inhibitor relates to treating the most active resynthesis compartment for that disease, in effect tailoring the dosing regimen of a BTK inhibitor to resynthesis rate in that compartment.

In rheumatoid arthritis (RA) and osteoarthritis, the inflammatory milleau of diseased joints results in development of lymphoid follicle-like structures in the tissues with high rates of proliferation and autoantigen-specific stimulation of B cell receptor signaling. At sites of inflammatory bone disease, osteoclasts stimulated by inflammatory factors such as receptor activator of nuclear factor kappa-β ligand (RANKL) induce BTK signals, resulting in an activated phenotype and secretion of osteolytic enzymes, further damaging the bone in this compartment. Treatment of patients with RA or osteolytic bone disease with a covalent inhibitor of BTK kinase requires sufficient delivery of the BTK inhibitor to the compartment of synovial fluid, diseased joints or bone. The method of use comprises the inhibition of BCR-mediated signaling by inhibiting BTK in these compartments to reduce the inflammation and progressive destruction of joints and bone tissue.

In lupus nephritis, cross-linking of autoreactive antibodies and deposition of immune complexes in the glomeruli of the kidney results in an inflammatory response that leads to endothelial and epithelial activation of tissues in the kidney cortex, extravasation of monocytes and activation of tissue macrophages, recruitment of neutrophils and activated fibroblasts, and the progressive loss of glomerular function. In systemic lupus erythematosus (SLE), the development of autoantibodies occurs in tissue compartments where following BCR stimulation, inappropriate survival of autoreactive B cell clones and maturation of autoreactive B cells into plasma cells occurs. The method of use comprises the inhibition of BTK in compartments where autoreactive B cells proliferate and/or produce autoantibodies, and the inhibition of BTK in compartments associated with tissue inflammation such as kidney, connective tissue and skin.

In chronic lymphocytic leukemia (CLL) and small lymphocytic lymphoma, activation of BTK through BCR signaling in neoplastic B cells within the compartment of the bone marrow drives proliferation of the tumor, induction of anti-apoptotic proteins, and release of malignant cells into the central blood compartment and the peripheral lymphoid tissues such as lymph nodes and spleen, which become sites of lymphadenopathy. Additionally, CLL and small lymphocytic lymphoma (SLL) cells may undergo further proliferation in sites of lymphadenopathy, as evidenced by the presence of Ki67, a proliferation marker, within these tissues. While absolute lymphocyte count (ALC) is monitored during treatment of CLL, responses at the sites of lymphadenopathy and in the bone marrow require the penetration of effective treatment into these compartments.

In diffuse large B-cell lymphoma (DLBCL), intra-patient diversity may exist in the proliferative rate of lymphadenopathic nodes or extranodal lesions exists. For example, higher metabolic activity is observed on positron emission tomography (PET) scans for a subset of lymphomatous lymph nodes within a patient's body. The proliferation rate of the distinct lesions represents different rates of de novo BTK synthesis and may be considered to be separate compartments with higher or lower rates of BTK resynthesis.

In DLBCL, inter-patient diversity in proliferative rate may be associated with specific mutations such as p53 inactivation, expression of the proto-oncogene c-Myc, and expression of antiapoptotic proteins such as Bcl-2 or Bcl-6, among other markers of aggressiveness. In such patient subsets, and others defined by the proliferative rate or BTK resynthesis rate, different therapeutic doses and/or regimens of a BTK inhibitor are identified.

Highly proliferative or aggressive DLBCL reflects enhanced BCR signaling, as designated in the “activated B cell” subset of tumors, which may contribute to a rapid resynthesis of BTK as well as dependency on the BTK signaling pathway to propogate the BCR-mediated growth signals.

In solid tumors, stromal components usually include a variable number of tumor-associated lymphocytes and myeloid cells such as tumor associated macrophages, which may exert pro-angiogenic and immunosuppressive effects within the tumor microenvironment. These cells have the ability to alter the phenotype and function of new infiltrating cells toward activation, surveillance and immune-mediated destruction of malignant cells, or toward an immunosuppressive phenotype. Thus, regulatory B and T lymphocytes (Bregs and Tregs), myeloid derived suppressor cells (MDSCs) and tissue resident histiocytes, dendritic cells and mast cells may provide stromal support and reduce innate and adaptive immune surveillance against transformed cells. The immune component of the tumor microenvironment is therefore also a tissue compartment of therapeutic interest when using a BTK inhibitor to treat solid tumors and hematologic malignancies characterized by infiltrating or stromal cells.

### SUMMARY OF THE INVENTION

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the target BTK occupancy is selected from the group consisting of greater than 85%, greater than 90%, greater than 91%, greater than 92%, greater than 93%, greater than 94%, greater than 95%, greater than 96%, greater than 97%, greater than 98%, and greater than 99%.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the BTK occupancy is estimated from a BTK resynthesis rate in a tumor lesion or a site of disease.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the BTK occupancy is estimated by a metabolic activity profile or a proliferative index in a tumor lesion or a site of disease. In an embodiment, the metabolic activity profile is measured using a method selected from the group consisting of magnetic resonance imaging and positron emission tomography.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the BTK occupancy is evaluated based on the binding of a BTK probe that binds to unoccupied BTK in a tumor lesions or site of disease. In an embodiment, the BTK probe is selected from the group consisting of a fluorescent probe and a positron emission tomography probe.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the BTK occupancy is evaluated based on the average BTK resynthesis rate in a population of patients with the BTK mediated disease.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the tissue compartment is selected from the group consisting of peripheral blood B cells, bone marrow B cells, lymph node B cells, autoreactive B cells, plasma cells, regulatory B cells, follicular dendritic cells, myeloid-derived dendritic cells, tumor stroma, tumor-associated macrophage, mast cells, alveolar macrophages, dust cells, plasmacytoid dendritic cells, cutaneous lymphocyte antigen (CLA)-positive T cells, lymphoid-inducer cells, Langerhans cells, monocytes, macrophages, histiocytes, Kupffer cells, glial cells, microglia, Schwann cells, Ito cells, hepatic stellate cells, pancreatic stellate cells, glioma cells, malignant B cells, adipocytes, sarcoid cells, granulocytes, neutrophils, eosinophils, hematopoietic stem cells, serous cells, mesenchymal stromal cells, osteoblasts, osteoclasts, infiltrating lymphocytes, immunocytes and inflammatory infiltrates.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the tissue compartment is selected from the group consisting of peripheral blood, bone marrow, germinal center, lymphoid follicle, gut-associated lymphoid tissue, tonsil, lymphoma lesion, ectopic lymphoid tissue, ectopic node, lymph node lesion, lymphadenopathy, spleen, solid tumor, tumor microenvironment, tumor stroma, bone, bone lesion, bone metastasis, synovial fluid, articular surface, joint, kidney, liver, lung, bronchus/bronchiole, mediastinum, pleura, peritoneum, cystadenocarcinoma, heart, pancreas, sinusoid, eye, nerve, brain, brain metastasis, brain lesion, central nervous system, skin, stomach, lamina propria, gut, colon, exocrine gland, salivary gland, lacrimal gland, breast, dermis, subdermis, epidermis, perivascular, inflammatory lesion, cutaneous lesion, granuloma, mastocytoma, papule, testis, ovary, and bladder.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the BTK inhibitor is selected from the group consisting of:

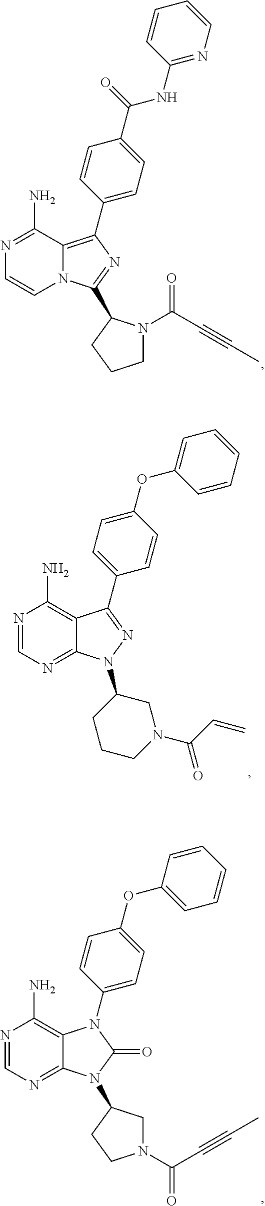 [SMILES: CC#CC(=O)N1CCC[C@H]1c1nc(-c2ccc(C(=O)Nc3ccccn3)cc2)c2c(N)nccn12 ; C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1]

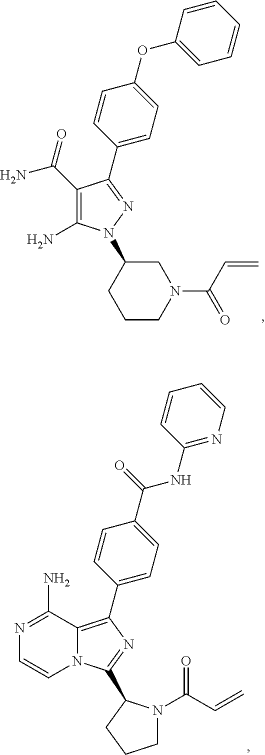 [SMILES: C=CC(=O)N1CCC[C@@H](n2nc(-c3ccc(Oc4ccccc4)cc3)c(C(N)=O)c2N)C1 ; C=CC(=O)N1CCC[C@H]1c1nc(-c2ccc(C(=O)Nc3ccccn3)cc2)c2c(N)nccn12]

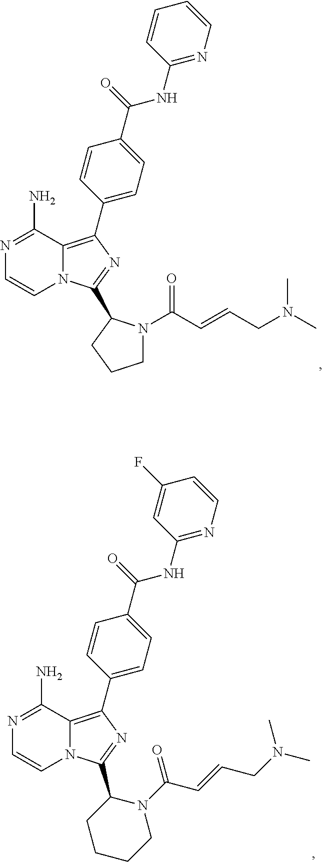 [SMILES: CN(C)C/C=C/C(=O)N1CCC[C@H]1c1nc(-c2ccc(C(=O)Nc3ccccn3)cc2)c2c(N)nccn12 ; CN(C)C/C=C/C(=O)N1CCCC[C@H]1c1nc(-c2ccc(C(=O)Nc3cc(F)ccn3)cc2)c2c(N)nccn12]

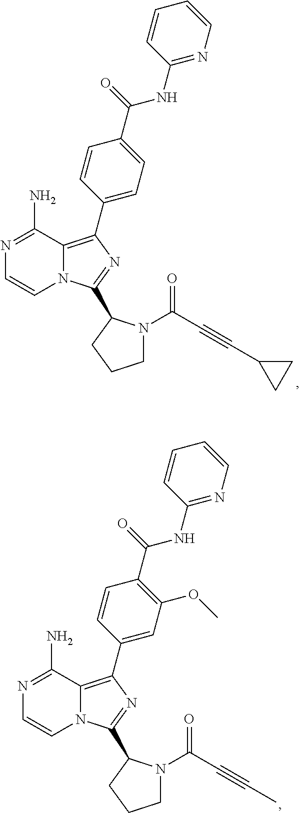 [SMILES: Nc1nccn2c([C@@H]3CCCN3C(=O)C#CC3CC3)nc(-c3ccc(C(=O)Nc4ccccn4)cc3)c12 ; CC#CC(=O)N1CCC[C@H]1c1nc(-c2ccc(C(=O)Nc3ccccn3)c(OC)c2)c2c(N)nccn12]

or a pharmaceutically-acceptable salt, cocrystal, hydrate, solvate, or prodrug thereof.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, further comprising the step of determining the target BTK occupancy in the tissue compartment using a relative resynthesis rate.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the first dose of the BTK inhibitor is administered once daily.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the first dose of the BTK inhibitor is administered twice daily.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the first dose of the BTK inhibitor is administered three times daily.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the second dose of the BTK inhibitor is administered once daily.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the second dose of the BTK inhibitor is administered twice daily.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the second dose of the BTK inhibitor is administered three times daily.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the first dose of the BTK inhibitor is selected from the group consisting of 5 mg, 10 mg, 15 mg, 20 mg, and 25 mg.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the second dose of the BTK inhibitor is selected from the group consisting of 5 mg, 10 mg, 15 mg, 20 mg, and 25 mg.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the first period is selected from the group consisting of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, and 21 days.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the second period is selected from the group consisting of 2 weeks, 1 month, 2 months, 3 months, 6 months, 9 months, 1 year, 2 years, 3 years, 4 years, 5 years, 6 years, 7 years, 8 years, 9 years, and 10 years.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the first dose of the BTK inhibitor is administered orally.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the first dose of the BTK inhibitor is administered topically.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the second dose of the BTK inhibitor is administered orally.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the second dose of the BTK inhibitor is administered topically.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the BTK mediated disease is a cancer selected from the group consisting of a cancer selected from the group consisting of non-Hodgkin's lymphoma, acute myeloid leukemia, chronic lymphocytic leukemia, small lymphocytic lymphoma, diffuse large B cell lymphoma, mantle cell lymphoma, MALT lymphoma, Waldenström's macroglobulinemia, follicular lymphoma, B cell acute lymphoblastic leukemia, Burkitt's leukemia, juvenile myelomonocytic leukemia, prolymphocytic leukemia, mast cell leukemia, hairy cell leukemia, Hodgkin's disease, multiple myeloma, thymus cancer, brain cancer, glioma, lung cancer, squamous cell cancer, skin cancer, melanoma, eye cancer, retinoblastoma, intraocular melanoma, oral cavity cancer, oropharyngeal cancer, bladder cancer, gastric cancer, stomach cancer, pancreatic cancer, breast cancer, cervical cancer, head cancer, neck cancer, renal cancer, kidney cancer, liver cancer, ovarian cancer, prostate cancer, colorectal cancer, bone cancer, esophageal cancer, testicular cancer, gynecological cancer, thyroid cancer, central nervous system cancer, cancer related to acquired immune deficiency syndrome, cervical carcinoma, nasopharyngeal carcinoma, Kaposi's sarcoma and, primary effusion lymphoma, adenocystic carcinoma, hepatocellular carcinoma, T-cell leukemia, and mastocytosis.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the BTK mediated disease is an inflammatory, immune, or autoimmune disorder selected from the group consisting of tumor angiogenesis, chronic inflammatory disease, rheumatoid arthritis, osteoarthritis, osteoporosis, atherosclerosis, inflammatory bowel disease, skin diseases such as psoriasis, eczema, and scleroderma, systemic sclerosis, diabetes, diabetic retinopathy, retinopathy of prematurity, age-related macular degeneration, hemangioma, ulcerative colitis, atopic dermatitis, pouchitis, spondylarthritis, uveitis, Behcet's disease, polymyalgia rheumatica, giant-cell arteritis, sarcoidosis, Kawasaki disease, juvenile idiopathic arthritis, hidratenitis suppurativa, Sjögren's syndrome, psoriatic arthritis, juvenile rheumatoid arthritis, ankylosing spoldylitis, asthma, Crohn's disease, lupus, lupus nephritis, and polycythemia vera.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the BTK mediated disease is an immune rejection associated with an organ or cell transplant selected from the group consisting of a disorder associated with anti-allogeneic antibodies, a disorder associated with allograft rejection prior to, during, or after organ or cell transplantation, pre-transplant conditioning of patients receiving solid organ transplant, a disorder associated with humoral acute rejection, a disorder associated with heart transplantation, a disorder associated with renal transplantation, a disorder associated with kidney transplantation, a disorder associated with lung transplantation, a disorder associated with liver transplantation, a disorder associated with ABO-incompatible transplantation, and a disorder associated with stem cell transplantation.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the BTK mediated disease is a graft-versus-host disease (GVHD), wherein the GVHD is selected from the group consisting of GVHD associated with stem cell transplant, GVHD associated with bone marrow transplant, thymus GVHD, skin GVHD, gastrointestinal GVHD, liver GVHD, acute GVHD, and chronic GVHD.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the BTK mediated disease is a dermatosis, wherein the dermatosis is selected from the group consisting of psoriasis vulgaris, guttate psoriasis, erythrodermic psoriasis, psoriatic nails, annular pustular psoriasis, pustular psoriasis, inverse psoriasis, psoriatic arthritis, keratoderma blennorrhagicum, parapsoriasis, erythema nodosum, palmoplantar hidradentitis, atopic dermatitis, atopic eczema, seborrheic eczema, seborrheic dermatitis, dyshidrosis, rosacea, cutaneous lupus erythematosus, acute cutaneous lupus erythematosus, subacute cutaneous lupus erythematosus, discoid lupus erythematosus, lupus erythromatosus tumidus, lupus nephritis, lupus erythematosus panniculitis, erythema multiforme, verruca, verrucous lupus erythematosus, vitiligo, alopecia areata, purigo nodularis, lichen planus, purigo pigmentosum, pemphigus vulgaris, bullous pemphigoid, pemphigus erythematosus, pemphigus nodularis, erythrodermic sarcoidosis, granulomatous dermatisis, scleroderma, systemic sclerosis, cutaneous manifestations of systemic sclerosis, diffuse cutaneous mastocytosis, erythrodermic mastocytosis, granuloma annulare, chondrodermatitis nodularis, contact dermatitis, drug eruptions, linear IgA bullous dermatosis, eosinophilic dermatitis, keratosis pilaris, lymphomatoid papulosis, pityriasis lichenoides et varioliformis acuta (PLEVA), lichenoides chronica (PLC), febrile ulceronecrotic Mucha-Habermann disease (FUMHD), chronic urticaria, rheumatoid neutrophilic dermatitis, cutaneous manifestations of graft-versus-host disease, cryoglobulinemic purpura, and purpura hyperglobulinemica.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein the BTK mediated disease is a dermatosis, wherein the dermatosis results from dermal manifestations of systemic diseases where sensitization, lymphocyte recruitment, lymphocyte skewing by local or lymph-node antigen presenting cells, activation of skin-resident or skin-homing lymphocytes, innate immune sensing, keratinocyte antimicrobial responses, activation of resident or infiltrating myeloid dendritic cells, plasmacytoid dendritic cells, macrophages, mast cells, neutrophils, and/or Langerhans cells, and wherein the dermatosis leads to the development of skin lesions.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein a diagnostic tool is used for an evaluation of BTK expression and/or resynthesis in the BTK mediated disease for determination of the optimal treatment regimen with the BTK inhibitor.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein a diagnostic tool is used for an evaluation of BTK expression and/or resynthesis in the BTK mediated disease for determination of the optimal treatment regimen with the BTK inhibitor, wherein the evaluation of BTK expression and/or resynthesis occurs prior to treatment of the BTK mediated disease.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein a diagnostic tool is used for an evaluation of BTK expression and/or resynthesis in the BTK mediated disease for determination of the optimal treatment regimen with the BTK inhibitor, wherein the evaluation of BTK expression and/or resynthesis occurs during the treatment of the BTK mediated disease.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein a diagnostic tool is used for an evaluation of BTK expression and/or resynthesis in the BTK mediated disease for determination of the optimal treatment regimen with the BTK inhibitor, wherein the evaluation of BTK expression and/or resynthesis is used to identify patients that are likely to benefit from treatment with Formula (II).

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein a diagnostic tool is used for an evaluation of BTK expression and/or resynthesis in the BTK mediated disease for determination of the optimal treatment regimen with the BTK inhibitor, wherein the evaluation of BTK expression and/or resynthesis is used to identify patients that are unlikely to benefit from treatment with Formula (II).

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein a diagnostic tool is used for an evaluation of BTK expression and/or resynthesis in the BTK mediated disease for determination of the optimal treatment regimen with the BTK inhibitor, wherein the evaluation of BTK expression and/or resynthesis is conducted using a member of the BTK pathway or a pharmacodynamic sequel of BTK pathway activation.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein a diagnostic tool is used for an evaluation of BTK expression and/or resynthesis in the BTK mediated disease for determination of the optimal treatment regimen with the BTK inhibitor, wherein the diagnostic tool is a kit.

In a preferred embodiment, the invention includes a method of treating a BTK mediated disease in a human comprising the steps of: (a) administering a Bruton's tyrosine kinase (BTK) inhibitor at a first dose for a first period sufficient to provide a target BTK occupancy in a tissue compartment; and (b) administering the BTK inhibitor at a second dose for a second period, wherein the second dose is less than the first dose and is sufficient to provide the target BTK occupancy in the tissue compartment, wherein a diagnostic tool is used for an evaluation of BTK expression and/or resynthesis in the BTK mediated disease for determination of the optimal treatment regimen with the BTK inhibitor, wherein the diagnostic tool is a laboratory-developed assay.

In a preferred embodiment, the invention includes a method of treating a disorder in a human comprising the step of administering a dose of a compound selected from the group consisting of:

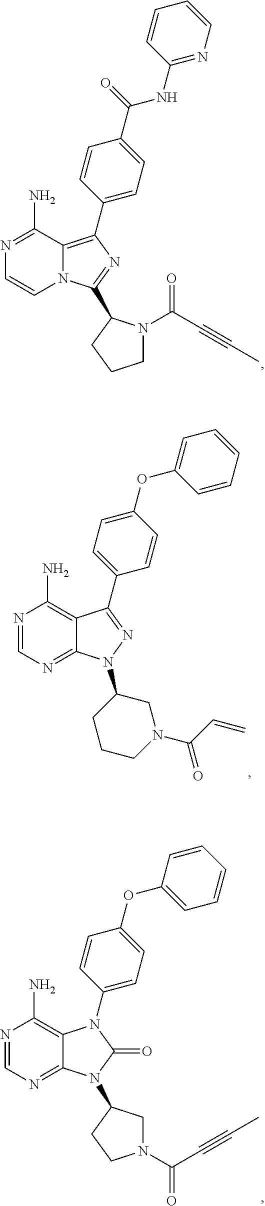 [SMILES: CC#CC(=O)N1CCC[C@H]1c1nc(-c2ccc(C(=O)Nc3ccccn3)cc2)c2c(N)nccn12 ; C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1]

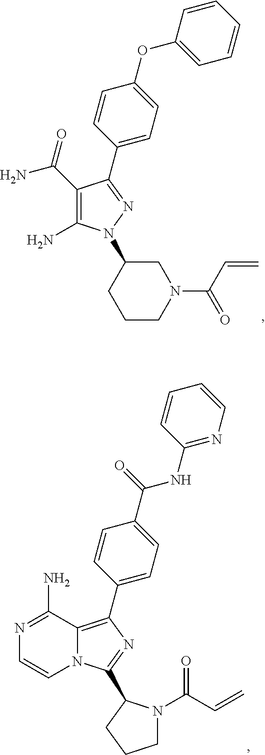 [SMILES: C=CC(=O)N1CCC[C@@H](n2nc(-c3ccc(Oc4ccccc4)cc3)c(C(N)=O)c2N)C1 ; C=CC(=O)N1CCC[C@H]1c1nc(-c2ccc(C(=O)Nc3ccccn3)cc2)c2c(N)nccn12]

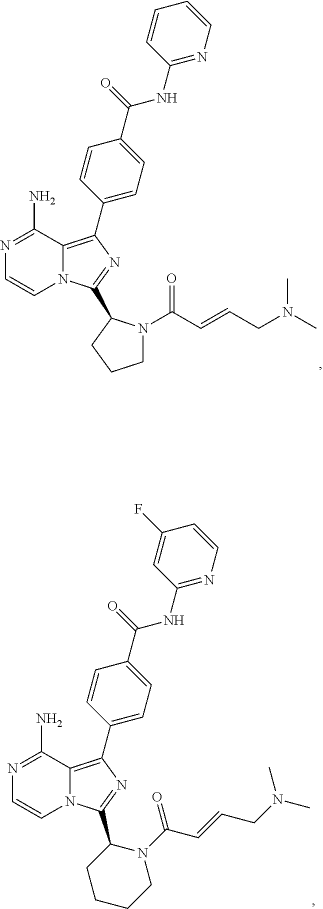 [SMILES: CN(C)C/C=C/C(=O)N1CCC[C@H]1c1nc(-c2ccc(C(=O)Nc3ccccn3)cc2)c2c(N)nccn12 ; CN(C)C/C=C/C(=O)N1CCCC[C@H]1c1nc(-c2ccc(C(=O)Nc3cc(F)ccn3)cc2)c2c(N)nccn12]

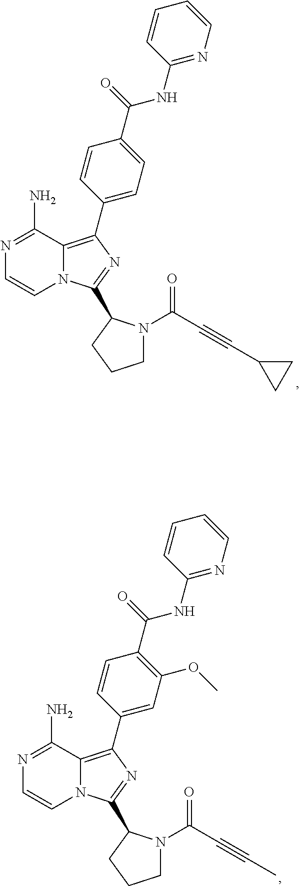 [SMILES: Nc1nccn2c([C@@H]3CCCN3C(=O)C#CC3CC3)nc(-c3ccc(C(=O)Nc4ccccn4)cc3)c12 ; CC#CC(=O)N1CCC[C@H]1c1nc(-c2ccc(C(=O)Nc3ccccn3)c(OC)c2)c2c(N)nccn12]

or a pharmaceutically-acceptable salt, cocrystal, hydrate, solvate, or prodrug thereof, wherein the dose is selected from the group consisting of 5 mg, 10 mg, 15 mg, 20 mg, and 25 mg, wherein the dose is administered once daily, twice daily, or three times daily, and wherein the dose is administered by a route of administration selected from the group consisting of oral administration, topical administration, and combinations thereof.

In a preferred embodiment, the invention includes a method of treating a disorder in a human comprising the step of administering a dose of a compound selected from the group consisting of:

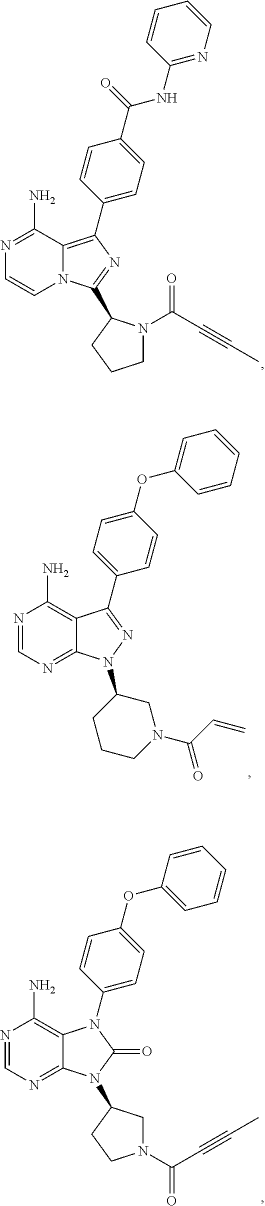 [SMILES: CC#CC(=O)N1CCC[C@H]1c1nc(-c2ccc(C(=O)Nc3ccccn3)cc2)c2c(N)nccn12 ; C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1]

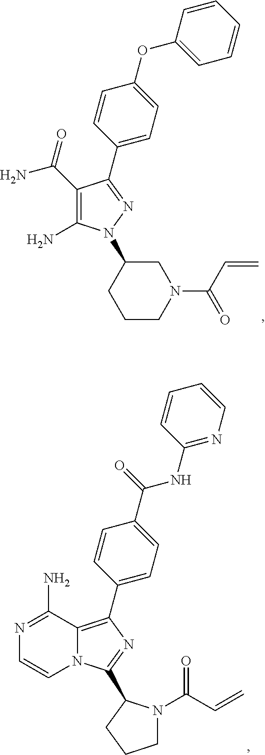 [SMILES: C=CC(=O)N1CCC[C@@H](n2nc(-c3ccc(Oc4ccccc4)cc3)c(C(N)=O)c2N)C1 ; C=CC(=O)N1CCC[C@H]1c1nc(-c2ccc(C(=O)Nc3ccccn3)cc2)c2c(N)nccn12]

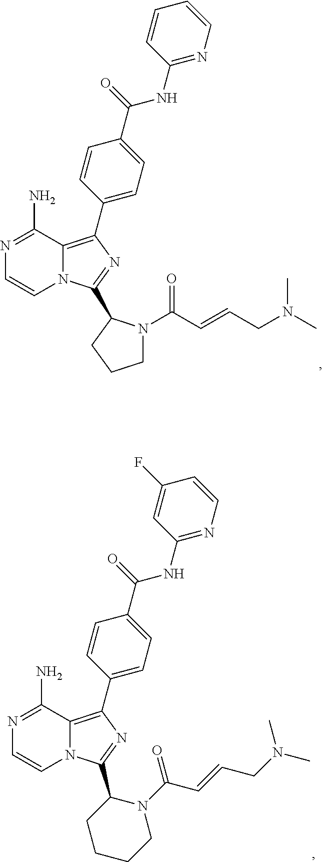 [SMILES: CN(C)C/C=C/C(=O)N1CCC[C@H]1c1nc(-c2ccc(C(=O)Nc3ccccn3)cc2)c2c(N)nccn12 ; CN(C)C/C=C/C(=O)N1CCCC[C@H]1c1nc(-c2ccc(C(=O)Nc3cc(F)ccn3)cc2)c2c(N)nccn12]

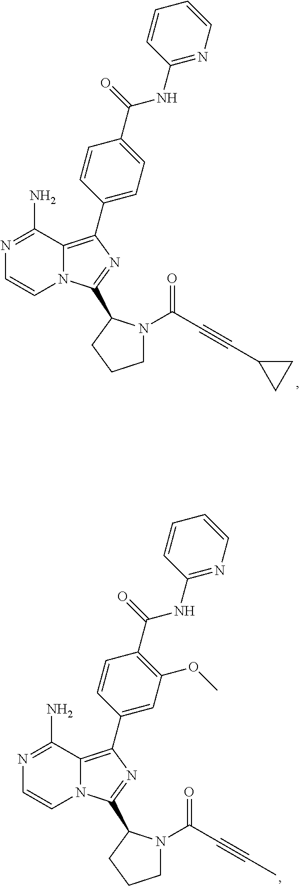 [SMILES: Nc1nccn2c([C@@H]3CCCN3C(=O)C#CC3CC3)nc(-c3ccc(C(=O)Nc4ccccn4)cc3)c12 ; CC#CC(=O)N1CCC[C@H]1c1nc(-c2ccc(C(=O)Nc3ccccn3)c(OC)c2)c2c(N)nccn12]

or a pharmaceutically-acceptable salt, cocrystal, hydrate, solvate, or prodrug thereof, wherein the dose is selected from the group consisting of 5 mg, 10 mg, 15 mg, 20 mg, and 25 mg, wherein the dose is administered once daily, twice daily, or three times daily, and wherein the dose is administered by a route of administration selected from the group consisting of oral administration, topical administration, and combinations thereof, wherein the disorder is a cancer, and wherein the cancer is selected from the group consisting of non-Hodgkin's lymphoma, acute myeloid leukemia, chronic lymphocytic leukemia, small lymphocytic lymphoma, diffuse large B cell lymphoma, mantle cell lymphoma, Waldenström's macroglobulinemia, follicular lymphoma, B cell acute lymphoblastic leukemia, Burkitt's leukemia, juvenile myelomonocytic leukemia, mast cell leukemia, hairy cell leukemia, Hodgkin's disease, multiple myeloma, thymus cancer, brain cancer, glioma, lung cancer, squamous cell cancer, skin cancer, melanoma, eye cancer, retinoblastoma, intraocular melanoma, oral cavity cancer, oropharyngeal cancer, adenocystic carcinoma, bladder cancer, gastric cancer, stomach cancer, pancreatic cancer, breast cancer, cervical cancer, head cancer, neck cancer, renal cancer, kidney cancer, liver cancer, ovarian cancer, prostate cancer, colorectal cancer, bone cancer, esophageal cancer, testicular cancer, gynecological cancer, thyroid cancer, central nervous system cancer, cancer related to acquired immune deficiency syndrome, cervical carcinoma, nasopharyngeal carcinoma, Kaposi's sarcoma and, primary effusion lymphoma, hepatocellular carcinoma, T-cell leukemia, and mastocytosis.

In a preferred embodiment, the invention includes a method of treating a disorder in a human comprising the step of administering a dose of a compound selected from the group consisting of:

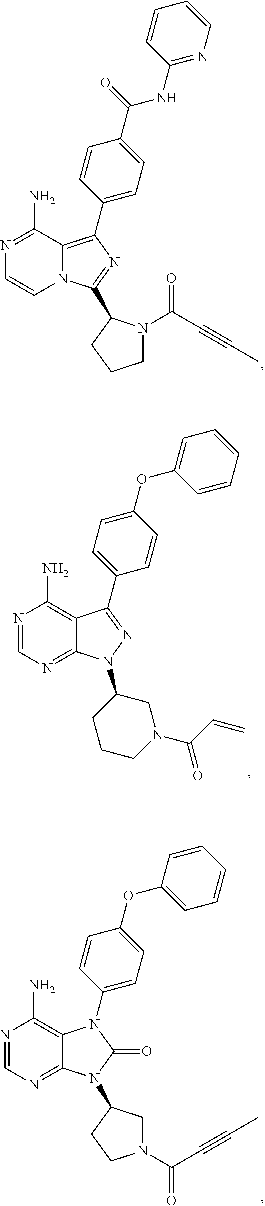 [SMILES: CC#CC(=O)N1CCC[C@H]1c1nc(-c2ccc(C(=O)Nc3ccccn3)cc2)c2c(N)nccn12 ; C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1]

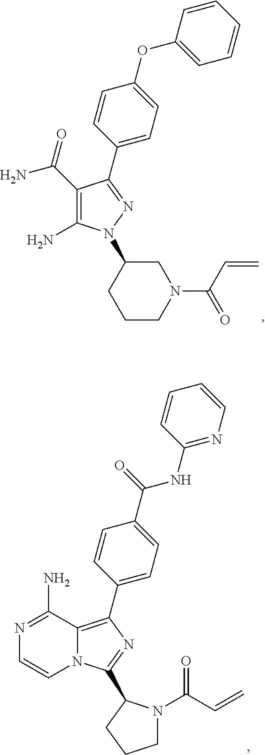 [SMILES: C=CC(=O)N1CCC[C@@H](n2nc(-c3ccc(Oc4ccccc4)cc3)c(C(N)=O)c2N)C1 ; C=CC(=O)N1CCC[C@H]1c1nc(-c2ccc(C(=O)Nc3ccccn3)cc2)c2c(N)nccn12]

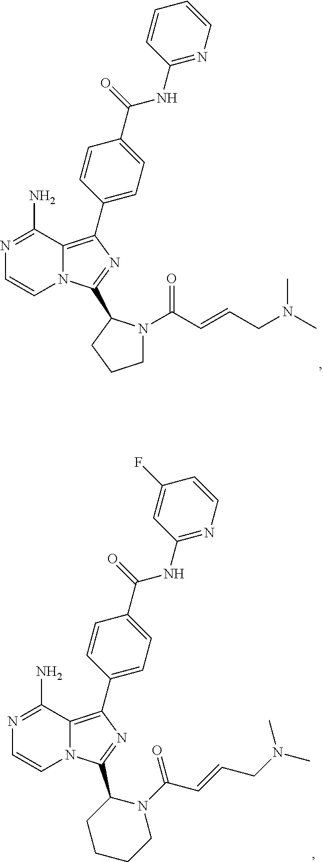 [SMILES: CN(C)C/C=C/C(=O)N1CCC[C@H]1c1nc(-c2ccc(C(=O)Nc3ccccn3)cc2)c2c(N)nccn12 ; CN(C)C/C=C/C(=O)N1CCCC[C@H]1c1nc(-c2ccc(C(=O)Nc3cc(F)ccn3)cc2)c2c(N)nccn12]

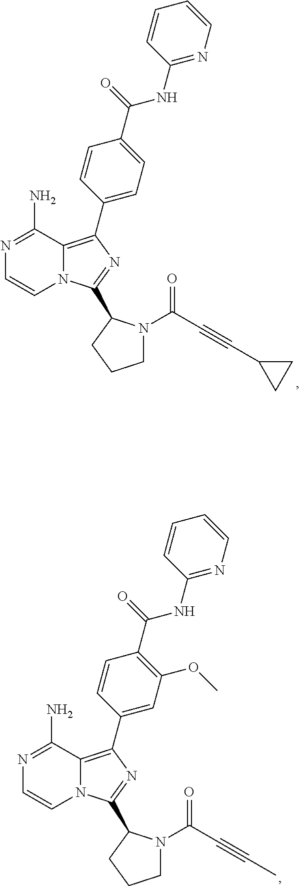 [SMILES: Nc1nccn2c([C@@H]3CCCN3C(=O)C#CC3CC3)nc(-c3ccc(C(=O)Nc4ccccn4)cc3)c12 ; CC#CC(=O)N1CCC[C@H]1c1nc(-c2ccc(C(=O)Nc3ccccn3)c(OC)c2)c2c(N)nccn12]

or a pharmaceutically-acceptable salt, cocrystal, hydrate, solvate, or prodrug thereof, wherein the dose is selected from the group consisting of 5 mg, 10 mg, 15 mg, 20 mg, and 25 mg, wherein the dose is administered once daily, twice daily, or three times daily, and wherein the dose is administered by a route of administration selected from the group consisting of oral administration, topical administration, and combinations thereof, wherein the disorder is an inflammatory, immune, or autoimmune disorder selected from the group consisting of tumor angiogenesis, chronic inflammatory disease, rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, ulcerative colitis, atopic dermatitis, pouchitis, spondylarthritis, uveitis, Behcet's disease, polymyalgia rheumatica, giant-cell arteritis, sarcoidosis, Kawasaki disease, juvenile idiopathic arthritis, hidratenitis suppurativa, Sjögren's syndrome, psoriatic arthritis, juvenile rheumatoid arthritis, ankylosing spoldylitis, asthma, Crohn's disease, lupus, lupus nephritis, and polycythemia vera.

In a preferred embodiment, the invention includes a method of treating a disorder in a human comprising the step of administering a dose of a compound selected from the group consisting of:

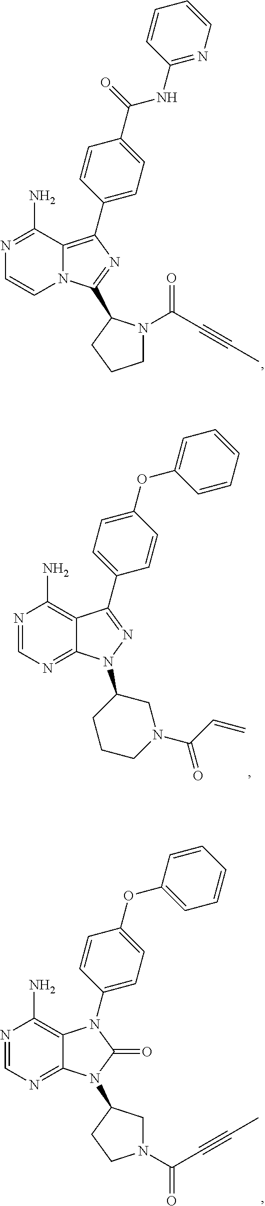 [SMILES: CC#CC(=O)N1CCC[C@H]1c1nc(-c2ccc(C(=O)Nc3ccccn3)cc2)c2c(N)nccn12 ; C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1]

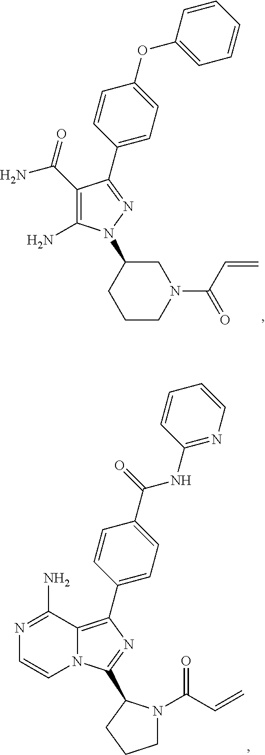 [SMILES: C=CC(=O)N1CCC[C@@H](n2nc(-c3ccc(Oc4ccccc4)cc3)c(C(N)=O)c2N)C1 ; C=CC(=O)N1CCC[C@H]1c1nc(-c2ccc(C(=O)Nc3ccccn3)cc2)c2c(N)nccn12]

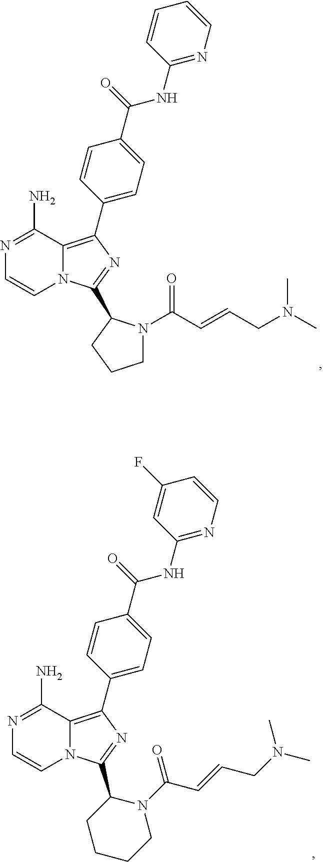 [SMILES: CN(C)C/C=C/C(=O)N1CCC[C@H]1c1nc(-c2ccc(C(=O)Nc3ccccn3)cc2)c2c(N)nccn12 ; CN(C)C/C=C/C(=O)N1CCCC[C@H]1c1nc(-c2ccc(C(=O)Nc3cc(F)ccn3)cc2)c2c(N)nccn12]

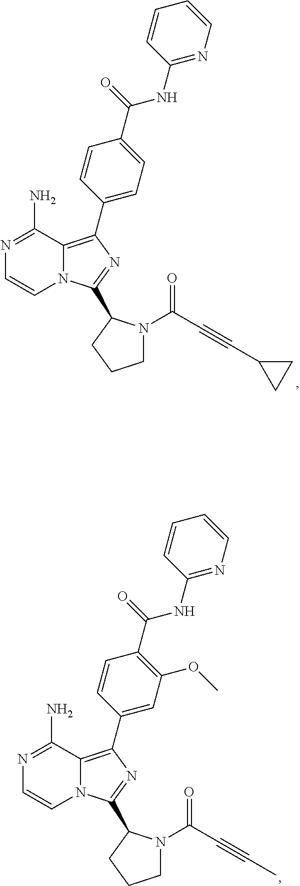 [SMILES: Nc1nccn2c([C@@H]3CCCN3C(=O)C#CC3CC3)nc(-c3ccc(C(=O)Nc4ccccn4)cc3)c12 ; CC#CC(=O)N1CCC[C@H]1c1nc(-c2ccc(C(=O)Nc3ccccn3)c(OC)c2)c2c(N)nccn12]

or a pharmaceutically-acceptable salt, cocrystal, hydrate, solvate, or prodrug thereof, wherein the dose is selected from the group consisting of 5 mg, 10 mg, 15 mg, 20 mg, and 25 mg, wherein the dose is administered once daily, twice daily, or three times daily, and wherein the dose is administered by a route of administration selected from the group consisting of oral administration, topical administration, and combinations thereof, wherein the disorder is an immune rejection associated with organ or cell transplant selected from the group consisting of a disorder associated with anti-allogeneic antibodies, a disorder associated with allograft rejection prior to, during, or after organ or cell transplantation, pre-transplant conditioning of patients receiving solid organ transplant, a disorder associated with humoral acute rejection, a disorder associated with heart transplantation, a disorder associated with renal transplantation, a disorder associated with kidney transplantation, a disorder associated with lung transplantation, a disorder associated with liver transplantation, a disorder associated with ABO-incompatible transplantation, and a disorder associated with stem cell transplantation.

In a preferred embodiment, the invention includes a method of treating a disorder in a human comprising the step of administering a dose of a compound selected from the group consisting of:

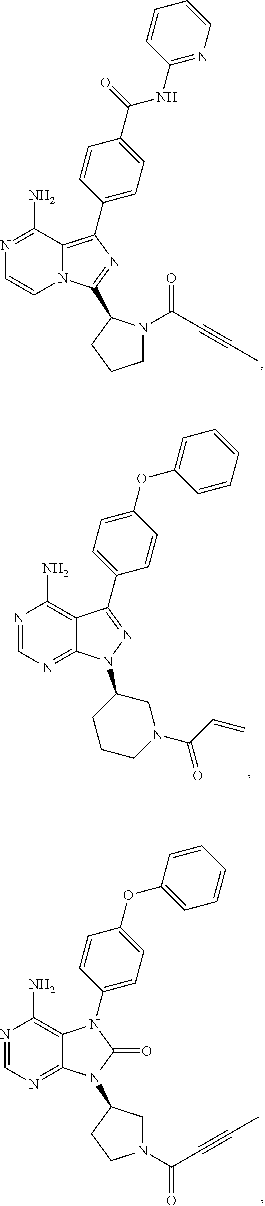 [SMILES: CC#CC(=O)N1CCC[C@H]1c1nc(-c2ccc(C(=O)Nc3ccccn3)cc2)c2c(N)nccn12 ; C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1]

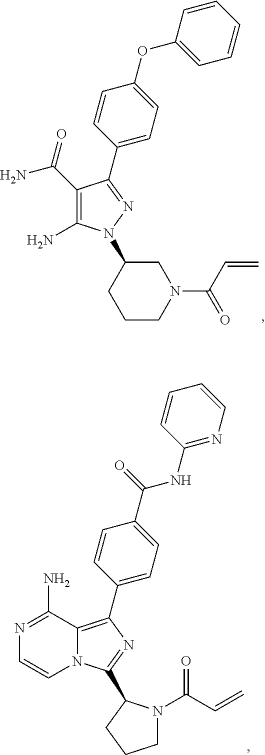 [SMILES: C=CC(=O)N1CCC[C@@H](n2nc(-c3ccc(Oc4ccccc4)cc3)c(C(N)=O)c2N)C1 ; C=CC(=O)N1CCC[C@H]1c1nc(-c2ccc(C(=O)Nc3ccccn3)cc2)c2c(N)nccn12]

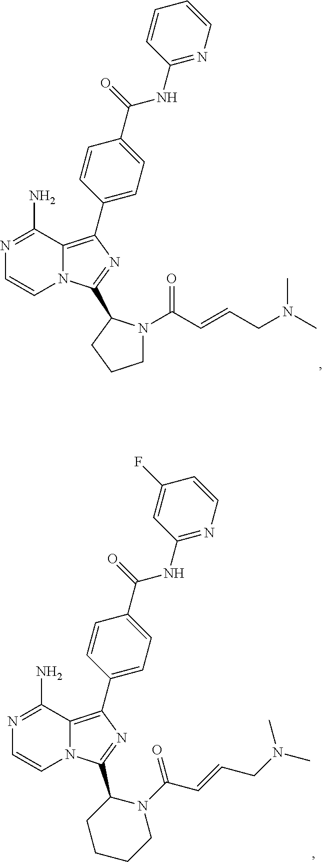 [SMILES: CN(C)C/C=C/C(=O)N1CCC[C@H]1c1nc(-c2ccc(C(=O)Nc3ccccn3)cc2)c2c(N)nccn12 ; CN(C)C/C=C/C(=O)N1CCCC[C@H]1c1nc(-c2ccc(C(=O)Nc3cc(F)ccn3)cc2)c2c(N)nccn12]

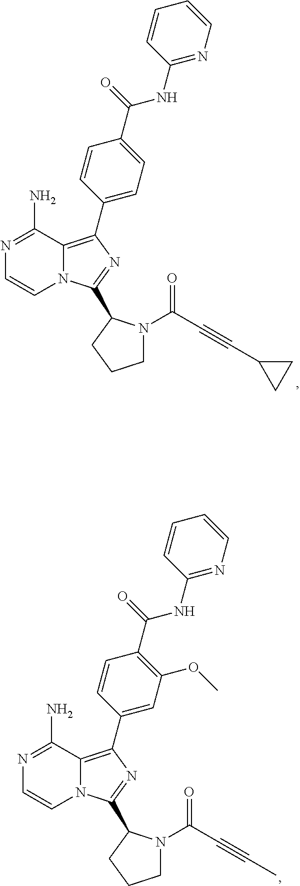 [SMILES: Nc1nccn2c([C@@H]3CCCN3C(=O)C#CC3CC3)nc(-c3ccc(C(=O)Nc4ccccn4)cc3)c12 ; CC#CC(=O)N1CCC[C@H]1c1nc(-c2ccc(C(=O)Nc3ccccn3)c(OC)c2)c2c(N)nccn12]

or a pharmaceutically-acceptable salt, cocrystal, hydrate, solvate, or prodrug thereof, wherein the dose is selected from the group consisting of 5 mg, 10 mg, 15 mg, 20 mg, and 25 mg, wherein the dose is administered once daily, twice daily, or three times daily, and wherein the dose is administered by a route of administration selected from the group consisting of oral administration, topical administration, and combinations thereof, wherein the disorder is a graft-versus-host disease (GVHD), wherein the GVHD is selected from the group consisting of GVHD associated with stem cell transplant, GVHD associated with bone marrow transplant, thymus GVHD, skin GVHD, gastrointestinal GVHD, liver GVHD, acute GVHD, and chronic GVHD.

In a preferred embodiment, the invention includes a method of treating a disorder in a human comprising the step of administering a dose of a compound selected from the group consisting of:

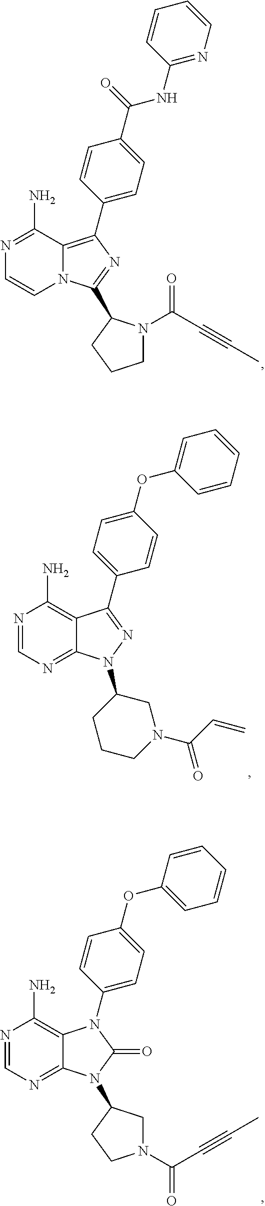 [SMILES: CC#CC(=O)N1CCC[C@H]1c1nc(-c2ccc(C(=O)Nc3ccccn3)cc2)c2c(N)nccn12 ; C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1]

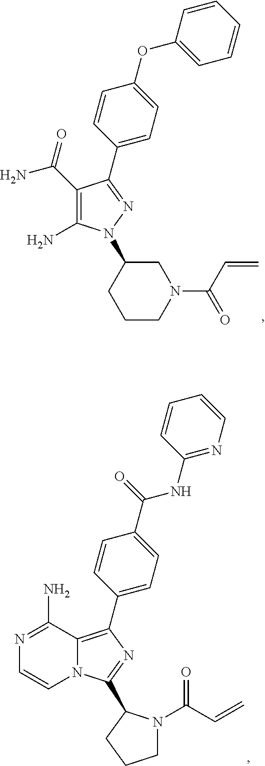 [SMILES: C=CC(=O)N1CCC[C@@H](n2nc(-c3ccc(Oc4ccccc4)cc3)c(C(N)=O)c2N)C1 ; C=CC(=O)N1CCC[C@H]1c1nc(-c2ccc(C(=O)Nc3ccccn3)cc2)c2c(N)nccn12]

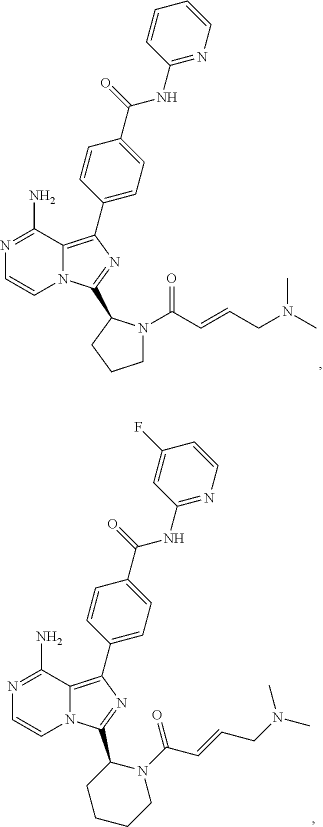 [SMILES: CN(C)C/C=C/C(=O)N1CCC[C@H]1c1nc(-c2ccc(C(=O)Nc3ccccn3)cc2)c2c(N)nccn12 ; CN(C)C/C=C/C(=O)N1CCCC[C@H]1c1nc(-c2ccc(C(=O)Nc3cc(F)ccn3)cc2)c2c(N)nccn12]

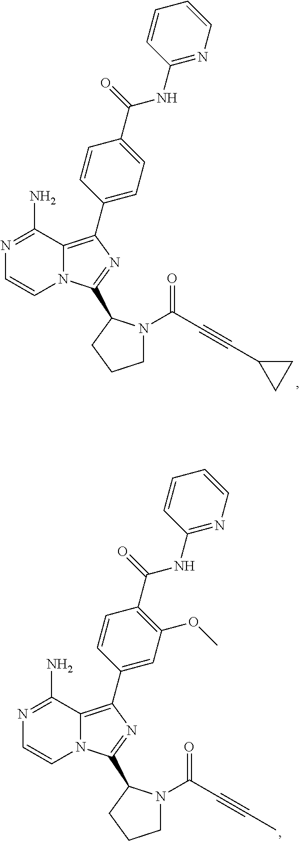 [SMILES: Nc1nccn2c([C@@H]3CCCN3C(=O)C#CC3CC3)nc(-c3ccc(C(=O)Nc4ccccn4)cc3)c12 ; CC#CC(=O)N1CCC[C@H]1c1nc(-c2ccc(C(=O)Nc3ccccn3)c(OC)c2)c2c(N)nccn12]

or a pharmaceutically-acceptable salt, cocrystal, hydrate, solvate, or prodrug thereof, wherein the dose is selected from the group consisting of 5 mg, 10 mg, 15 mg, 20 mg, and 25 mg, wherein the dose is administered once daily, twice daily, or three times daily, and wherein the dose is administered by a route of administration selected from the group consisting of oral administration, topical administration, and combinations thereof, wherein the disorder is a dermatosis, wherein the dermatosis is selected from the group consisting of psoriasis vulgaris, guttate psoriasis, erythrodermic psoriasis, psoriatic nails, annular pustular psoriasis, pustular psoriasis, inverse psoriasis, psoriatic arthritis, keratoderma blennorrhagicum, parapsoriasis, erythema nodosum, palmoplantar hidradentitis, atopic dermatitis, atopic eczema, seborrheic eczema, seborrheic dermatitis, dyshidrosis, rosacea, cutaneous lupus erythematosus, acute cutaneous lupus erythematosus, subacute cutaneous lupus erythematosus, discoid lupus erythematosus, lupus erythromatosus tumidus, lupus nephritis, lupus erythematosus panniculitis, erythema multiforme, verruca, verrucous lupus erythematosus, vitiligo, alopecia areata, purigo nodularis, lichen planus, purigo pigmentosum, pemphigus vulgaris, bullous pemphigoid, pemphigus erythematosus, pemphigus nodularis, erythrodermic sarcoidosis, granulomatous dermatisis, scleroderma, systemic sclerosis, cutaneous manifestations of systemic sclerosis, diffuse cutaneous mastocytosis, erythrodermic mastocytosis, granuloma annulare, chondrodermatitis nodularis, contact dermatitis, drug eruptions, linear IgA bullous dermatosis, eosinophilic dermatitis, keratosis pilaris, lymphomatoid papulosis, pityriasis lichenoides et varioliformis acuta (PLEVA), lichenoides chronica (PLC), febrile ulceronecrotic Mucha-Habermann disease (FUMHD), chronic urticaria, rheumatoid neutrophilic dermatitis, cutaneous manifestations of graft-versus-host disease, cryoglobulinemic purpura, and purpura hyperglobulinemica.

In a preferred embodiment, the invention includes a method of treating a disorder in a human comprising the step of administering a dose of a compound selected from the group consisting of:

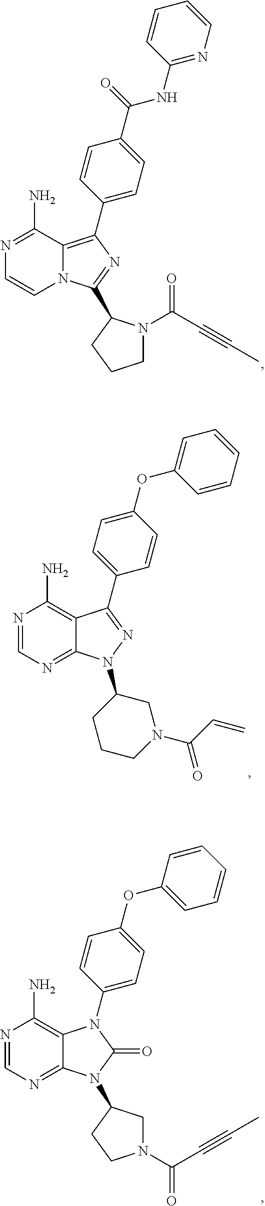 [SMILES: CC#CC(=O)N1CCC[C@H]1c1nc(-c2ccc(C(=O)Nc3ccccn3)cc2)c2c(N)nccn12 ; C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1]

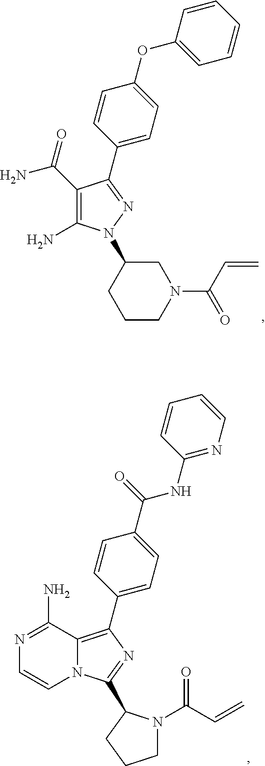 [SMILES: C=CC(=O)N1CCC[C@@H](n2nc(-c3ccc(Oc4ccccc4)cc3)c(C(N)=O)c2N)C1 ; C=CC(=O)N1CCC[C@H]1c1nc(-c2ccc(C(=O)Nc3ccccn3)cc2)c2c(N)nccn12]

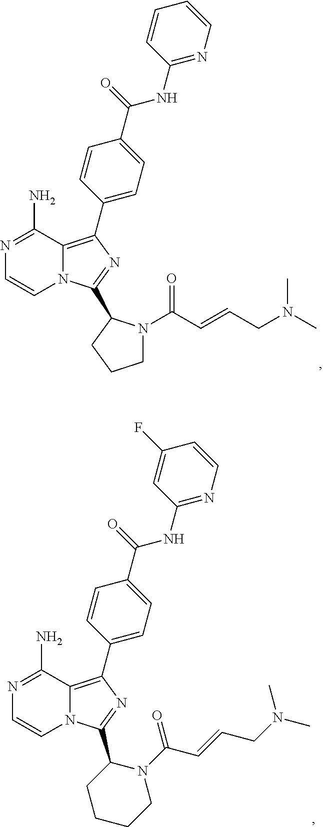 [SMILES: CN(C)C/C=C/C(=O)N1CCC[C@H]1c1nc(-c2ccc(C(=O)Nc3ccccn3)cc2)c2c(N)nccn12 ; CN(C)C/C=C/C(=O)N1CCCC[C@H]1c1nc(-c2ccc(C(=O)Nc3cc(F)ccn3)cc2)c2c(N)nccn12]

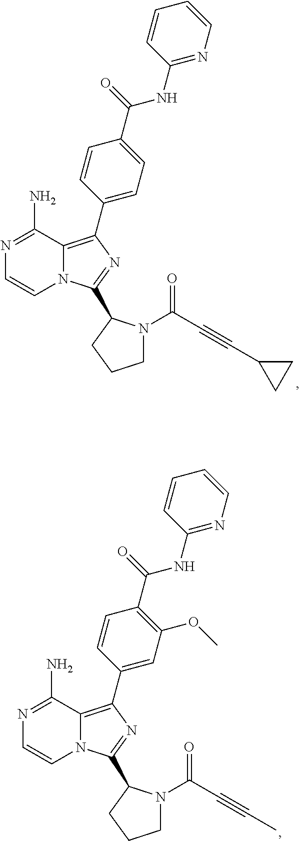 [SMILES: Nc1nccn2c([C@@H]3CCCN3C(=O)C#CC3CC3)nc(-c3ccc(C(=O)Nc4ccccn4)cc3)c12 ; CC#CC(=O)N1CCC[C@H]1c1nc(-c2ccc(C(=O)Nc3ccccn3)c(OC)c2)c2c(N)nccn12]

or a pharmaceutically-acceptable salt, cocrystal, hydrate, solvate, or prodrug thereof, wherein the dose is selected from the group consisting of 5 mg, 10 mg, 15 mg, 20 mg, and 25 mg, wherein the dose is administered once daily, twice daily, or three times daily, and wherein the dose is administered by a route of administration selected from the group consisting of oral administration, topical administration, and combinations thereof, wherein the disorder is a dermatosis, wherein the dermatosis results from dermal manifestations of systemic diseases where sensitization, lymphocyte recruitment, lymphocyte skewing by local or lymph-node antigen presenting cells, activation of skin-resident or skin-homing lymphocytes, innate immune sensing, keratinocyte antimicrobial responses, activation of resident or infiltrating myeloid dendritic cells, plasmacytoid dendritic cells, macrophages, mast cells, neutrophils, and/or Langerhans cells, and wherein the dermatosis leads to the development of skin lesions.

In a preferred embodiment, the invention includes a method of treating a disorder in a human comprising the step of administering a dose of a compound selected from the group consisting of:

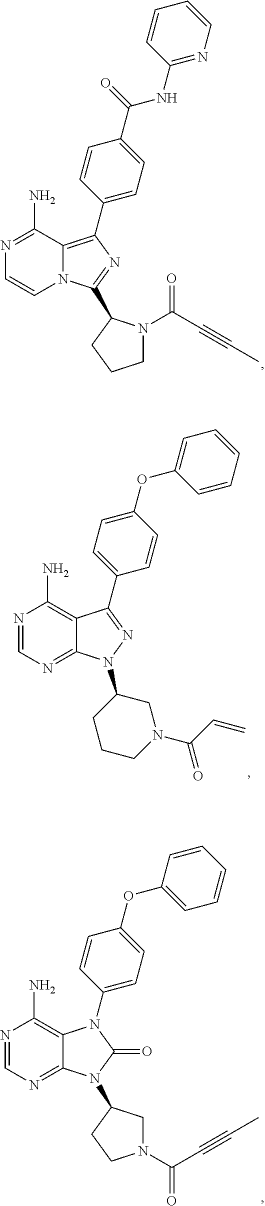 [SMILES: CC#CC(=O)N1CCC[C@H]1c1nc(-c2ccc(C(=O)Nc3ccccn3)cc2)c2c(N)nccn12 ; C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1]

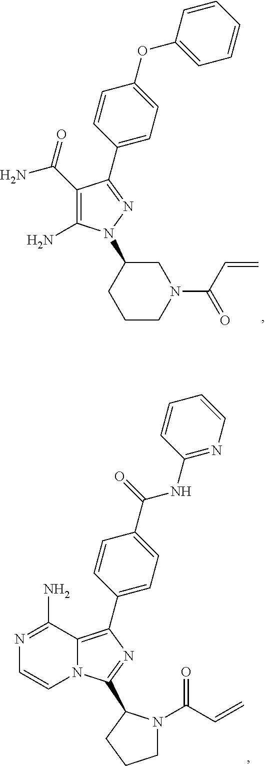 [SMILES: C=CC(=O)N1CCC[C@@H](n2nc(-c3ccc(Oc4ccccc4)cc3)c(C(N)=O)c2N)C1 ; C=CC(=O)N1CCC[C@H]1c1nc(-c2ccc(C(=O)Nc3ccccn3)cc2)c2c(N)nccn12]

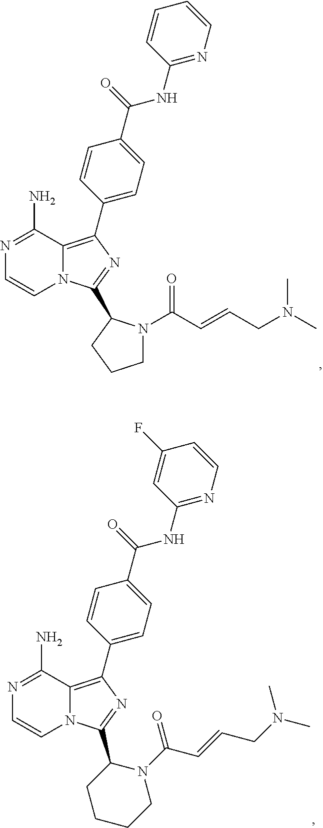 [SMILES: CN(C)C/C=C/C(=O)N1CCC[C@H]1c1nc(-c2ccc(C(=O)Nc3ccccn3)cc2)c2c(N)nccn12 ; CN(C)C/C=C/C(=O)N1CCCC[C@H]1c1nc(-c2ccc(C(=O)Nc3cc(F)ccn3)cc2)c2c(N)nccn12]

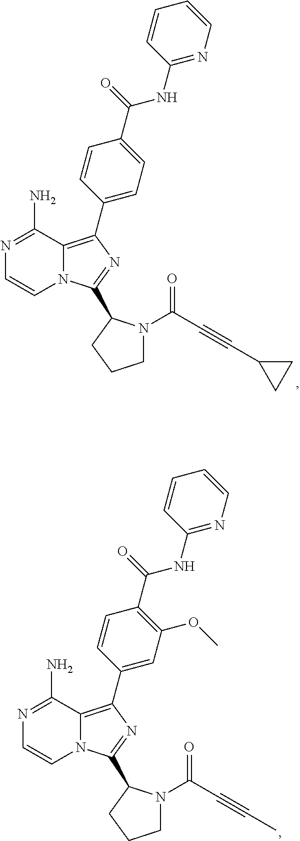 [SMILES: Nc1nccn2c([C@@H]3CCCN3C(=O)C#CC3CC3)nc(-c3ccc(C(=O)Nc4ccccn4)cc3)c12 ; CC#CC(=O)N1CCC[C@H]1c1nc(-c2ccc(C(=O)Nc3ccccn3)c(OC)c2)c2c(N)nccn12]

or a pharmaceutically-acceptable salt, cocrystal, hydrate, solvate, or prodrug thereof, wherein the dose is selected from the group consisting of 5 mg, 10 mg, 15 mg, 20 mg, and 25 mg, wherein the dose is administered once daily, twice daily, or three times daily, and wherein the dose is administered by a route of administration selected from the group consisting of oral administration, topical administration, and combinations thereof, wherein the human is a member of a special population, and wherein the special population is selected from the group consisting of children, juveniles, infants, adolescents, nursing mothers, pregnant women, elderly/frail individuals, patients requiring polypharmacy, patients with hepatic impairment, slow metabolizers, or intolerant and/or sensitive individuals.

In a preferred embodiment, the invention includes a method of treating a BTK mediated disease in a human comprising the step of: (a) administering a BTK inhibitor at a dose and schedule sufficient to provide a target BTK occupancy in a tissue compartment over the course of sub-chronic or chronic administration. In an embodiment, the BTK mediated disease is a cancer selected from the group consisting of a cancer selected from the group consisting of non-Hodgkin's lymphoma, acute myeloid leukemia, chronic lymphocytic leukemia, small lymphocytic lymphoma, diffuse large B cell lymphoma, mantle cell lymphoma, Waldenstrom's macroglobulinemia, follicular lymphoma, B cell acute lymphoblastic leukemia, Burkitt's leukemia, juvenile myelomonocytic leukemia, mast cell leukemia, hairy cell leukemia, Hodgkin's disease, multiple myeloma, thymus cancer, brain cancer, glioma, lung cancer, squamous cell cancer, skin cancer, melanoma, eye cancer, retinoblastoma, intraocular melanoma, oral cavity cancer, oropharyngeal cancer, adenocystic carcinoma, bladder cancer, gastric cancer, stomach cancer, pancreatic cancer, breast cancer, cervical cancer, head cancer, neck cancer, renal cancer, kidney cancer, liver cancer, ovarian cancer, prostate cancer, colorectal cancer, bone cancer, esophageal cancer, testicular cancer, gynecological cancer, thyroid cancer, central nervous system cancer, cancer related to acquired immune deficiency syndrome, cervical carcinoma, nasopharyngeal carcinoma, Kaposi's sarcoma and, primary effusion lymphoma, hepatocellular carcinoma, T-cell leukemia, and mastocytosis. In an embodiment, the BTK mediated disease is an inflammatory, immune, or autoimmune disorder selected from the group consisting of tumor angiogenesis, chronic inflammatory disease, rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, ulcerative colitis, atopic dermatitis, pouchitis, spondylarthritis, uveitis, Behcet's disease, polymyalgia rheumatica, giant-cell arteritis, sarcoidosis, Kawasaki disease, juvenile idiopathic arthritis, hidratenitis suppurativa, Sjögren's syndrome, psoriatic arthritis, juvenile rheumatoid arthritis, ankylosing spoldylitis, asthma, Crohn's disease, lupus, lupus nephritis, and polycythemia vera. In an embodiment, the BTK mediated disease is an immune rejection associated with an organ or cell transplant selected from the group consisting of a disorder associated with anti-allogeneic antibodies, a disorder associated with allograft rejection prior to, during, or after organ or cell transplantation, pre-transplant conditioning of patients receiving solid organ transplant, a disorder associated with humoral acute rejection, a disorder associated with heart transplantation, a disorder associated with renal transplantation, a disorder associated with kidney transplantation, a disorder associated with lung transplantation, a disorder associated with liver transplantation, a disorder associated with ABO-incompatible transplantation, and a disorder associated with stem cell transplantation. In an embodiment, the BTK mediated disease is a graft-versus-host disease (GVHD), wherein the GVHD is selected from the group consisting of GVHD associated with stem cell transplant, GVHD associated with bone marrow transplant, thymus GVHD, skin GVHD, gastrointestinal GVHD, liver GVHD, acute GVHD, and chronic GVHD. In an embodiment, the BTK mediated disease is a dermatosis, wherein the dermatosis is selected from the group consisting of psoriasis vulgaris, guttate psoriasis, erythrodermic psoriasis, psoriatic nails, annular pustular psoriasis, pustular psoriasis, inverse psoriasis, psoriatic arthritis, keratoderma blennorrhagicum, parapsoriasis, erythema nodosum, palmoplantar hidradentitis, atopic dermatitis, atopic eczema, seborrheic eczema, seborrheic dermatitis, dyshidrosis, rosacea, cutaneous lupus erythematosus, acute cutaneous lupus erythematosus, subacute cutaneous lupus erythematosus, discoid lupus erythematosus, lupus erythromatosus tumidus, lupus nephritis, lupus erythematosus panniculitis, erythema multiforme, verruca, verrucous lupus erythematosus, vitiligo, alopecia areata, purigo nodularis, lichen planus, purigo pigmentosum, pemphigus vulgaris, bullous pemphigoid, pemphigus erythematosus, pemphigus nodularis, erythrodermic sarcoidosis, granulomatous dermatisis, scleroderma, systemic sclerosis, cutaneous manifestations of systemic sclerosis, diffuse cutaneous mastocytosis, erythrodermic mastocytosis, granuloma annulare, chondrodermatitis nodularis, contact dermatitis, drug eruptions, linear IgA bullous dermatosis, eosinophilic dermatitis, keratosis pilaris, lymphomatoid papulosis, pityriasis lichenoides et varioliformis acuta (PLEVA), lichenoides chronica (PLC), febrile ulceronecrotic Mucha-Habermann disease (FUMHD), chronic urticaria, rheumatoid neutrophilic dermatitis, cutaneous manifestations of graft-versus-host disease, cryoglobulinemic purpura, and purpura hyperglobulinemica. In an embodiment, the BTK mediated disease is a dermatosis, wherein the dermatosis results from dermal manifestations of systemic diseases where sensitization, lymphocyte recruitment, lymphocyte skewing by local or lymph-node antigen presenting cells, activation of skin-resident or skin-homing lymphocytes, innate immune sensing, keratinocyte antimicrobial responses, activation of resident or infiltrating myeloid dendritic cells, plasmacytoid dendritic cells, macrophages, mast cells, neutrophils, and/or Langerhans cells, and wherein the dermatosis leads to the development of skin lesions. In any of the foregoing embodiments, the treated human is part of a special population, wherein the special population can be selected from the group consisting of children, juveniles, infants, adolescents, nursing mothers, pregnant women, elderly/frail individuals, patients requiring polypharmacy, patients with hepatic impairment, slow metabolizers, or intolerant and/or sensitive individuals. In any of the foregoing embodiments, the target BTK occupancy is selected from the group consisting of greater than 85%, greater than 90%, greater than 91%, greater than 92%, greater than 93%, greater than 94%, greater than 95%, greater than 96%, greater than 97%, greater than 98%, and greater than 99%. In any of the foregoing embodiments, the tissue compartment is selected from the group consisting of peripheral blood B cells, bone marrow B cells, lymph node B cells, autoreactive B cells, plasma cells, regulatory B cells, follicular dendritic cells, myeloid-derived dendritic cells, tumor stroma, tumor-associated macrophage, mast cells, alveolar macrophages, dust cells, plasmacytoid dendritic cells, cutaneous lymphocyte antigen (CLA)-positive T cells, lymphoid-inducer cells, Langerhans cells, monocytes, macrophages, histiocytes, Kupffer cells, glial cells, microglia, Schwann cells, Ito cells, hepatic stellate cells, pancreatic stellate cells, glioma cells, malignant B cells, adipocytes, sarcoid cells, granulocytes, neutrophils, eosinophils, hematopoietic stem cells, serous cells, mesenchymal stromal cells, osteoblasts, osteoclasts, infiltrating lymphocytes, immunocytes and inflammatory infiltrates. In any of the foregoing embodiments, the tissue compartment is selected from the group consisting of peripheral blood, bone marrow, germinal center, lymphoid follicle, gut-associated lymphoid tissue, tonsil, lymphoma lesion, ectopic lymphoid tissue, ectopic node, lymph node lesion, lymphadenopathy, spleen, solid tumor, tumor microenvironment, tumor stroma, bone, bone lesion, bone metastasis, synovial fluid, articular surface, joint, kidney, liver, lung, bronchus/bronchiole, mediastinum, pleura, peritoneum, cystadenocarcinoma, heart, pancreas, sinusoid, eye, nerve, brain, brain metastasis, brain lesion, central nervous system, skin, stomach, lamina propria, gut, colon, exocrine gland, salivary gland, lacrimal gland, breast, dermis, subdermis, epidermis, perivascular, inflammatory lesion, cutaneous lesion, granuloma, mastocytoma, papule, testis, ovary, and bladder.

In a preferred embodiment, the invention includes a method of treating a BTK mediated disease in a human comprising the step of: (a) administering a BTK inhibitor in a dosage form that provides controlled release of the active pharmaceutical agent over time, wherein the release is sufficient to provide a target BTK occupancy in a tissue compartment over the course of sub-chronic or chronic administration. In an embodiment, the BTK mediated disease is a cancer selected from the group consisting of a cancer selected from the group consisting of non-Hodgkin's lymphoma, acute myeloid leukemia, chronic lymphocytic leukemia, small lymphocytic lymphoma, diffuse large B cell lymphoma, mantle cell lymphoma, Waldenstrom's macroglobulinemia, follicular lymphoma, B cell acute lymphoblastic leukemia, Burkitt's leukemia, juvenile myelomonocytic leukemia, mast cell leukemia, hairy cell leukemia, Hodgkin's disease, multiple myeloma, thymus cancer, brain cancer, glioma, lung cancer, squamous cell cancer, skin cancer, melanoma, eye cancer, retinoblastoma, intraocular melanoma, oral cavity cancer, oropharyngeal cancer, adenocystic carcinoma, bladder cancer, gastric cancer, stomach cancer, pancreatic cancer, breast cancer, cervical cancer, head cancer, neck cancer, renal cancer, kidney cancer, liver cancer, ovarian cancer, prostate cancer, colorectal cancer, bone cancer, esophageal cancer, testicular cancer, gynecological cancer, thyroid cancer, central nervous system cancer, cancer related to acquired immune deficiency syndrome, cervical carcinoma, nasopharyngeal carcinoma, Kaposi's sarcoma and, primary effusion lymphoma, hepatocellular carcinoma, T-cell leukemia, and mastocytosis. In an embodiment, the BTK mediated disease is an inflammatory, immune, or autoimmune disorder selected from the group consisting of tumor angiogenesis, chronic inflammatory disease, rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, ulcerative colitis, atopic dermatitis, pouchitis, spondylarthritis, uveitis, Behcet's disease, polymyalgia rheumatica, giant-cell arteritis, sarcoidosis, Kawasaki disease, juvenile idiopathic arthritis, hidratenitis suppurativa, Sjögren's syndrome, psoriatic arthritis, juvenile rheumatoid arthritis, ankylosing spoldylitis, asthma, Crohn's disease, lupus, lupus nephritis, and polycythemia vera. In an embodiment, the BTK mediated disease is an immune rejection associated with an organ or cell transplant selected from the group consisting of a disorder associated with anti-allogeneic antibodies, a disorder associated with allograft rejection prior to, during, or after organ or cell transplantation, pre-transplant conditioning of patients receiving solid organ transplant, a disorder associated with humoral acute rejection, a disorder associated with heart transplantation, a disorder associated with renal transplantation, a disorder associated with kidney transplantation, a disorder associated with lung transplantation, a disorder associated with liver transplantation, a disorder associated with ABO-incompatible transplantation, and a disorder associated with stem cell transplantation. In an embodiment, the BTK mediated disease is a graft-versus-host disease (GVHD), wherein the GVHD is selected from the group consisting of GVHD associated with stem cell transplant, GVHD associated with bone marrow transplant, thymus GVHD, skin GVHD, gastrointestinal GVHD, liver GVHD, acute GVHD, and chronic GVHD. In an embodiment, the BTK mediated disease is a dermatosis, wherein the dermatosis is selected from the group consisting of psoriasis vulgaris, guttate psoriasis, erythrodermic psoriasis, psoriatic nails, annular pustular psoriasis, pustular psoriasis, inverse psoriasis, psoriatic arthritis, keratoderma blennorrhagicum, parapsoriasis, erythema nodosum, palmoplantar hidradentitis, atopic dermatitis, atopic eczema, seborrheic eczema, seborrheic dermatitis, dyshidrosis, rosacea, cutaneous lupus erythematosus, acute cutaneous lupus erythematosus, subacute cutaneous lupus erythematosus, discoid lupus erythematosus, lupus erythromatosus tumidus, lupus nephritis, lupus erythematosus panniculitis, erythema multiforme, verruca, verrucous lupus erythematosus, vitiligo, alopecia areata, purigo nodularis, lichen planus, purigo pigmentosum, pemphigus vulgaris, bullous pemphigoid, pemphigus erythematosus, pemphigus nodularis, erythrodermic sarcoidosis, granulomatous dermatisis, scleroderma, systemic sclerosis, cutaneous manifestations of systemic sclerosis, diffuse cutaneous mastocytosis, erythrodermic mastocytosis, granuloma annulare, chondrodermatitis nodularis, contact dermatitis, drug eruptions, linear IgA bullous dermatosis, eosinophilic dermatitis, keratosis pilaris, lymphomatoid papulosis, pityriasis lichenoides et varioliformis acuta (PLEVA), lichenoides chronica (PLC), febrile ulceronecrotic Mucha-Habermann disease (FUMHD), chronic urticaria, rheumatoid neutrophilic dermatitis, cutaneous manifestations of graft-versus-host disease, cryoglobulinemic purpura, and purpura hyperglobulinemica. In an embodiment, the BTK mediated disease is a dermatosis, wherein the dermatosis results from dermal manifestations of systemic diseases where sensitization, lymphocyte recruitment, lymphocyte skewing by local or lymph-node antigen presenting cells, activation of skin-resident or skin-homing lymphocytes, innate immune sensing, keratinocyte antimicrobial responses, activation of resident or infiltrating myeloid dendritic cells, plasmacytoid dendritic cells, macrophages, mast cells, neutrophils, and/or Langerhans cells, and wherein the dermatosis leads to the development of skin lesions. In any of the foregoing embodiments, the treated human is part of a special population. In any of the foregoing embodiments, the special population can be selected from the group consisting of children, juveniles, infants, adolescents, nursing mothers, pregnant women, elderly/frail individuals, patients requiring polypharmacy, patients with hepatic impairment, slow metabolizers, or intolerant and/or sensitive individuals. In any of the foregoing embodiments, the target BTK occupancy is selected from the group consisting of greater than 85%, greater than 90%, greater than 91%, greater than 92%, greater than 93%, greater than 94%, greater than 95%, greater than 96%, greater than 97%, greater than 98%, and greater than 99%. In any of the foregoing embodiments, the tissue compartment is selected from the group consisting of peripheral blood B cells, bone marrow B cells, lymph node B cells, autoreactive B cells, plasma cells, regulatory B cells, follicular dendritic cells, myeloid-derived dendritic cells, tumor stroma, tumor-associated macrophage, mast cells, alveolar macrophages, dust cells, plasmacytoid dendritic cells, cutaneous lymphocyte antigen (CLA)-positive T cells, lymphoid-inducer cells, Langerhans cells, monocytes, macrophages, histiocytes, Kupffer cells, glial cells, microglia, Schwann cells, Ito cells, hepatic stellate cells, pancreatic stellate cells, glioma cells, malignant B cells, adipocytes, sarcoid cells, granulocytes, neutrophils, eosinophils, hematopoietic stem cells, serous cells, mesenchymal stromal cells, osteoblasts, osteoclasts, infiltrating lymphocytes, immunocytes and inflammatory infiltrates. In any of the foregoing embodiments, the tissue compartment is selected from the group consisting of peripheral blood, bone marrow, germinal center, lymphoid follicle, gut-associated lymphoid tissue, tonsil, lymphoma lesion, ectopic lymphoid tissue, ectopic node, lymph node lesion, lymphadenopathy, spleen, solid tumor, tumor microenvironment, tumor stroma, bone, bone lesion, bone metastasis, synovial fluid, articular surface, joint, kidney, liver, lung, bronchus/bronchiole, mediastinum, pleura, peritoneum, cystadenocarcinoma, heart, pancreas, sinusoid, eye, nerve, brain, brain metastasis, brain lesion, central nervous system, skin, stomach, lamina propria, gut, colon, exocrine gland, salivary gland, lacrimal gland, breast, dermis, subdermis, epidermis, perivascular, inflammatory lesion, cutaneous lesion, granuloma, mastocytoma, papule, testis, ovary, and bladder.

In a preferred embodiment, the invention includes a pharmaceutical composition comprising a dose of a compound selected from the group consisting of:

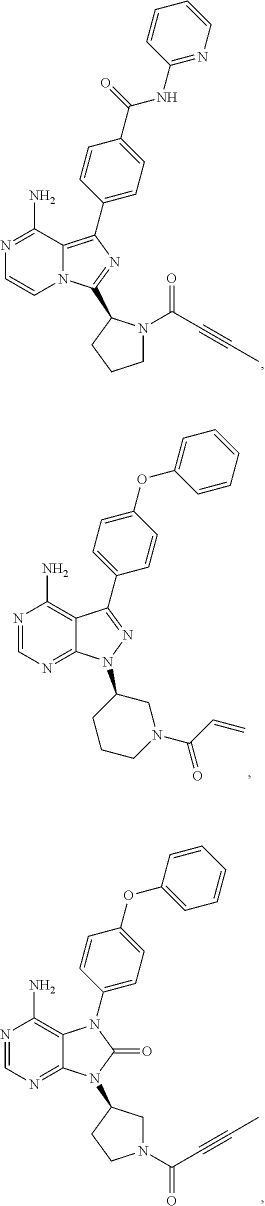 [SMILES: CC#CC(=O)N1CCC[C@H]1c1nc(-c2ccc(C(=O)Nc3ccccn3)cc2)c2c(N)nccn12 ; C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1]

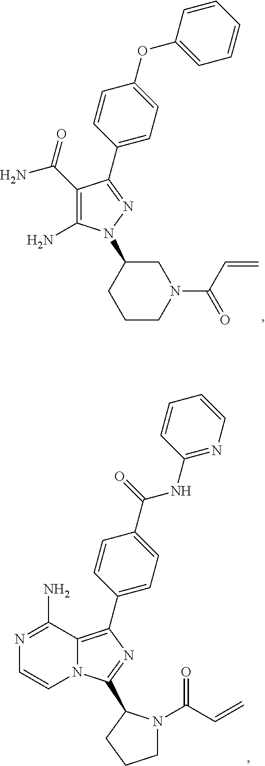 [SMILES: C=CC(=O)N1CCC[C@@H](n2nc(-c3ccc(Oc4ccccc4)cc3)c(C(N)=O)c2N)C1 ; C=CC(=O)N1CCC[C@H]1c1nc(-c2ccc(C(=O)Nc3ccccn3)cc2)c2c(N)nccn12]

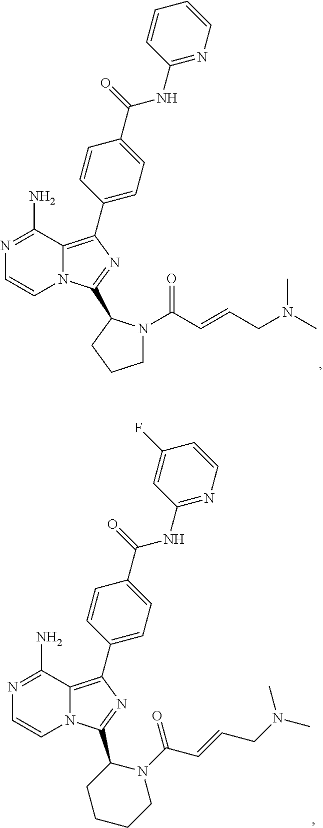 [SMILES: CN(C)C/C=C/C(=O)N1CCC[C@H]1c1nc(-c2ccc(C(=O)Nc3ccccn3)cc2)c2c(N)nccn12 ; CN(C)C/C=C/C(=O)N1CCCC[C@H]1c1nc(-c2ccc(C(=O)Nc3cc(F)ccn3)cc2)c2c(N)nccn12]

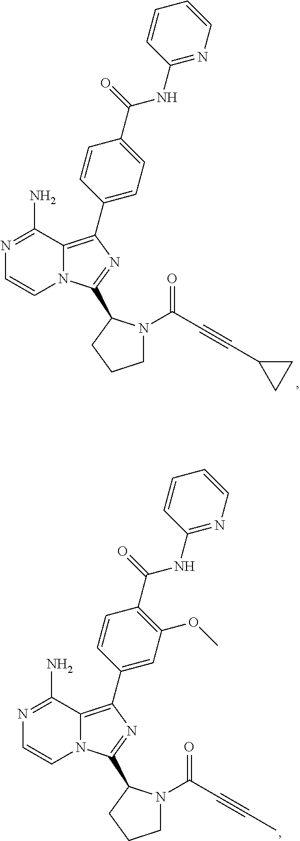 [SMILES: Nc1nccn2c([C@@H]3CCCN3C(=O)C#CC3CC3)nc(-c3ccc(C(=O)Nc4ccccn4)cc3)c12 ; CC#CC(=O)N1CCC[C@H]1c1nc(-c2ccc(C(=O)Nc3ccccn3)c(OC)c2)c2c(N)nccn12]

or a pharmaceutically-acceptable salt, cocrystal, hydrate, solvate, or prodrug thereof, and a pharmaceutically-acceptable excipient, wherein the dose is selected from the group consisting of 5 mg, 10 mg, 15 mg, 20 mg, and 25 mg, and wherein the composition is suitable for topical delivery. In a preferred embodiment, the invention provides a use of the pharmaceutical composition of any of the foregoing embodiments in the manufacture of a medicament for the treatment of a dermatosis. In a preferred embodiment, the dermatosis is selected from the group consisting of psoriasis vulgaris, guttate psoriasis, erythrodermic psoriasis, psoriatic nails, annular pustular psoriasis, pustular psoriasis, inverse psoriasis, psoriatic arthritis, keratoderma blennorrhagicum, parapsoriasis, erythema nodosum, palmoplantar hidradentitis, atopic dermatitis, atopic eczema, seborrheic eczema, seborrheic dermatitis, dyshidrosis, rosacea, cutaneous lupus erythematosus, acute cutaneous lupus erythematosus, subacute cutaneous lupus erythematosus, discoid lupus erythematosus, lupus erythromatosus tumidus, lupus nephritis, lupus erythematosus panniculitis, erythema multiforme, verruca, verrucous lupus erythematosus, vitiligo, alopecia areata, purigo nodularis, lichen planus, purigo pigmentosum, pemphigus vulgaris, bullous pemphigoid, pemphigus erythematosus, pemphigus nodularis, erythrodermic sarcoidosis, granulomatous dermatisis, scleroderma, systemic sclerosis, cutaneous manifestations of systemic sclerosis, diffuse cutaneous mastocytosis, erythrodermic mastocytosis, granuloma annulare, chondrodermatitis nodularis, contact dermatitis, drug eruptions, linear IgA bullous dermatosis, eosinophilic dermatitis, keratosis pilaris, lymphomatoid papulosis, pityriasis lichenoides et varioliformis acuta (PLEVA), lichenoides chronica (PLC), febrile ulceronecrotic Mucha-Habermann disease (FUMHD), chronic urticaria, rheumatoid neutrophilic dermatitis, cutaneous manifestations of graft-versus-host disease, cryoglobulinemic purpura, and purpura hyperglobulinemica. In a preferred embodiment, the dermatosis results from dermal manifestations of systemic diseases where sensitization, lymphocyte recruitment, lymphocyte skewing by local or lymph-node antigen presenting cells, activation of skin-resident or skin-homing lymphocytes, innate immune sensing, keratinocyte antimicrobial responses, activation of resident or infiltrating myeloid dendritic cells, plasmacytoid dendritic cells, macrophages, mast cells, neutrophils, and/or Langerhans cells, and wherein the dermatosis leads to the development of skin lesions.

In a preferred embodiment, the invention includes a method of treating a dermatosis comprising the step of administering a therapeutically-effective amount of a BTK inhibitor. In an embodiment, the dermatosis is selected from the group consisting of psoriasis vulgaris, guttate psoriasis, erythrodermic psoriasis, psoriatic nails, annular pustular psoriasis, pustular psoriasis, inverse psoriasis, psoriatic arthritis, keratoderma blennorrhagicum, parapsoriasis, erythema nodosum, palmoplantar hidradentitis, atopic dermatitis, atopic eczema, seborrheic eczema, seborrheic dermatitis, dyshidrosis, rosacea, cutaneous lupus erythematosus, acute cutaneous lupus erythematosus, subacute cutaneous lupus erythematosus, discoid lupus erythematosus, lupus erythromatosus tumidus, lupus nephritis, lupus erythematosus panniculitis, erythema multiforme, verruca, verrucous lupus erythematosus, vitiligo, alopecia areata, purigo nodularis, lichen planus, purigo pigmentosum, pemphigus vulgaris, bullous pemphigoid, pemphigus erythematosus, pemphigus nodularis, erythrodermic sarcoidosis, granulomatous dermatisis, scleroderma, systemic sclerosis, cutaneous manifestations of systemic sclerosis, diffuse cutaneous mastocytosis, erythrodermic mastocytosis, granuloma annulare, chondrodermatitis nodularis, contact dermatitis, drug eruptions, linear IgA bullous dermatosis, eosinophilic dermatitis, keratosis pilaris, lymphomatoid papulosis, pityriasis lichenoides et varioliformis acuta (PLEVA), lichenoides chronica (PLC), febrile ulceronecrotic Mucha-Habermann disease (FUMHD), chronic urticaria, rheumatoid neutrophilic dermatitis, cutaneous manifestations of graft-versus-host disease, cryoglobulinemic purpura, and purpura hyperglobulinemica. In an embodiment, the dermatosis results from dermal manifestations of systemic diseases where sensitization, lymphocyte recruitment, lymphocyte skewing by local or lymph-node antigen presenting cells, activation of skin-resident or skin-homing lymphocytes, innate immune sensing, keratinocyte antimicrobial responses, activation of resident or infiltrating myeloid dendritic cells, plasmacytoid dendritic cells, macrophages, mast cells, neutrophils, and/or Langerhans cells, and wherein the dermatosis leads to the development of skin lesions. In a preferred embodiment, the dermatosis is selected from the group consisting of psoriasis, scleroderma, atopic dermatitis, and cutaneous lupus erythematosus. In any of the foregoing embodiments, the BTK inhibitor is selected from the group consisting of:

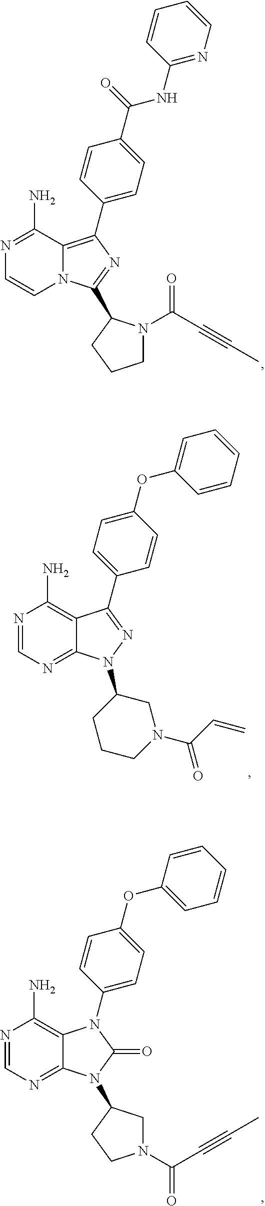 [SMILES: CC#CC(=O)N1CCC[C@H]1c1nc(-c2ccc(C(=O)Nc3ccccn3)cc2)c2c(N)nccn12 ; C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1]

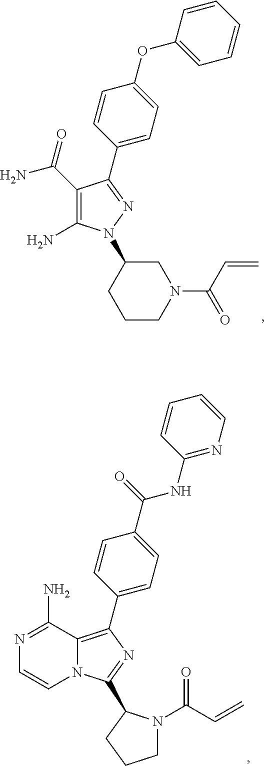 [SMILES: C=CC(=O)N1CCC[C@@H](n2nc(-c3ccc(Oc4ccccc4)cc3)c(C(N)=O)c2N)C1 ; C=CC(=O)N1CCC[C@H]1c1nc(-c2ccc(C(=O)Nc3ccccn3)cc2)c2c(N)nccn12]

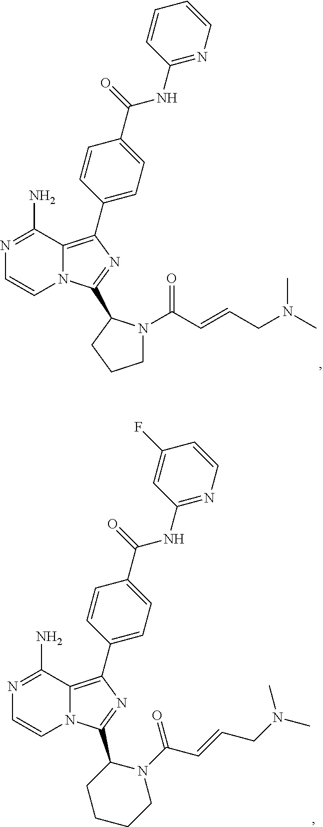 [SMILES: CN(C)C/C=C/C(=O)N1CCC[C@H]1c1nc(-c2ccc(C(=O)Nc3ccccn3)cc2)c2c(N)nccn12 ; CN(C)C/C=C/C(=O)N1CCCC[C@H]1c1nc(-c2ccc(C(=O)Nc3cc(F)ccn3)cc2)c2c(N)nccn12]

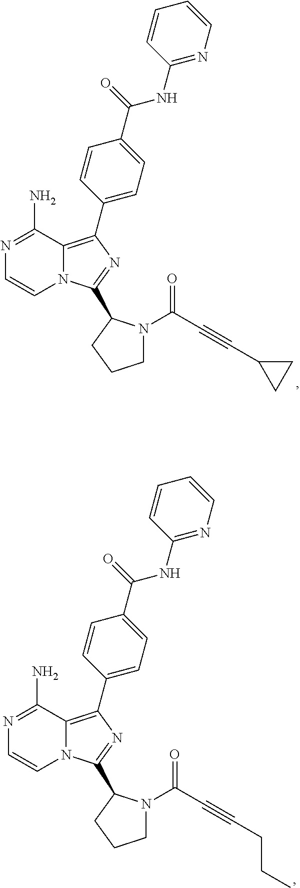 [SMILES: Nc1nccn2c([C@@H]3CCCN3C(=O)C#CC3CC3)nc(-c3ccc(C(=O)Nc4ccccn4)cc3)c12 ; CCCC#CC(=O)N1CCC[C@H]1c1nc(-c2ccc(C(=O)Nc3ccccn3)cc2)c2c(N)nccn12]

or a pharmaceutically-acceptable salt, cocrystal, hydrate, solvate, or prodrug thereof. In any of the foregoing embodiments, the therapeutically effective dose of the BTK inhibitor is selected from the group consisting of 5 mg, 10 mg, 15 mg, 20 mg, 25 mg, 30 mg, 45 mg, 60 mg, 75 mg, 90 mg, and 100 mg, wherein the therapeutically effective dose is administered at an interval selected from the group consisting of once daily, twice daily, three times daily, and four times daily, and wherein the therapeutically effective dose is administered by a route of administration selected from the group consisting of oral administration, topical administration, and combinations thereof.

In an embodiment, the invention includes compositions and methods of treating a leukemic cancer that exhibits a higher rate of BTK resynthesis in leukemic bone marrow B cells relative to the BTK resynthesis rate in leukemic blood B cells, comprising the step of administering a dose of a compound to reduce the rate of BTK resynthesis, wherein the compound is a covalent BTK inhibitor. In an embodiment, the invention includes compositions and methods of treating a leukemic cancer that exhibits a higher rate of BTK resynthesis in leukemic bone marrow B cells relative to the BTK resynthesis rate in leukemic blood B cells, comprising the step of administering a dose of a compound to inhibit BTK and reduce the rate of BTK resynthesis, wherein the compound is a compound of Formula (I) to Formula (XXV), the dose is administered once daily, twice daily, or three times daily, and the leukemic cancer is chronic lymphocytic leukemia (CLL), acute lymphoblastic leukemia (ALL), small lymphocytic lymphoma (SLL), diffuse large B cell lymphoma (DLBCL), Richter's transformation (RT), mantle cell lymphoma (MCL), Burkitt lymphoma (BL), or Waldenstrom's macroglobulinemia (WM).

In an embodiment, the invention includes a method of treating a leukemic cancer that exhibits a higher rate of BTK resynthesis in leukemic bone marrow B cells relative to the BTK resynthesis rate in leukemic lymph node B cells, comprising the step of administering a dose of a compound to inhibit BTK and reduce the rate of BTK resynthesis, wherein the compound is a compound of Formula (I) to Formula (XXV), the dose is administered once daily, twice daily, or three times daily, and the leukemic cancer is CLL, SLL, DLBCL, RT, MCL, BL, or WM.

In an embodiment, the invention includes a method of treating an acute leukemic cancer that exhibits a higher rate of BTK resynthesis in acute leukemic blood B cells than the BTK resynthesis rate in chronic leukemic blood B cells, comprising the step of administering a dose of a compound to inhibit BTK and reduce the rate of BTK resynthesis, wherein the compound is a compound of Formula (I) to Formula (XXV), the dose is administered once daily, twice daily, or three times daily, and the leukemic cancer is B cell acute lymphoblastic leukemia (B-ALL), BL, prolymphocytic leukemia or Richter's Transformation.

In an embodiment, the invention includes a method of treating a B cell malignancy that exhibits a higher rate of BTK resynthesis in peripheral lymph nodes with lymphadenopathy than the BTK resynthesis rate in circulating tumor cells or in bone marrow, comprising the step of administering a dose of a compound to inhibit BTK and reduce the rate of BTK resynthesis, wherein the compound is a compound of Formula (I) to Formula (XXV), the dose is administered once daily, twice daily, or three times daily, and the B cell malignancy is DLBCL, RT, MCL, BL, WM, follicular lymphoma (FL), T-cell/histiocyte rich large B cell lymphoma, EBV positive DLBCL of the elderly, primary cutaneous DLBCL, primary DLBCL of the central nervous system, primary mediastinal large B cell lymphoma, transformations of Castleman's disease, an unclassifyable B cell lymphoma with features of DLBCL and Hodgkin disease, or Hodgkin's lymphoma.

In an embodiment, the invention includes a method of treating a B cell disorder that exhibits a higher rate of BTK resynthesis in peripheral lymph nodes with lymphadenopathy than the BTK resynthesis rate in circulating B cells or in normally developing bone marrow, comprising the step of administering a dose of a compound to inhibit BTK and reduce the rate of BTK resynthesis, wherein the compound is a compound of Formula (I) to Formula (XXV), the dose is administered once daily, twice daily, or three times daily, and the treated disease is a post-transplant lymphoproliferative disorder, lymphomatous granulomatosis, or chronic fatigue syndrome.

In an embodiment, the invention includes a method of treating an autoimmune disease that exhibits a higher rate of BTK resynthesis in tissue disease sites than the BTK resynthesis rate in circulating peripheral blood B cells or in normally developing bone marrow B cells, comprising of the step of administering a dose of a compound to inhibit BTK and reduce the rate of BTK resynthesis, wherein the compound is a compound of Formula (I) to Formula (XXV), the dose is administered once daily, twice daily, or three times daily, and the autoimmune disease is rheumatoid arthritis, juvenile RA, osteoarthritis, ankylosing spondylitis, psoriatic arthritis, psoriasis vulgaris, pemphigus vulgaris, bullous pemphigoid, Sjogren's syndrome (SS), systemic lupus erythromatosus (SLE), discoid lupus erythromatosus (discoid LE), LE tumidus, lupus nephritis (LN), antiphospholipidosis, whipple, dermatomyositis, polymyositis, autoimmune thrombocytopenia, idiopathic thrombocytopenia purpura, thrombotic thrombocytopenia purpura, autoimmune (cold) agglutinin disease, autoimmune hemolytic anemia, cryoglobulinemia, autoimmune vasculitis, ANCA-associated vasculitis, scleroderma, systemic sclerosis, multiple sclerosis (MS), chronic focal encephalitis, Guillian-Barre syndrome, chronic fatigue syndrome, mononucleosis, neuromyelitis optica, autoimmune uveitis, Grave's disease, thyroid associated opthalmopathy, granulomatosis with microscopic polyangitis, Wegener's granulomatosis, idiopathic pulmonary fibrosis, sarcoidosis, idiopathic membranous nephropathy, IgA nephropathy, glomerulosclerosis, pancreatitis, type I diabetes, and type II diabetes.

In an embodiment, the invention includes a method of treating an autoimmune disease that exhibits a rate of BTK resynthesis, which can be measured in cells from diseased tissue or peripheral blood using a suitable assay to quantify the presence of unoccupied BTK target sites at certain times following administration of an agent that covalently inactivates the BTK kinase. The presence of unoccupied BTK target sites in relevant cells may be measured using an enzyme-linked immunosorbent assay (ELISA), flow cytometry, ligand-binding assay on beads, immunohistochemistry, or other in vitro diagnostic techniques with relevant detection methodology. The method of treating a specific disease based on the regeneration rate of BTK in diseased tissues comprises the step of measuring the BTK resynthesis rate in a patient or group of patients with the specific disease and administering a dose of a compound to inhibit BTK and reduce the rate of BTK resynthesis wherein the compound is a compound of Formula (I) to Formula (XXV), and the dose is administered once daily, twice daily, or three times daily, depending on the measured BTK resynthesis rate.

In an embodiment, the invention includes a method of treating cancer, a method of treating inflammatory, immune, and autoimmune diseases, and a method of surpressing immune responses for organ or cell transplants, wherein the cancer, disease, or immune response to be suppressed exhibits a rate of BTK resynthesis, which can be measured in sites of disease using specific imaging agents to detect the presence of unoccupied BTK target sites when combined with CT scans, positron emission tomography (PET) imaging, magnetic resonance imaging (MRI), or near infrared fluorescence imaging, or other in vivo imaging modalities, to customize the treatment of a specific disease based on the regeneration rate of BTK in diseased tissues.

In an embodiment, the method comprises the step of measuring the BTK resynthesis rate in a patient or group of patients with the specific disease and administering a dose of a compound to inhibit BTK and reduce the rate of BTK resynthesis wherein the compound is a compound of Formula (I) to Formula (XXV), and the dose is administered once daily, twice daily, or three times daily, depending on the measured BTK resynthesis rate.

In an embodiment, the invention includes a method of treating a B cell malignancy that exhibits a rate of BTK resynthesis, which can be measured in cells from affected lymph nodes, in bone marrow, peripheral blood, or other sites of lesions such as metastases, using a suitable assay to quantify the presence of unoccupied BTK target sites at certain times following administration of an agent that covalently binds to, and inactivates BTK. The presence of unoccupied BTK target sites in relevant cells may be measured using ELISA, flow cytometry, ligand-binding assay on beads, immunohistochemistry, or other in vitro diagnostic technique with relevant detection methodology. The method of treating a specific B cell malignancy based on the regeneration rate of BTK in tumor cells comprises the step of measuring the BTK resynthesis rate in a subject or group of subjects with the malignancy and administering a dose of a compound to inhibit BTK and reduce the rate of BTK resynthesis wherein the compound is a compound of Formula (I) to Formula (XXV), and the dose is administered once daily, twice daily, or three times daily, depending on the measured BTK resynthesis rate.

In an embodiment, the invention includes a method of treating a B cell malignancy that exhibits a rate of BTK resynthesis, which can be measured in tumor bearing tissues and bone marrow using specific imaging agents to detect the presence of unoccupied BTK target sites when combined with CT scans, PET imaging, MRI, or NMR imaging to evaluate disease activity, or other in vivo imaging modalities, to customize the treatment of a B cell malignancy based on the regeneration rate of BTK in tumor bearing tissues. The method comprises the step of measuring the BTK resynthesis rate in a subject or group of subjects with the specific disease and administering a dose of a compound to inhibit BTK and reduce the rate of BTK resynthesis wherein the compound is a compound of Formula (I) to Formula (XXV), and the dose is administered once daily, twice daily, or three times daily, depending on the measured BTK resynthesis rate.

In an embodiment, the the invention includes a method of treating a B cell malignancy that exhibits different rates of BTK resynthesis in different lesions, which can be measured using specific imaging agents to detect the presence of unoccupied BTK target sites when combined with CT scans, PET imaging, MRI, or NMR imaging to evaluate disease activity, or other in vivo imaging modalities, to customize the treatment of a B cell malignancy based on the regeneration rate of BTK in a subset of tumor lesions within the human body. The method comprises the step of measuring the BTK resynthesis rate in several lesions, such as index lesions, lesions with rapid metabolism, and newly arising lesions within the body, and administering a dose of a compound to inhibit BTK and reduce the rate of BTK resynthesis wherein the compound is a compound of Formula (I) to Formula (XXV), and the dose is administered once daily, twice daily, or three times daily, depending on the most rapid measured BTK resynthesis rate in an individual patient or in a group of patients or patient subset in a malignant disease.

In an embodiment, the invention includes a method of treating BTK positive diseases in which the resynthesis of BTK is monitored at the sites of diseased tissue by means of in vitro or in vivo measurements following administration of a covalent inhibitor of BTK wherein the compound is a compound of Formula (I) to Formula (XXV), and the diseased tissue site is a compartment containing BTK with a resynthesis rate that differs from other compartments within the body, such as the peripheral blood compartment or bone marrow compartment, and the resynthesis rate in the compartment comprising the diseased tissue is used to define the dose level, dose schedule or dosage form of the inhibitor.

In an embodiment, the invention includes a method of treating BTK positive diseases in which the resynthesis of BTK is monitored at the sites of diseased tissue by means of in vitro or in vivo measurements following administration of a covalent inhibitor of BTK wherein the compound is a compound of Formula (I) to Formula (XXV), and a rapidly growing tumor lesion is a compartment containing BTK with a resynthesis rate that differs from other compartments within the body, such as the peripheral blood compartment or a compartment associated with more indolent tumor lesions, and the resynthesis rate in the compartment comprising the rapidly growing tumor lesion is used to define the dose level, dose schedule or dosage form of the inhibitor.

In an embodiment, the invention includes a method of treating CLL in which the resynthesis of BTK is monitored at the sites of diseased tissue by means of in vitro or in vivo measurements following administration of a covalent inhibitor of BTK wherein the compound is a compound of Formula (I) to Formula (XXV), and the bone marrow is a compartment containing BTK with a resynthesis rate that differs from other compartments within the body, such as the peripheral blood compartment or the compartment of CLL cells that are lodged within lymphoid tissues or other tissues of the body including bone marrow, and the resynthesis rate in the compartment comprising the the bone marrow is used to define the dose level, dose schedule or dosage form of the inhibitor.

In an embodiment, the invention includes a method of treating RA in which the resynthesis of BTK is monitored at the sites of diseased tissue by means of in vitro or in vivo measurements following administration of a covalent inhibitor of BTK wherein the compound is a compound of Formula (I) to Formula (XXV), and the synovial fluid is a compartment containing BTK with a resynthesis rate that differs from other compartments within the body, such as the peripheral blood compartment or the compartment comprising lymphoid tissues, and the resynthesis rate in the compartment comprising the synovial fluid is used to define the dose level, dose schedule or dosage form of the inhibitor.

In an embodiment, the invention includes a method of treating autoimmune diseases in which the resynthesis of BTK is monitored at the sites of diseased tissue by means of in vitro or in vivo measurements following administration of a covalent inhibitor of BTK wherein the compound is a compound of Formula (I) to Formula (XXV), and the tissues affected by autoimmune disease activity comprise a compartment with a BTK resynthesis rate that differs from other compartments within the body, such as the peripheral blood compartment or the compartment comprising lymphoid tissues, and the resynthesis rate in the compartment comprising the diseased tissues is used to define the dose level, dose schedule or dosage form of the inhibitor.

In an embodiment, the invention includes a method of treating patients receiving HLA-mismatched or incompletely matched transplants in which the resynthesis of BTK is monitored at the sites of transplant or tissue affected by anti-allogen immunity, by means of in vitro or in vivo measurements following administration of a covalent inhibitor of BTK wherein the compound is a compound of Formula (I) to Formula (XXV), and the tissues affected by anti-allogen immune activity comprise a compartment with a BTK resynthesis rate that differs from other compartments within the body, such as the peripheral blood compartment or the compartment comprising unstimulated immunocytes, and the resynthesis rate in the compartment comprising the diseased tissues is used to define the dose level, dose schedule or dosage form of the inhibitor.

In an embodiment, the invention includes a method for treating BTK positive diseases with a controlled release formulation of a covalent inhibitor of BTK wherein the compound is a compound of Formula (I) to Formula (XXV), and the controlled release formulation providing sufficient strength to be absorbed from the drug delivery point into the primary compartment (peripheral blood) and pass into the compartment of diseased tissue and therein inhibit BTK and reduce the rate of BTK synthesis during the entire dosing interval. Controlled release can include extended release and delayed release or combinations of extended, delayed, and immediate release formulations in a single dosage unit or in separate dosage units. Controlled release formulations include formulations in which the compound is released in a single bolus targeted at a single section of the gastrointestinal (GI) tract, a single long bolus or in multiple boluses targeting different specific sections of the mammalian GI tract or segments of the section, including but not limited to the stomach, duodenum, jejunum, ileum, cecum, colon, rectum or anal canal. Controlled release may be based on polymers or excipients that dissolve or form pores at particular pH, swell to inhibit GI transit or retard release, react at different pH to reduce density of the formulation and cause the unit to be retained by buoyancy, and/or have specific chemical or physical properties that allow them to react to particular conditions in different sections of the GI tract including but not limited to the action of bile salts, ionic strength, enzymes, pH, volume, microflora, or time.

In an embodiment, the invention includes a method for treating BTK positive diseases with a regimen that includes a higher loading dose followed after a period of time, sufficient to reduce the rate of BTK resynthesis in the tissue compartment of interest, with a maintenance dose that is sufficient to inhibit BTK during an extended or chronic dosing phase. The loading dose results in rapid attainment of steady state BTK inhibition and the maintenance dose results in sustained inhibition of BTK following the reduction of the BTK resynthesis rate in the tissue compartment of interest.

### BRIEF DESCRIPTION OF THE DRAWINGS

The foregoing summary, as well as the following detailed description of the invention, will be better understood when read in conjunction with the appended drawings.

FIG. 1A illustrates a two-compartment PK model with a delay for oral absorption which was used to fit concentration versus time data from healthy volunteers dosed with 15 mg QD Formula (II) for seven days. The model is a two-compartment PK model with a delay d(1,3) for oral absorption. The q1 compartment represents the primary compartment (i.e., the bloodstream or circulatory system), the q2 compartment represents the drug delivery point (i.e., the gut generally, the stomach, and/or the duodenum), the q4 compartment represents peripheral compartments, the rates k(3,2), k(4,1), and k(1,4) represent the intercompartment rates, the rate k(0,1) represents the output rate (i.e., degradation of BTK), and s1 represents the sampling point (i.e., the bloodstream or circulatory system). FIG. 1B and FIG. 1C show observed (solid symbols) versus model (solid line) mean concentration-time profiles for Formula (II) after a dose of 15 mg. The Day 7 profile was derived from the Day 1 model fit (FIG. 1B), overlaid with Day 7 data (FIG. 1C). Unweighted data were not used in the model fit.

FIG. 2 illustrates a compartmental biophase PD model used to fit Formula (II) BTK occupancy data, wherein the q7 compartment represents un-modified BTK (i.e., BTK that is not covalently bound with Formula (II)), the q6 compartment represents BTK covalently bound to Formula (II), and each compartment has a turnover rate (input rate output rate). Output rates k(0,7) and k(0,6) were assumed to be equal. The rate constant k(6,7) is a saturable rate constant representing irreversible inactivation of BTK by Formula (II). The PK model and the PD model were linked by the rate constant k(6,7) which was saturable and contained a drug concentration term C (the concentration of drug in compartment q1 (FIG. 1A)). Occupation of the receptor was determined by the ratio: q6/(q6+q7). The symbol s2 represents the sampling point (i.e., the bloodstream or central compartment), which captures both functional (unbound) BTK and inactivated BTK as a percentage target occupancy.

FIG. 3 illustrates BTK occupancy in healthy volunteers following repeat dose 15 mg administration for 7 days, fitted to a PK/PD model. The presence of inactivated BTK in peripheral blood B lymphocytes was measured using a BTK active-site specific probe in an ELISA assay and expressed as a percentage of pre-study levels. Model turnover rate changes with time; unweighted data were not used in the model fit.

FIG. 4A and FIG. 4B illustrate the effect of change in BTK resynthesis rate over time during treatment with Formula (II) on initial model fits of the Day 1 and Day 7 steady state data. The presence of inactivated BTK in peripheral blood B lymphocytes was measured using a BTK active-site specific probe in an ELISA assay. The percentage BTK target occupancy during the treatment and post-dosing intervals was measured and the model fits using the initial post-dose BTK resynthesis rate (t_{1/2}=20 hours, FIG. 4A) and the later post-dose BTK resynthesis rate (t_{1/2}=119 hours, FIG. 4B) were applied.

FIG. 5 illustrates the PK/PD model fit for inhibition of BTK phosphorylation in healthy human volunteers dosed at 15 mg QD of Formula (II). The percentage of BTK functional activation was measured using phospho-flow cytometry following ex vivo BCR stimulation of peripheral blood B cells. Fitted estimates (line) and data from the healthy volunteer study are overlaid.

FIG. 6A and FIG. 6B illustrate predicted plasma concentration time profile for Formula (II) when delivered as a single oral 25 mg dose on Day 1 (FIG. 6A) and Day 7 (FIG. 6B), based on the PK/PD model derived from a healthy volunteer study. The actual data from 40 healthy human volunteers treated with 25 mg Formula (II) on Study Day 1 and Study Day 7 are overlaid.

FIG. 7A, FIG. 7B, and FIG. 7C illustrate BTK target occupancy in healthy volunteers and two PK/PD model estimates. FIG. 7A illustrates the BTK target occupancy in peripheral blood B lymphocytes from healthy human volunteers (n=40) after two single oral doses of Formula (II) at 25 mg separated by 1 week. The presence of inactivated BTK in peripheral blood B lymphocytes was measured using a BTK active-site specific probe in an ELISA assay. The percentage BTK target occupancy during the treatment and post-dosing intervals, and linear regressions depicting the rate of decline in BTK target occupancy from the peak values at T=4 hours post-dose are shown. Linear correlations for BTK target occupancy terminal phase decline were statistically significant (p<0.0001), although the linear curve-fit only approximates the elimination phase kinetics of BTK target occupancy as determined by the PK/PD model. The PK/PD model estimates for doses of 25 mg Formula (II) showed that BTK occupancy after the second dose was well fitted at Study Day 1 (FIG. 7B) and Study Day 7 (FIG. 7C), whereas the rate of BTK resynthesis was faster after the initial dose.

FIG. 8 illustrates simulated percentage BTK target occupancy using the PK/PD model to estimate PD effect of dosing with Formula (II) at 15 mg BID and 30 mg QD.

FIG. 9 illustrates simulated percentage BTK occupancy using the PK/PD model to estimate PD effect of dosing with Formula (II) at 15, 30, and 45 mg QD.

FIG. 10 illustrates simulated percentage BTK occupancy using the PK/PD model to estimate PD effect of dosing with Formula (II) at 15, 30, and 45 mg BID.

FIG. 11 illustrates PK/PD simulated percentage inhibition of BTK phosphorylation using the PK/PD model estimate effect of dosing with Formula (II) on pBTK inhibition with dose regimens of 15 mg BID versus 30 mg QD.

FIG. 12 illustrates the model fit of the Formula (II) concentration versus time profile in subjects treated with a 50 mg dose of Formula (II) by oral administration. To model dosages higher than 25 mg, the model k(4,1) constant rate was decreased. Data from healthy volunteers treated with 50 mg Formula (II) are overlaid.

FIG. 13 illustrates simulated BTK occupancy from the final PK/PD model with the BTK resynthesis stepped to a lower rate after Day 2 of dose administration at 100 mg QD of Formula (II). Mean BTK percentage occupancy data from patients with CLL treated with this dose regimen are overlaid. The presence of inactivated BTK in CLL tumor cells was measured using a BTK active-site specific probe in an ELISA assay.

FIG. 14 illustrates simulated BTK occupancy from the final PK/PD model with the BTK resynthesis stepped to a lower rate after Day 2 of dose administration at 100 mg BID of Formula (II). Mean BTK percentage occupancy data from human subjects with CLL treated with this dose regimen are overlaid. The presence of inactivated BTK in CLL tumor cells was measured using a BTK active-site specific probe in an ELISA assay.

FIG. 15 illustrates simulated BTK occupancy from the final PK/PD model with the BTK resynthesis stepped to a lower rate after Day 2 of dose administration at 250 mg QD of Formula (II). Mean BTK percentage occupancy data from patients with CLL treated with this dose regimen are overlaid. The presence of inactivated BTK in CLL tumor cells was measured using a BTK active-site specific probe in an ELISA assay.

FIG. 16 illustrates simulated BTK occupancy from the final PK/PD model with the BTK resynthesis stepped to a lower rate after Day 2 of dose administration at 400 mg QD of Formula (II). Mean BTK percentage occupancy data from human subjects with CLL treated with this dose regimen are overlaid. The presence of inactivated BTK in CLL tumor cells was measured using a BTK active-site specific probe in an ELISA assay.

FIG. 17 illustrates PK/PD simulated BTK occupancy at Formula (II) dosing regimens of 30 mg QD versus 15 mg BID.

FIG. 18 illustrates PK/PD simulated BTK occupancy at a Formula (II) loading-dose, maintenance dose regimen of 60 mg BID loading dose for 7 days followed by a 30 mg QD maintenance dose.

FIG. 19 illustrates PK/PD simulated BTK occupancy at a Formula (II) loading-dose, maintenance dose regimen of 60 mg BID loading dose for 7 days followed by a 15 mg QD maintenance dose.

FIG. 20 illustrates PK/PD simulated BTK occupancy at a Formula (II) loading-dose, maintenance dose regimen of 60 mg BID loading dose for 7 days followed by a 7.5 mg QD maintenance dose.

FIG. 21 illustrates the BTK target occupancy data and model fit in peripheral blood B lymphocytes from healthy human volunteers (n=40) during 7 days of dosing with oral Formula (II) at 15 mg QD. The presence of inactivated BTK in peripheral blood B lymphocytes was measured using a BTK active-site specific probe in an ELISA assay. The percentage BTK target occupancy during the treatment and post-dosing intervals, and modeled estimates based on a k_{4,1} of 0.91 and a BTK resynthesis rate of 0.1 for the first 48 hours and 0.04 thereafter. The unweighted data point was not used in the modeled estimate.

FIG. 22 illustrates the effect of oral administration of Formula (II) at 15 mg QD for seven consecutive days on the intracellular levels of total BTK protein in peripheral blood B lymphocytes. The percentage of pre-study BTK protein level was determined in cryopreserved B cells by flow cytometry analysis of Mean Fluorescence Intensity. Decreased BTK levels were observed after 48 hours.

FIG. 23A and FIG. 23B illustrate the rate of resynthesis of BTK in healthy human volunteers treated with a single oral administration of Formula (II) at doses of 50, 75 and 100 mg (QD), or two doses of 25 and 50 mg separated by 12 hours (BID). The presence of inactivated BTK in peripheral blood B lymphocytes was measured using a BTK active-site specific probe in an ELISA assay. The percentage BTK target occupancy during the treatment and post-dosing intervals is shown in the FIG. 23A. Linear regressions of the decline in BTK target occupancy from the sample taken 3 hours after the last dose, until the end of the monitoring interval, were calculated using GraphPad Prism (FIG. 23B).

FIG. 24A, FIG. 24B, FIG. 24C, FIG. 24D, FIG. 24E, and FIG. 24F illustrate the effects of oral dosing with Formula (II) on BCR-mediated signaling function in healthy volunteers at 12 hours after administration of the following doses: 2.5 mg BID, 5 mg BID, 25 mg BID, 50 mg BID, 50 mg QD, 75 mg QD, 100 mg QD. Individual Cmax and AUC levels are plotted against the percentage of BTK target occupancy (FIG. 24A and FIG. 24B) and the percentage of inhibition of BCR stimulated (FIG. 24C and FIG. 24D) CD86 and CD69 (FIG. 24E and FIG. 24F).

FIG. 25 illustrates return of B cell function in healthy human volunteers following treatment with the last of 7 daily oral doses of 15 mg Formula (II) for seven days. Unmodified BTK was measured using a BTK active-site specific probe in an ELISA assay. Phosphorylated BTK and S6 protein were measured by phospho-flow cytometry at 15 minutes after BCR stimulation; surface up-regulation of CD69 and CD86 and down-regulation of CXCR4 were measured by flow cytometry at 24 hours after BCR stimulation in B lymphocytes from cryopreserved PBMC preparations sampled at the indicated times.

FIG. 26A, FIG. 26B, FIG. 26C, FIG. 26D, and FIG. 26E illustrate the concentration versus time profile for Formula (II) when dosed via oral gavage at 30 mg/kg/day to rats, compared with dietary administration at concentrations of 100 and 500 ppm in rat chow, after 14 days of dosing. The percentage of BTK target occupancy in the spleens of the rats was evaluated on Day 14. The mean pharmacokinetic parameters from dosing groups of 6 male rats are shown in inset; BTK target occupancy was evaluated (n=3) using a BTK active-site specific probe in an ELISA assay.

FIG. 27A and FIG. 27B illustrate return of B cell function after treatment of mice with three BTK inhibitors. Expression of CD86 and CD69 following stimulation of splenocytes with anti-IgM was evaluated at the noted times post-dose after oral administration of BTK inhibitors to mice. The percentage of BTK target occupancy is noted in FIG. 27C, demonstrating resynthesis rate of unmodified BTK in this mouse model.

FIG. 28A and FIG. 28B illustrate return of functional signaling through the BCR following stimulation of splenocytes with anti-IgM was evaluated at the noted times post dose after oral administration of BTK inhibitors to mice. The basal levels and stimulated levels of phosphorylated S6 protein were monitored over time.

FIG. 29A and FIG. 29B illustrate return of BCR-mediated signaling function after treatment with two BTK inhibitors. Expression of CD86 and CD69 following stimulation of splenocytes with anti-IgM was evaluated at the noted times post-dose after oral administration of BTK inhibitors to mice.

FIG. 30 illustrates the BTK target occupancy in dogs with spontaneously occurring canine lymphoma following oral administration of Formula (II), in samples of peripheral blood CD21+B cells and in fine needle aspirates from lymphoma lesions at the indicated times.

FIG. 31A, FIG. 31B, FIG. 31C, and FIG. 31D illustrate the BTK target occupancy in CD5⁺/CD19⁺ tumor cells in patients with relapsed/refractory CLL treated with oral administration of Formula (II) at the indicated doses. Peripheral blood samples were obtained over time and BTK target occupancy was evaluated using a BTK active-site specific probe in an ELISA assay.

FIG. 32 illustrates the level of BCR-mediated signaling via BTK in patients with chronic lymphocytic leukemia treated with oral administration of Formula (II) at the indicated times. Peripheral blood samples were obtained and BTK activity was evaluated in CD19⁺/CD5⁺ tumor cells by phospho-flow cytometry of p-BTK at 15-minutes after BCR stimulation. The BCR-mediated signaling through BTK was significantly inhibited following treatment with Formula (II).

FIG. 33 illustrates the per cell level of BTK in patients with chronic lymphocytic leukemia treated with oral administration of 100 mg BID Formula (II) for the indicated times. Peripheral blood samples were obtained and BTK protein levels were evaluated in CD19+/CD5+ tumor cells by flow cytometry. Expression of BTK protein was decreased by a median of 26% of the pre-dose values after 4 weeks of treatment, demonstrating that treatment with Formula (II) inhibited not only the functional activity of BTK in tumor cells but also its resynthesis rate in the therapeutically relevant compartment.

FIG. 34 illustrates the rate of resynthesis of BTK in patients with chronic lymphocytic leukemia treated with oral administration of 100 mg QD and 100 mg BID of Formula (II). The presence of unmodified BTK at 4 hours post-dosing and at the end of the dosing interval was measured using a BTK active-site specific probe in an ELISA assay and expressed as a percentage of pre-study values for each patient. The slopes of the two lines represent the relative rates of BTK regeneration on Day 8 of dosing (after steady-state was achieved).

FIG. 35A and FIG. 35B illustrate the effects of vehicle (left) and Formula (II) (right) on flux at two timepoints, in the ID8 syngeneic orthotropic ovarian cancer model.

FIG. 36 illustrates tumor microenvironment responses to treatment with the BTK inhibitor of Formula (II), with a significant reduction in immunosuppressive tumor associated lymphocytes and myeloid cells, and an increase in cytolytic lymphocytes in tumor-bearing mice, in comparison to a control (vehicle).

FIG. 37 illustrates that treatment with the BTK inhibitor of Formula (II) impairs ID8 ovarian cancer growth in the ID8 syngeneic murine model in comparison to a control (vehicle).

FIG. 38A and FIG. 38B illustrate that treatment with the BTK inhibitor of Formula (II) induces a tumor response that correlates with a significant reduction in total B cells and regulatory B cells (Bregs) in the ID8 tumor microenvironment.

FIG. 39A and FIG. 39B illustrate that treatment with the BTK inhibitor of Formula (II) induces a tumor response that correlates with an increase in tumor infiltrating CD8⁺ T cells and a reduction in immunosuppressive tumor infiltrating Tregs in the ID8 syngeneic murine model.

FIG. 40A, FIG. 40B, FIG. 40C, FIG. 40D, FIG. 40E, FIG. 40F, and FIG. 40G illustrate the bone density in hind limbs of nude rats (n=6 per group) implanted with intratibial MDA-MB231 tumor allografts and treated for up to 41 days with vehicle control, zoledronate (active control) or varying doses of Formula (II). Oral gavage administration of Formula (II) was scheduled as 3 days of initial QD dosing, followed by 30 days of BID dosing at the following dose levels: 3/3, 30/30, and 180/90 mg/kg/day. An untreated group of rats, without tibial implants (n=3) represents bone density of concurrently obtained normal rat tibial images. Statistical significance was determined by two-way ANOVA-Dunnett: ns=not significant, *=P<0.05, **=P<0.01, ***=P<0.001, versus Group 1.

FIG. 41A, FIG. 41B, FIG. 41C, FIG. 41D, FIG. 41E, and FIG. 41F illustrate the effect of Formula (II) treatment on the development of anti-keyhole limpet hemocyanin (KLH) T cell dependent antibody responses in male rats. Sixteen males per group were inoculated with antigen by subcutaneous injection on Day 50 of treatment with Formula (II). Peripheral blood was sampled at 1, 2, and 3 weeks after KLH inoculation and the KLH-specific IgM and IgG levels were measured by ELISA. Raw serum concentration data from treatment groups were compared against vehicle-treated controls using the non-parametric Kruskal-Wallis ANOVA with post-hoc Dunn's tests. Significance levels noted: *p<0.05; **p<0.01.

FIG. 42A, FIG. 42B, FIG. 42C, FIG. 42D, FIG. 42E, and FIG. 42F illustrates the effect of Formula (II) treatment on the development of anti-keyhole limpet hemocyanin (KLH) T cell dependent antibody responses in female rats. Sixteen females per group were inoculated with antigen by subcutaneous injection on Day 50 of treatment with Formula (II). Peripheral blood was sampled at 1, 2, and 3 weeks after KLH inoculation and the KLH-specific IgM and IgG levels were measured by ELISA. Raw serum concentration data from treatment groups were compared against vehicle-treated controls using the non-parametric Kruskal-Wallis ANOVA with post-hoc Dunn's tests. Significance levels noted: *p<0.05; **p<0.01; ***p<0.001; ****p<0.0001.

FIG. 43 illustrates the relative protein expression of BTK in various cell types and tissues. The figure is taken from GeneCard entry for BTK (available at: genecard.org).

FIG. 44 illustrates BTK target occupancy data. Mouse splenocytes were isolated from spleens from mice that were part of the mouse CIA study semi-therapeutic protocol, 3 hr after the last dosing on day 14 of the treatment and cryopreserved. After thawing, BTK target occupancy was measured on splenocyte cell pellets for the treatment groups indicated. BTK target occupancy is calculated based on the luminescence signal versus the vehicle control. Error bars represent the standard deviation for the 3 sets of splenocytes analyzed.

FIG. 45 illustrates changes in a functional measure of BCR signalling through the CD86 marker. Mouse splenocytes were isolated from spleens from mice that were part of the mouse CIA study semi-therapeutic protocol, 3 hr after the last dosing on day 14 of the treatment and cryopreserved. After thawing, inhibition of anti-IgM-induced PD markers CD86 and CD69 on mouse splenocyte B cells were measured. Error bars represent the standard deviation for the 3 sets of splenocytes analyzed.

FIG. 46 illustrates changes in a functional measure of BCR signalling through the CD69 marker. Mouse splenocytes were isolated from spleens from mice that were part of the mouse CIA study semi-therapeutic protocol, 3 hr after the last dosing on day 14 of the treatment and cryopreserved. After thawing, inhibition of anti-IgM-induced PD markers CD86 and CD69 on mouse splenocyte B cells were measured. Error bars represent the standard deviation for the 3 sets of splenocytes analyzed.

### DETAILED DESCRIPTION OF THE INVENTION

While preferred embodiments of the invention are shown and described herein, such embodiments are provided by way of example only and are not intended to otherwise limit the scope of the invention. Various alternatives to the described embodiments of the invention may be employed in practicing the invention.

### DEFINITIONS

Unless defined otherwise, all technical and scientific terms used herein have the same meaning as is commonly understood by one of skill in the art to which this invention belongs. All patents and publications referred to herein are incorporated by reference in their entireties.

The term “BTK mediated disease” or “BTK mediated disorder” means any disease or disorder wherein modulation of the BTK signaling pathway in a cell may modulate the disease or disorder, such that the disease or disorder may be treated or prevented, or such that the symptoms of the disease or disorder may be reduced or ameliorated.

The terms “co-administration” and “administered in combination with” as used herein, encompass administration of two or more agents to a subject so that both agents and/or their metabolites are present in the subject at the same time. Co-administration includes simultaneous administration in separate compositions, administration at different times in separate compositions, or administration in a composition in which both agents are present.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc., which can readily be determined by one of ordinary skill in the art. The term also applies to a dose that will induce a particular response in target cells (e.g., the reduction of platelet adhesion and/or cell migration) or in these cells within a specific compartment in the body (e.g., tumor bearing lymph nodes or bone marrow, the microenvironment of a solid tumor, or sites of autoimmune disease activity, and sites of inflammatory responses). The specific dose will vary depending on the particular compound and dosage form chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “pharmaceutically acceptable salt” refers to salts derived from a variety of organic and inorganic counter ions known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In selected embodiments, the pharmaceutically acceptable base addition salt is chosen from ammonium, potassium, sodium, calcium, and magnesium salts.

“Pharmaceutically acceptable carrier” or “pharmaceutically acceptable excipient” is intended to include any and all solvents, dispersion media, coatings, antibacterial and antifungal agents, isotonic and absorption delaying agents. The use of such media and agents for pharmaceutically active substances is well known in the art. Except insofar as any conventional media or agent is incompatible with the active ingredient, its use in the therapeutic compositions of the invention is contemplated. Supplementary active ingredients can also be incorporated into the described compositions.

“Prodrug” is intended to describe a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers the advantages of solubility, tissue compatibility or delayed release in a mammalian organism, as described in, e.g., Bundgaard, Design of Prodrugs, Elsevier, 1985. The term “prodrug” is also intended to include any covalently bonded carriers, which release the active compound in vivo when administered to a subject. Prodrugs of an active compound, as described herein, may be prepared by modifying functional groups present in the active compound in such a way that the modifications are cleaved, either in routine manipulation or in vivo, to yield the active parent compound. Prodrugs include, for example, compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetates, formates and benzoate derivatives of an alcohol, various ester derivatives of a carboxylic acid, or acetamide, formamide and benzamide derivatives of an amine functional group in the active compound.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary from, for example, between 1% and 15% of the stated number or numerical range.

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range, e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (iso-propyl), n-butyl, n-pentyl, 1,1-dimethylethyl (t-butyl) and 3-methylhexyl. Unless stated otherwise specifically in the specification, an alkyl group is optionally substituted by one or more of substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, alkyl, fluoroalkyl, carbocyclyl, carbocyclylalkyl, aryl, aralkyl, heterocycloalkyl, heterocycloalkylalkyl, heteroaryl, or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocycyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl, or heteroarylalkyl.

“Alkenyl-cycloalkyl” refers to an -(alkenyl)cycloalkyl radical where alkenyl and cyclo alkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc. . . . , up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e., (C₃₋₁₀)cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloseptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein “R” is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the “R” of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R₂ of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in sources such as Greene et al., Protective Groups in Organic Synthesis, 4th Ed., John Wiley & Sons, 2007, which is incorporated herein by reference in its entirety.

“Aromatic” or “aryl” or “Ar” refers to an aromatic radical with six to ten ring atoms (e.g., (C₆₋₁₀)aromatic or C₆₋₁₀ aromatic, or (C₆₋₁₀)aryl or C₆₋₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in sources such as Greene et al., Protective Groups in Organic Synthesis, 4th^{rd} Ed., John Wiley & Sons, 2007. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” include optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., (C₁₋₄)heteroalkyl or C₁₋₄ heteroalkyl which refers to the chain length in total, which in this example is 4 atoms long. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” refers to a 5- to 18-membered aromatic radical (e.g., (C₅₋₁₈)heteroaryl or C₅₋₁₈ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. An N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Moiety” refers to a specific segment or functional group of a molecule. Chemical moieties are often recognized chemical entities embedded in or appended to a molecule.

“Nitro” refers to the —NO₂ radical.

“Oxa” refers to the —O— radical.

“Oxo” refers to the ═O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or the Pirkle alcohol, or derivatization of a compounds using a chiral compound such as Mosher's acid followed by chromatography or nuclear magnetic resonance spectroscopy.

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically enriched” and “non-racemic,” as used herein, refer to compositions in which the percent by weight of one enantiomer is greater than the amount of that one enantiomer in a control mixture of the racemic composition. The terms “enantiomerically pure” or “substantially enantiomerically pure” refers to a composition that comprises at least 98% of a single enantiomer and less than 2% of the opposite enantiomer.

In preferred embodiments, an enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions (Wiley Interscience, New York, 1981); Eliel, Stereochemistry of Carbon Compounds (McGraw-Hill, N Y, 1962); and Eliel and Wilen, Stereochemistry of Organic Compounds (Wiley-Interscience, New York, 1994).

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g. in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

“Substituted” means that the referenced group may have attached one or more groups, radicals, or additional moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)-(optionally substituted heteroaryl) and —S(O)-(optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

Compounds of the invention also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline forms of the compound, including, for example, polymorphs, pseudopolymorphs, solvates, hydrates, unsolvated polymorphs (including anhydrates), conformational polymorphs, and mixtures thereof. “Solvate” refers to a compound in physical association with one or more molecules of a pharmaceutically acceptable solvent. “Hydrate” refers to a compound in physical association with one or more molecules of water.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

### BTK Inhibitors

The BTK inhibitor may be any BTK inhibitor known in the art. In particular, it is one of the BTK inhibitors described in more detail in the following paragraphs.

In an embodiment, the BTK inhibitor is a compound of Formula (I):

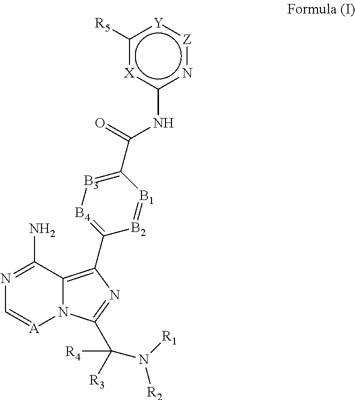

or a pharmaceutically acceptable salt, ester, solvate, hydrate, cocrystal, or prodrug thereof, wherein:
X is CH, N, O or S;
Y is C(R₆), N, O or S;
Z is CH, N or bond;
A is CH or N;
B₁ is N or C(R₇);
   B₂ is N or C(R₈);
B₃ is N or C(R₉);
B₄ is N or C(R₁₀);
R₁ is R₁₁C(═O), R₁₂S(═O), R₁₃S(═O)₂ or (C₁₋₆)alkyl optionally substituted with R₁₄;
R₂ is H, (C₁₋₃)alkyl or (C₃₋₇)cycloalkyl;
R₃ is H, (C₁₋₆)alkyl or (C₃₋₇)cycloalkyl); or
R₂ and R₃ form, together with the N and C atom they are attached to, a (C₃₋₇)heterocycloalkyl optionally substituted with one or more fluorine, hydroxyl, (C₁₋₃)alkyl, (C₁₋₃)alkoxy or oxo;
R₄ is H or (C₁₋₃)alkyl;
R₅ is H, halogen, cyano, (C₁₋₃)alkoxy, (C₃₋₆)cycloalkyl, any alkyl group of which is optionally substituted with one or more halogen; or R₅ is (C₆₋₁₀)aryl or (C₂₋₆)heterocycloalkyl;
R₆ is H or (C₁₋₃)alkyl; or
R₅ and R₆ together may form a (C₃₋₇)cycloalkenyl or (C₂₋₆)heterocycloalkenyl, each optionally substituted with (C₁₋₃)alkyl or one or more halogens;
R₇ is H, halogen, CF₃, (C₁₋₃)alkyl or (C₁₋₃)alkoxy;
R₈ is H, halogen, CF₃, (C₁₋₃)alkyl or (C₁₋₃)alkoxy; or
R₇ and R₈ together with the carbon atoms they are attached to, form (C₆₋₁₀)aryl or (C₁₋₉)heteroaryl;
R₉ is H, halogen, (C₁₋₃)alkyl or (C₁₋₃)alkoxy;
R₁₀ is H, halogen, (C₁₋₃)alkyl or (C₁₋₃)alkoxy;
R₁₁ is independently selected from the group consisting of (C₁₋₆)alkyl, (C₂₋₆)alkenyl and (C₂₋₆)alkynyl, where each alkyl, alkenyl or alkynyl is optionally substituted with one or more substituents selected from the group consisting of hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl and (C₃₋₇)heterocycloalkyl; or R₁₁ is (C₁₋₃)alkyl-C(O)—S—(C₁₋₃)alkyl; or
R₁₁ is (C₁₋₅)heteroaryl optionally substituted with one or more substituents selected from the group consisting of halogen or cyano;
R₁₂ and R₁₃ are independently selected from the group consisting of (C₂₋₆)alkenyl or (C₂₋₆)alkynyl, both optionally substituted with one or more substituents selected from the group consisting of hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl and (C₃₋₇)heterocycloalkyl; or a (C₁₋₅)heteroaryl optionally substituted with one or more substituents selected from the group consisting of halogen and cyano; and
R₁₄ is independently selected from the group consisting of halogen, cyano, (C₂₋₆)alkenyl and (C₂₋₆)alkynyl, both optionally substituted with one or more substituents selected from the group consisting of hydroxyl, (C₁₋₄)alkyl, (C₃₋₇)cycloalkyl, (C₁₋₄)alkylamino, di[(C₁₋₄)alkyl]amino, (C₁₋₃)alkoxy, (C₃₋₇)cycloalkoxy, (C₆₋₁₀)aryl, (C₁₋₅)heteroaryl and (C₃₋₇)heterocycloalkyl;
   with the proviso that:
   0 to 2 atoms of X, Y, Z can simultaneously be a heteroatom;
when one atom selected from X, Y is O or S, then Z is a bond and the other atom selected from X, Y can not be O or S;
when Z is C or N then Y is C(R₆) or N and X is C or N;
0 to 2 atoms of B₁, B₂, B₃ and B₄ are N;
with the terms used having the following meanings:
(C₁₋₂)alkyl means an alkyl group having 1 to 2 carbon atoms, being methyl or ethyl,
(C₁₋₃)alkyl means a branched or unbranched alkyl group having 1-3 carbon atoms, being methyl, ethyl, propyl or isopropyl;
(C₁₋₄)alkyl means a branched or unbranched alkyl group having 1-4 carbon atoms, being methyl, ethyl, propyl, isopropyl, butyl, isobutyl, sec-butyl and tert-butyl, (C₁₋₃)alkyl groups being preferred;
(C₁₋₅)alkyl means a branched or unbranched alkyl group having 1-5 carbon atoms, for example methyl, ethyl, propyl, isopropyl, butyl, isobutyl, sec-butyl, tert-butyl, pentyl and isopentyl, (C₁₋₄)alkyl groups being preferred. (C₁₋₆)Alkyl means a branched or unbranched alkyl group having 1-6 carbon atoms, for example methyl, ethyl, propyl, isopropyl, butyl, tert-butyl, n-pentyl and n-hexyl. (C₁₋₅)alkyl groups are preferred, (C₁₋₄)alkyl being most preferred;
(C₁₋₂)alkoxy means an alkoxy group having 1-2 carbon atoms, the alkyl moiety having the same meaning as previously defined;
(C₁₋₃)alkoxy means an alkoxy group having 1-3 carbon atoms, the alkyl moiety having the same meaning as previously defined. (C₁₋₂)alkoxy groups are preferred;
(C₁₋₄)alkoxy means an alkoxy group having 1-4 carbon atoms, the alkyl moiety having the same meaning as previously defined. (C₁₋₃)alkoxy groups are preferred, (C₁₋₂)alkoxy groups being most preferred;
(C₂₋₄)alkenyl means a branched or unbranched alkenyl group having 2-4 carbon atoms, such as ethenyl, 2-propenyl, isobutenyl or 2-butenyl;
(C₂₋₆)alkenyl means a branched or unbranched alkenyl group having 2-6 carbon atoms, such as ethenyl, 2-butenyl, and n-pentenyl, (C₂₋₄)alkenyl groups being most preferred;
(C₂₋₄)alkynyl means a branched or unbranched alkynyl group having 2-4 carbon atoms, such as ethynyl, 2-propynyl or 2-butynyl;
(C₂₋₆)alkynyl means a branched or unbranched alkynyl group having 2-6 carbon atoms, such as ethynyl, propynyl, n-butynyl, n-pentynyl, isopentynyl, isohexynyl or n-hexynyl. (C₂₋₄)alkynyl groups are preferred; (C₃₋₆)cycloalkyl means a cycloalkyl group having 3-6 carbon atoms, being cyclopropyl, cyclobutyl, cyclopentyl or cyclohexyl;
(C₃₋₇)cycloalkyl means a cycloalkyl group having 3-7 carbon atoms, being cyclopropyl, cyclobutyl, cyclopentyl, cyclohexyl or cycloheptyl;
(C₂₋₆)heterocycloalkyl means a heterocycloalkyl group having 2-6 carbon atoms, preferably 3-5 carbon atoms, and one or two heteroatoms selected from N, O and/or S, which may be attached via a heteroatom if feasible, or a carbon atom; preferred heteroatoms are N or O; also preferred are piperidine, morpholine, pyrrolidine and piperazine; with the most preferred
(C₂₋₆)heterocycloalkyl being pyrrolidine; the heterocycloalkyl group may be attached via a heteroatom if feasible;
(C₃₋₇)heterocycloalkyl means a heterocycloalkyl group having 3-7 carbon atoms, preferably 3-5 carbon atoms, and one or two heteroatoms selected from N, O and/or S. Preferred heteroatoms are N or O; preferred (C₃₋₇) heterocycloalkyl groups are azetidinyl, pyrrolidinyl, piperidinyl, homopiperidinyl or morpholinyl; more preferred (C₃₋₇)heterocycloalkyl groups are piperidine, morpholine and pyrrolidine; and the heterocycloalkyl group may be attached via a heteroatom if feasible;
(C₃₋₇)cycloalkoxy means a cycloalkyl group having 3-7 carbon atoms, with the same meaning as previously defined, attached via a ring carbon atom to an exocyclic oxygen atom;
(C₆₋₁₀)aryl means an aromatic hydrocarbon group having 6-10 carbon atoms, such as phenyl, naphthyl, tetrahydronaphthyl or indenyl; the preferred (C₆₋₁₀)aryl group is phenyl;
(C₁₋₅)heteroaryl means a substituted or unsubstituted aromatic group having 1-5 carbon atoms and 1-4 heteroatoms selected from N, O and/or S; the (C₁₋₅)heteroaryl may optionally be substituted; preferred (C₁₋₅)heteroaryl groups are tetrazolyl, imidazolyl, thiadiazolyl, pyridyl, pyrimidyl, triazinyl, thienyl or furyl, a more preferred (C₁₋₅)heteroaryl is pyrimidyl;
(C₁₋₉)heteroaryl means a substituted or unsubstituted aromatic group having 1-9 carbon atoms and 1-4 heteroatoms selected from N, O and/or S; the (C₁₋₉)heteroaryl may optionally be substituted; preferred (C₁₋₉)heteroaryl groups are quinoline, isoquinoline and indole;
[(C₁₋₄)alkyl]amino means an amino group, monosubstituted with an alkyl group containing 1-4 carbon atoms having the same meaning as previously defined; preferred [(C₁₋₄)alkyl]amino group is methylamino;
di[(C₁₋₄)alkyl]amino means an amino group, disubstituted with alkyl group(s), each containing 1-4 carbon atoms and having the same meaning as previously defined; preferred di[(C₁₋₄)alkyl]amino group is dimethylamino;
halogen means fluorine, chlorine, bromine or iodine;
(C₁₋₃)alkyl-C(O)—S—(C₁₋₃)alkyl means an alkyl-carbonyl-thio-alkyl group, each of the alkyl groups having 1 to 3 carbon atoms with the same meaning as previously defined;
(C₃₋₇)cycloalkenyl means a cycloalkenyl group having 3-7 carbon atoms, preferably 5-7 carbon atoms; preferred (C₃₋₇)cycloalkenyl groups are cyclopentenyl or cyclohexenyl; cyclohexenyl groups are most preferred;
(C₂₋₆)heterocycloalkenyl means a heterocycloalkenyl group having 2-6 carbon atoms, preferably 3-5 carbon atoms; and 1 heteroatom selected from N, O and/or S; preferred (C₂₋₆)heterocycloalkenyl groups are oxycyclohexenyl and azacyclohexenyl group.
   In the above definitions with multifunctional groups, the attachment point is at the last group.
When, in the definition of a substituent, it is indicated that “all of the alkyl groups” of said substituent are optionally substituted, this also includes the alkyl moiety of an alkoxy group.
   A circle in a ring of Formula (I) indicates that the ring is aromatic.
   Depending on the ring formed, the nitrogen, if present in X or Y, may carry a hydrogen.

In a preferred embodiment, the BTK inhibitor is a compound of Formula (I) or a pharmaceutically acceptable salt thereof, wherein:
X is CH or S;
Y is C(R₆);
Z is CH or bond;
A is CH;
B₁ is N or C(R₇);
B₂ is N or C(R₈);
B₃ is N or CH;
B₄ is N or CH;
R₁ is R₁₁C(═O),
R₂ is (C₁₋₃)alkyl;
R₃ is (C₁₋₃)alkyl; or
R₂ and R₃ form, together with the N and C atom they are attached to, a (C₃₋₇)heterocycloalkyl ring selected from the group consisting of azetidinyl, pyrrolidinyl, piperidinyl, and morpholinyl, optionally substituted with one or more fluorine, hydroxyl, (C₁₋₃)alkyl, or (C₁₋₃)alkoxy;
R₄ is H;
R₅ is H, halogen, cyano, (C₁₋₃)alkoxy, (C₃₋₆)cycloalkyl, or an alkyl group which is optionally substituted with one or more halogen;
R₆ is H or (C₁₋₃)alkyl;
R₇ is H, halogen or (C₁₋₃)alkoxy;
R₈ is H or (C₁₋₃)alkyl; or
R₇ and R₈ form, together with the carbon atom they are attached to a (C₆₋₁₀)aryl or (C₁₋₉)heteroaryl;
R₅ and R₆ together may form a (C₃₋₇)cycloalkenyl or (C₂₋₆)heterocycloalkenyl, each optionally substituted with (C₁₋₃)alkyl or one or more halogen;
R₁₁ is independently selected from the group consisting of (C₂₋₆)alkenyl and (C₂₋₆)alkynyl, where each alkenyl or alkynyl is optionally substituted with one or more substituents selected from the group consisting of hydroxyl, (C₁₋₄ alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl and (C₃₋₇)heterocycloalkyl;
   with the proviso that 0 to 2 atoms of B₁, B₂, B₃ and B₄ are N.

In an embodiment of Formula (I), B₁ is C(R₇); B₂ is C(R₈); B₃ is C(R₉); B₄ is C(R₁₀); R₇, R₉, and R₁₀ are each H; and R₈ is hydrogen or methyl.

In an embodiment of Formula (I), the ring containing X, Y and Z is selected from the group consisting of pyridyl, pyrimidyl, pyridazyl, triazinyl, thiazolyl, oxazolyl and isoxazolyl.

In an embodiment of Formula (I), the ring containing X, Y and Z is selected from the group consisting of pyridyl, pyrimidyl and pyridazyl.

In an embodiment of Formula (I), the ring containing X, Y and Z is selected from the group consisting of pyridyl and pyrimidyl.

In an embodiment of Formula (I), the ring containing X, Y and Z is pyridyl.

In an embodiment of Formula (I), R₅ is selected from the group consisting of hydrogen, fluorine, methyl, methoxy and trifluoromethyl.

In an embodiment of Formula (I), R₅ is hydrogen.

In an embodiment of Formula (I), R₂ and R₃ together form a heterocycloalkyl ring selected from the group consisting of azetidinyl, pyrrolidinyl, piperidinyl, homopiperidinyl and morpholinyl, optionally substituted with one or more of fluoro, hydroxyl, (C₁₋₃)alkyl and (C₁₋₃)alkoxy.

In an embodiment of Formula (I), R₂ and R₃ together form a heterocycloalkyl ring selected from the group consisting of azetidinyl, pyrrolidinyl and piperidinyl.

In an embodiment of Formula (I), R₂ and R₃ together form a pyrrolidinyl ring.

In an embodiment of Formula (I), R₁ is independently selected from the group consisting of (C₁₋₆)alkyl, (C₂₋₆)alkenyl or (C₂₋₆)alkynyl, each optionally substituted with one or more substituents selected from the group consisting of hydroxyl, (C₁₋₄)alkyl, (C₃₋₇)cycloalkyl, [(C₁₋₄)alkyl]amino, di[(C₁₋₄)alkyl] amino, (C₁₋₃)alkoxy, (C₃₋₇)cycloalkoxy, (C₆₋₁₀)aryl and (C₃₋₇)heterocycloalkyl.

In an embodiment of Formula (I), B₁, B₂, B₃ and B₄ are CH; X is N; Y and Z are CH; R₅ is CH₃; A is N; R₂, R₃ and R₄ are H; and R₁ is CO—CH₃.

In an embodiment of Formula (I), B₁, B₂, B₃ and B₄ are CH; X and Y are N; Z is CH; R₅ is CH₃; A is N; R₂, R₃ and R₄ are H; and R₁ is CO—CH₃.

In an embodiment of Formula (I), B₁, B₂, B₃ and B₄ are CH; X and Y are N; Z is CH; R₅ is CH₃; A is CH; R₂ and R₃ together form a piperidinyl ring; R₄ is H; and R₁ is CO-ethenyl.

In an embodiment of Formula (I), B₁, B₂, B₃ and B₄ are CH; X, Y and Z are CH; R₅ is H; A is CH; R₂ and R₃ together form a pyrrolidinyl ring; R₄ is H; and R₁ is CO-propynyl.

In an embodiment of Formula (I), B₁, B₂, B₃ and B₄ are CH; X, Y and Z are CH; R₅ is CH₃; A is CH; R₂ and R₃ together form a piperidinyl ring; R₄ is H; and R₁ is CO-propynyl.

In an embodiment of Formula (I), B₁, B₂, B₃ and B₄ are CH; X and Y are N; Z is CH; R₅ is H; A is CH; R₂ and R₃ together form a morpholinyl ring; R₄ is H; and R₁ is CO-ethenyl.

In an embodiment of Formula (I), B₁, B₂, B₃ and B₄ are CH; X and Y are N; Z is CH; R₅ is CH₃; A is CH; R₂ and R₃ together form a morpholinyl ring; R₄ is H; and R₁ is CO-propynyl.

In a preferred embodiment, the BTK inhibitor is a compound of Formula (II):

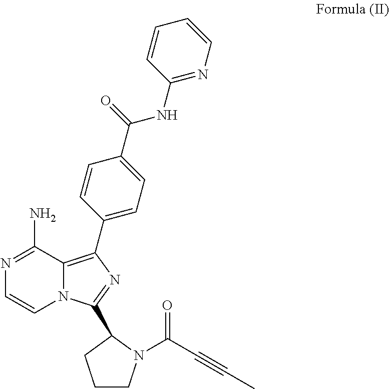 [SMILES: CC#CC(=O)N1CCC[C@H]1c1nc(-c2ccc(C(=O)Nc3ccccn3)cc2)c2c(N)nccn12]

or a pharmaceutically acceptable salt, ester, solvate, hydrate, cocrystal, or prodrug thereof. The preparation of this compound is described in U.S. Patent Application Publication No. 2014/0155385 A1, the disclosure of which is incorporated herein by reference.

In a preferred embodiment, the BTK inhibitor is (S)-4-(8-amino-3-(1-(but-2-ynoyl)pyrrolidin-2-yl)imidazo[1,5-a]pyrazin-1-yl)-N-(pyridin-2-yl)benzamide or pharmaceutically acceptable salt, solvate, hydrate, cocrystal, or prodrug thereof.

In a preferred embodiment, the BTK inhibitor is a compound of Formula (III):

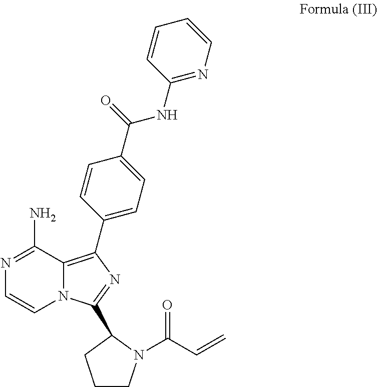 [SMILES: C=CC(=O)N1CCC[C@H]1c1nc(-c2ccc(C(=O)Nc3ccccn3)cc2)c2c(N)nccn12]

or a pharmaceutically acceptable salt, ester, solvate, hydrate, cocrystal, or prodrug thereof. The preparation of this compound is described in U.S. Patent Application Publication No. 2014/0155385 A1, the disclosure of which is incorporated herein by reference.

In a preferred embodiment, the BTK inhibitor is a compound of Formula (IV):

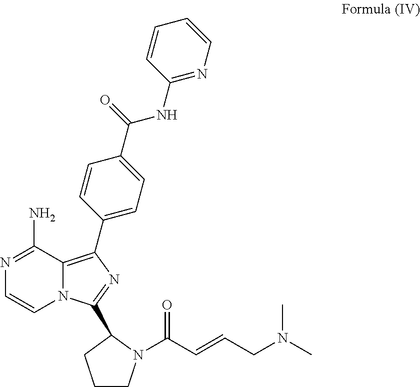 [SMILES: CN(C)C/C=C/C(=O)N1CCC[C@H]1c1nc(-c2ccc(C(=O)Nc3ccccn3)cc2)c2c(N)nccn12]

or a pharmaceutically acceptable salt, ester, solvate, hydrate, cocrystal, or prodrug thereof. The preparation of this compound is described in U.S. Patent Application Publication No. 2014/0155385 A1, the disclosure of which is incorporated herein by reference.

In a preferred embodiment, the BTK inhibitor is a compound of Formula (V):

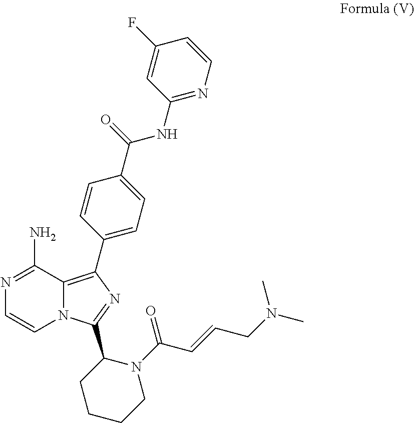 [SMILES: CN(C)C/C=C/C(=O)N1CCCC[C@H]1c1nc(-c2ccc(C(=O)Nc3cc(F)ccn3)cc2)c2c(N)nccn12]

or a pharmaceutically acceptable salt, ester, solvate, hydrate, cocrystal, or prodrug thereof. The preparation of this compound is described in U.S. Patent Application Publication No. 2014/0155385 A1, the disclosure of which is incorporated herein by reference.

In a preferred embodiment, the BTK inhibitor is a compound of Formula (VI):

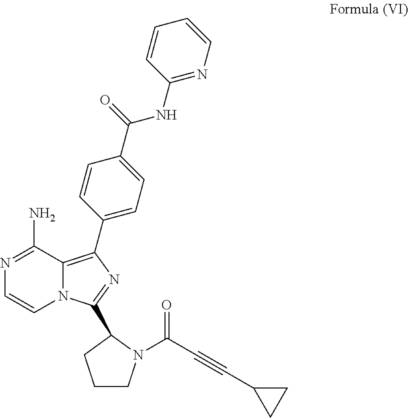 [SMILES: Nc1nccn2c([C@@H]3CCCN3C(=O)C#CC3CC3)nc(-c3ccc(C(=O)Nc4ccccn4)cc3)c12]

or a pharmaceutically acceptable salt, ester, solvate, hydrate, cocrystal, or prodrug thereof. The preparation of this compound is described in U.S. Patent Application Publication No. 2014/0155385 A1, the disclosure of which is incorporated herein by reference.

In a preferred embodiment, the BTK inhibitor is a compound of Formula (VII):

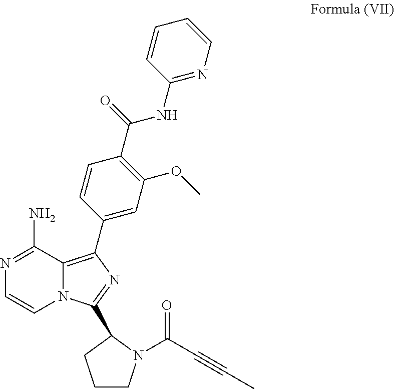 [SMILES: CC#CC(=O)N1CCC[C@H]1c1nc(-c2ccc(C(=O)Nc3ccccn3)c(OC)c2)c2c(N)nccn12]

or a pharmaceutically acceptable salt, ester, solvate, hydrate, cocrystal, or prodrug thereof. The preparation of this compound is described in U.S. Patent Application Publication No. 2014/0155385 A1, the disclosure of which is incorporated herein by reference.

In other embodiments, the BTK inhibitors include, but are not limited to, those compounds described in U.S. Patent Application Publication No. 2014/0155385 A1, the disclosures of each of which are specifically incorporated by reference herein.

In an embodiment, the BTK inhibitor is a compound of Formula (VIII):

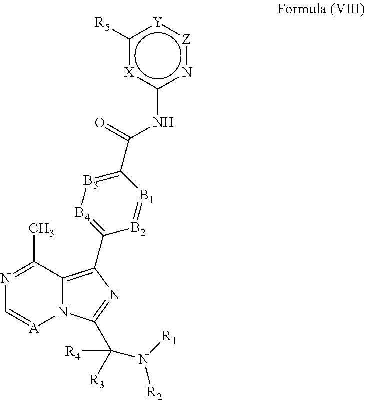

or a pharmaceutically acceptable salt, ester, solvate, hydrate, cocrystal, or prodrug thereof, wherein:
X is CH, N, O or S;
Y is C(R₆), N, O or S;
Z is CH, N or bond;
A is CH or N;
B₁ is N or C(R₇);
B₂ is N or C(R₈);
B₃ is N or C(R₉);
B₄ is N or C(R₁₀);
R₁ is R₁₁C(O), R₁₂S(O), R₁₃SO₂ or (C₁₋₆)alkyl optionally substituted with R₁₄;
R₂ is H, (C₁₋₃)alkyl or (C₃₋₇)cycloalkyl;
R₃ is H, (C₁₋₆)alkyl or (C₃₋₇)cycloalkyl); or
R₂ and R₃ form, together with the N and C atom they are attached to, a (C₃₋₇)heterocycloalkyl optionally substituted with one or more fluorine, hydroxyl, (C₁₋₃)alkyl, (C₁₋₃)alkoxy or oxo;
R₄ is H or (C₁₋₃)alkyl;
R₅ is H, halogen, cyano, (C₁₋₃)alkoxy, (C₃₋₆)cycloalkyl; all alkyl groups of R5 are optionally substituted with one or more halogen; or R₅ is (C₆₋₁₀)aryl or (C₂₋₆)heterocycloalkyl;
R₆ is H or (C₁₋₃)alkyl; or R₅ and R₆ together may form a (C₃₋₇)cycloalkenyl, or (C₂₋₆)heterocycloalkenyl; each optionally substituted with (C₁₋₃)alkyl, or one or more halogen;
R₇ is H, halogen, CF₃, (C₁₋₃)alkyl or (C₁₋₃)alkoxy;
R₈ is H, halogen, CF₃, (C₁₋₃)alkyl or (C₁₋₃)alkoxy; or
R₇ and R₈ together with the carbon atoms they are attached to, form (C₆₋₁₀)aryl or (C₁₋₅)heteroaryl;
R₉ is H, halogen, (C₁₋₃)alkyl or (C₁₋₃)alkoxy;
R₁₀ is H, halogen, (C₁₋₃)alkyl or (C₁₋₃)alkoxy;
R₁₁ is independently selected from a group consisting of (C₁₋₆)alkyl, (C₂₋₆)alkenyl and (C₂₋₆)alkynyl each alkyl, alkenyl or alkynyl optionally substituted with one or more groups selected from hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl or (C₃₋₇)heterocycloalkyl, or
R₁₁ is (C₁₋₃)alkyl-C(O)—S—(C₁₋₃)alkyl; or
R₁₁ is (C₁₋₅)heteroaryl optionally substituted with one or more groups selected from halogen or cyano.
R₁₂ and R₁₃ are independently selected from a group consisting of (C₂₋₆)alkenyl or (C₂₋₆)alkynyl both optionally substituted with one or more groups selected from hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl, or (C₃₋₇)heterocycloalkyl; or
(C₁₋₅)heteroaryl optionally substituted with one or more groups selected from halogen or cyano;
R₁₄ is independently selected from a group consisting of halogen, cyano or (C₂₋₆)alkenyl or (C₂₋₆)alkynyl both optionally substituted with one or more groups selected from hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl, (C₁₋₅)heteroaryl or (C₃₋₇)heterocycloalkyl;
   with the proviso that
   0 to 2 atoms of X, Y, Z can simultaneously be a heteroatom;
   when one atom selected from X, Y is O or S, then Z is a bond and the other atom selected from X, Y can not be O or S;
   when Z is C or N then Y is C(R₆) or N and X is C or N;
   0 to 2 atoms of B₁, B₂, B₃ and B₄ are N;
      with the terms used having the following meanings:
(C₁₋₃)alkyl means a branched or unbranched alkyl group having 1-3 carbon atoms, being methyl, ethyl, propyl or isopropyl;
(C₁₋₄)alkyl means a branched or unbranched alkyl group having 1-4 carbon atoms, being methyl, ethyl, propyl, isopropyl, butyl, isobutyl, sec-butyl and tert-butyl, (C₁₋₃)alkyl groups being preferred;
(C₁₋₆)alkyl means a branched or unbranched alkyl group having 1-6 carbon atoms, for example methyl, ethyl, propyl, isopropyl, butyl, tert-butyl, n-pentyl and n-hexyl. (C₁₋₅)alkyl groups are preferred, (C₁₋₄)alkyl being most preferred;
(C₁₋₂)alkoxy means an alkoxy group having 1-2 carbon atoms, the alkyl moiety having the same meaning as previously defined;
(C₁₋₃)alkoxy means an alkoxy group having 1-3 carbon atoms, the alkyl moiety having the same meaning as previously defined, with (C₁₋₂)alkoxy groups preferred;
(C₂₋₃)alkenyl means an alkenyl group having 2-3 carbon atoms, such as ethenyl or 2-propenyl;
(C₂₋₄)alkenyl means a branched or unbranched alkenyl group having 2-4 carbon atoms, such as ethenyl, 2-propenyl, isobutenyl or 2-butenyl;
(C₂₋₆)alkenyl means a branched or unbranched alkenyl group having 2-6 carbon atoms, such as ethenyl, 2-butenyl, and n-pentenyl, with (C₂₋₄)alkenyl groups preferred, and (C₂₋₃)alkenyl groups even more preferred;
(C₂₋₄)alkynyl means a branched or unbranched alkynyl group having 2-4 carbon atoms, such as ethynyl, 2-propynyl or 2-butynyl;
(C₂₋₃)alkynyl means an alkynyl group having 2-3 carbon atoms, such as ethynyl or 2-propynyl;
(C₂₋₆)alkynyl means a branched or unbranched alkynyl group having ₂₋₆ carbon atoms, such as ethynyl, propynyl, n-butynyl, n-pentynyl, isopentynyl, isohexynyl or n-hexynyl, with (C₂₋₄)alkynyl groups preferred, and (C₂₋₃)alkynyl groups more preferred;
(C₃₋₆)cycloalkyl means a cycloalkyl group having 3-6 carbon atoms, being cyclopropyl, cyclobutyl, cyclopentyl or cyclohexyl;
(C₃₋₇)cycloalkyl means a cycloalkyl group having 3-7 carbon atoms, being cyclopropyl, cyclobutyl, cyclopentyl, cyclohexyl or cycloheptyl;
(C₂₋₆)heterocycloalkyl means a heterocycloalkyl group having 2-6 carbon atoms, preferably 3-5 carbon atoms, and one or two heteroatoms selected from N, O and/or S, which may be attached via a heteroatom if feasible, or a carbon atom; preferred heteroatoms are N or O; preferred groups are piperidine, morpholine, pyrrolidine and piperazine; a most preferred (C₂₋₆)heterocycloalkyl is pyrrolidine; and the heterocycloalkyl group may be attached via a heteroatom if feasible;
(C₃₋₇)heterocycloalkyl means a heterocycloalkyl group having 3-7 carbon atoms, preferably 3-5 carbon atoms, and one or two heteroatoms selected from N, O and/or S; preferred heteroatoms are N or O; preferred (C₃₋₇) heterocycloalkyl groups are azetidinyl, pyrrolidinyl, piperidinyl, homopiperidinyl or morpholinyl; more preferred (C₃₋₇)heterocycloalkyl groups are piperidine, morpholine and pyrrolidine; even more preferred are piperidine and pyrrolodine; and the heterocycloalkyl group may be attached via a heteroatom if feasible;
(C₃₋₇)cycloalkoxy means a cycloalkyl group having 3-7 carbon atoms, with the same meaning as previously defined, attached via a ring carbon atom to an exocyclic oxygen atom;
(C₆₋₁₀)aryl means an aromatic hydrocarbon group having 6-10 carbon atoms, such as phenyl, naphthyl, tetrahydronaphthyl or indenyl; the preferred (C₆₋₁₀)aryl group is phenyl;
(C₁₋₅)heteroaryl means a substituted or unsubstituted aromatic group having 1-5 carbon atoms and 1-4 heteroatoms selected from N, O and/or S, wherein the (C₁₋₅)heteroaryl may optionally be substituted; preferred (C₁₋₅)heteroaryl groups are tetrazolyl, imidazolyl, thiadiazolyl, pyridyl, pyrimidyl, triazinyl, thienyl or furyl, and the more preferred (C₁₋₅)heteroaryl is pyrimidyl;
[(C₁₋₄)alkyl]amino means an amino group, monosubstituted with an alkyl group containing 1-4 carbon atoms having the same meaning as previously defined; the preferred [(C₁₋₄)alkyl]amino group is methylamino;
di[(C₁₋₄)alkyl]amino means an amino group, disubstituted with alkyl group(s), each containing 1-4 carbon atoms and having the same meaning as previously defined; the preferred di[(C₁₋₄)alkyl]amino group is dimethylamino;
halogen means fluorine, chlorine, bromine or iodine;
(C₁₋₃)alkyl-C(O)—S—(C₁₋₃)alkyl means an alkyl-carbonyl-thio-alkyl group, each of the alkyl groups having 1 to 3 carbon atoms with the same meaning as previously defined;
(C₃₋₇)cycloalkenyl means a cycloalkenyl group having 3-7 carbon atoms, preferably 5-7 carbon atoms; preferred (C₃₋₇)cycloalkenyl groups are cyclopentenyl or cyclohexenyl; and cyclohexenyl groups are most preferred;
(C₂₋₆)heterocycloalkenyl means a heterocycloalkenyl group having 2-6 carbon atoms, preferably 3-5 carbon atoms; and 1 heteroatom selected from N, O and/or S; the preferred (C₂₋₆)heterocycloalkenyl groups are oxycyclohexenyl and azacyclohexenyl groups.
   In the above definitions with multifunctional groups, the attachment point is at the last group.
When, in the definition of a substituent, is indicated that “all of the alkyl groups” of said substituent are optionally substituted, this also includes the alkyl moiety of an alkoxy group.
   A circle in a ring of Formula (VIII) indicates that the ring is aromatic.
   Depending on the ring formed, the nitrogen, if present in X or Y, may carry a hydrogen.

In a preferred embodiment, the invention relates to a compound according to Formula (VIII) wherein B₁ is C(R₇); B₂ is C(R₈); B₃ is C(R₉) and B₄ is C(R₁₀).

In other embodiments, the BTK inhibitors include, but are not limited to, those compounds described in International Patent Application Publication No. WO 2013/010869, the disclosures of each of which are specifically incorporated by reference herein.

In an embodiment, the BTK inhibitor is a compound of Formula (IX):

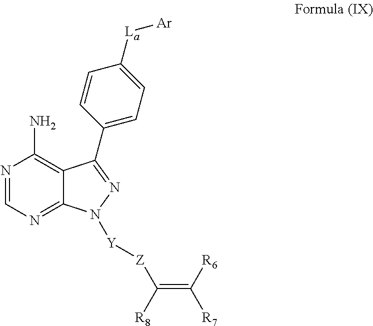

or a pharmaceutically acceptable salt, ester, solvate, hydrate, cocrystal, or prodrug thereof, wherein:
Lₐ is CH₂, O, NH or S;
Ar is a substituted or unsubstituted aryl, or a substituted or unsubstituted heteroaryl;
Y is an optionally substituted group selected from the group consisting of alkyl, heteroalkyl, cycloalkyl, heterocycloalkyl, aryl and heteroaryl;
Z is C(═O), OC(═O), NRC(═O), C(═S), S(═O), OS(═O)ₓ, or NRS(═O)ₓ, where x is 1 or 2;
R⁷ and R⁸ are each independently H; or R⁷ and R⁸ taken together form a bond;
R⁶ is H; and
R is H or (C₁₋₆)alkyl.

In a preferred embodiment, the BTK inhibitor is ibrutinib, also known as PCI-32765, or a pharmaceutically acceptable salt, ester, solvate, hydrate, cocrystal, or prodrug thereof. In an exemplary embodiment, the BTK inhibitor is (R)-1-(3-(4-amino-3-(4-phenoxyphenyl)-1H-pyrazolo[3,4-d]pyrimidin-1-yl)piperidin-1-yl)prop-2-en-1-one, or a pharmaceutically acceptable salt, ester, solvate, hydrate, cocrystal, or prodrug thereof. In an embodiment, the BTK inhibitor is 1-[(3R)-3-[4-amino-3-(4-phenoxyphenyl)-1H-pyrazolo[3,4-d]pyrimidin-1-yl]piperidin-1-yl]prop-2-en-1-one, or a pharmaceutically acceptable salt, ester, solvate, hydrate, cocrystal, or prodrug thereof. In an embodiment, the BTK inhibitor is (S)-1-(3-(4-amino-3-(4-phenoxyphenyl)-1H-pyrazolo[3,4-d]pyrimidin-1-yl)piperidin-1-yl)prop-2-en-1-one, or a pharmaceutically acceptable salt, ester, solvate, hydrate, cocrystal, or prodrug thereof. In a preferred embodiment, the BTK inhibitor has the structure of Formula (X), or an enantiomer thereof, or a pharmaceutically acceptable salt, ester, solvate, hydrate, cocrystal, or prodrug thereof:

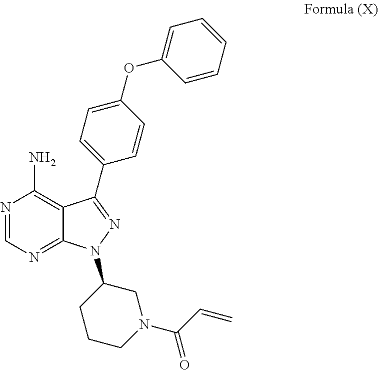 [SMILES: C=CC(=O)N1CCC[C@@H](n2nc(-c3ccc(Oc4ccccc4)cc3)c3c(N)ncnc32)C1]

In an embodiment, the BTK inhibitor is a compound of Formula (XI):

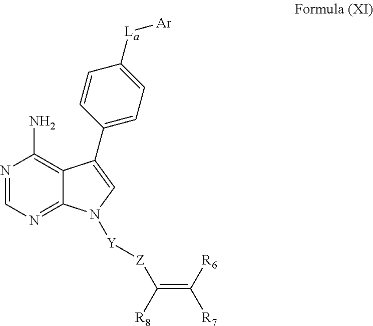

or a pharmaceutically acceptable salt, ester, solvate, hydrate, cocrystal, or prodrug thereof, wherein:
Lₐ is CH₂, O, NH or S;
Ar is a substituted or unsubstituted aryl, or a substituted or unsubstituted heteroaryl;
Y is an optionally substituted group selected from the group consisting of alkyl, heteroalkyl, cycloalkyl, heterocycloalkyl, aryl and heteroaryl;
Z is C(═O), OC(═O), NRC(═O), C(═S), S(═O), OS(═O) or NRS(═O), where x is 1 or 2;
R⁷ and R⁸ are each H; or R⁷ and R⁸ taken together form a bond;
R⁶ is H; and
R is H or (C₁₋₆)alkyl.

In an embodiment, the BTK inhibitor is a compound of Formula (XII):

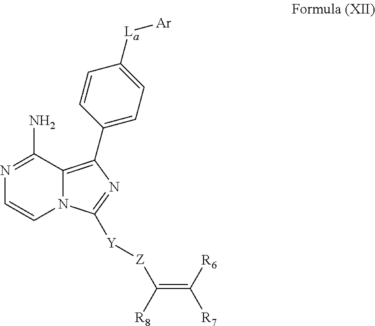

or a pharmaceutically acceptable salt, ester, solvate, hydrate, cocrystal, or prodrug thereof, wherein:
Lₐ is CH₂, O, NH or S;
Ar is a substituted or unsubstituted aryl, or a substituted or unsubstituted heteroaryl;
Y is an optionally substituted group selected from the group consisting of alkyl, heteroalkyl, cycloalkyl, heterocycloalkyl, aryl and heteroaryl;
Z is C(═O), OC(═O), NRC(═O), C(═S), S(═O)ₓ, OS(═O)ₓ or NRS(═O)ₓ, where x is 1 or 2;
R⁷ and R⁸ are each H; or R⁷ and R⁸ taken together form a bond;
R⁶ is H; and
R is H or (C₁₋₆)alkyl.

In an embodiment, the BTK inhibitor is a compound of Formula (XIII):

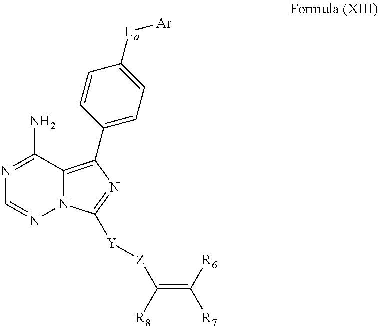

or a pharmaceutically acceptable salt, ester, solvate, hydrate, cocrystal, or prodrug thereof, wherein:
Lₐ is CH₂, O, NH or S;
Ar is a substituted or unsubstituted aryl, or a substituted or unsubstituted heteroaryl;
Y is an optionally substituted group selected from the group consisting of alkyl, heteroalkyl, cycloalkyl, heterocycloalkyl, aryl and heteroaryl;
Z is C(═O), OC(═O), NRC(═O), C(═S), S(═O)ₓ, OS(═O)ₓ or NRS(═O)ₓ, where x is 1 or 2;
R⁷ and R⁸ are each H; or R⁷ and R⁸ taken together form a bond;
R⁶ is H; and
R is H or (C₁₋₆)alkyl.

In an embodiment, the BTK inhibitor is a compound disclosed in U.S. Pat. No. 7,459,554, the disclosure of which is specifically incorporated herein by reference. In an embodiment, the BTK inhibitor is a compound of Formula (XIV):

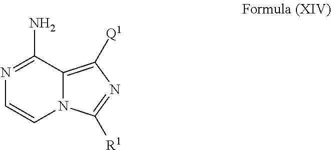

or a pharmaceutically acceptable salt, ester, solvate, hydrate, cocrystal, or prodrug thereof, wherein:
Q¹ is aryl¹, heteroaryl¹, cycloalkyl, heterocyclyl, cycloalkenyl, or heterocycloalkenyl, any of which is optionally substituted by one to five independent G¹ substituents;
R¹ is alkyl, cycloalkyl, bicycloalkyl, aryl, heteroaryl, aralkyl, heteroaralkyl, heterocyclyl, or heterobicycloalkyl, any of which is optionally substituted by one or more independent G¹¹ substituents;
G¹ and G⁴¹ are each independently halo, oxo, —CF₃, —OCF₃, —OR², —NR²R³(R^{3a})ⱼ₁, —C(O)R², —CO₂R², —CONR²R³, —NO₂, —CN, —S(O)ⱼ₁R², —SO₂NR²R³, NR²(C═O)R³, NR²(C═O)OR³, NR²(C═O)NR²R³, NR²S(O)ⱼ₁R³, —(C═S)OR², —(C═O)SR², —NR²(C═NR³)NR^{2a}R^{3a}, —NR²(C═NR³)OR^{2a}, —NR²(C═NR³)SR^{3a}, —O(C═O)OR², —O(C═O)NR²R³, —O(C═O)SR², —S(C═O)OR², —S(C═O)NR²R³, (C₀₋₁₀)alkyl, (C₂₋₁₀)alkenyl, (C₂₋₁₀)alkynyl, (C₁₋₁₀)alkoxy(C₁₋₁₀)alkyl, (C₁₋₁₀)alkoxy(C₂₋₁₀)alkenyl, (C₁₋₁₀)alkoxy(C₂₋₁₀)alkynyl, (C₁₋₁₀)alkylthio(C₁₋₁₀)alkyl, (C₁₋₁₀)alkylthio(C₂₋₁₀)alkenyl, (C₁₋₁₀)alkylthio(C₂₋₁₀)alkynyl, cyclo(C₃₋₈)alkyl, cyclo(C₃₋₈)alkenyl, cyclo(C₃₋₈)alkyl(C₁₋₁₀)alkyl, cyclo(C₃₋₈)alkenyl(C₁₋₁₀)alkyl, cyclo(C₃₋₈) alkyl(C₂₋₁₀)alkenyl, cyclo(C₃₋₈)alkenyl(C₂₋₁₀)alkenyl, cyclo(C₃₋₈)alkyl(C₂₋₁₀)alkynyl, cyclo(C₃₋₈)alkenyl(C₂₋₁₀)alkynyl, heterocyclyl-(C₀₋₁₀)alkyl, heterocyclyl-(C₂₋₁₀)alkenyl, or heterocyclyl-(C₂₋₁₀)alkynyl, any of which is optionally substituted with one or more independent halo, oxo, —CF₃, —OCF₃, —OR²²², —NR²²²R³³³(R³³³a)ⱼ₁ₐ, —C(O)R²²², —CO₂R²²², —CONR²²²R³³³, —NO₂, —CN, —S(O)ⱼ₁ₐR²²², —SO₂NR²²²R³³³, NR²²²(C═O)R³³³, NR²²²(C═O)OR³³³, NR²²²(C═O)NR²²²R³³³, NR²²²S(O)ⱼ₁ₐR³³³, —(C═S)OR²²², —(C═O)SR²²², —NR²²²(C═NR³³³)NR^{222a}R^{333a}, —NR²²²(C═NR³³³)OR^{222a}, —NR²²²(C═NR³³³)SR^{333a}, —O(C═O)OR²²², —O(C═O)NR²²²R³³³, —O(C═O)SR²²², —S(C═O)OR²²², or —S(C═O)NR²²²R³³³ substituents; or —(X¹)ₙ—(Y¹)ₘ—R⁴; or aryl-(C₀₋₁₀)alkyl, aryl-(C₂₋₁₀)alkenyl, or aryl-(C₂₋₁₀) alkynyl, any of which is optionally substituted with one or more independent halo, —CF₃, —OCF₃, —OR²²², —NR²²²R³³³(R^{333a})ⱼ₂ₐ, —C(O)R²²², —CO₂R²²², —CONR²²²R³³³, —NO₂, —CN, —S(O)ⱼ₂ₐR²²², —SO₂NR²²²R³³³, NR²²²(C═O)R³³³, NR²²²(C═O)OR³³³, NR²²²(C═O)NR²²²R³³³, NR²²²S(O)ⱼ₂ₐR³³³, —(C═S)OR²²², —(C═O)SR²²², —NR²²²(C═NR³³³)NR^{222a}R^{333a}, —NR²²²(C═NR³³³)OR^{222a}, —NR²²²(C═NR³³³)SR^{333a}, —O(C═O)OR²²², —O(C═O)NR²²²R³³³, —O(C═O)SR²²², —S(C═O)OR²²², or —S(C═O)NR²²²R³³³ substituents; or hetaryl-(C₀₋₁₀)alkyl, hetaryl-(C₂₋₁₀)alkenyl, or hetaryl-(C₂₋₁₀)alkynyl, any of which is optionally substituted with one or more independent halo, —CF₃, —OCF₃, —OR²²², —NR²²², R³³³(R^{333a})ⱼ₃ₐ, —C(O)R²²², —CO₂R²²², —CONR²²²R³³³, —NO₂, —CN, —S(O)ⱼ₃ₐR²²², —SO₂NR²²²R³³³, NR²²²(C═O)R³³³, NR²²²(C═O)OR³³³, NR²²²(C═O)NR²²²R³³³, NR²²²S(O)ⱼ₃ₐR³³³, —(C═S)OR²²², —(C═O)SR²²², —NR²²²(C═NR³³³)NR²²²aR³³³a, —NR²²²(C═NR³³³)OR^{222a}, NR²²²(C═NR³³³)SR³³³a, —O(C═O)OR²²², —O(C═O)NR²²²R³³³, —O(C═O)SR²²², —S(C═O)OR²²², or —S(C═O)NR²²²R³³³ substituents;
G¹¹ is halo, oxo, —CF₃, —OCF₃, —OR²¹, —NR²¹R³¹(R^{3a1})ⱼ₄, —C(O)R²¹, —CO₂R²¹, —CONR²¹R³¹, —NO₂, —CN, —S(O)ⱼ₄R²¹, —SO₂NR²¹R³¹, NR²¹(C═O)R³¹, NR²¹(C═O)OR³¹, NR²¹(C═O)NR²¹R³¹, NR²¹S(O)ⱼ₄R³¹, —(C═S)OR²¹, —(C═O)SR²¹, —NR²¹ (C═NR³¹)NR^{2a1}R^{3a1}, —NR²¹(C═NR³¹)OR^{2a1}, —NR²¹(C═NR³¹)SR^{3a1}, —O(C═O)OR²¹, —O(C═O)NR²¹R³¹, —O(C═O)SR²¹, —S(C═O)OR²¹, —S(C═O)NR²¹R³¹, —P(O)OR²¹OR³¹, (C₀₋₁₀)alkyl, (C₂₋₁₀)alkenyl, (C₂₋₁₀)alkynyl, (C₁₋₁₀) alkoxy(C₁₋₁₀)alkyl, (C₁₋₁₀)alkoxy(C₂₋₁₀)alkenyl, (C₁₋₁₀)alkoxy(C₂₋₁₀)alkynyl, (C₁₋₁₀) alkylthio(C₁₋₁₀)alkyl, (C₁₋₁₀)alkylthio(C₂₋₁₀)alkenyl, (C₁₋₁₀)alkylthio(C₂₋₁₀)alkynyl, cyclo(C₃₋₈)alkyl, cyclo(C₃₋₈)alkenyl, cyclo(C₃₋₈)alkyl(C₁₋₁₀)alkyl, cyclo(C₃₋₈)alkenyl(C₁₋₁₀) alkyl, cyclo(C₃₋₈)alkyl(C₂₋₁₀)alkenyl, cyclo(C₃₋₈)alkenyl(C₂₋₁₀)alkenyl, cyclo(C₃₋₈) alkyl(C₂₋₁₀) alkynyl, cyclo(C₃₋₈)alkenyl(C₂₋₁₀)alkynyl, heterocyclyl-(C₀₋₁₀)alkyl, heterocyclyl-(C₂₋₁₀) alkenyl, or heterocyclyl-(C₂₋₁₀)alkynyl, any of which is optionally substituted with one or more independent halo, oxo, —CF₃, —OCF₃, —OR²²²¹, —NR²²²¹R³³³¹(R^{333a1})ⱼ₄ₐ, —C(O)R²²²¹, —CO₂R²²²¹, —CONR²²²¹R³³³¹, —NO₂, —CN, —S(O)ⱼ₄ₐR²²²¹, —SO₂NR²²²¹R³³³¹, NR²²²¹(C═O)R³³³¹, NR²²²¹(C═O)OR³³³¹, NR²²²¹(C═O)NR²²²¹R³³³¹, NR²²²¹S(O)ⱼ₄ₐR³³³¹, —(C═S)OR²²²¹, —(C═O)SR²²²¹, —NR²²²¹(C═NR³³³¹)NR²²²¹R^{333a1}, —NR²²²¹(C═NR³³³¹)OR^{222a1}, —NR²²²¹(C═NR³³³¹)SR^{333a1}, —O(C═O)OR²²²¹, —O(C═O)NR²²²¹R³³³¹, —O(C═O)SR²²²¹, —S(C═O)OR²²²¹, —P(O)OR²²²¹OR³³³¹, or —S(C═O)NR²²²¹R³³³¹ substituents; or aryl-(C₀₋₁₀)alkyl, aryl-(C₂₋₁₀)alkenyl, or aryl-(C₂₋₁₀)alkynyl, any of which is optionally substituted with one or more independent halo, —CF₃, —OCF₃, —OR²²²¹, —NR²²²¹R³³³¹(R^{333a1})ⱼ₅ₐ, —C(O)R²²²¹, —CO₂R²²²¹, —CONR²²²¹R³³³¹, —NO₂, —CN, —S(O)ⱼ₅ₐR²²²¹, —SO₂NR²²²¹R³³³¹, NR²²²¹(C═O)R³³³¹, NR²²²¹(C═O)OR³³³¹, NR²²²¹(C═O)NR²²²¹R³³³¹, NR²²²¹S(O)ⱼ₅ₐR³³³¹, —(C═S)OR²²²¹, —(C═O)SR²²²¹, —NR²²²¹(C═NR³³³¹)NR^{222a1}R^{333a1}, —NR²²²¹(C═NR³³³¹)OR^{222a1}, —NR²²²¹(C═NR³³³¹)SR^{333a1}, —O(C═O)OR²²²¹, —O(C═O)NR²²²¹R³³³¹, —O(C═O)SR²²²¹, —S(C═O)OR²²²¹, —P(O)OR²²²¹R³³³¹, or —S(C═O)NR²²²¹R³³³¹ substituents; or hetaryl-(C₀₋₁₀) alkyl, hetaryl-(C₂₋₁₀)alkenyl, or hetaryl-(C₂₋₁₀)alkynyl, any of which is optionally substituted with one or more independent halo, —CF₃, —OCF₃, —OR²²²¹, —NR²²²¹R³³³¹(R^{333a1})ⱼ₆ₐ, —C(O)R²²²¹, —CO₂R²²²¹, —CONR²²²¹R³³³¹, —NO₂, —CN, —S(O)ⱼ₆ₐR²²²¹, —SO₂NR²²²¹R³³³¹, NR²²²¹(C═O)R³³³¹, NR²²²¹(C═O)OR³³³¹, NR²²²¹(C═O)NR²²²¹R³³³¹, NR²²²¹S(O)ⱼ₆ₐR³³³¹, —(C═S)OR²²²¹, —(C═O)SR²²²¹, —NR²²²¹(C═NR³³³¹)NR^{222a1}R^{333a1}, —NR₂₂₂₁(C═NR³³³¹)OR^{222a1}, —NR²²²¹(C═NR³³³¹)SR^{333a1}, —O(C═O)OR²²²¹, —O(C═O)NR²²²¹R³³³¹, —O(C═O)SR²²²¹, —S(C═O)OR²²²¹, —P(O)OR²²²¹OR³³³¹, or —S(C═O)NR²²²¹R³³³¹ substituents; or G¹¹ is taken together with the carbon to which it is attached to form a double bond which is substituted with R⁵ and G¹¹¹;
R², R^{2a}, R³, R^{3a}, R²²², R²²²a, R³³³, R^{333a}, R²¹, R^{2a1}, R³¹, R^{3a1}, R²²²¹, R^{222a1}, R³³³¹, and R^{333a} are each independently equal to (C₀₋₁₀)alkyl, (C₂₋₁₀)alkenyl, (C₂₋₁₀)alkynyl, (C₁₋₁₀)alkoxy(C₁₋₁₀)alkyl, (C₁₋₁₀)alkoxy(C₂₋₁₀)alkenyl, (C₁₋₁₀)alkoxy(C₂₋₁₀)alkynyl, (C₁₋₁₀)alkylthio(C₁₋₁₀)alkyl, (C₁₋₁₀)alkylthio(C₂₋₁₀)alkenyl, (C₁₋₁₀)alkylthio(C₂₋₁₀)alkynyl, cyclo(C₃₋₈)alkyl, cyclo(C₃₋₈)alkenyl, cyclo(C₃₋₈)alkyl(C₁₋₁₀)alkyl, cyclo(C₃₋₈)alkenyl(C₁₋₁₀)alkyl, cyclo(C₃₋₈)alkyl(C₂₋₁₀)alkenyl, cyclo(C₃₋₈)alkenyl(C₂₋₁₀)alkenyl, cyclo(C₃₋₈)alkyl(C₂₋₁₀)alkynyl, cyclo(C₃₋₈)alkenyl(C₂₋₁₀)alkynyl, heterocyclyl-(C₀₋₁₀)alkyl, heterocyclyl-(C₂₋₁₀)alkenyl, or heterocyclyl-(C₂₋₁₀)alkynyl, any of which is optionally substituted by one or more G¹¹¹ substituents; or aryl-(C₀₋₁₀)alkyl, aryl-(C₂₋₁₀)alkenyl, or aryl-(C₂₋₁₀)alkynyl, hetaryl-(C₀₋₁₀)alkyl, hetaryl-(C₂₋₁₀)alkenyl, or hetaryl-(C₂₋₁₀)alkynyl, any of which is optionally substituted by one or more G¹¹¹ substituents; or in the case of —NR²R³(R^{3a})ⱼ₁ or —NR²²²R³³³(R³³³a)ⱼ₁ₐ or —NR²²²R³³³(R³³³a)ⱼ₂ₐ or —NR²²²¹R³³³¹(R^{333a1})ⱼ₃ₐ or —NR²²²¹R³³³¹(R^{333a1})ⱼ₄ₐ or —NR²²²¹R³³³¹(R^{333a1})ⱼ₅ₐ or —NR²²²¹R³³³¹(R^{333a1})ⱼ₆ₐ, R² and R³ or R²²² and R³³³3 or R²²²¹ and R³³³¹ taken together with the nitrogen atom to which they are attached form a 3-10 membered saturated ring, unsaturated ring, heterocyclic saturated ring, or heterocyclic unsaturated ring, wherein said ring is optionally substituted by one or more G¹¹¹ substituents;
X¹ and Y¹ are each independently —O—, —NR⁷—, —S(O)ⱼ₇—, —CR⁵R⁶—, —N(C(O)OR⁷)—, —N(C(O)R⁷)—, —N(SO₂R⁷)—, —CH₂O—, —CH₂S—, —CH₂N(R⁷)—, —CH(NR⁷)—, —CH₂N(C(O)R⁷)—, —CH₂N(C(O)OR⁷)—, —CH₂N(SO₂R⁷)—, —CH(NHR⁷)—, —CH(NHC(O)R⁷)—, —CH(NHSO₂R⁷)—, —CH(NHC(O)OR⁷)—, —CH(OC(O)R⁷)—, —CH(OC(O)NHR⁷)—, —CH═CH—, —C.ident.C—, —C(═NOR⁷)—, —C(O)—, —CH(OR⁷)—, —C(O)N(R⁷)—, —N(R⁷)C(O)—, —N(R⁷)S(O)—, —N(R⁷)S(O)₂—, —OC(O)N(R⁷)—, —N(R⁷)C(O)N(R⁷)—, —NR⁷C(O)O—, —S(O)N(R⁷)—, —S(O)₂N(R⁷)—, —N(C(O)R⁷)S(O)—, —N(C(O)R⁷)S(O)₂—, —N(R⁷)S(O)N(R⁷)—, —N(R⁷)S(O)₂N(R⁷)—, —C(O)N(R⁷)C(O)—, —S(O)N(R⁷)C(O)—, —S(O)₂N(R⁷)C(O)—, —OS(O)N(R⁷)—, —OS(O)₂N(R⁷)—, —N(R⁷)S(O)O—, —N(R⁷)S(O)₂O—, —N(R⁷)S(O)C(O)—, —N(R⁷)S(O)₂C(O)—, —SON(C(O)R⁷)—, —SO₂N(C(O)R⁷)—, —N(R⁷)SON(R⁷)—, —N(R⁷)SO₂N(R⁷)—, —C(O)O—, —N(R⁷)P(OR⁸)O—, —N(R⁷)P(OR⁸)—, —N(R⁷)P(O)(OR⁸)O—, —N(R⁷)P(O)(OR⁸)—, —N(C(O)R⁷)P(OR⁸)O—, —N(C(O)R⁷)P(OR⁸)—, —N(C(O)R⁷)P(O)(OR⁸)O—, —N(C(O)R⁷)P(OR⁸)—, —CH(R⁷)S(O)—, —CH(R⁷)S(O)₂—, —CH(R⁷)N(C(O)OR⁷)—, —CH(R⁷)N(C(O)R⁷)—, —CH(R⁷)N(SO₂R⁷)—, —CH(R⁷)O—, —CH(R⁷)S—, —CH(R⁷)N(R⁷)—, —CH(R⁷)N(C(O)R⁷)—, —CH(R⁷)N(C(O)OR⁷)—, —CH(R⁷)N(SO₂R⁷)—, —CH(R⁷)C(═NOR⁷)—, —CH(R⁷)C(O)—, —CH(R⁷)CH(OR⁷)—, —CH(R⁷)C(O)N(R⁷)—, —CH(R⁷)N(R⁷)C(O)—, —CH(R⁷)N(R⁷)S(O)—, —CH(R⁷)N(R⁷)S(O)₂—, —CH(R⁷)OC(O)N(R⁷)—, —CH(R⁷)N(R⁷)C(O)N(R⁷)—, —CH(R⁷)NR⁷C(O)O—, —CH(R⁷)S(O)N(R⁷)—, —CH(R⁷)S(O)₂N(R⁷)—, —CH(R⁷)N(C(O)R⁷)S(O)—, —CH(R⁷)N(C(O)R⁷)S(O)—, —CH(R⁷)N(R⁷)S(O)N(R⁷)—, —CH(R⁷)N(R⁷)S(O)₂N(R⁷)—, —CH(R⁷)C(O)N(R⁷)C(O)—, —CH(R⁷)S(O)N(R⁷)C(O)—, —CH(R⁷)S(O)₂N(R⁷)C(O)—, —CH(R⁷)OS(O)N(R⁷)—, —CH(R⁷)OS(O)₂N(R⁷)—, —CH(R⁷)N(R⁷)S(O)O—, —CH(R⁷)N(R⁷)S(O)₂O—, —CH(R⁷)N(R⁷)S(O)C(O)—, —CH(R⁷)N(R⁷)S(O)₂C(O)—, —CH(R⁷)SON(C(O)R⁷)—, —CH(R⁷)SO₂N(C(O)R⁷)—, —CH(R⁷)N(R⁷)SON(R⁷)—, —CH(R⁷)N(R⁷)SO₂N(R⁷)—, —CH(R⁷)C(O)O—, —CH(R⁷)N(R⁷)P(OR⁸)O—, —CH(R⁷)N(R⁷)P(OR⁸)—, —CH(R⁷)N(R⁷)P(O)(OR⁸)O—, —CH(R⁷)N(R⁷)P(O)(OR⁸)—, —CH(R⁷)N(C(O)R⁷)P(OR⁸)O—, —CH(R⁷)N(C(O)R⁷)P(OR⁸)—, —CH(R⁷)N(C(O)R⁷)P(O)(OR⁸)O—, or —CH(R⁷)N(C(O)R⁷)P(OR⁸)—;
or X¹ and Y¹ are each independently represented by one of the following structural formulas:

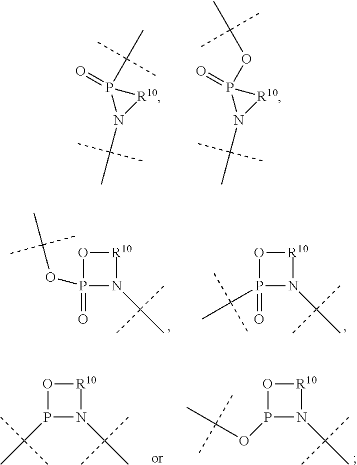

R¹⁰, taken together with the phosphinamide or phosphonamide, is a 5-, 6-, or 7-membered aryl, heteroaryl or heterocyclyl ring system;
R⁵, R⁶, and are each independently a (C₀₋₁₀)alkyl, (C₂₋₁₀)alkenyl, (C₂₋₁₀)alkynyl, (C₁₋₁₀)alkoxy(C₁₋₁₀)alkyl, (C₁₋₁₀)alkoxy(C₂₋₁₀)alkenyl, (C₁₋₁₀)alkoxy(C₂₋₁₀)alkynyl, (C₁₋₁₀)alkylthio(C₁₋₁₀)alkyl, (C₁₋₁₀)alkylthio(C₂₋₁₀)alkenyl, (C₁₋₁₀)alkylthio(C₂₋₁₀)alkynyl, cyclo(C₃₋₈)alkyl, cyclo(C₃₋₈)alkenyl, cyclo(C₃₋₈)alkyl(C₁₋₁₀)alkyl, cyclo(C₃₋₈)alkenyl(C₁₋₁₀)alkyl, cyclo(C₃₋₈)alkyl(C₂₋₁₀)alkenyl, cyclo(C₃₋₈)alkenyl(C₂₋₁₀)alkenyl, cyclo(C₃₋₈)alkyl(C₂₋₁₀)alkynyl, cyclo(C₃₋₈)alkenyl(C₂₋₁₀)alkynyl, heterocyclyl-(C₀₋₁₀)alkyl, heterocyclyl-(C₂₋₁₀)alkenyl, or heterocyclyl-(C₂₋₁₀)alkynyl, any of which is optionally substituted with one or more independent halo, —CF₃, —OCF₃, —OR⁷⁷, —NR⁷⁷R⁸⁷, —C(O)R⁷⁷, —CO₂R⁷⁷, —CONR⁷⁷R⁸⁷, —NO₂, —CN, —S(O)ⱼ₅ₐR⁷⁷, —SO₂NR⁷⁷R⁸⁷, NR⁷⁷(C═O)R⁸⁷, NR⁷⁷(C═O)OR⁸⁷, NR⁷⁷(C═O)NR⁷⁸R⁸⁷, NR⁷⁷S(O)ⱼ₅ₐR⁸⁷, —(C═S)OR⁷⁷, —(C═O)SR⁷⁷, —NR⁷⁷(C═NR⁸⁷)NR⁷⁸R⁸⁸, —NR⁷⁷(C═NR⁸⁷)OR⁷⁸, —NR⁷⁷(C═NR⁸⁷)SR⁷⁸, —O(C═O)OR⁷⁷, —O(C═O)NR⁷⁷R⁸⁷, —O(C═O)SR⁷⁷, —S(C═O)OR⁷⁷, —P(O)OR⁷⁷OR⁸⁷, or —S(C═O)NR⁷⁷R⁸⁷ substituents; or aryl-(C₀₋₁₀)alkyl, aryl-(C₂₋₁₀)alkenyl, or aryl-(C₂₋₁₀)alkynyl, any of which is optionally substituted with one or more independent halo, —CF₃, —OCF₃, —OR⁷⁷, —NR⁷⁷R⁸⁷, —C(O)R⁷⁷, —CO₂R⁷⁷, —CONR⁷⁷R⁸⁷, —NO₂, —CN, —S(O)ⱼ₅ₐR⁷⁷, —SO₂NR⁷⁷R⁸⁷, NR⁷⁷(C═O)R⁸⁷, NR⁷⁷(C═O)OR⁸⁷, NR⁷⁷(C═O)NR⁷⁸R⁸⁷, NR⁷⁷S(O)ⱼ₅ₐR⁸⁷, —(C═S)OR⁷⁷, —(C═O)SR⁷⁷, —NR⁷⁷(C═NR⁸⁷)NR⁷⁸R⁸⁸, —NR⁷⁷(C═NR⁸⁷)OR⁷⁸, —NR⁷⁷(C═NR⁸⁷)SR⁷⁸, —O(C═O)OR⁷⁷, —O(C═O)NR⁷⁷R⁸⁷, —O(C═O)SR⁷⁷, —S(C═O)OR⁷⁷, —P(O)OR⁷⁷R⁸⁷, or —S(C═O)NR⁷⁷R⁸⁷ substituents; or hetaryl-(C₀₋₁₀)alkyl, hetaryl-(C₂₋₁₀)alkenyl, or hetaryl-(C₂₋₁₀)alkynyl, any of which is optionally substituted with one or more independent halo, —CF₃, —OCF₃, —OR⁷⁷, —NR⁷⁷R⁸⁷, —C(O)R⁷⁷, —CO₂R⁷⁷, —CONR⁷⁷R⁸⁷, —NO₂, —CN, —S(O)ⱼ₅ₐR⁷⁷, —SO₂NR⁷⁷R⁸⁷, NR⁷⁷(C═O)R⁸⁷, NR⁷⁷(C═O)OR⁸⁷, NR⁷⁷(C═O)NR⁷⁸R⁸⁷, NR⁷⁷S(O)ⱼ₅ₐR⁸⁷, —(C═S)OR⁷⁷, —(C═O)SR⁷⁷, —NR⁷⁷(C═NR⁸⁷)NR⁷⁸R⁸⁸, —NR⁷⁷(C═NR⁸⁷)OR⁷⁸, —NR⁷⁷(C═NR⁸⁷)SR⁷⁸, —O(C═O)OR⁷⁷, —O(C═O)NR⁷⁷R⁸⁷, —O(C═O)SR⁷⁷, —S(C═O)OR⁷⁷, —P(O)OR⁷⁷OR⁸⁷, or —S(C═O)NR⁷⁷R⁸⁷ substituents; or R⁵ with R⁶ taken together with the respective carbon atom to which they are attached, form a 3-10 membered saturated or unsaturated ring, wherein said ring is optionally substituted with R⁶⁹; or R⁵ with R⁶ taken together with the respective carbon atom to which they are attached, form a 3-10 membered saturated or unsaturated heterocyclic ring, wherein said ring is optionally substituted with R⁶⁹;
R⁷ and R⁸ are each independently H, acyl, alkyl, alkenyl, aryl, heteroaryl, heterocyclyl or cycloalkyl, any of which is optionally substituted by one or more G¹¹¹ substituents;
R⁴ is H, alkyl, alkenyl, alkynyl, aryl, heteroaryl, cycloalkyl, heterocyclyl, cycloalkenyl, or heterocycloalkenyl, any of which is optionally substituted by one or more G⁴¹ substituents;
R⁶⁹ is equal to halo, —OR⁷⁸, —SH, —NR⁷⁸R⁸⁸, —CO₂R⁷⁸, —CONR⁷⁸R⁸⁸, —NO₂, —CN, —S(O)ⱼ₈R⁷⁸, —SO₂NR⁷⁸R⁸⁸, (C₀₋₁₀)alkyl, (C₂₋₁₀)alkenyl, (C₂₋₁₀)alkynyl, (C₁₋₁₀)alkoxy(C₁₋₁₀)alkyl, (C₁₋₁₀)alkoxy(C₂₋₁₀)alkenyl, (C₁₋₁₀)alkoxy(C₂₋₁₀)alkynyl, (C₁₋₁₀)alkylthio(C₁₋₁₀)alkyl, (C₁₋₁₀)alkylthio(C₂₋₁₀)alkenyl, (C₁₋₁₀)alkylthio(C₂₋₁₀)alkynyl, cyclo(C₃₋₈)alkyl, cyclo(C₃₋₈)alkenyl, cyclo(C₃₋₈)alkyl(C₁₋₁₀)alkyl, cyclo(C₃₋₈)alkenyl(C₁₋₁₀)alkyl, cyclo(C₃₋₈)alkyl(C₂₋₁₀)alkenyl, cyclo(C₃₋₈)alkenyl(C₂₋₁₀)alkenyl, cyclo(C₃₋₈)alkyl(C₂₋₁₀)alkynyl, cyclo(C₃₋₈)alkenyl(C₂₋₁₀)alkynyl, heterocyclyl-(C₀₋₁₀)alkyl, heterocyclyl-(C₂₋₁₀)alkenyl, or heterocyclyl-(C₂₋₁₀)alkynyl, any of which is optionally substituted with one or more independent halo, cyano, nitro, —OR⁷⁷⁸, —SO₂NR⁷⁷⁸R⁸⁸⁸, or —NR⁷⁷⁸R⁸⁸⁸ substituents; or aryl-(C₀₋₁₀)alkyl, aryl-(C₂₋₁₀)alkenyl, or aryl-(C₂₋₁₀)alkynyl, any of which is optionally substituted with one or more independent halo, cyano, nitro, —OR⁷⁷⁸, (C₁₋₁₀)alkyl, (C₂₋₁₀)alkenyl, (C₂₋₁₀)alkynyl, halo(C₁₋₁₀)alkyl, halo(C₂₋₁₀)alkenyl, halo(C₂₋₁₀)alkynyl, —COOH, (C₁₋₄)alkoxycarbonyl, —CONR⁷⁷⁸R⁸⁸⁸, —SO₂NR⁷⁷⁸R⁸⁸⁸, or —NR⁷⁷⁸R⁸⁸⁸ substituents; or hetaryl-(C₀₋₁₀)alkyl, hetaryl-(C₂₋₁₀)alkenyl, or hetaryl-(C₂₋₁₀)alkynyl, any of which is optionally substituted with one or more independent halo, cyano, nitro, —OR⁷⁷⁸, (C₁₋₁₀)alkyl, (C₂₋₁₀)alkenyl, (C₂₋₁₀)alkynyl, halo(C₁₋₁₀)alkyl, halo(C₂₋₁₀)alkenyl, halo(C₂₋₁₀)alkynyl, —COOH, (C₁₋₄)alkoxycarbonyl, —CONR⁷⁷⁸R⁸⁸⁸, —SO₂NR⁷⁷⁸R⁸⁸⁸, or —NR⁷⁷⁸R⁸⁸⁸ substituents; or mono(C₁₋₆alkyl)amino(C₁₋₆)alkyl, di((C₁₋₆)alkyl)amino(C₁₋₆)alkyl, mono(aryl)amino(C₁₋₆)alkyl, di(aryl)amino(C₁₋₆)alkyl, or —N((C₁₋₆)alkyl)-(C₁₋₆)alkyl-aryl, any of which is optionally substituted with one or more independent halo, cyano, nitro, —OR⁷⁷⁸, (C₁₋₁₀)alkyl, (C₂₋₁₀)alkenyl, (C₂₋₁₀)alkynyl, halo(C₁₋₁₀)alkyl, halo(C₂₋₁₀)alkenyl, halo(C₂₋₁₀)alkynyl, —COOH, (C₁₋₄)alkoxycarbonyl, —CONR⁷⁷⁸R⁸⁸⁸ SO₂NR⁷⁷⁸R⁸⁸⁸, or —NR⁷⁷⁸R⁸⁸⁸ substituents; or in the case of —NR⁷⁸R⁸⁸, R⁷⁸ and R⁸⁸ taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, (C₁₋₁₀)alkoxy, —SO₂NR⁷⁷⁸R⁸⁸⁸, or —NR⁷⁷⁸R⁸⁸⁸ substituents;
R⁷⁷, R⁷⁸, R⁸⁷, R⁸⁸, R⁷⁷⁸, and R⁸⁸⁸ are each independently (C₀₋₁₀)alkyl, (C₂₋₁₀)alkenyl, (C₂₋₁₀)alkynyl, (C₁₋₁₀)alkoxy(C₁₋₁₀)alkyl, (C₁₋₁₀)alkoxyC₂₋₁₀)alkenyl, (C₁₋₁₀)alkoxy(C₂₋₁₀)alkynyl, (C₁₋₁₀)alkylthio(C₁₋₁₀)alkyl, (C₁₋₁₀)alkylthio(C₂₋₁₀)alkenyl, (C₁₋₁₀)alkylthio(C₂₋₁₀)alkynyl, cyclo(C₃₋₈)alkyl, cyclo(C₃₋₈)alkenyl, cyclo(C₃₋₈)alkyl(C₁₋₁₀)alkyl, cyclo(C₃₋₈)alkenyl(C₁₋₁₀)alkyl, cyclo(C₃₋₈)alkyl(C₂₋₁₀)alkenyl, cyclo(C₃₋₈)alkenyl(C₂₋₁₀)alkenyl, cyclo(C₃₋₈)alkyl(C₂₋₁₀)alkynyl, cyclo(C₃₋₈)alkenyl(C₂₋₁₀)alkynyl, heterocyclyl-(C₀₋₁₀)alkyl, heterocyclyl-(C₂₋₁₀)alkenyl, heterocyclyl-(C₂₋₁₀)alkynyl, (C₁₋₁₀)alkylcarbonyl, (C₂₋₁₀)alkenylcarbonyl, (C₂₋₁₀)alkynylcarbonyl, (C₁₋₁₀)alkoxycarbonyl, (C₁₋₁₀)alkoxycarbonyl(C₁₋₁₀)alkyl, mono(C₁₋₆)alkylaminocarbonyl, di(C₁₋₆)alkylaminocarbonyl, mono(aryl)aminocarbonyl, di(aryl)aminocarbonyl, or (C₁₋₁₀)alkyl(aryl)aminocarbonyl, any of which is optionally substituted with one or more independent halo, cyano, hydroxy, nitro, (C₁₋₁₀)alkoxy, —SO₂N((C₀₋₄)alkyl)((C₀₋₄)alkyl), or —N((C₀₋₄)alkyl)((C₀₋₄)alkyl) substituents; or aryl-(C₀₋₁₀)alkyl, aryl-(C₂₋₁₀)alkenyl, or aryl-(C₂₋₁₀)alkynyl, any of which is optionally substituted with one or more independent halo, cyano, nitro, —O((C₀₋₄)alkyl), (C₁₋₁₀)alkyl, (C₂₋₁₀)alkenyl, (C₂₋₁₀)alkynyl, halo(C₁₋₁₀)alkyl, halo(C₂₋₁₀)alkenyl, halo(C₂₋₁₀)alkynyl, —COOH, (C₁₋₄)alkoxycarbonyl, —CON((C₀₋₄)alkyl)((C₀₋₁₀)alkyl), —SO₂N((C₀₋₄)alkyl)((C₀₋₄)alkyl), or —N((C₀₋₄)alkyl)((C₀₋₄)alkyl) substituents; or hetaryl-(C₀₋₁₀)alkyl, hetaryl-(C₂₋₁₀)alkenyl, or hetaryl-(C₂₋₁₀)alkynyl, any of which is optionally substituted with one or more independent halo, cyano, nitro, —O((C₀₋₄)alkyl), (C₁₋₁₀)alkyl, (C₂₋₁₀)alkenyl, (C₂₋₁₀)alkynyl, halo(C₁₋₁₀)alkyl, halo(C₂₋₁₀)alkenyl, halo(C₂₋₁₀)alkynyl, —COOH, (C₁₋₄)alkoxycarbonyl, —CON((C₀₋₄)alkyl)((C₀₋₄)alkyl), —SO₂N((C₀₋₄)alkyl)((C₀₋₄)alkyl), or —N((C₀₋₄)alkyl)((C₀₋₄)alkyl) substituents; or mono((C₁₋₆)alkyl)amino(C₁₋₆)alkyl, di((C₁₋₆)alkyl)amino(C₁₋₆)alkyl, mono(aryl)amino(C₁₋₆)alkyl, di(aryl)amino(C₁₋₆)alkyl, or —N((C₁₋₆)alkyl)-(C₁₋₆)alkyl-aryl, any of which is optionally substituted with one or more independent halo, cyano, nitro, —O((C₀₋₄)alkyl), (C₂₋₁₀)alkenyl, (C₂₋₁₀)alkynyl, halo(C₁₋₁₀)alkyl, halo(C₂₋₁₀)alkenyl, halo(C₂₋₁₀)alkynyl, —COOH, (C₁₋₄)alkoxycarbonyl, —CON((C₀₋₄)alkyl)((C₀₋₄)alkyl), —SO₂N((C₀₋₄)alkyl)((C₀₋₄)alkyl), or —N((C₀₋₄)alkyl)((C₀₋₄)alkyl) substituents; and n, m, j1, j1a, j2a, j3a, j4, j4a, j5a, j6a, j7, and j8 are each independently equal to 0, 1, or 2.

In an embodiment, the BTK inhibitor is a compound selected from the structures disclosed in U.S. Pat. Nos. 8,450,335 and 8,609,679, and U.S. Patent Application Publication Nos. 2010/0029610 A1, 2012/0077832 A1, 2013/0065879 A1, 2013/0072469 A1, and 2013/0165462 A1, the disclosures of which are incorporated by reference herein. In an embodiment, the BTK inhibitor is a compound of Formula (XV) or Formula (XVI):

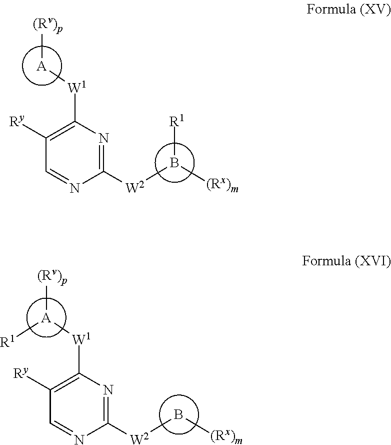

or a pharmaceutically acceptable salt, ester, solvate, hydrate, cocrystal, or prodrug thereof, wherein:
Ring A is an optionally substituted group selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 4-7 membered saturated or partially unsaturated heterocyclic ring having 1-3 heteroatoms independently selected from nitrogen, oxygen, or sulfur, an optionally substituted 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
Ring B is an optionally substituted group selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 4-7 membered saturated or partially unsaturated heterocyclic ring having 1-3 heteroatoms independently selected from nitrogen, oxygen, or sulfur, an optionally substituted 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
R¹ is a warhead group;
R^{y} is hydrogen, halogen, —CN, —CF₃, C₁₋₄ aliphatic, C₁₋₄ haloaliphatic, —OR, —C(O)R, or —C(O)N(R)₂;
each R group is independently hydrogen or an optionally substituted group selected from C₁₋₆ aliphatic, phenyl, an optionally substituted 4-7 membered heterocyclic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur;
W¹ and W² are each independently a covalent bond or a bivalent C₁₋₃ alkylene chain wherein one methylene unit of W¹ or W² is optionally replaced by —NR²—, —N(R²)C(O)—, —C(O)N(R²)—, —N(R²)SO₂—, —SO₂N(R²)—, —O—, —C(O)—, —OC(O)—, —C(O)O—, —S—, —SO— or —SO₂—,
R² is hydrogen, optionally substituted C₁₋₆ aliphatic, or —C(O)R, or:
R² and a substituent on Ring A are taken together with their intervening atoms to form a 4-6 membered saturated, partially unsaturated, or aromatic fused ring, or:
R² and R^{y} are taken together with their intervening atoms to form an optionally substituted 4-7 membered partially unsaturated or aromatic fused ring;
m and p are independently 0-4; and
R^{x} and R^{v} are independently selected from —R, halogen, —OR, —O(CH₂)_{q}OR, —CN, —NO₂, —SO₂R, —SO₂N(R)₂, —SOR, —C(O)R, —CO₂R, —C(O)N(R)₂, —NRC(O)R, —NRC(O)NR₂, —NRSO₂R, or —N(R)₂, wherein q is 1-4; or:
R^{x} and R¹ when concurrently present on Ring B are taken together with their intervening atoms to form an optionally substituted 5-7 membered saturated, partially unsaturated, or aryl ring having 0-3 heteroatoms independently selected from nitrogen, oxygen, or sulfur, wherein said ring is substituted with a warhead group and 0-3 groups independently selected from oxo, halogen, —CN, or C₁₋₆ aliphatic; or
R^{v} and R¹ when concurrently present on Ring A are taken together with their intervening atoms to form an optionally substituted 5-7 membered saturated, partially unsaturated, or aryl ring having 0-3 heteroatoms independently selected from nitrogen, oxygen, or sulfur, wherein said ring is substituted with a warhead group and 0-3 groups independently selected from oxo, halogen, —CN, or C₁₋₆ aliphatic.

In an embodiment, the BTK inhibitor is a compound of Formula (XV) or Formula (XVI), wherein:
Ring A is selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, an optionally substituted 4-7 membered saturated or partially unsaturated heterocyclic ring having 1-3 heteroatoms independently selected from nitrogen, oxygen, or sulfur, an optionally substituted 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
Ring B is selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, an optionally substituted 4-7 membered saturated or partially unsaturated heterocyclic ring having 1-3 heteroatoms independently selected from nitrogen, oxygen, or sulfur, an optionally substituted 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
R¹ is -L-Y, wherein:
L is a covalent bond or a bivalent C₁₋₈ saturated or unsaturated, straight or branched, hydrocarbon chain, wherein one, two, or three methylene units of L are optionally and independently replaced by cyclopropylene, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(N₂)—;
Y is hydrogen, C₁₋₆ aliphatic optionally substituted with oxo, halogen, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with at 1-4 groups independently selected from -Q-Z, oxo, NO₂, halogen, CN, or C₁₋₆ aliphatic, wherein:
Q is a covalent bond or a bivalent C₁₋₆ saturated or unsaturated, straight or branched, hydrocarbon chain, wherein one or two methylene units of Q are optionally and independently replaced by —NR—, —S—, —O—, —C(O)—, —SO—, or —SO₂—; and
Z is hydrogen or C₁₋₆ aliphatic optionally substituted with oxo, halogen, or CN;
R^{y} is hydrogen, halogen, —CN, —CF₃, C₁₋₄ aliphatic, C₁₋₄ haloaliphatic, —OR, —C(O)R, or —C(O)N(R)₂;
each R group is independently hydrogen or an optionally substituted group selected from C₁₋₆ aliphatic, phenyl, an optionally substituted 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur;
W¹ and W² are each independently a covalent bond or a bivalent C₁₋₃ alkylene chain wherein one methylene unit of W¹ or W² is optionally replaced by —NR²—, —N(R²)C(O)—, —C(O)N(R²)—, —N(R²)SO₂—, —SO₂N(R²)—, —O—, —C(O)—, —OC(O)—, —C(O)O—, —S—, —SO— or —SO₂—;
R² is hydrogen, optionally substituted C₁₋₆ aliphatic, or —C(O)R, or:
R² and a substituent on Ring A are taken together with their intervening atoms to form a 4-6 membered partially unsaturated or aromatic fused ring; or
R² and R^{y} are taken together with their intervening atoms to form a 4-6 membered saturated, partially unsaturated, or aromatic fused ring;
m and p are independently 0-4; and
R^{x} and R^{y} are independently selected from —R, halogen, —OR, —O(CH₂)_{q}OR, —CN, —NO₂, —SO₂R, —SO₂N(R)₂, —SOR, —C(O)R, —CO₂R, —C(O)N(R)₂, —NRC(O)R, —NRC(O)NR₂, —NRSO₂R, or —N(R)₂, wherein R is independently selected from the group consisting of hydrogen, cycloalkyl, alkenyl, cycloalkenyl, alkynyl, aryl, heteroaryl, and heterocycly; or:
R^{x} and R¹ when concurrently present on Ring B are taken together with their intervening atoms to form a 5-7 membered saturated, partially unsaturated, or aryl ring having 0-3 heteroatoms independently selected from nitrogen, oxygen, or sulfur, wherein said ring is substituted with a warhead group and 0-3 groups independently selected from oxo, halogen, —CN, or C₁₋₆ aliphatic; or
R^{v} and R¹ when concurrently present on Ring A are taken together with their intervening atoms to form a 5-7 membered saturated, partially unsaturated, or aryl ring having 0-3 heteroatoms independently selected from nitrogen, oxygen, or sulfur, wherein said ring is substituted with a warhead group and 0-3 groups independently selected from oxo, halogen, —CN, or C₁₋₆ aliphatic.

As defined generally above, Ring A is selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, an optionally substituted 4-7 membered saturated or partially unsaturated heterocyclic ring having 1-3 heteroatoms independently selected from nitrogen, oxygen, or sulfur, an optionally substituted 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur.

In preferred embodiments, Ring A is an optionally substituted phenyl group. In some embodiments, Ring A is an optionally substituted naphthyl ring or an optionally substituted bicyclic 8-10 membered heteroaryl ring having 1-4 heteroatoms independently selected from nitrogen, oxygen, or sulfur. In certain other embodiments, Ring A is an optionally substituted 3-7 membered carbocyclic ring. In yet other embodiments, Ring A is an optionally substituted 4-7 membered heterocyclic ring having 1-3 heteroatoms independently selected from nitrogen, oxygen, or sulfur. In preferred embodiments, Ring B is an optionally substituted phenyl group.

In certain embodiments, Ring A in Formula (XV) or Formula (XVI) is substituted as defined herein. In some embodiments, Ring A is substituted with one, two, or three groups independently selected from halogen, R^{o}, or —(CH₂)₀₋₄OR^{o}, or —O(CH₂)₀₋₄R^{o}, wherein each R^{o} is independently selected from the group consisting of cycloalkyl, alkenyl, cycloalkenyl, alkynyl, aryl, heteroaryl, and heterocyclyl. Exemplary substituents on Ring A include Br, I, Cl, methyl, —CF₃, —C≡CH₂phenyl, —OCH₂(fluorophenyl), or —OCH₂pyridyl.

In a preferred embodiment, the BTK inhibitor is CC-292 (also known as AVL-292), or a pharmaceutically acceptable salt, ester, solvate, hydrate, cocrystal, or prodrug thereof, most preferably a hydrochloride salt or a besylate salt thereof. In a preferred embodiment, the BTK inhibitor is a compound of Formula (XVII):

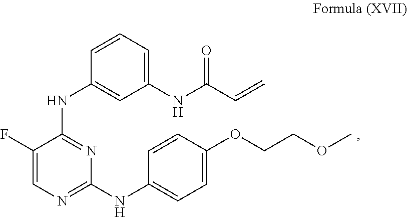 [SMILES: C=CC(=O)Nc1cccc(Nc2nc(Nc3ccc(OCCOC)cc3)ncc2F)c1]

which is N-(3-((5-fluoro-2-((4-(2-methoxyethoxy)phenyl)amino)pyrimidin-4-yl)amino)phenyl)acrylamide, or a pharmaceutically acceptable salt, ester, solvate, hydrate, cocrystal, or prodrug thereof, or in an preferred embodiment is a hydrochloride salt or a besylate salt thereof. The preparation of this compound is described in U.S. Patent Application Publication No. 2010/0029610 A1 at Example 20, the disclosure of which is incorporated by reference herein. The preparation of the besylate salt (i.e., the benzenesulfonic acid salt) of this compound is described in U.S. Patent Application Publication No. 2012/0077832 A1, the disclosure of which is incorporated by reference herein. In an embodiment, the BTK inhibitor is a compound selected from the structures disclosed in U.S. Patent Application Publication No. 2010/0029610 A1 or No. 2012/0077832 A1, the disclosures of which are incorporated by reference herein.

In a preferred embodiment, the BTK inhibitor is N-(3-((5-fluoro-2-((4-(2-methoxyethoxy)phenyl)amino)pyrimidin-4-yl)amino)phenyl)acrylamide or a pharmaceutically acceptable salt, ester, solvate, hydrate, cocrystal, or prodrug thereof, or more preferably a hydrochloride salt or besylate salt thereof. The preparation of this compound is described in U.S.

Patent Application Publication Nos. 2010/0029610 A1 and 2012/0077832 A1, the disclosure of which is incorporated by reference herein. The preparation of this compound is described in U.S. Patent Application Publication No. 2010/0029610 A1 at Example 20, the disclosure of which is incorporated by reference herein. The preparation of its besylate salt of this compound is described in U.S. Patent Application Publication No. 2012/0077832 A1, the disclosure of which is incorporated by reference herein.

In an embodiment, the BTK inhibitor is a compound of Formula (XVIII):

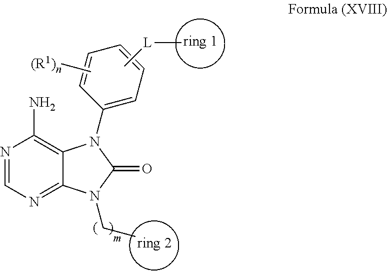

or a pharmaceutically acceptable salt, ester, solvate, hydrate, cocrystal, or prodrug thereof, wherein:
L represents (1) —O—, (2) —S—, (3) —SO—, (4) —SO₂— (5) —NH—, (6) —C(O)—, (7) —CH₂O—, (8) —O—CH₂—, (9) —CH₂—, or (10) —CH(OH)—;
R¹ represents (1) a halogen atom, (2) a C₁₋₄ alkyl group, (3) a C₁₋₄ alkoxy group, (4) a C₁₋₄ haloalkyl group, or (5) a C₁₋₄ haloalkoxy group;
ring1 represents a 4- to 7-membered cyclic group, which may be substituted by from one to five substituents each independently selected from the group consisting of (1) halogen atoms, (2) C₁₋₄ alkyl groups, (3) C₁₋₄ alkoxy groups, (4) nitrile, (5) C₁₋₄ haloalkyl groups, and (6) C₁₋₄ haloalkoxy groups, wherein when two or more substituents are present on ring1, these substituents may form a 4- to 7-membered cyclic group together with the atoms in ring1 to which these substituents are bound;
ring2 represents a 4- to 7-membered saturated heterocycle, which may be substituted by from one to three —K—R²; K represents (1) a bond, (2) a C₁₋₄ alkylene, (3) —C(O)—, (4) —C(O)—CH₂—, (5) —CH₂—C(O)—, (6) —C(O)O—, or (7) —SO₂— (wherein the bond on the left is bound to the ring2);
R² represents (1) a C₁₋₄ alkyl, (2) a C₂₋₄ alkenyl, or (3) a C₂₋₄ alkynyl group, each of which may be substituted by from one to five substituents each independently selected from the group consisting of (1) NR³R⁴, (2) halogen atoms, (3) CONR⁵R⁶, (4) CO₂R⁷, and (5) OR⁸;
R³ and R⁴ each independently represent (1) a hydrogen atom, or (2) a C₁₋₄ alkyl group which may be substituted by OR⁹ or CONR¹⁰R¹¹; R³ and R⁴ may, together with the nitrogen atom to which they are bound, form a 4- to 7-membered nitrogenous saturated heterocycle, which may be substituted by an oxo group or a hydroxyl group;
R⁵ and R⁶ each independently represent (1) a hydrogen atom, (2) a C₁₋₄ alkyl group, or (3) a phenyl group;
R⁷ represents (1) a hydrogen atom or (2) a C₁₋₄ alkyl group;
R⁸ represents (1) a hydrogen atom, (2) a C₁₋₄ alkyl group, (3) a phenyl group, or (4) a benzotriazolyl group; R⁹ represents (1) a hydrogen atom or (2) a C₁₋₄ alkyl group;
R¹⁰ and R¹¹ each independently represent (1) a hydrogen atom or (2) a C₁₋₄ alkyl group;
n represents an integer from 0 to 4;
m represents an integer from 0 to 2; and
when n is two or more, the R¹'s may be the same as each other or may differ from one another).

In an embodiment, the BTK inhibitor is a compound of Formula (XIX):

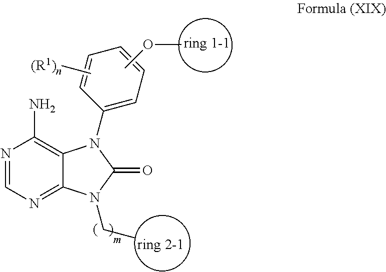

or a pharmaceutically acceptable salt, ester, solvate, hydrate, cocrystal, or prodrug thereof, wherein:
R¹ represents (1) a halogen atom, (2) a C₁₋₄ alkyl group, (3) a C₁₋₄ alkoxy group, (4) a C₁₋₄ haloalkyl group, or (5) a C₁₋₄ haloalkoxy group;
ring1-1 represents a benzene, cyclohexane, or pyridine ring, each of which may be substituted by from one to five substituents each independently selected from the group consisting of (1) halogen atoms, (2) C₁₋₄ alkyl groups, (3) C₁₋₄ alkoxy groups, (4) nitrile, (5) CF₃;
ring2-1 represents a 4- to 7-membered nitrogenous saturated heterocycle, which may be substituted by from one to three —K—R²; wherein K represents (1) a bond, (2) a C₁₋₄ alkylene, (3) —C(O)—, (4) —C(O)—CH₂—, (5) —CH₂—C(O)—, (6) —C(O)O—, or (7) —SO₂— (wherein the bond on the left is bound to the ring2);
R² represents (1) a C₁₋₄ alkyl, (2) a C₂₋₄ alkenyl, or (3) a C₂₋₄ alkynyl group, each of which may be substituted by from one to five substituents each independently selected from the group consisting of (1) NR³R⁴, (2) halogen atoms, (3) CONR⁵R⁶, (4) CO₂R⁷, and (5) OR⁸;
R³ and R⁴ each independently represent (1) a hydrogen atom, or (2) a C₁₋₄ alkyl group which may be substituted by OR⁹ or CONR¹⁰R¹¹; R³ and R⁴ may, together with the nitrogen atom to which they are bound, form a 4- to 7-membered nitrogenous saturated heterocycle, which may be substituted by an oxo group or a hydroxyl group;
R⁵ and R⁶ each independently represent (1) a hydrogen atom, (2) a C₁₋₄ alkyl group, or (3) a phenyl group;
R⁷ represents (1) a hydrogen atom or (2) a C₁₋₄ alkyl group;
R⁸ represents (1) a hydrogen atom, (2) a C₁₋₄ alkyl group, (3) a phenyl group, or (4) a benzotriazolyl group; R⁹ represents (1) a hydrogen atom or (2) a C₁₋₄ alkyl group;
R¹⁰ and R¹¹ each independently represent (1) a hydrogen atom or (2) a C₁₋₄ alkyl group;
n represents an integer from 0 to 4;
m represents an integer from 0 to 2; and
when n is two or more, the R¹'s may be the same as each other or may differ from one another).

In a preferred embodiment, the BTK inhibitor is a compound of Formula (XX):

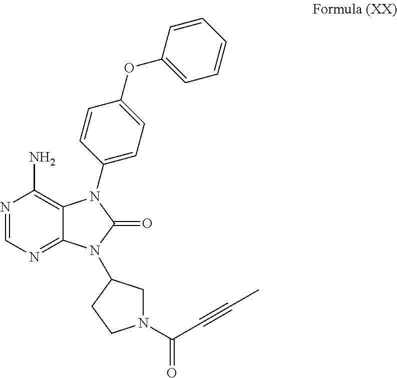 [SMILES: CC#CC(=O)N1CCC(n2c(=O)n(-c3ccc(Oc4ccccc4)cc3)c3c(N)ncnc32)C1]

or a pharmaceutically acceptable salt, ester, solvate, hydrate, cocrystal, or prodrug thereof, preferably a hydrochloride salt thereof. The preparation of this compound is described in U.S. Patent Application Publication No. 2014/0330015 A1, the disclosure of which is incorporated by reference herein. In an embodiment, the BTK inhibitor is 6-amino-9-(1-(but-2-ynoyl)pyrrolidin-3-yl)-7-(4-phenoxyphenyl)-7,9-dihydro-8H-purin-8-one or a pharmaceutically acceptable salt, ester, solvate, hydrate, cocrystal, or prodrug thereof, or preferably a hydrochloride salt thereof. In an embodiment, the BTK inhibitor is 6-amino-9-[(3S)-1-(2-butynoyl)-3-pyrrolidinyl]-7-(4-phenoxyphenyl)-7,9-dihydro-8H-purin-8-one or a pharmaceutically acceptable salt, ester, solvate, hydrate, cocrystal, or prodrug thereof, or a hydrochloride salt thereof.

The R-enantiomer of Formula (XX) is also known as ONO-4059, and is given by Formula (XXI). In a preferred embodiment, the BTK inhibitor is a compound of Formula (XXI):

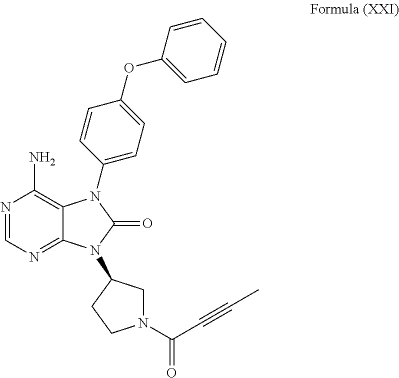 [SMILES: CC#CC(=O)N1CC[C@@H](n2c(=O)n(-c3ccc(Oc4ccccc4)cc3)c3c(N)ncnc32)C1]

or a pharmaceutically acceptable salt, ester, solvate, hydrate, cocrystal, or prodrug thereof, preferably a hydrochloride salt thereof.

In an embodiment, the BTK inhibitor is 6-amino-9-[(3R)-1-(2-butynoyl)-3-pyrrolidinyl]-7-(4-phenoxyphenyl)-7,9-dihydro-8H-purin-8-one or or a pharmaceutically acceptable salt, ester, solvate, hydrate, cocrystal, or prodrug thereof, preferably a hydrochloride salt thereof.

The preparation of Formula (XXI) is described in International Patent Application Publication No. WO 2013/081016 A1 and U.S. Patent Application Publication No. 2014/0330015 A1, the disclosure of each of which is incorporated by reference herein. In brief, the BTK inhibitor of Formula (XXI) can be prepared by the following procedure.

Step 1: A solution of dibenzylamine (10.2 g) in dichloromethane (30 mL) is dripped into a solution of 4,6-dichloro-5-nitropyrimidine (10 g) in dichloromethane (70 mL) on an ice bath. Then triethylamine (14.4 mL) is added, and the mixture is stirred for 1 hour. Water is added to the reaction mixture, the organic layer is washed with a saturated aqueous sodium chloride solution and dried over anhydrous sodium sulfate, and the solvent is concentrated under reduced pressure to obtain N,N-dibenzyl-6-chloro-5-nitropyrimidine-4-amine (19.2 g).

Step 2: The compound prepared in Step 1 (19 g) and tert-butyl (3R)-3-aminopyrrolidine-1-carboxylate (10.5 g) are dissolved in dioxane (58 mL). Triethylamine (8.1 mL) is added, and the mixture is stirred for 5 hours at 50° C. The reaction mixture is returned to room temperature, the solvent is distilled off, water is added, and extraction is performed with ethyl acetate. The organic layer is washed with saturated aqueous sodium chloride solution, then dried over anhydrous sodium sulfate, and the solvent is distilled off. The residue is purified by silica gel column chromatography to obtain tert-butyl (3R)-3-{[6-(dibenzylamino)-5-nitropyrimidin-4-yl]amino}pyrrolidine-1-carboxylate (27.0 g).

Step 3: An ethyl acetate (360 mL) solution of the compound prepared in Step 2 (17.5 g) is dripped into a mixture of zinc (23.3 g) and a 3.0 M aqueous ammonium chloride solution (11.4 g) on an ice bath, and the temperature is immediately raised to room temperature. After stirring for 2 hours, the reaction mixture is filtered through CELITE and the solvent is distilled off. The residue is purified by silica gel column chromatography to obtain tert-butyl (3R)-3-{[5-amino-6-(dibenzylamino)pyrimidin-4-yl]amino}pyrrolidine-1-carboxylate (12.4 g).

Step 4: The compound prepared in Step 3 (8.4 g) and 1,1′-carbonyl diimidazole (5.9 g) are dissolved in tetrahydrofuran (120 mL) and the solution is stirred for 15 hours at 60° C. The solvent is distilled off from the reaction mixture, water is added, and extraction with ethyl acetate is performed. The organic layer is washed with saturated aqueous sodium chloride solution, dried over anhydrous sodium sulfate, and the solvent is distilled off. The residue is purified by silica gel column chromatography to obtain tert-butyl (3R)-3-[6-(dibenzylamino)-8-oxo-7,8-dihydro-9H-purin-9-yl]pyrrolidin-1-carboxylate (7.8 g).

Step 5: The compound prepared in Step 4 (7.8 g) is dissolved in methanol (240 mL) and ethyl acetate (50 mL), 20% Pearlman's catalyst (Pd(OH)₂/C) (8.0 g, 100 wt %) is added, hydrogen gas replacement is carried out, and stirring is performed for 7.5 hours at 60° C. The reaction mixture is filtered through CELITE and the solvent is distilled off to obtain tert-butyl (3R)-3-(6-amino-8-oxo-7,8-dihydro-9H-purin-9-yl)pyrrolidine-1-carboxylate (5.0 g).

Step 6: At room temperature p-phenoxy phenyl boronic acid (2.1 g), copper(II) acetate (1.48 g), molecular sieve 4A (2.5 g), and pyridine (0.82 mL) are added to a dichloromethane suspension (200 mL) of the compound prepared in Step 5 (2.5 g), followed by stirring for 21 hours. The reaction mixture is filtered through CELITE and the residue is purified by silica gel column chromatography to obtain tert-butyl (3R)-3-[6-amino-8-oxo-7-(4-phenoxyphenyl)-7,8-dihydro-9H-purin-9-yl]pyrrolidine-1-carboxylate (1.3 g).

Step 7: At room temperature 4 N HCl/dioxane (13 mL) is added to a methanol (13 mL) suspension of the compound prepared in Step 6 (1.3 g 2.76 mmol, 1.0 equivalent), and the mixture is stirred for 1 hour. The solvent is then distilled off to obtain (3R)-6-amino-9-pyrrolidin-3-yl-7-(4-phenoxyphenyl)-7,9-dihydro-8H-purin-8-one dihydrochloride (1.5 g).

Step 8: After 2-butylnoic acid (34 mg), 1-ethyl-3-(3-dimethylaminopropyl) carbodiimide hydrochloride (EDC) (78 mg), 1-hydroxybenzotriazole (HOBt) (62 mg), and triethylamine (114 mL) are added to a solution of the compound prepared in Step 7 (100 mg) in dimethyl formamide (3 mL), the mixture is stirred at room temperature for 3 hours. Water is added to the reaction mixture and extraction with ethyl acetate is performed. The organic layer is washed with saturated sodium carbonate solution and saturated aqueous sodium chloride solution, then dried over anhydrous sodium sulfate, and the solvent is distilled off. The residue is purified by thin layer chromatography (dichloromethane:methanol:28% ammonia water=90:10:1) to obtain 6-amino-9-[(3R)-1-(2-butynoyl)-3-pyrrolidinyl]-7-(4-phenoxyphenyl)-7,9-dihydro-8H-purin-8-one (Formula (XXI)) (75 mg).

The hydrochloride salt of the compound of Formula (XXI) can be prepared as follows: 6-amino-9-[(3R)-1-(2-butynoyl)-3-pyrrolidinyl]-7-(4-phenoxyphenyl)-7,9-dihydro-8H-purin-8-one (3.0 g) (which may be prepared as described above) is placed in a 300 mL 3-neck pear-shaped flask, ethyl acetate (30 mL) and 1-propanol (4.5 mL) are added, and the external temperature is set at 70° C. (internal temperature 61° C.). After it is confirmed that the compound prepared in Step 8 has dissolved completely, 10% HO/methanol (3.5 mL) is added, and after precipitation of crystals is confirmed, the crystals are ripened by the following sequence: external temperature 70° C. for 30 min, external temperature 60° C. for 30 min, external temperature 50° C. for 60 min, external temperature 40° C. for 30 min, room temperature for 30 min, and an ice bath for 30 min. The resulting crystals are filtered, washed with ethyl acetate (6 mL), and dried under vacuum at 50° C. to obtain white crystals of 6-amino-9-[(3R)-1-(2-butynoyl)-3-pyrrolidinyl]-7-(4-phenoxyphenyl)-7,9-dihydro-8H-purin-8-one hydrochloride (2.76 g).

In an embodiment, the BTK inhibitor is a compound selected from the structures disclosed in International Patent Application Publication No. WO 2013/081016 A1 and U.S. Patent Application Publication No. US 2014/0330015 A1, the disclosure of each of which is incorporated by reference herein.

In an embodiment, the BTK inhibitor is a compound of Formula (XXII):

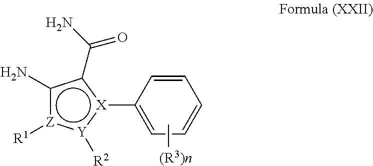

or a pharmaceutically acceptable salt, ester, solvate, hydrate, cocrystal, or prodrug thereof, wherein:
X—Y—Z is N—C—C and R² is present, or C—N—N and R² is absent;
R¹ is a 3-8 membered, N-containing ring, wherein the N is unsubstituted or substituted with R⁴;
R² is H or lower alkyl, particularly methyl, ethyl, propyl or butyl; or
R¹ and R² together with the atoms to which they are attached, form a 4-8 membered ring, preferably a 5-6 membered ring, selected from cycloalkyl, saturated or unsaturated heterocycle, aryl, and heteroaryl rings unsubstituted or substituted with at least one substituent L-R⁴;
R³ is in each instance, independently halogen, alkyl, 5-alkyl, CN, or OR⁵;
n is 1, 2, 3, or 4, preferably 1 or 2;
L is a bond, NH, heteroalkyl, or heterocyclyl;
R⁴ is COR′, CO₂R′, or SO₂R′, wherein R′ is substituted or unsubstituted alkyl, substituted or unsubstituted alkenyl, substituted or unsubstituted alkynyl;
R⁵ is H or unsubstituted or substituted heteroalkyl, alkyl, cycloalkyl, saturated or unsaturated heterocyclyl, aryl, or heteroaryl.

In some embodiments, the BTK inhibitor is one of the following particular embodiments of Formula (XXII):
X—Y—Z is C—N—N and R² is absent; and R¹ is 3-8 membered, N-containing ring, N-substituted with R⁴;
X—Y—Z is N—C—C and R² is present, R¹ is 3-8 membered, N-containing ring, N-substituted with R⁴; and R² is H or lower alkyl;
X—Y—Z is N—C—C and R² is present; and R¹ and R² together with the atoms to which they are attached, form a 4-8 membered ring selected from cycloalkyl, saturated or unsaturated heterocycle, aryl, and heteroaryl rings unsubstituted or substituted with at least one substituent L-R⁴, wherein preferred rings of R¹ and R² are 5-6-membered, particularly dihydropyrrole, tetrahydropyridine, tetrahydroazepine, phenyl, or pyridine;
X—Y—Z is N—C—C and R² is present; and R¹ and R² together with the atoms to which they are attached, form a 5-6 membered ring, preferably (a) phenyl substituted with a single -L-R⁴, or (b) dihydropyrrole or tetrahydropyridine, N-substituted with a single -L-R⁴ wherein L is bond;
R¹ is piperidine or azaspiro[3.3]heptane, preferably N-substituted with R⁴;
R⁴ is COR′ or SO₂R′, particularly wherein R′ is substituted or unsubstituted alkenyl, particularly substituted or unsubstituted ethenyl; or
R⁵ is unsubstituted or substituted alkyl or aryl, particularly substituted or unsubstituted phenyl or methyl, such as cyclopropyl-substituted methyl with or tetrabutyl-substituted phenyl.

In some embodiments, the BTK inhibitor is one of the following particular embodiments of Formula (XXII):
R¹ is piperidine or azaspiro[3.3]heptane, N-substituted with R⁴, wherein R⁴ is H, COR′ or SO₂R′, and R′ is substituted or unsubstituted alkenyl, particularly substituted or unsubstituted ethenyl;
R³ is —OR⁵, R⁵ is phenyl, and n is 1;
R¹ and R², together with the atoms to which they are attached, form a 5-6 membered ring, preferably (a) phenyl substituted with a single -L-R⁴, or (b) dihydropyrrole or tetrahydropyridine, N-substituted with a single -L-R⁴ wherein L is bond; R³ is —OR⁵; n is 1; R⁴ is COR′, and R′ is ethenyl; and R⁵ is phenyl; and
X—Y—Z is C—N—N and R² is absent; R¹ is piperidine, N-substituted with R⁴; R³ is —OR⁵; n is 1; R⁴ is COR′, and R′ is unsubstituted or substituted alkenyl, particularly ethenyl; and R⁵ is substituted or unsubstituted aryl, particularly phenyl.

In a preferred embodiment, the BTK inhibitor is a compound selected from the group consisting of Formula (XXIII), Formula (XXIV), and Formula (XXV):

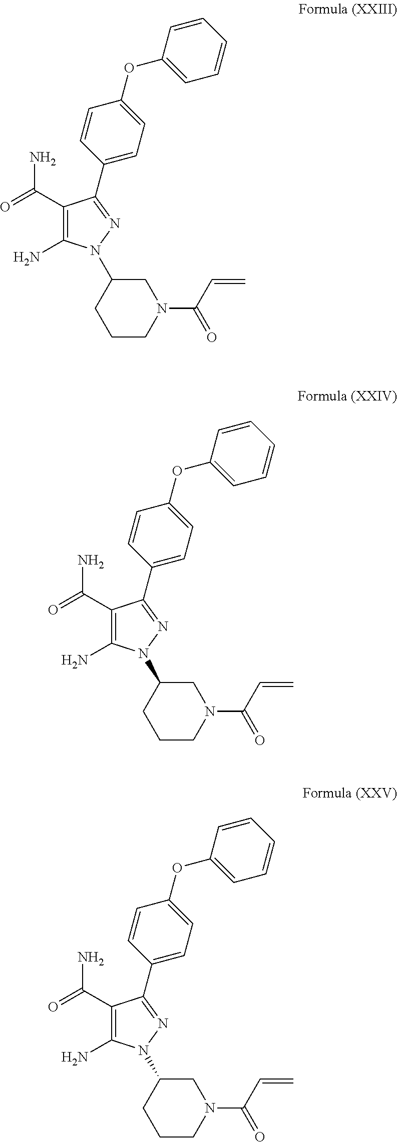 [SMILES: C=CC(=O)N1CCCC(n2nc(-c3ccc(Oc4ccccc4)cc3)c(C(N)=O)c2N)C1 ; C=CC(=O)N1CCC[C@@H](n2nc(-c3ccc(Oc4ccccc4)cc3)c(C(N)=O)c2N)C1 ; C=CC(=O)N1CCC[C@H](n2nc(-c3ccc(Oc4ccccc4)cc3)c(C(N)=O)c2N)C1]

or a pharmaceutically acceptable salt, ester, solvate, hydrate, cocrystal, or prodrug thereof. Formula (XXIV) is also known as BGB-3111. The preparation of these compounds is described in International Patent Application Publication No. WO 2014/173289 A1 and U.S. Patent Application Publication No. US 2015/0005277 A1, the disclosures of which are incorporated by reference herein.

In brief, the BTK inhibitor of Formula (XXIII) can be prepared by the following procedure.

### Step 1. Preparation of 2-(hydroxy(4-phenoxyphenyl)methylene)malononitrile

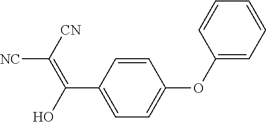 [SMILES: N#CC(C#N)=C(O)c1ccc(Oc2ccccc2)cc1]

A solution of 4-phenoxybenzoic acid (300 g, 1.4 mol) in SOCl₂ (1.2 L) is stirred at 80° C. under N₂ for 3 hours. The mixture is concentrated in vacuum to give the intermediate (315 g) which is used for next step without further purification.

To a solution of propanedinitrile (89.5 g, 1355 mmol) and DIEA (350 g, 2710 mmol) in THF (800 mL) is dropwise a solution of the intermediate (315 g) in toluene (800 mL) at 0-5° C. over 2 hours. The resultant mixture is allowed to warm to RT and stirred for 16 hours. The reaction is quenched with water (2.0 L) and extracted with of EA (2.0 L×3). The combined organic layers are washed with 1000 mL of 3 N HCl aqueous solution, brine (2.0 L×3), dried over Na₂SO₄ and concentrated to give the crude product (330 g, 93%).

### Step 2. Preparation of 2-(Methoxy(4-phenoxyphenyl)methylene)malononitrile

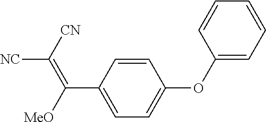 [SMILES: COC(=C(C#N)C#N)c1ccc(Oc2ccccc2)cc1]

A solution of 2-(hydroxy(4-phenoxyphenyl)methylene)malononitrile (50 g, 190.8 mmol) in CH(OMe₃) (500 mL) is heated to 75° C. for 16 hours. Then the mixture is concentrated to a residue and washed with MeOH (50 mL) to give 25 g (47.5%) of 2-(methoxy(4-phenoxyphenyl)methylene)malononitrile as a yellow solid.

### Step 3. Preparation of 5-amino-3-(4-phenoxyphenyl)-1H-pyrazole-4-carbonitrile

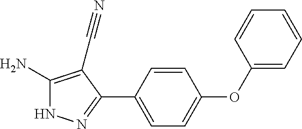 [SMILES: N#Cc1c(-c2ccc(Oc3ccccc3)cc2)n[nH]c1N]

To a solution of 2-(methoxy(4-phenoxyphenyl)methylene)malononitrile (80 g, 290 mmol) in ethanol (200 mL) is added hydrazine hydrate (20 mL). The mixture is stirred at RT for 16 hours then is concentrated to give the crude product and washed with MeOH (30 mL) to afford 55 g (68.8%) of 5-amino-3-(4-phenoxyphenyl)-1H-pyrazole-4-carbonitrile as a off-white solid.

### Step 4. Preparation of tert-butyl 3-(tosyloxy)piperidine-1-carboxylate

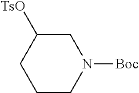 [SMILES: Cc1ccc(S(=O)(=O)OC2CCCN(C(=O)OC(C)(C)C)C2)cc1]

wherein “Boc” represents a tert-butyloxycarbonyl protecting group.

To a solution of tert-butyl 3-hydroxypiperidine-1-carboxylate (1.05 g, 5.0 mmol) in pyridine (8 mL) is added TsCl (1.425 g, 7.5 mmol). The mixture is stirred at RT under N₂ for two days. The mixture is concentrated and partitioned between 100 mL of EA and 100 mL of HCl (1 N) aqueous solution. The organic layer is separated from aqueous layer, washed with saturated NaHCO₃ aqueous solution (100 mL×2), brine (100 mL×3) and dried over Na₂SO₄. The organic layer is concentrated to afford 1.1 g (60%) of tert-butyl 3-(tosyloxy)piperidine-1-carboxylate as a colorless oil.

### Step 5. Preparation of tert-butyl 3-(5-amino-4-cyano-3-(4-phenoxyphenyl)-1H-pyrazol-1-yl)piperidine-1-carboxylate

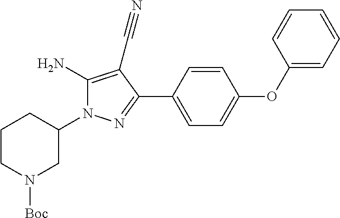 [SMILES: CC(C)(C)OC(=O)N1CCCC(n2nc(-c3ccc(Oc4ccccc4)cc3)c(C#N)c2N)C1]

To a solution of tert-butyl 3-(tosyloxy)piperidine-1-carboxylate (355 mg, 1.0 mmol) and 5-amino-3-(4-phenoxyphenyl)-1H-pyrazole-4-carbonitrile (276 mg, 1.0 mmol) in 5 mL of DMF is added Cs₂CO₃ (650 mg, 2.0 mmol). A tosyloxy leaving group is employed in this reaction. The mixture is stirred at RT for 16 hours, 75° C. for 3 hours and 60° C. for 16 hours. The mixture is concentrated washed with brine (100 mL×3) and dried over Na₂SO₄. The material is concentrated and purified by chromatography column on silica gel (eluted with petroleum ether/ethyl actate=3/1) to afford 60 mg (13%) of tert-butyl 3-(5-amino-4-cyano-3-(4-phenoxyphenyl)-1H-pyrazol-1-yl)piperidine-1-carboxylate as a yellow oil.

### Step 6. Preparation of tert-butyl 3-(5-amino-4-carbamoyl-3-(4-phenoxyphenyl)-1H-pyrazol-1-yl)piperidine-1-carboxylate

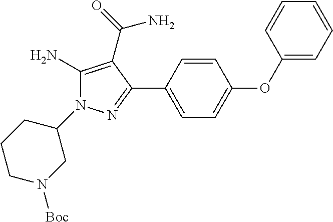 [SMILES: CC(C)(C)OC(=O)N1CCCC(n2nc(-c3ccc(Oc4ccccc4)cc3)c(C(N)=O)c2N)C1]

To a solution of tert-butyl 3-(5-amino-4-cyano-3-(4-phenoxyphenyl)-1H-pyrazol-1-yl)piperidine-1-carboxylate (100 mg, 0.22 mmol) in DMSO (2 mL) and ethanol (2 mL) was added the solution of NaOH (200 mg, 5 mmol) in water (1 mL) and H₂O₂ (1 mL). The mixture is stirred at 60° C. for 15 min and concentrated to remove EtOH, after which 10 mL of water and 50 mL of ethyl acetate are added. The organic layer is separated from aqueous layer, washed with brine (30 mL×3) and dried over Na₂SO₄. After concentratation, 50 mg of residue is used directly in the next step, wherein 50 mg of residue is purified by pre-TLC (eluted with petroleum ether/ethyl actate=1/1) to afford 12 mg (30%) of tert-butyl 3-(5-amino-4-carbamoyl-3-(4-phenoxyphenyl)-1H-pyrazol-1-yl)piperidine-1-carboxylate as a white solid.

### Step 7. Preparation of 5-amino-3-(4-phenoxyphenyl)-1-(piperidin-3-yl)-1H-pyrazole-4-carboxamide

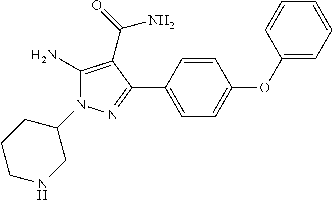 [SMILES: NC(=O)c1c(-c2ccc(Oc3ccccc3)cc2)nn(C2CCCNC2)c1N]

To a solution of tert-butyl 3-(5-amino-4-carbamoyl-3-(4-phenoxyphenyl)-1H-pyrazol-1-yl)piperidine-1-carboxylate (50 mg, 0.11 mmol) in ethyl acetate (1 mL) is added concentrated HCl (0.75 mL). The mixture is stirred at RT for 1 hour. Then saturated NaHCO₃ is added until pH>7, followed by ethyl acetate (50 mL). The organic layer is separated from aqueous layer, washed with brine (50 mL×3) and dried over Na₂SO₄. The resulting product is concentrated and purified by Pre-TLC (eluted with dichloromethane/MeOH/NH₃—H₂O=5/1/0.01) to afford 10 mg (25%) of 5-amino-3-(4-phenoxyphenyl)-1-(piperidin-3-yl)-1H-pyrazole-4-carboxamide as a white solid.

### Step 8. Preparation of 1-(1-acryloylpiperidin-3-yl)-5-amino-3-(4-phenoxyphenyl)-1H-pyrazole-4-carboxamide

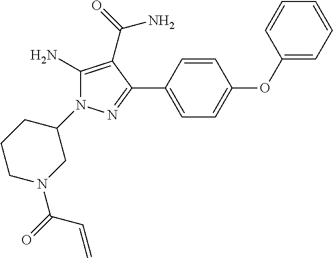 [SMILES: C=CC(=O)N1CCCC(n2nc(-c3ccc(Oc4ccccc4)cc3)c(C(N)=O)c2N)C1]

To a solution of 5-amino-3-(4-phenoxyphenyl)-1-(piperidin-3-yl)-1H-pyrazole-4-carboxamide (63 mg, 0.17 mmol) in dichloromethane (4 mL) is added pyridine (27 mg, 0.34 mmol). Then a solution of acryloyl chloride (12 mg, 0.17 mmol) in dichloromethane (1 mL) is added dropwise. After stirring at RT for 4 hours, the mixture is partitioned between 100 mL of dichloromethane and 100 mL of brine. The organic layer is separated from aqueous layer, washed with brine (100 mL×2) and dried over Na₂SO₄. The material is concentrated and purified by Pre-TLC (eluted with dichloromethane/MeOH=10/1) to afford 4 mg (5.5%) of 1-(1-acryloylpiperidin-3-yl)-5-amino-3-(4-phenoxyphenyl)-1H-pyrazole-4-carboxamide as a white solid.

The enantiomers of Formula (XXIII) provided by the procedure above may be prepared from 5-amino-3-(phenoxyphenyl)-1H-pyrazole-4-carbonitrile and (S)-tert-butyl 3-hydroxypiperidine-1-carboxylate using a similar procedure (step 4 to 8) for Formula (XXIV), or from (R)-tert-butyl 3-hydroxypiperidine-1-carboxylate using a similar procedure (step 4 to 8) for Formula (XXV). Under appropriate conditions recognized by one of ordinary skill in the art, a racemic mixture of Formula (XXIII) may be separated by chiral HPLC, the crystallization of chiral salts, or other means described above to yield Formula (XXIV) and Formula (XXV) of high enantiomeric purity.

In an embodiment, the BTK inhibitor is a compound selected from the structures disclosed in U.S. Patent Application Publication No. US 2015/0005277A1, the disclosure of which is incorporated by reference herein.

In an embodiment, the BTK inhibitor is a compound selected from the structures disclosed in U.S. Pat. No. 8,957,065, the disclosure of which is incorporated by reference herein. In an embodiment, the BTK inhibitor is HM-71224 (Hanmi Pharm. Co.), or a pharmaceutically acceptable salt, solvate, hydrate, cocrystal, or prodrug thereof. In an embodiment, the BTK inhibitor is N-(3-(2-(4-(4-methylpiperazin-1-yl)phenylamino)thieno[3,2-d]pyrimidine-4-yloxy)phenyl)acrylamide, or a pharmaceutically acceptable salt, solvate, hydrate, cocrystal, or prodrug thereof. In an embodiment, the BTK inhibitor is N-(3-((2-((2-methoxy-4-(4-methylpiperazin-1-yl)phenyl)amino)thieno[3,2-d]pyrimidin-4-yl)oxy)phenyl)acrylamide, or a pharmaceutically acceptable salt, solvate, hydrate, cocrystal, or prodrug thereof.

In an embodiment, the BTK inhibitor is 7-acryloyl-2-(4-phenoxyphenyl)-5,6,7,8-tetrahydro-4H-pyrazolo [5′,1′: 2,3]imidazo[4,5-c]pyridine-3-carboxamide, or a pharmaceutically acceptable salt, solvate, hydrate, cocrystal, or prodrug thereof.

Other BTK inhibitors suitable for use in the present methods, uses, and compositions also include, but are not limited to, those described in, for example, International Patent Application Publication Nos. WO 2013/010868, WO 2012/158843, WO 2012/135944, WO 2012/135937, U.S. Patent Application Publication Nos. 2011/0177011, 2014/0155385, 2014/0323464, and 2014/0045833, and U.S. Pat. Nos. 8,501,751, 8,476,284, 8,008,309, 7,960,396, 7,825,118, 7,732,454, 7,514,444, 7,459,554, 7,405,295, and 7,393,848, the disclosures of each of which are incorporated herein by reference.

### Pharmaceutical Compositions

In some embodiments, the invention provides pharmaceutical compositions and methods for treating solid tumor cancers, lymphomas, leukemias, and blood dyscrasias.

In some embodiments, the invention provides pharmaceutical compositions of a BTK inhibitor for the treatment of a disease associated with BTK activity selected from inflammatory disorders, hyperproliferative disorders, and cancers that include but are not limited to acute myeloid leukemia, chronic lymphocytic leukemia, rheumatoid arthritis, psoriatic arthritis, infectious arthritis, progressive chronic arthritis, deforming arthritis, osteoarthritis, traumatic arthritis, gouty arthritis, Reiter's syndrome, polychondritis, acute synovitis, spondylitis, glomerulonephritis (with or without nephrotic syndrome), autoimmune hematologic disorders, hemolytic anemia, aplastic anemia, idiopathic thrombocytopenia, and neutropenia, autoimmune gastritis, and autoimmune inflammatory bowel diseases, ulcerative colitis, Crohn's disease, host versus graft disease, allograft rejection, chronic thyroiditis, Graves' disease, schleroderma, diabetes (type I and type II), active hepatitis (acute and chronic), pancreatitis, primary biliary cirrhosis, myasthenia gravis, multiple sclerosis, systemic lupus erythematosis, psoriasis, atopic dermatitis, contact dermatitis, eczema, skin sunburns, vasculitis (e.g. Behcet's disease) chronic renal insufficiency, Stevens-Johnson syndrome, inflammatory pain, idiopathic sprue, cachexia, sarcoidosis, Guillain-Barré syndrome, uveitis, conjunctivitis, kerato conjunctivitis, otitis media, periodontal disease, pulmonary interstitial fibrosis, asthma, bronchitis, rhinitis, sinusitis, pneumoconiosis, pulmonary insufficiency syndrome, pulmonary emphysema, pulmonary fibrosis, silicosis, chronic inflammatory pulmonary disease, chronic obstructive pulmonary disease, a proliferative diseases, non-Hodgkin lymphoma, diffuse large B-cell lymphoma (DLBCL), activated B cell diffuse large B-cell lymphoma (ABC-DLBCL), germinal center B cell diffuse large B-cell lymphoma (GCB-DLBCL), mantle cell lymphoma (MCL), B cell chronic lymphocytic leukemia, acute lymphoblastic leukemia, acute lymphoblastic leukemia with mature B cell, and B cell lymphoma, a proliferative mast cell disease, a bone disorder related to multiple myeloma, rheumatoid arthritis, psoriatic arthritis, osteoarthritis, proliferative diseases, non-Hodgkin's lymphoma, post-transplant lymphoproliferative disorders, blood dyscrasias, monoclonal gammopathy of undetermined source, light chain amyloidosis, acute lymphoblastic leukemia, acute lymphoblastic leukemia with mature B cell, or B cell lymphoma, a proliferative mast cell disease, a bone disorder related to multiple myeloma, AIDS-related (e.g., lymphoma and Kaposi's sarcoma) cancer, viral-induced cancer, or non-cancerous hyperproliferative disorders such as monoclonal B cell lymphocytosis, benign hyperplasia of the skin (e.g., psoriasis), restenosis, or prostate (e.g., benign prostatic hypertrophy (BPH)).

In some embodiments, the invention provides pharmaceutical compositions of a BTK inhibitor for the treatment of a solid tumor cancer selected from the group consisting of bladder cancer, squamous cell carcinoma, head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity cancer, oropharyngeal cancer, gastric cancer, stomach cancer, cervical cancer, renal cancer, kidney cancer, liver cancer, ovarian cancer, prostate cancer, colorectal cancer, esophageal cancer, testicular cancer, gynecological cancer, thyroid cancer, acquired immune deficiency syndrome (AIDS)-related cancers (e.g., lymphoma and Kaposi's sarcoma), viral-induced cancers such as cervical carcinoma (human papillomavirus), B-cell lymphoproliferative disease, nasopharyngeal carcinoma (Epstein-Barr virus), Kaposi's sarcoma and primary effusion lymphomas (Kaposi's sarcoma herpesvirus), hepatocellular carcinoma (hepatitis B and hepatitis C viruses), and T-cell leukemias (Human T-cell leukemia virus-1), glioblastoma, esophogeal tumors, head and neck tumor, metastatic colon cancer, head and neck squamous cell carcinoma, ovary tumor, pancreas tumor, renal cell carcinoma, hematological neoplasms, small-cell lung cancer, non-small-cell lung cancer, stage IV melanoma, and glioma.

The pharmaceutical compositions are typically formulated to provide a therapeutically effective amount of a covalent BTK inhibitor as the active ingredients, or a pharmaceutically acceptable salt, ester, prodrug, solvate, hydrate or derivative thereof. Where desired, the pharmaceutical compositions contain a pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants. Where desired, other agent(s) may be mixed into a preparation or both components may be formulated into separate preparations for use in combination separately or at the same time.

In some embodiments, the concentration of the BTK inhibitors provided in the pharmaceutical compositions and methods disclosed herein is independently less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the whole pharmaceutical composition or dosage form.

In some embodiments, the concentration of BTK inhibitors provided in the pharmaceutical compositions and methods disclosed herein is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the whole pharmaceutical composition or dosage form.

In some embodiments, the concentration of the BTK inhibitor in the compositions and methods disclosed herein is independently in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12% or approximately 1% to approximately 10% w/w, w/v or v/v of the whole pharmaceutical composition or dosage form.

In some embodiments, the concentration of the BTK inhibitor of the invention is independently in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v of the whole pharmaceutical composition or dosage form.

In some embodiments, the dose or amount of the BTK inhibitor of the invention is independently equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g or 0.0001 g present as the active ingredient in the whole pharmaceutical composition or dosage form.

In some embodiments, the dose or amount of the BTK inhibitor of the invention is independently more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g or 10 g present as the active ingredient in the whole pharmaceutical composition or dosage form.

The BTK inhibitor according to the invention is effective over a wide dosage range. For example, in the treatment of adult humans, dosages independently range from 0.01 to 1000 mg, from 0.5 to 100 mg, from 1 to 50 mg per day, and from 5 to 40 mg per day are examples of dosages that may be used. The exact dosage will depend upon the amount of BTK resynthesis in the human subject in any particular tissue compartment, and also route of administration, the form in which the compound is administered, the gender and age of the subject to be treated, the body weight of the subject to be treated, and the preference and experience of the attending physician.

Efficacy of the compounds and combinations of compounds described herein in treating, preventing and/or managing the indicated diseases or disorders can be tested using various animal or human models known in the art.

Described below are non-limiting exemplary pharmaceutical compositions and methods for preparing the same.

### Pharmaceutical Compositions for Oral Administration

In some embodiments, the invention provides a pharmaceutical composition for oral administration containing a covalent BTK inhibitor, and at least one pharmaceutical excipient suitable for oral administration.

In some embodiments, the invention provides a solid pharmaceutical composition for oral administration containing: (i) an effective amount of a BTK inhibitor and (ii) a pharmaceutical excipient suitable for oral administration. In selected embodiments, the composition further contains (iii) an effective amount of another active compound.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The invention further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Multiple BTK inhibitors can be used as active ingredients and can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

The methods of present invention may be achieved by formulation of the compositions into any suitable pharmaceutical dosage forms to provide a variety of drug release profiles, including immediate release, sustained release, and delayed release. In this respect various dosage forms are contemplated herein. These include, without limitation, pulsating release formulations including compositions of the present invention (wherein individual doses of the therapeutic agent is released at repeated intervals); extended release (ER) formulations including compositions of the present invention (in which slow release of the therapeutic agent provides therapeutic concentrations for 8-12 hours); controlled release (CR) formulations including compositions of the present invention (wherein the therapeutica gent is released at a constant rate); modified release (MR) formulations including compositions of the present invention (which provides gives drug release characteristic of time and/or location that are chosen to obtain therapeutic or convenience objective).

Therefore, in some embodiments, the pharmaceutical dosage form is formulated to prevent a therapeutic active agent release after administration until a predetermined interval of time has passed (timed release). In some embodiments, the pharmaceutical dosage form is formulated to provide substantially continuous release of a therapeutically active agent over a predetermined time period (sustained release). In some embodiments, the pharmaceutical dosage form is formulated to provide release of a therapeutically active agent immediately following administration of the pharmaceutical composition (immediate release).

In some embodiments, the pharmaceutical dosage form is formulated to release a therapeutically active agent in pulses, wherein a single pharmaceutical dosage form provides for an initial dose of a therapeutically active agent followed by a release-free time interval, after which a second dose of the therapeutically active agent is released, which may in turn be followed by one or more additional release-free time intervals and therapeutically active agent release pulses (e.g., pulsatile release).

In some embodiments, a pharmaceutical dosage form formulated to provide pulsatile release is useful, for example, with therapeutically active agents that have short half-lives and must be administered two or three times daily. In some embodiments, a pharmaceutical dosage form formulated to provide pulsatile release is useful to obtain a target BTK occupany in a tissue compartment or cell compartment based on BTK resynthesis rate. In some embodiments, a pharmaceutical dosage form formulated to provide pulsatile release is useful with therapeutically active agents that exhibit “first-pass effect” (also known as “first-pass metabolism” or “presystemic metabolism”), e.g., therapeutically active agents that are extensively metabolized and therefore whose concentration is greatly reduced before the therapeutically active agents reach the systemic circulation. In some embodiments, a pharmaceutical dosage form formulated to provide pulsatile release is useful with active agents which lose the desired therapeutic effect when constant blood levels are maintained. In some embodiments, a pharmaceutical dosage form formulated to provide pulsatile release is useful for minimizing the abuse potential of certain types of therapeutically active agents, e.g., therapeutically active agents for which tolerance, addiction and deliberate overdose can be problematic.

Any of the pharmaceutical dosage forms disclosed herein may be administered to a subject via any suitable route of administration, including, without limitation, oral, rectal, nasal, pulmonary, epidural, ocular, otic, intra-arterial, intracardiac, intracerebroventricular, intradermal, intravenous, intramuscular, intraperitoneal, intraosseous, intrathecal, intravesical, subcutaneous, topical, transdermal, transmucosal, sublingual, buccal, vaginal, and inhalational routes of administration. In some preferred embodiments, routes of delivery for MR formulations include, without limitation, injections, implants; topical plasters, tablet, capsule, ovule, suppository, film, vaginal ring, tampon, and osmotic pump system.

In a preferred embodiment, the pharmaceutical dosage form of the present invention is a controlled release pharmaceutical preparation comprising a core containing a compound of the present invention and a coating layer on the surface of the core. In some embodiments, the pharmaceutical dosage form comprises an immediate release core containing a therapeutic agent and one or more pharmaceutically acceptable excipients. In some embodiments, the pharmaceutical dosage form comprises a coating comprising a rate controlling polymer on the immediate release core. Any suitable rate controlling polymer may be used in the dosage forms of the present invention. In some preferred embodiments, examples of the rate controlling polymer include, without limitation, hydroxypropyl methyl cellulose acetate succinate, hydroxy propyl methylcellulose phthalate, methylcellulose, ethylcellulose, cellulose acetate, cellulose acetate phthalate, cellulose acetate trimellitate, carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins; vinyl polymers and copolymers such as polyvinyl pyrrolidone, vinyl acetate, vinylacetate phthalate, vinyl acetate crotonic acid copolymer, ethylene-vinyl acetate copolymer; enzymatically degradable polymers such as azo polymers, pectin, chitosan, amylase, guar gum, zein, shellac or a combination thereof.

In some embodiments, examples of the rate controlling polymer include, without limitation, cellulose acetate, cellulose triacetate, agar acetate, amylose triacetate, beta glucan acetate, acetaldehyde dimethyl acetate, cellulose acetate methyl carbamate, cellulose acetate phthalate, cellulose acetate succinate, cellulose acetate dimethylamino acetate, cellulose acetate ethyl carbonate, cellulose acetate chloroacetate, cellulose acetate ethyl oxalate, cellulose acetate butyl sulfonate, cellulose acetate propionate, poly(vinylmethylether) copolymers, cellulose acetate butyl sulfonate, cellulose acetate octate, cellulose acetate laurate, cellulose acetate p-toluene sulfonate, triacetate of locust gum bean, hydroxylated ethylene-vinyl acetate, cellulose acetate butyrate, ethyl cellulose and combinations thereof.

In some embodiments, the controlled release formulation comprises a multi-layered inner core, and/or a multi-layered coat.

Examples of pulsatile release formulation that may be adapted for use with the compositions of the present invention include, without limitation, those formulations described in: U.S. Pat. Nos. 5,413,777; 5,260,068; 4,777,049; 5,391,381; 5,472,708; and 5,260,069; and International Patent Application Publication No. WO 1998/32424, the disclosures of which are incorporated by reference herein.

In a preferred embodiment, the pharmaceutical dosage form of the present invention is sustained release solid dosage form. Examples of sustained release solid dosage forms include, without limitation, those described in: U.S. Pat. Nos. 6,056,977; 8,277,840; 4,690,682; 5,767,153; 4,889,721; 4,753,801; 5,773,031; 6,197,344; 7,422,758; 5,480,868; 6,087,324; 4,261,970; 4,869,904; 3,344,029; 6,852,724; 4,178,361; 2,951,792; 3,065,143; 4,837,032; 6,376,461; 8,067,020; 7,323,169; 5,136,968; 8,920,837; 5,330,767; 3,901,969; 6,528,093; 4,765,990; 6,355,236; 6,503,911; 3,911,100; 3,147,187; 2,805,977; 7,838,032; 5,261,896; 6,011,011; 5,593,694; 8,197,846; 4,968,508; 5,002,774; 5,601,844; 6,007,843; 4,990,340; 6,458,387; 4,988,679; 7,179,490; 3,901,968; 3,374,146; 5,238,686; 6,426,091; 4,781,919; 3,773,920; 7,662,408; 8,470,359; 5,972,891; 8,197,839; 8,877,242; 6,756,049; 8,992,979; 5,688,530; 6,447,796; 8,034,379; 7,883,718; 7,838,024; 8,361,052; 6,991,808; 7,833,545; 8,921,326; 8,529,542; 6,964,781; 6,506,410; 6,756,058; 8,318,210; 5,795,882; 6,419,961; 8,012,508; 6,410,052; 6,740,634; 4,889,720; 7,884,071; 8,574,613; 7,820,200; and 4,845,123; and International Patent Application Publication Nos. WO 2011/162413, 2011/078394, 2006/032089, 2005/117934, 2003/002102, 2005/123120, 2003/009833, 2012/063257, 2003/061634, 2003/009800, 2010/007623, 2003/051335, 2011/007353, 2013/024051, 2001/012233, 2001/072318, 2014/078486, 2007/147861, 2001/005430, 2002/026214, 2003/022242, 1998/032423, 2006/116565, 2006/116565, 2014/147526, 2003/009829, 2007/115033, 2007/115033, 2008/022146, 2008/022146, 2008/058288, 2008/058288, 2015/021153, 2008/075762, 2011/094431, 2012/003479, 2010/075072, 2013/173657, 2009/060322, 2010/102071, 2006/093838, 2006/093838, 2006/004167, 1998/029105, 2003/002091, 2011/024168, 2015/025312, 2014/036534, 2013/058838, 2013/175507, 1997/011681, 2012/131669, 2007/050294, 2008/086492, 2014/113377, and 2011/14348, the disclosures of which are incorporated by reference herein.

Examples of immediate release formulation that may be adapted for use with the compositions of the present invention include, without limitation, those formulations described in: U.S. Pat. Nos. 7,108,859; 8,895,058; 4,674,480; 8,580,298; 9,011,905; and 8,197,839; U.S. Patent Application Publication Nos. US 2014/0066447, 2007/0141140, 2006/0275365, and 2007/0059359; and International Patent Application Publication Nos. WO 2013/064900, 2004/073592, 2006/131394, and 2006/131393, the disclosures of which are incorporated by reference herein.

Examples of delayed release formulations that may be adapted for use with the compositions of the present invention include, without limitation, those formulations described in: U.S. Pat. Nos. 5,108,758; 7,105,174; 7,108,859; 6,677,319; 8,883,201; and 7,704,977; U.S. Patent Application Publication Nos. US 2002/0004070, 2003/0104054, 2003/0104058, 2014/0100283, 2010/0022480, 2008/0193590, 2010/0247640, 2007/0238707, 2007/0292512, 2007/0148228, 2008/0311223, 2010/0203163, 2006/0280795, 2011/0287094, and 2009/0324741; and International Patent Application Publication Nos. WO 1989/011269, 2015/089326, 2004/012717, 2007/117706, 2007/146234, 2011/107755, 2008/151433, and 2014/130678.

In some preferred embodiments, the pharmaceutical dosage forms comprise dosage units housed in a closed capsule. In some preferred embodiments, the pharmaceutical dosage forms comprise compressed tablets. In some preferred embodiments, the pharmaceutical dosage forms comprise a single tablet of which the drug-containing dosage units represent integral but discrete segments. In some preferred embodiments, the pharmaceutical dosage forms comprise drug-containing particles or beads. The drug-containing particle or bead (wherein a drug-containing particle or bead refers to drug-coated inert supports, e.g., lactose beads coated with drug) may release drug substantially immediately following ingestion of the dosage form, or follow a sustained release profile or delayed release profile.

In some embodiments, the pharmaceutical dosage forms comprise individual dosage units that are compacted in a single tablet, and represent integral but discrete segments thereof (e.g., layers). For example, drug-containing particles or drug-containing beads can be compressed together into a single tablet using conventional tabletting means.

### Pharmaceutical Compositions for Injection

In selected embodiments, the invention provides a pharmaceutical composition for injection containing a covalent BTK inhibitor and a pharmaceutical excipient suitable for injection. Components and amounts of agents in the compositions are as described herein.

The forms in which the compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, and thimerosal.

Sterile injectable solutions are prepared by incorporating the BTK inhibitor in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Eleventh Edition, McGraw-Hill, 2010.

Administration of covalent BTK inhibitors or pharmaceutical compositions of these compounds can be effected by any method that enables delivery of the compounds to the desired tissue compartment. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The combination of compounds can also be administered intraadiposally or intrathecally.

The invention also provides kits. The kits include a covalent BTK inhibitor in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another agent. In selected embodiments, the BTK inhibitor and the agent are provided as separate compositions in separate containers within the kit. In selected embodiments, the BTK inhibitor and the agent are provided as a single composition within a container in the kit. Suitable packaging and additional articles for use (e.g., measuring cup for liquid preparations, foil wrapping to minimize exposure to air, and the like) are known in the art and may be included in the kit.

### BTK Occupancy and Resynthesis

BTK occupancy (or BTK target occupancy) measures the amount of BTK enzyme that has been covalently bound to a BTK inhibitor at the active site kinase. Methods for the measurement of BTK occupancy in B cell lysates include, for example, use of selective probe molecules linked to biotin or other probes that may used for detection in various assay platforms. In case of biotin-labeled occupancy probes, the target occupancy can be measured and assayed by streptavidin pull down methods that bind the biotinylated probe molecule and BTK protein with standard enzyme-linked immunosorbent assay (ELISA) methods using streptavidin coated plates, as described, e.g., in Evans, et al., J. Pharmacol. Exp. Ther. 2013, 346, 219-228, or following capture using antibodies against BTK with detection using streptavadin conjugated to enzymes or probes for detection. When an appropriately standardized method is used, the BTK occupancy may be reported as a percentage of available BTK that is covalently bound, with 100% occupancy indicating that all BTK is covalently bound. BTK occupancy may also be reported as free BTK per mass of total protein (e.g., pg free BTK/ng total protein or ng free BTK/ng total protein), or may be reported as the percentage of free BTK that is available for detection by the BTK active site probe. Other suitable methods for determining BTK occupancy are described in U.S. Patent Application Publication No. US 2015/0260723 A1, the disclosure of which is incorporated by reference herein.

In an embodiment, the invention provides a method of treating a disorder caused by cellular BTK activity (i.e., a BTK mediated disorder) comprising the step of administering a covalent BTK inhibitor at a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%. In an embodiment, the invention provides a method of treating a BTK mediated disorder, wherein the disorder is a cancer, comprising the step of administering a BTK inhibitor at a dose effective to obtain a BTK occupancy selected from the group consisting of greater than 90%, greater than 91%, greater than 92%, greater than 93%, greater than 94%, greater than 95%, greater than 96%, greater than 97%, greater than 98%, and greater than 99%.

In an embodiment, the invention provides a method of treating a BTK mediated disorder comprising the step of administering a BTK inhibitor at a dose effective to obtain an average BTK occupancy selected from the group consisting of 80%, 81%, 82%, 83%, 84%, 85%, 86%, 87%, 88%, 89%, 90%, 91%, 92%, 93%, 94%, 95%, 96%, 97%, 98%, 99%, 99.5%, and 100%. In an embodiment, the invention provides a method of treating a BTK mediated disorder, wherein the disorder is an inflammatory, immune, or autoimmune disorder, comprising the step of administering a BTK inhibitor at a dose effective to obtain an average BTK occupancy selected from the group consisting of about 80%, about 85%, about 90%, about 95%, about 96%, about 97%, about 98%, and about 99%.

In an embodiment, the invention provides a method of treating a BTK mediated disorder comprising the step of administering a BTK inhibitor at a dose effective to obtain a BTK occupancy selected from the group consisting of between 80% and 85%, between 82.5% and 87.5%, between 85% and 90%, between 87.5% and 92.5%, between 90% and 95%, between 92.5% and 97.5%, and between 95% and 100%. In an embodiment, the invention provides a method of treating a BTK mediated disorder comprising the step of administering a BTK inhibitor at a dose effective to obtain a BTK occupancy selected from the group consisting of between 95% and 97%, between 96% and 98%, between 97% and 99%, and between 98% and 100%.

BTK resynthesis refers to the process by which new BTK enzyme is produced after existing BTK enzyme becomes occupied by covalent attachment to a BTK inhibitor. This can occur within a viable cell over time, or can occur during the generation of new cells upon proliferation or transit from a tissue compartment of therapeutic interest (e.g., bone marrow) into the assayed compartment. The BTK resynthesis rate can be measured by determining BTK occupancy over a period of time in a specific compartment; or by determining the presence of free BTK in a specimen sampled from a compartment of interest at a certain time after administration of a fully occupying dose of a covalent BTK inhibitor. The BTK resynthesis rate can also be obtained as an average rate.

BTK resynthesis rate may be determined by fitting BTK inhibitor occupancy data against a suitable biochemical kinetics model. For example, if the target occupancy assay determines free protein, and assuming full occupancy of the protein after dosing, free BTK may be determined after dosing by applying a pharmacokinetic-pharmacodynamic (PK/PD) model that estimates the t1/2 of BTK target occupancy as a function of the BTK resynthesis rate. Another approach is to use the following equation: free BTK=new BTK/h*h (where h refers to hours), or apply a linear extrapolation from data observing the decline in BTK target occupancy and the return of BTK signaling function during the washout period after dosing with a BTK inhibitor. The latter approaches assumes a linear synthesis rate for BTK over time, whereas the former approach is more nuanced and predicts a first or second order terminal elimination phase for BTK target occupancy. The target occupancy assay may be performed such that the free BTK is not an absolute value but is based on 100% free BTK in the individual prior to dosing. A value of 100% occupancy of BTK is determined by incubation of the same test sample with a high dose of an exogenous covalent BTK inhibitor. The BTK resynthesis rate (new BTK/hour) may be expressed as % per hour, as a percentage of predose free BTK. Alternately, if the expression of BTK protein is quantified, the BTK resynthesis rate may be expressed quantitatively, as pg/μg tissue/hour or pg/μg total protein/hour.

In an embodiment, the invention includes a method of treating cancer, a method of treating inflammatory, immune, and autoimmune diseases, and a method of surpressing immune responses for organ or cell transplants, wherein the cancer, disease, or immune response to be suppressed exhibits a rate of BTK resynthesis, which can be measured in sites of disease using specific imaging agents to detect the presence of unoccupied BTK target sites when combined with CT scans, positron emission tomography (PET) imaging, magnetic resonance imaging (MRI), or near infrared fluorescence imaging, or other in vivo imaging modalities, to customize the treatment of a specific disease based on the regeneration rate of BTK in diseased tissues. In an embodiment, the PET probe is a ¹¹C-labeled BTK inhibitor. In an embodiment, the PET probe is a ¹¹C-labeled BTK inhibitor. In an embodiment, the PET probe is a ¹¹C-labeled BTK inhibitor, such as the BTK inhibitors of Formulas (I) to (XXV), labeled at a specific carbon position, such as an exocylic carbon position, which may be prepared by synthetic methods known to those of ordinary skill in the art. In an embodiment, the PET probe is a ¹⁸F-labeled BTK inhibitor, such as the BTK inhibitors of Formulas (I) to (XXV), wherein a hydrogen is substituted by a ¹⁸F nucleus, such as an substitution at an aryl position, which may be prepared by synthetic methods known to those of ordinary skill in the art. Preparation of organic molecules containing ¹¹C, ¹⁸F, ¹³N and ¹⁵O labels for PET imaging is described, e.g., in Miller, et al., Angewandte Chemie Int. Ed., 2008, 47, 8998-9033.

In an embodiment, the BTK resynthesis rate is selected from the group consisting of about 0.1 pg free BTK/μg total protein/hour, about 0.5 pg free BTK/μg total protein/hour, about 1 pg free BTK/μg total protein/hour, about 2 pg free BTK/μg total protein/hour, about 3 pg free BTK/μg total protein/hour, about 4 pg free BTK/μg total protein/hour, about 5 pg free BTK/μg total protein/hour, about 6 pg free BTK/μg total protein/hour, about 7 pg free BTK/μg total protein/hour, about 8 pg free BTK/μg total protein/hour, about 9 pg free BTK/μg total protein/hour, about 10 pg free BTK/μg total protein/hour, about 20 pg free BTK/μg total protein/hour, and about 50 pg free BTK/μg total protein/hour.

In an embodiment, the BTK resynthesis half-life is selected from the group consisting of 2 hours, 4 hours, 6 hours, 8 hours, 10 hours, 12 hours, 14 hours, 16 hours, 18 hours, 20 hours, 22 hours, 24 hours, 48 hours, and 72 hours.

### Diseases Exhibiting Different BTK Resynthesis Rates in Different Tissues

Leukemias and lymphomas may show different relative rates of BTK resynthesis in B cells within, for example, the bone marrow, lymph nodes, and blood. A number of subsets of B cells are produced in the human body, as described in Perez-Andres, et al., Cytometry B (Clinical Cytometry), 2013, 78B (Suppl. 1), S47-S60 and Allman, et al., Curr. Opin. Immunol. 2008, 20, 149-157. For example, follicular B cells can mature in both the bone marrow and spleen, and can occupy at least two distinct niches. Certain B cell subsets can recirculate between different tissue compartments through peripherial blood. Tissue compartments include secondary lymphoid tissues (such as lymph nodes and mucosa-associated lymphoid tissues), bone marrow, and spleen, as described in Perez-Andres, et al., Cytometry B (Clinical Cytometry), 2013, 78B (Suppl. 1), S47-S60. Other tissue compartments may include sites of primary or metastatic disease such as occurs in primary central nervous system lymphoma, primary testicular lymphoma, and mucosa-associated lymphoid tissue (MALT) lymphomas. Knowledge of the recirculation of BTK bearing tumor cells through these compartments is important in the effective treatment of immune and lymphoproliferative diseases such as leukemia. The BTK target occupancy and resynthesis rate (or half-life) can be determined for different tissue compartments of interest using established sampling techniques, such as blood draws, fine needle aspirates and bone marrow biopsies.

BTK occupancies that approximate absolute values can be measured using methods such as that described in Evans, et al., J. Pharmacol. Exp. Ther. 2013, 346, 219-228. Other relative methods for measurement BTK occupancies may also be used with appropriate correction for total BTK in the sample, such as the Western blot method described in: Advani, et al., J. Clin. Oncol. 2013, 31, 88-94. Pulse chase methods, also known as pulse chase analysis, may also be used to assess BTK resynthesis rates. Pulse chase methods make use of pulsed exposure of the cell to a labeled compound (e.g., a radiolabeled amino acid) that is incorporated by the cell into the BTK protein, followed by exposure of the cell to unlabeled compound as a chase, after which the labeled BTK protein may be tracked until it degrades. The pulse may also be achieved using a labeled covalent BTK inhibitor, such as radiolabeled Formula (II), after which degradation of the protein-BTK inhibitor product may be tracked. Suitable pulse chase methods are described, for example, in Jansens and Braakman, Pulse-Chase Labeling Techniques for the Analysis of Protein Maturation and Degradation, In Protein Misfolding and Disease (Methods in Molecular Biology), Vol. 232, 2003, pp. 133-145.

In an embodiment, the invention includes methods of treatment of hematological malignancies and solid tumor cancers that exhibit different BTK resynthesis rates in the malignant cells in at least two different tissue compartments. For example, leukemias including chronic lymphocytic leukemia, small lymphocytic lymphoma, prolymphocytic leukemia, promyelocytic leukemia, diffuse large B cell lymphoma, mantle cell lymphoma, or B cell acute lymphoblastic leukemia may exhibit malignant cells in at least two different tissue compartments with different BTK resynthesis rates.

In an embodiment, the invention includes methods of treating inflammatory, immune, and autoimmune diseases, including dermatoses, which exhibit different BTK resynthesis rates in at least two different tissue compartments. For example, a dermatosis may exhibit a different BTK resynthesis rate (e.g., in a cutaneous lesion) in comparison to the BTK resynthesis rate in non-inflamed, normal skin.

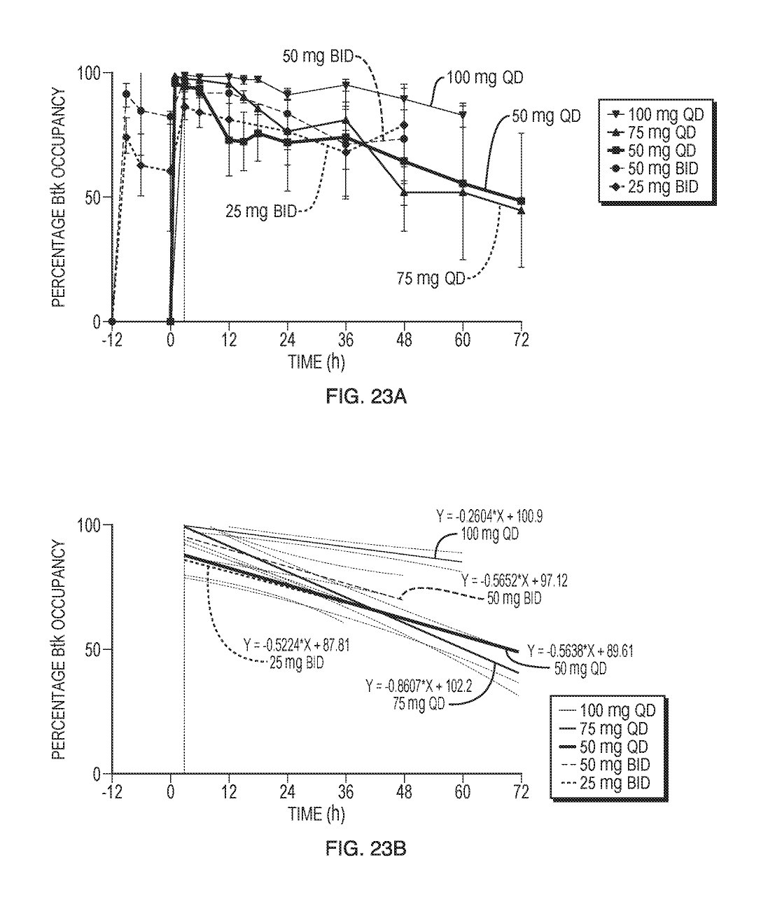

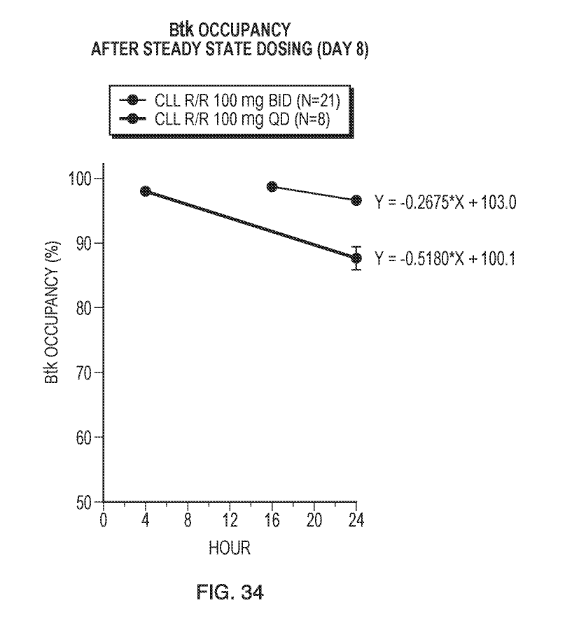

In an embodiment, the BTK resynthesis ratio between two tissue compartments is selected from the group consisting of 0.01 to 1, 0.1 to 1, 0.5 to 1, 1 to 1, 1 to 1.5, 1 to 2, 1 to 5, 1 to 10, and 1 to 100. In the peripheral blood B cells of healthy volunteers treated with a covalent inhibitor of BTK, the resynthesis rate of BTK was higher among those treated with doses that led to incomplete BTK target occupancy, as illustrated in FIG. 23. In comparison, in the tissue compartment comprising CLL tumor cells, the resynthesis rate of BTK was higher among those treated with 100 mg QD, compared with resynthesis following a 100 mg BID dose. Of interest, the resynthesis rate in the compartment of CLL tumor cells after dosing with 100 mg BID for 8 days (i.e., at steady state, FIG. 34) was similar to that observed in the normal B lymphocytes from healthy volunteers. Since treatment with a BTK inhibitor induces the release of B lymphocytes including CLL tumor cells from tissues into the peripheral blood (see Advani, et al., J. Clin. Oncol. 2013, 31, 88-94), one component of the BTK resynthesis rate observed in the blood is the contribution of peripheral (tissue-based) lymphocytes that transit into the central compartment.

### Dosages and Dosing Regimens

The amount of the BTK inhibitor administered will be dependent on the human subject being treated, the severity of the disorder or condition, the rate of administration, the disposition of the compounds and the discretion of the prescribing physician. For each disease setting and for subsets of patients within each disease setting, the resynthesis rate of BTK in target cells/tissues of interest, and the desired percentage of inhibition of BTK function, will also influence the amount of the BTK inhibitor administered. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day.

In an embodiment, the BTK inhibitor is administered in a single dose. Typically, such administration will be by injection—e.g., intravenous injection, in order to introduce the agents quickly. However, other routes may be used as appropriate. A single dose of the BTK inhibitor may also be used for treatment of an acute condition.

In selected embodiments, the BTK inhibitor is administered in multiple doses. Dosing may be about once, twice, three times, four times, five times, six times, or more than six times per day. Dosing may be about once a month, once every two weeks, once a week, or once every other day. In other embodiments, the BTK inhibitor is administered about once per day to about 6 times per day. In another embodiment the administration of the BTK inhibitor continues for less than about 7 days. In yet another embodiment the administration continues for more than about 6, 10, 14, 28 days, two months, six months, or one year. In some cases, continuous dosing is achieved and maintained as long as necessary.

Administration of the BTK inhibitor may continue as long as necessary. In selected embodiments, the BTK inhibitor is administered for more than 1, 2, 3, 4, 5, 6, 7, 14, or 28 days. In some embodiments, the BTK inhibitor is administered for less than 28, 14, 7, 6, 5, 4, 3, 2, or 1 day. In selected embodiments, the BTK inhibitor is administered on an ongoing basis—e.g., for the treatment of chronic effects.

An effective amount of the inhibitor may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The effective amount of a BTK inhibitor may be determined according to an aspect of the present invention by comparing and interpreting the BTK occupancy or BTK resynthesis rate obtained from B cells in different tissue compartments. In an embodiment, a leukemia, including chronic lymphocytic leukemia, small lymphocytic lymphoma, prolymphocytic leukemia, diffuse large B cell lymphoma, mantle cell lymphoma, or B cell acute lymphoblastic leukemia, shows a difference in BTK occupancy or BTK resynthesis rate between tumor cells in blood and tumor cells in tissue compartments (including lymph nodes and bone marrow), wherein the BTK occupancy or BTK resynthesis rate is greater in the tissue compartment by an amount selected from the group consisting of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In an embodiment, a leukemia, including chronic lymphocytic leukemia, small lymphocytic lymphoma, diffuse large B cell lymphoma, mantle cell lymphoma, or B cell acute lymphoblastic leukemia, shows a difference in BTK occupancy or BTK resynthesis rate between tumor cells in blood and tumor cells in tissue compartments (including lymph nodes, bone marrow, and sites of primary and/or metastatic lymphoma), wherein the BTK occupancy or BTK resynthesis rate is greater in the tissue compartment by an amount in the range selected from the group consisting of 0 to 10%, 10 to 20%, 20% to 30%, 30% to 40%, 40% to 50%, 50% to 60%, 60% to 70%, 70% to 80%, 80% to 90%, or 90% to 100%.

In an embodiment, the invention includes a method of treating a leukemic cancer that exhibits a higher rate of BTK resynthesis in leukemic bone marrow B cells relative to the BTK resynthesis rate in leukemic blood B cells, comprising the step of administering a dose of a compound to reduce the rate of BTK resynthesis, wherein the compound is selected from the group consisting of Formula (I), Formula (II), Formula (III), Formula (IV), Formula (V), and Formula (VI), or a pharmaceutically-acceptable salt, cocrystal, hydrate, solvate, or prodrug thereof, wherein the dose is administered once daily, twice daily, or three times daily, and wherein the leukemic cancer is chronic lymphocytic leukemia, small lymphocytic lymphoma, diffuse large B cell lymphoma, or mantle cell lymphoma.

In an embodiment, the invention includes a method of treating a leukemic cancer that exhibits a higher rate of BTK resynthesis in leukemic bone marrow B cells relative to the BTK resynthesis rate in leukemic lymph node B cells, comprising the step of administering a dose of a compound to reduce the rate of BTK resynthesis, wherein the compound is selected from the group consisting of Formula (I), Formula (II), Formula (III), Formula (IV), Formula (V), and Formula (VI), or a pharmaceutically-acceptable salt, cocrystal, hydrate, solvate, or prodrug thereof, wherein the dose is administered once daily, twice daily, or three times daily, and wherein the leukemic cancer is chronic lymphocytic leukemia, small lymphocytic lymphoma, diffuse large B cell lymphoma, or mantle cell lymphoma.

In an embodiment, the invention includes a method of treating an acute leukemic cancer that exhibits a higher rate of BTK resynthesis in acute leukemic blood B cells than the BTK resynthesis rate in chronic leukemic blood B cells, comprising the step of administering a dose of a compound to reduce the rate of BTK resynthesis, wherein the compound is selected from the group consisting of Formula (I), Formula (II), Formula (III), Formula (IV), Formula (V), and Formula (VI), or a pharmaceutically-acceptable salt, cocrystal, hydrate, solvate, or prodrug thereof, wherein the dose is administered once daily, twice daily, or three times daily, and wherein the leukemic cancer is B cell acute lymphoblastic leukemia.

### Methods of Treating Cancers

In some embodiments, the invention provides a method of treating a BTK-mediated disease or a hyperproliferative disorder in a subject, wherein the hyperproliferative disorder is a cancer. In an embodiment, the subject is a mammal. In an embodiment, the subject is a human. In an embodiment, the subject is a mammal, wherein the mammal is a companion animal, such as a canine, feline, or equine.

In some embodiments, the invention provides a method of treating a cancer in a human subject, wherein the cancer is a leukemia, a lymphoma, or a solid tumor cancer, comprising the step of administering to said human subject a therapeutically effective amount of a BTK inhibitor, or a pharmaceutically acceptable salt, ester, prodrug, solvate, cocrystal, or hydrate of the BTK inhibitor. In some embodiments, the invention provides a method of treating a cancer selected from the group consisting of non-Hodgkin's lymphoma, acute myeloid leukemia, chronic lymphocytic leukemia (CLL), small lymphocytic lymphoma (SLL), diffuse large B cell lymphoma (DLBCL), mantle cell lymphoma (MCL), Waldenström's macroglobulinemia, follicular lymphoma, B cell acute lymphoblastic leukemia, Burkitt's leukemia, juvenile myelomonocytic leukemia, mast cell leukemia, hairy cell leukemia, Hodgkin's disease, multiple myeloma, thymus cancer, brain cancer, glioma, lung cancer, squamous cell cancer, skin cancer, melanoma, eye cancer, retinoblastoma, intraocular melanoma, oral cavity cancer, oropharyngeal cancer, bladder cancer, gastric cancer, stomach cancer, pancreatic cancer, breast cancer, cervical cancer, head cancer, neck cancer, renal cancer, kidney cancer, liver cancer, ovarian cancer, prostate cancer, colorectal cancer, bone cancer (e.g., bone metastases), esophageal cancer, testicular cancer, gynecological cancer, thyroid cancer, central nervous system cancer, cancer related to acquired immune deficiency syndrome (e.g., lymphoma and Kaposi's sarcoma), viral-induced cancers such as cervical carcinoma (human papillomavirus), B cell lymphoproliferative disease and nasopharyngeal carcinoma (Epstein-Barr virus), Kaposi's sarcoma and primary effusion lymphomas (Kaposi's sarcoma herpesvirus), hepatocellular carcinoma (hepatitis B and hepatitis C viruses), and T-cell leukemias (human T-cell leukemia virus-1), or mastocytosis. In some embodiments, the invention provides a method of treating a non-cancerous hyperproliferative disorder such as benign hyperplasia of the skin (e.g., psoriasis), restenosis, or prostate conditions (e.g., benign prostatic hypertrophy (BPH)). In some embodiments, the invention provides a method of treating a proliferative disorder in myeloid lineage cells, such as acute myeloid leukemia and chronic myelogenous leukemia.

In an embodiment, the invention provides a method of treating a subtype of CLL in a human that comprises the step of administering to said human subject a therapeutically effective amount of a BTK inhibitor, or a pharmaceutically acceptable salt, ester, prodrug, cocrystal, solvate or hydrate thereof. A number of subtypes of CLL have been characterized. CLL may be classified for immunoglobulin heavy-chain variable-region (IgV_{H}) mutational status in leukemic cells. Damle, et al., Blood 1999, 94, 1840-47; Hamblin, et al., Blood 1999, 94, 1848-54. Patients with IgV_{H} mutations generally survive longer than patients without IgV_{H} mutations. ZAP70 expression (positive or negative) is also used to characterize CLL. Rassenti, et al., N. Engl. J. Med. 2004, 351, 893-901. The methylation of ZAP-70 at CpG3 is also used to characterize CLL, for example by pyrosequencing. Claus, et al., J. Clin. Oncol. 2012, 30, 2483-91; Woyach, et al., Blood 2014, 123, 1810-17. CLL is also classified by stage of disease under the Binet or Rai criteria. Binet, et al., Cancer 1977, 40, 855-64; K. R. Rai, T. Han, Hematol. Oncol. Clin. North Am. 1990, 4, 447-56. Other common mutations, such as 11p deletion, 13q deletion, and 17p deletion can be assessed using well-known techniques such as fluorescence in situ hybridization (FISH). In an embodiment, the invention provides a method of treating a CLL in a human that comprises the step of administering to said human a therapeutically effective amount of a BTK inhibitor of Formula (II), or a pharmaceutically acceptable salt or ester, prodrug, cocrystal, solvate or hydrate thereof, wherein the CLL is selected from the group consisting of IgV_{H} mutation negative CLL, ZAP-70 positive CLL, ZAP-70 methylated at CpG3 CLL, CD38 positive CLL, chronic lymphocytic leukemia characterized by a 17p13.1 (17p) deletion, and CLL characterized by a 11q22.3 (11q) deletion.

In an embodiment, the invention provides a method of treating a CLL in a human that comprises the step of administering to said mammal a therapeutically effective amount of a BTK inhibitor, or a pharmaceutically acceptable salt, ester, prodrug, cocrystal, solvate, or hydrate thereof, wherein the CLL has undergone a Richter's transformation. Methods of assessing Richter's transformation, which is also known as Richter's syndrome, are described in Jain and O'Brien, Oncology, 2012, 26, 1146-52. Richter's transformation is a subtype of CLL that is observed in 5-10% of patients. It involves the development of aggressive lymphoma from CLL and has a generally poor prognosis.

In an embodiment, the invention provides a method of treating a hematological malignancy in a human comprising the step of administering to said human a therapeutically effective amount of a BTK inhibitor, or a pharmaceutically acceptable salt, ester, prodrug, cocrystal, solvate, or hydrate thereof. Hematological malignancies include CLL and SLL, as well as other cancers of the blood, including B cell malignancies. In an embodiment, the invention relates to a method of treating a hematological malignancy selected from the group consisting of non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Waldenstrom's macroglobulinemia (WM), Burkitt's lymphoma, multiple myeloma, or myelofibrosis in a human that comprises the step of administering a therapeutically effective amount of a BTK inhibitor, or a pharmaceutically acceptable salt, ester, prodrug, cocrystal, solvate or hydrate thereof. In an embodiment, the invention relates to a method of treating a NHL selected from the group consisting of indolent NHL and aggressive NHL comprising the step of administering a therapeutically effective amount of a BTK inhibitor, or a pharmaceutically acceptable salt, ester, prodrug, cocrystal, solvate or hydrate thereof.

In an embodiment, the invention provides a method of treating a DLBCL selected from the group consisting of activated B-cell like diffuse large B-cell lymphoma (DLBCL-ABC) and germinal center B-cell like diffuse large B-cell lymphoma (DLBCL-GCB), comprising the step of administering a therapeutically effective amount of a BTK inhibitor, or a pharmaceutically acceptable salt or ester, prodrug, cocrystal, solvate or hydrate thereof.

In an embodiment, the invention provides a method of treating an MCL selected from the group consisting of mantle zone MCL, nodular MCL, diffuse MCL, and blastoid MCL (also known as blastic variant MCL), comprising the step of administering a therapeutically effective amount of a BTK inhibitor, or a pharmaceutically acceptable salt or ester, prodrug, cocrystal, solvate or hydrate thereof.

In an embodiment, the invention provides a method of treating a B-ALL selected from the group consisting of early pre-B cell B-ALL, pre-B cell B-ALL, mature B cell B-ALL (also known as Burkitt's leukemia), and prolymphocytic leukemia comprising the step of administering a therapeutically effective amount of a BTK inhibitor, or a pharmaceutically acceptable salt or ester, prodrug, cocrystal, solvate or hydrate thereof.

In an embodiment, the invention provides a method of treating a Burkitt's lymphoma selected from the group consisting of sporadic Burkitt lymphoma, endemic Burkitt lymphoma, and human immunodeficiency virus-associated Burkitt lymphoma, comprising the step of administering a therapeutically effective amount of a BTK inhibitor, or a pharmaceutically acceptable salt or ester, prodrug, cocrystal, solvate or hydrate thereof.

In an embodiment, the invention provides a method of treating a multiple myeloma selected from the group consisting of hyperdiploid multiple myeloma and non-hyperdiploid multiple myeloma, plasmacytoma, monoclonal gammopathy of undetermined significance (MGUS), or amyloidosis comprising the step of administering a therapeutically effective amount of a BTK inhibitor, or a pharmaceutically acceptable salt or ester, prodrug, cocrystal, solvate or hydrate thereof.

In an embodiment, the invention provides a method of treating a myelofibrosis selected from the group consisting of primary myelofibrosis (also known as chronic idiopathic myelofibrosis) and myelofibrosis secondary to polycythemia vera or essential thrombocythaemia, comprising the step of administering a therapeutically effective amount of a BTK inhibitor, or a pharmaceutically acceptable salt or ester, prodrug, cocrystal, solvate or hydrate thereof. In an embodiment, the invention provides a method of treating myeloproliferative disorders, myeloproliferative neoplasms, polycythemia vera, essential thrombocythemia, myelodysplastic syndrome, chronic myelogenous leukemia (e.g., BCR-ABL1-positive), chronic neutrophilic leukemia, or chronic eosinophilic leukemia.

Efficacy of the methods and compositions described herein in treating, preventing and/or managing the indicated diseases or disorders can be tested using various models known in the art. For example, models for determining efficacy of treatments for pancreatic cancer are described in Herreros-Villanueva, et al., World J. Gastroenterol. 2012, 18, 1286-1294. Models for determining efficacy of treatments for breast cancer are described, e.g., in Fantozzi, Breast Cancer Res. 2006, 8, 212. Models for determining efficacy of treatments for ovarian cancer are described, e.g., in Mullany, et al., Endocrinology 2012, 153, 1585-92; and Fong, et al., J. Ovarian Res. 2009, 2, 12. Models for determining efficacy of treatments for melanoma are described, e.g., in Damsky, et al., Pigment Cell & Melanoma Res. 2010, 23, 853-859. Models for determining efficacy of treatments for lung cancer are described, e.g., in Meuwissen, et al., Genes & Development, 2005, 19, 643-664. Models for determining efficacy of treatments for lung cancer are described, e.g., in Kim, Clin. Exp. Otorhinolaryngol. 2009, 2, 55-60; and Sano, Head Neck Oncol. 2009, 1, 32.

### Methods of Treating Inflammatory, Immune, and Autoimmune Diseases, Including Dermatoses

In some embodiments, the invention provides a method of treating a BTK-mediated disease or hyperproliferative disorder in a subject, wherein the hyperproliferative disorder is an inflammatory, immune, or autoimmune disorder. In an embodiment, the subject is a mammal. In an embodiment, the subject is a human. In an embodiment, the subject is a mammal, wherein the mammal is a companion animal, such as a canine, feline, or equine.

In some embodiments, the invention provides a method of treating an inflammatory, immune, or autoimmune disorder in a human that comprises administering to said human a therapeutically effective amount of a BTK inhibitor, or a pharmaceutically acceptable salt or ester, prodrug, solvate or hydrate of the BTK inhibitor.

In some embodiments, the invention provides a method of treating an inflammatory, immune, or autoimmune disorder selected from the group consisting of tumor angiogenesis, chronic inflammatory disease, rheumatoid arthritis, atherosclerosis, inflammatory bowel disease, skin diseases such as psoriasis, atopic dermatitis, bullous pemphigoid, eczema, and scleroderma, diabetes, diabetic retinopathy, retinopathy of prematurity, age-related macular degeneration, hemangioma, ulcerative colitis, atopic dermatitis, pouchitis, spondylarthritis, uveitis, Behcet's disease, polymyalgia rheumatica, post-herpetic neuralgia, systemic exertion intolerance disease, giant-cell arteritis, sarcoidosis, Kawasaki disease, juvenile idiopathic arthritis, hidratenitis suppurativa, Sjögren's syndrome, psoriatic arthritis, juvenile rheumatoid arthritis, ankylosing spoldylitis, asthma, Crohn's disease, lupus, lupus nephritis, and polycythemia vera.

In some embodiments, the invention provides a method of treating an inflammatory, immune, or autoimmune disorder that is a chronic B cell disorder in which BCR signaling leads to the inappropriate production of autoimmune antibodies or release of pro-inflammatory cytokines and activation of immune cells including inflammatory T cells. In diseases of this type, reducing BCR signaling by inhibition of BTK may lead to therapeutic benefit. In some embodiments, the invention provides a method of treating an inflammatory, immune, or autoimmune disorder selected from the group consisting of rheumatoid arthritis (RA), juvenile RA, juvenile idiopathic arthritis, osteoarthritis, psoriatic arthritis, psoriasis vulgaris, pemphigus, bullous pemphigoid, osteoarthritis, infectious arthritis, progressive chronic arthritis, polymyalgia rheumatic, deforming arthritis, traumatic arthritis, gouty arthritis, Reiter's syndrome, polychrondritis, acute synovitis, ankylosing spondylitis, spondylitis, Sjogren's syndrome (SS), systemic lupus erythromatosus (SLE), discoid lupus erythromatosus (discoid LE), LE tumidus, lupus nephritis (LN), antiphospholipidosis, dermatomyositis, polymyositis, autoimmune hematologic disorders, thrombocytopenia, idiopathic thrombocytopenia purpura, thrombotic thrombocytopenia purpura, autoimmune (cold) agglutinin disease, autoimmune hemolytic anemia, cryoglobulinemia, aplastic anemia, neutropenia, autoimmune vasculitis, Behcet's disease, anti-neutrophil cytoplasmic antibody (ANCA)-associated vasculitis, scleroderma, systemic sclerosis, myasthenia gravis, multiple sclerosis (MS), chronic focal encephalitis, Guillian-Barre syndrome, chronic fatigue syndrome, systemic exertion intolerance disease, neuromyelitis optica, autoimmune uveitis, conjunctivitis, keratoconjuctivitis, Grave's disease, thyroid associated opthalmopathy, chronic thyroiditis, granulomatosis with microscopic polyangitis, Wegener's granulomatosis, autoimmune gastritis, autoimmune inflammatory bowel diseases, ulcerative colitis, Crohn's disease, graft versus host disease, idiopathic sprue, autoimmune hepatitis, active hepatitis (acute and chronic), idiopathic pulmonary fibrosis, bronchitis, pulmonary interstitial fibrosis, chronic inflammatory pulmonary disease, sarcoidosis, idiopathic membranous nephropathy, IgA nephropathy, glomerulosclerosis, glomerulonephritis (with or without nephrotic syndrome), pancreatitis and type 1 or type 2 diabetes.

In some embodiments, the invention provides a method of treating an inflammatory, immune, or autoimmune disorder, wherein the inflammatory, immune, or autoimmune disorder is a chronic autoimmune and inflammatory disorder in which BTK signaling in myeloid cells and mast cells leads to the inappropriate release of pro-inflammatory cytokines and activation of immune cells including inflammatory T cells, autoreactive B cells, activated tissue macrophages, activated mast cells, infiltrating monocytes and granulocytic inflammatory infiltrates, and activation of tissue-resident dendritic cell populations. In diseases of this nature, reducing BTK signaling through surface or endocytic receptors on the myeloid cells may lead to therapeutic benefit. In some embodiments, the invention provides a method of treating an inflammatory, immune, or autoimmune disorder selected from the group consisting of diabetic retinopathy, giant cell arteritis, Kawasaki disease, inflammatory bowel disease, irritable bowel disease, idiopathic sprue, enteropathy, post-herpetic neuralgia, polymyalgia rheumatic, primary biliary cirrhosis, myasthenia gravis, inflammatory pain, cachexia, periodontal disease, otitis media, pneumoconiosis, mononucleosis, pulmonary emphysema, pulmonary fibrosis, silicosis, chronic inflammatory pulmonary disease, chronic obstructive pulmonary disease, pulmonary insufficiency, pulmonary interstitial fibrosis, whipple, benign hyperplasia of the skin (e.g., psoriasis), myalgias caused by infections, cachexia secondary to infections, systemic exertion intolerance disease, atherosclerosis, granulomatosis, granulomatosis with microscopic polyangitis, hidradenitis suppurativa, age-related macular degeneration, and amyloidosis.

In some embodiments, the invention provides a method of treating an inflammatory, immune, or autoimmune disorder selected from the group consisting tumor angiogenesis, chronic inflammatory disease, rheumatoid arthritis, atherosclerosis, inflammatory bowel disease, skin diseases such as psoriasis, eczema, and scleroderma, Type 1 diabetes, Type 2 diabetes, diabetic retinopathy, retinopathy of prematurity, age-related macular degeneration, hemangioma, ulcerative colitis, atopic dermatitis, pouchitis, spondylarthritis, uveitis, Behcet's disease, polymyalgia rheumatica, giant-cell arteritis, sarcoidosis, Kawasaki disease, juvenile idiopathic arthritis, hidratenitis suppurativa, Sjögren's syndrome, psoriatic arthritis, juvenile rheumatoid arthritis, ankylosing spondylitis, Crohn's disease, lupus, lupus nephritis, human leukocyte antigen (HLA) associated diseases, autoantibodies, immunotherapy, Addison's disease, autoimmune polyendocrine syndrome type 1 (APS-1), autoimmune polyendocrine syndrome type 2 (APS-2), Grave's disease, Hashimoto's thyroiditis, polyendocrine autoimmunity, iatrogenic autoimmunity, idiopathic hypoparathyroidism, and vitilago.

In some embodiments, the invention provides a method of treating an inflammatory, immune, or autoimmune disorder, wherein the inflammatory, immune, or autoimmune disorder is a dermatosis in which BTK-mediated signals are involved with the recruitment, activation and/or proliferation of inflammatory cells and production of inflammatory mediators and antimicrobial peptides in the skin. In some embodiments, the invention provides a method of treating a dermatosis wherein the dermatosis results from dermal manifestations of systemic diseases where sensitization, lymphocyte recruitment, lymphocyte skewing by local or lymph-node antigen presenting cells, activation of skin-resident or skin-homing lymphocytes, innate immune sensing, keratinocyte antimicrobial responses, activation of resident or infiltrating myeloid dendritic cells, plasmacytoid dendritic cells, macrophages, mast cells, neutrophils, eosinophils, and/or Langerhans cells leads to development of skin lesions. In some embodiments, the invention provides a method of treating a dermatosis selected from the group consisting of psoriasis vulgaris, guttate psoriasis, erythrodermic psoriasis, psoriatic nails, annular pustular psoriasis, pustular psoriasis, inverse psoriasis, psoriatic arthritis, keratoderma blennorrhagicum, parapsoriasis, erythema nodosum, palmoplantar hidradentitis, atopic dermatitis, atopic eczema, seborrheic eczema, seborrheic dermatitis, dyshidrosis, rosacea, cutaneous lupus erythematosus, acute cutaneous lupus erythematosus, subacute cutaneous lupus erythematosus, discoid lupus erythematosus, lupus erythromatosus tumidus, lupus nephritis (LN), lupus erythematosus panniculitis, erythema multiforme, verruca, verrucous lupus erythematosus, vitiligo, alopecia areata, purigo nodularis, lichen planus, purigo pigmentosum, pemphigus vulgaris, bullous pemphigoid, pemphigus erythematosus, pemphigus nodularis, erythrodermic sarcoidosis, granulomatous dermatisis, scleroderma, systemic sclerosis, cutaneous manifestations of systemic sclerosis, diffuse cutaneous mastocytosis, erythrodermic mastocytosis, granuloma annulare, chondrodermatitis nodularis, contact dermatitis, drug eruptions, linear IgA bullous dermatosis, eosinophilic dermatitis, keratosis pilaris, lymphomatoid papulosis, pityriasis lichenoides et varioliformis acuta (PLEVA), lichenoides chronica (PLC), febrile ulceronecrotic Mucha-Habermann disease (FUMHD), chronic urticaria, rheumatoid neutrophilic dermatitis, cryoglobulinemic purpura, and purpura hyperglobulinemica.

In some embodiments, the invention relates to a method of treating, with a BTK inhibitor, a dermatosis, wherein the dermatosis is a condition that results from deposition of antibodies or autoantibodies within the dermal/epidermal junction and the accumulation of innate and adaptive immunocytes to these regions within the skin.

In some embodiments, the invention relates to a method of treating, with a BTK inhibitor, a dermatosis that results from an allergic reaction.

In some embodiments, the invention relates to a method of treating, with a BTK inhibitor, a dermatosis that features an increased proliferation of keratinocytes in the epidermis and the dysregulation of differentiation events through the strata of epidermal layers, and progressive loss of barrier functions.

In some embodiments, the invention relates to a method of treating, with a BTK inhibitor, an inflammatory dermatosis that occurs in genetically pre-disposed individuals.

In some embodiments, the invention relates to a method of treating, with a BTK inhibitor, a skin manifestation of an underlying autoimmune, allergic, or inflammatory disorder.

In some embodiments, the invention relates to a method of treating, with a BTK inhibitor, a dermatosis affecting palmar, plantar, nail, axillary, or genitocrural regions, or scalp, or other localized cutaneous manifestation of an inflammatory disorder.

In some embodiments, the invention provides a method of treating a hyperproliferative disorder, wherein the hyperproliferative disorder is a chronic autoimmune and inflammatory disorder of the bone in which BTK signaling in osteoclasts, mast cells, and myeloid cells is involved in osteolysis, osteoclastic processes, imbalance of bone remodeling processes, or loss of bone density. Diseases of this nature, which often have an autoimmune component as well, include osteoarthritis, bone loss due to metastases, osteolytic lesions, osteoporosis, ankylosing spondylitis, spondylarthritis, diffuse idiopathic skeletal hyperostosis, gouty arthritis, and bone disorders related to multiple myeloma. In some embodiments, the invention provides a method of treating a hyperproliferative disorder, wherein the hyperproliferative disorder is selected from the group consisting of osteoarthritis, bone loss due to metastases, osteolytic lesions, osteoporosis, ankylosing spondylitis, spondylarthritis, diffuse idiopathic skeletal hyperostosis, gouty arthritis, and bone disorders related to multiple myeloma.

In some embodiments, the invention provides a method treating allergic and atopic diseases in which activated B cells produce IgE antibodies and mast cells degranulate following engagement of the FcεR leading to release of pro-inflammatory factors and acute activation of local tissue responses as well as chronic changes to endothelial cells, neuroreceptors and other proximal structures which govern organ function. Such conditions include atopic dermatitis, contact dermatitis, eczema, atopic eczema, pemphigus vulgaris, bullous pemphigus, prurigo nodularis, Stevens-Johnson syndrome, asthma, airway hypersensitivity, bronchospasm, bronchitis, reactive asthma, chronic obstructive pulmonary disease, type 1 hypersensitivity, type 2 hypersensitivity, allergic rhinitis, allergic conjunctivitis, and other inflammatory or obstructive disease on airways. Allergies that can be treated or prevented include, among others, allergies to foods, food additives, insect poisons, dust mites, pollen, animal materials, metals, and certain drugs.

Efficacy of the methods described herein in treating, preventing and/or managing the indicated diseases or disorders can be tested using various animal models known in the art. Efficacy in treating, preventing and/or managing arthritis (e.g., rheumatoid or psoriatic arthritis) can be assessed using the autoimmune animal models described in, for example, Williams, et al., Chem. Biol. 2010,17, 123-34, WO 2009/088986, WO 2009/088880, and WO 2011/008302. Efficacy in treating, preventing and/or managing psoriasis can be assessed using transgenic or knockout mouse model with targeted mutations in epidermis, vasculature or immune cells, mouse model resulting from spontaneous mutations, and immuno-deficient mouse model with xenotransplantation of human skin or immune cells, all of which are described, for example, in Boehncke, et al., Clinics in Dermatology, 2007, 25, 596-605. Efficacy in treating, preventing and/or managing fibrosis or fibrotic conditions can be assessed using the unilateral ureteral obstruction model of renal fibrosis, which is described, for example, in Chevalier, et al., Kidney International 2009, 75, 1145-1152; the bleomycin induced model of pulmonary fibrosis described in, for example, Moore, et al., Am. J. Physiol. Lung. Cell. Mol. Physiol. 2008, 294, L152-L160; a variety of liver/biliary fibrosis models described in, for example, Chuang, et al., Clin. Liver Dis. 2008, 12, 333-347 and Omenetti, et al., Laboratory Investigation, 2007, 87, 499-514 (biliary duct-ligated model); or any of a number of myelofibrosis mouse models such as described in Varicchio, et al., Expert Rev. Hematol. 2009, 2(3), 315-334. Efficacy in treating, preventing and/or managing scleroderma can be assessed using a mouse model induced by repeated local injections of bleomycin described, for example, in Yamamoto, et al., J. Invest. Dermatol. 1999, 112, 456-462. Efficacy in treating, preventing and/or managing dermatomyositis can be assessed using a myositis mouse model induced by immunization with rabbit myosin as described, for example, in Phyanagi, et al., Arthritis & Rheumatism, 2009, 60(10), 3118-3127. Efficacy in treating, preventing and/or managing lupus can be assessed using various animal models described, for example, in Ghoreishi, et al., Lupus, 2009, 19, 1029-1035; Ohl, et al., J. Biomed. Biotechnol., 2011, Article ID 432595; Xia, et al., Rheumatology, 2011, 50, 2187-2196; Pau, et al., PLoS ONE, 2012, 7(5), e36761; Mustafa, et al., Toxicology, 2011, 290, 156-168; Ichikawa, et al., Arthritis & Rheumatism, 2012, 62(2), 493-503; Rankin, et al., J. Immunology, 2012, 188, 1656-1667. Efficacy in treating, preventing and/or managing Sjögren's syndrome can be assessed using various mouse models described, for example, in Chiorini, et al., J. Autoimmunity, 2009, 33, 190-196. Efficacy in treating, preventing and/or managing autoimmune cytopenias can be assessed using mouse models induced by intravenous administration of erythrocytes and/or platelets from the rat as described, for example, in Musaji et al., Exp. Hematol. 2004, 32, 87-94; or from HLA mismatched donor mice as described for example in Yabe et al., Bone Marrow Transplant. 1996, 17, 985-91.

In an embodiment, provided herein is a method of treating, preventing and/or managing asthma in a human subject comprising administering to said human subject a therapeutically effective amount of a BTK inhibitor, or a pharmaceutically acceptable salt or ester, prodrug, solvate or hydrate of the BTK inhibitor. As used herein, “asthma” encompasses airway constriction associated with inflammation. Common triggers of asthma include, but are not limited to, exposure to an environmental stimulants (e.g., allergens), cold air, warm air, perfume, moist air, exercise or exertion, and emotional stress. Also provided herein is a method of treating, preventing and/or managing one or more symptoms associated with asthma. Examples of the symptoms include, but are not limited to, severe coughing, airway constriction and mucus production. Efficacy in treating, preventing and/or managing asthma can be assessed using the ovalbumin induced asthma model described, for example, in Lee, et al., J. Allergy Clin. Immunol. 2006, 118, 403-9.

In an embodiment, provided herein is a method of treating, preventing and/or managing atopic dermatitis, and other atopic diseases in a human subject comprising administering to said human subject a therapeutically effective amount of a BTK inhibitor, or a pharmaceutically acceptable salt or ester, prodrug, solvate or hydrate of the BTK inhibitor. As used herein, “atopic dermatitis” encompasses atopic skin diseases, including eczema, prurigo nodularis, ichthyosis vulgaris, psoriasis and other dermatoses that constitute persistent or bothersome skin rashes observed in juveniles or adults. Atopic skin disorders are often observed and difficult to treat in companion animals, especially dogs. Common triggers of atopic dermatitis include, but are not limited to, exposure to environmental stimulants (e.g., allergens), infections (i.e., with S. aureus), activation of mast cells, and inadequate barrier function due to genetic disposition, skin dryness, viral infections and/or emotional stress. Also provided herein is a method of treating, preventing and/or managing one or more symptoms associated with atopic dermatitis. Examples of the symptoms include, but are not limited to, reddening, cracking and ichthyoses, pruritis, lichenification and excorciations. Pathological thickening of the epidermis and rete ridges, subdermal perivascular inflammatory foci are seen acutely, and in chronic cases pronounced acanthosis and hyperkeratosis are observed microscopically. Efficacy in treating, preventing and/or managing atopic dermatitis can be assessed using the dust mite antigen (Dermatophagoides farinae) induced skin model in the Nc/nga mouse as described, for example, in Yamamoto et al., Allergol Int. 2007, 56(2), 139-48.

### Methods of Suppressing Immune Responses for Organ or Cell Transplantation

In some embodiments, the invention provides a method of suppressing an immune response before or after organ or cell transplantation in a subject. In an embodiment, the subject is a mammal. In an embodiment, the subject is a human. In an embodiment, the subject is a mammal, wherein the mammal is a companion animal, such as a canine, feline, or equine.

In an embodiment, the invention provides a method of suppressing an immune response before or after organ or cell transplantation in a human subject comprising administering to said human subject a therapeutically effective amount of a BTK inhibitor, or a pharmaceutically acceptable salt or ester, prodrug, solvate or hydrate of the BTK inhibitor. In an embodiment, the invention provides a method of suppressing an immune response before or during organ or cell transplantation in a human subject, wherein the human subject is the donor of the transplant, comprising administering to said human subject a therapeutically effective amount of a BTK inhibitor, or a pharmaceutically acceptable salt or ester, prodrug, solvate or hydrate of the BTK inhibitor. In an embodiment, the invention provides a method of suppressing an immune response before or after organ or cell transplantation in a human subject, wherein the human subject is the recipient of the transplant, comprising administering to said human subject a therapeutically effective amount of a BTK inhibitor, or a pharmaceutically acceptable salt or ester, prodrug, solvate or hydrate of the BTK inhibitor. In an embodiment, the invention provides a method of treating patients with high levels of anti-allo-HLA antibodies with a BTK inhibitor prior to transplant to reduce the anti-allo-HLA burden as part of the transplant conditioning treatment. In some embodiments, the invention provides a method of treating patients with a BTK during, or after transplant to reduce de novo generation of anti-allo antibodies. In an embodiment, the invention provides a method of suppressing allograft rejection prior to, during, or after organ or cell transplantation in a human subject comprising administering to said human subject a therapeutically effective amount of a BTK inhibitor, or a pharmaceutically acceptable salt or ester, prodrug, solvate or hydrate of the BTK inhibitor. In an embodiment, the invention provides a method of the pre-transplant conditioning regimen of patients receiving solid organ transplant using a BTK inhibitor. In an embodiment, the invention provides a method of suppressing humoral acute rejection with a BTK inhibitor prior to, during, or after organ transplantation during the early post-operative stages of engraftment in a human subject comprising administering to said human subject a therapeutically effective amount of a BTK inhibitor, or a pharmaceutically acceptable salt or ester, prodrug, solvate or hydrate of the BTK inhibitor. In an embodiment, the invention provides a method of suppressing the infiltration of myeloid cells into the tissue allograft by inhibition of BTK prior to, during or after organ transplantation. In an embodiment, the invention provides a method of reducing the physiological changes associated with ischemia/reperfusion in organs following transplantation and thus reducing the pro-inflammatory signals that result in leukocyte migration. In an embodiment, the invention provides a method of inhibiting effective B cell antigen presentation to T lymphocytes during the post-engraftment phase of organ transplantation, and therefore reduces the development of allograft-specific cytotoxic and helper T cell populations, including CD8 T cells, Th1 T cells, Th2 T cells and Th17 T cells, and other pro-inflammatory T cell populations. In an embodiment, the invention provides a method of preventing de novo activation of B cells after transplantation by treatment with a BTK inhibitor at a dose that prevents signaling through the BCR in the compartment described by the transplanted organ. In an embodiment, the invention provides a method of preventing de novo activation of B cells after transplantation by treatment with a BTK inhibitor at a dose that prevents signaling through the BCR in the compartment described by the draining lymph nodes from the transplanted organ. In an embodiment, the invention provides a method of treating acute or chronic graft rejection with a BTK inhibitor after organ transplantation at a dose that prevents signaling through the BCR in the compartment described by the inflamed tissue within the transplanted organ. In any of the foregoing embodiments, the organ or cell transplantation is selected from the group consisting of heart transplantation, renal transplantation, kidney transplantation, lung transplantation, liver transplantation, ABO-incompatible transplantation, and stem cell transplantation. In some embodiments, the invention provides a method of treating a human subject wherein the human subject is a transplant recipient, comprising the step of administering a BTK inhibitor.

In an embodiment, the invention provides a method of treating a human wherein the human is a transplant recipient, comprising the step of administering a BTK inhibitor in combination with a therapy selected from the group consisting of corticosteroids, rituximab, cyclosporine, motefil mycophenylate, cyclophosphamide, belimumab, other immunosuppressive drugs, and combinations thereof. In an embodiment, the invention provides a method of treating a mammal wherein the mammal is a transplant recipient, comprising the step of administering a BTK inhibitor in combination with a therapy selected from the group consisting of corticosteroids, rituximab, cyclosporine, motefil mycophenylate, cyclophosphamide, belimumab, other immunosuppressive drugs, and combinations thereof. In an embodiment, the administration of a BTK inhibitor reduces the dosage of a therapy selected from the group consisting of corticosteroids, rituximab, cyclosporine, motefil mycophenylate, cyclophosphamide, belimumab, other immunosuppressive drugs, and combinations thereof. In any of the foregoing embodiments, the human is an adult. In any of the foregoing embodiments, the human is a pediatric patient.

In an embodiment, the invention provides a method of treating graft-versus-host disease (GVHD), comprising the step of administering a BTK inhibitor, wherein the GVHD is selected from the group consisting of GVHD associated with stem cell transplant, GVHD associated with bone marrow transplant, thymus GVHD, skin GVHD, gastrointestinal GVHD, liver GVHD, acute GVHD, and chronic GVHD.

### EXAMPLES

### Example 1. A Phase 1, Single-Center, Open-Label, Single-Treatment Study

A Phase 1, single-center, open-label, single-treatment study in healthy volunteers was conducted to assess the BTK occupancy of Formula (II) after multiple-dose administration in healthy adult subjects under fasting conditions. The study primarily focused on characterizing the pharmacodynamics (PD) of Formula (II) by assessing the BTK occupancy profile of Formula (II) in peripheral blood mononuclear cells (PBMCs) and by measuring the expression of lymphocyte B activation markers, CD69 and CD86, during and after multiple oral dose administration of 15 mg daily in healthy subjects. As a secondary objective, the study evaluated the pharmacokinetic (PK) profile, safety, and tolerability after multiple-dose administrations of Formula (II) in the healthy subjects. Moreover, the study determined the effects of Formula (II) on peripheral blood T cells and myeloid-derived suppressor cells (MDSCs).

Forty, healthy adult, non-tobacco using men and women were enrolled in the study. A 15 mg Formula (II) dose (1×15 mg capsule) was administered once daily (QD) to each subject for 7 consecutive days (Days 1 to 7) with a washout period (6 days). PD blood samples were collected from each subject before dosing on Day 1, throughout the study, and up to 144 hours after dosing on Day 7 to characterize Formula (II) PD effects. PK sampling for Formula (II) was also collected before dosing on Day 1, throughout the study, and for 24 hours after dosing on Day 7. Additionally, potential Formula (II) safety issues were monitored through physical examination, vital sign measurements, 12 lead electrocardiograms (ECGs), AEs and clinical laboratory tests.

The occupancy of BTK by Formula (II) was measured in PBMCs with the aid of a biotin tagged Formula (II) analogue probe. The effect of Formula (II) on functional activation of B cells following BCR stimulation (measured via the phorphorylation of BTK and upregulation of CD69 and CD86 activation markers) was also evaluated.

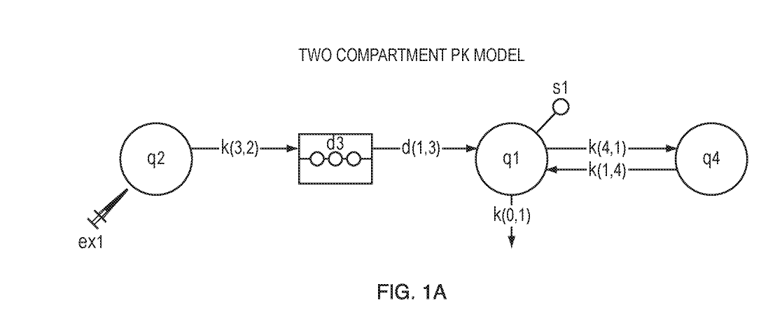

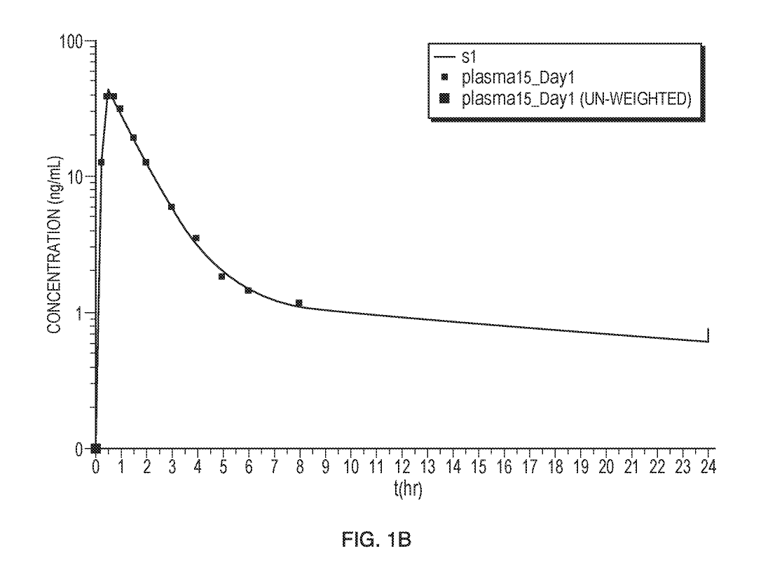

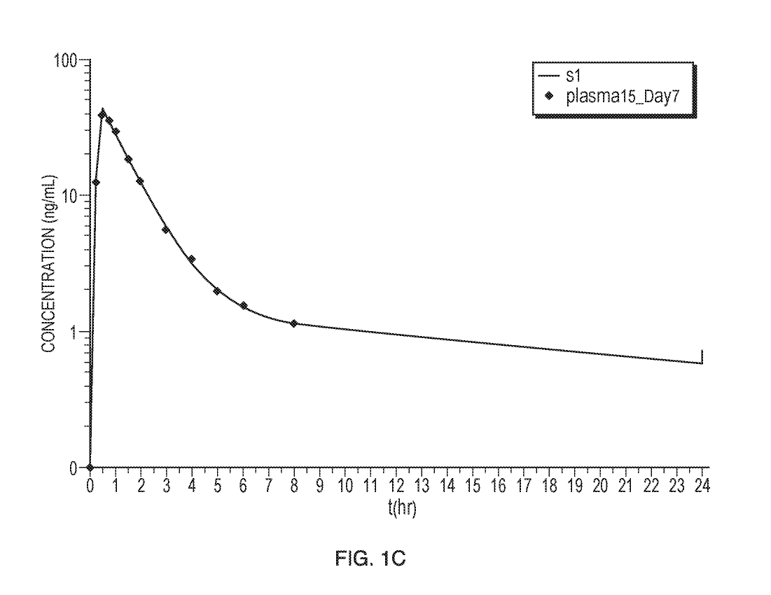

The mean measured plasma concentration of Formula (II) versus time data (PK data) and mean occupancy of BTK by Formula (II) (PD data) were fitted with biophase compartment PK and PD models, respectively to enable prediction of PK and PD for different doses, dose regimens and dose durations. The biophase compartment PK model used to fit Formula (II) concentration versus time data is depicted in FIG. 1(A). The model is a naïve, mean data, two-compartment PK model with a delay d(1,3) for oral absorption. Fitting (optimization) was accomplished via weighed least squares. The q1 compartment represents the primary compartment (i.e., the bloodstream or circulatory system), the q2 compartment represents the drug delivery point (i.e., the gut generally, the stomach, and/or the duodenum), the q4 compartment represents peripheral compartments, the rates k(3,2), k(4,1), and k(1,4) represent the intercompartment rates, the rate k(0,1) represents the output rate (i.e., clearance of Formula (II)), and s1 represents the sampling point (i.e., the bloodstream or circulatory system).

After a dose of 15 mg Formula (II) on Day 1, the model closely fit the observed mean PK concentration data on Day 1 (FIG. 1(B)). FIG. 1(C) shows the fit of the same model, with parameters fixed from Day 1, overlaid with Day 7 PK concentration data. Visually, the Day 1 model fit closely described the mean Day 7 PK data. Thus, after repeat QD dosing, the PK on Day 1 was similar to the PK on Day 7 with no apparent accumulation.

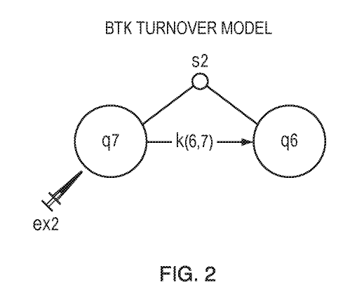

FIG. 2 illustrates a compartmental biophase BTK turnover PD model used to fit Formula (II) BTK occupancy data, wherein the q7 compartment represents un-modified BTK (i.e., BTK that is not covalently bound with Formula (II), the q6 compartment represents BTK covalently bound to Formula (II), and each compartment has a turnover rate (input rate—output rate). Output rates k(0,7) and k(0,6) were assumed to be equal. The rate constant k(6,7) is a saturable rate constant representing irreversible inactivation of BTK by Formula (II). Occupancy of the BTK kinase active site was determined by the ratio: q6/(q6+q7). The symbol s2 represents the sampling point (i.e., the central compartment or bloodstream). A reworking of this same model would allow for a different sampling point (i.e., q4, a compartment that could represent bone marrow, lymph node or other tissue compartment of interest, with different rates of BTK resynthesis than the central compartment).

The PK/PD model links the models in depicted in FIG. 1(A) and FIG. 2 and predicts the BTK target occupancy in the central compartment based on the BTK resynthesis rates empirically determined by sampling in humans treated with Formula (II), a covalent inhibitor of BTK. The flux of unmodified (new) BTK target binding sites into the central compartment reflects de novo synthesis of BTK within target cells and generation of new BTK containing target cells, in addition to the movement of target cells between peripheral and central compartments.

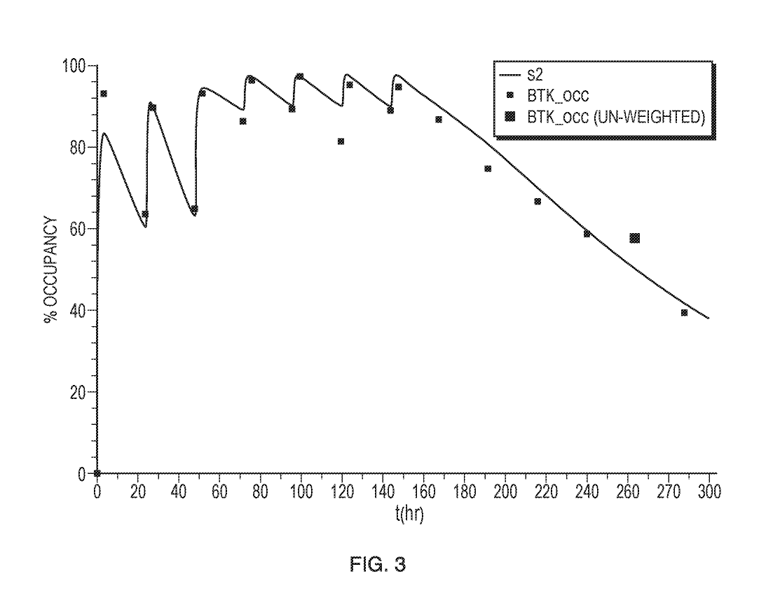

FIG. 3 illustrates the final model fit to the mean percentage BTK occupancy data obtained during the healthy volunteer study (shown as data points). During the development of the model, it was discovered that the BTK resynthesis rate was higher following administration of initial doses that resulted in a lower percentage of BTK target occupancy, as compared with the rate of BTK resynthesis after subsequent fully occupying doses. For example, during Days 1 and 2 of the 15 mg repeat dose study, the BTK target occupancy at the end of the dosing interval was approximately 60%, and the estimated half-life of occupied BTK was ˜20 hours (FIG. 3).

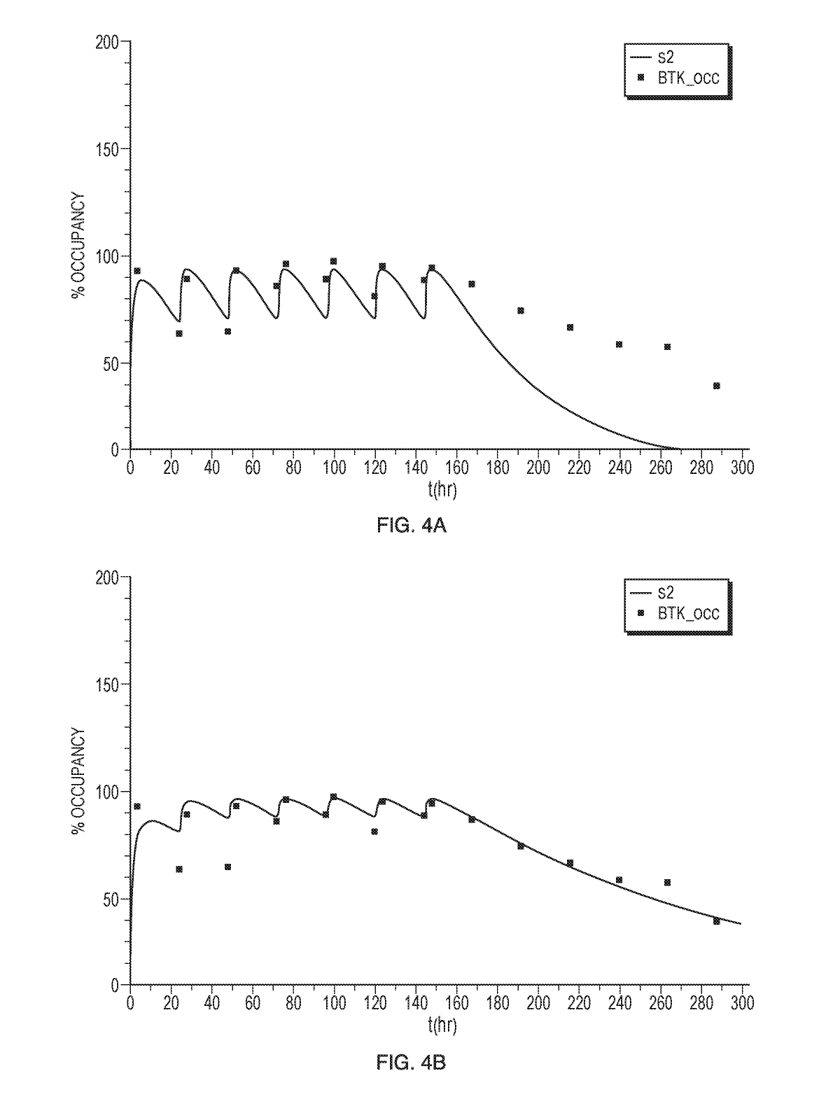

Surprisingly, repeated oral administration of the 15 mg QD dose of Formula (II) led to a high and sustained degree of BTK occupancy at steady state. The estimated half-life of occupied BTK (i.e., the time required for BTK occupancy to fall to 50% of an initial value via resynthesis of BTK) was ˜119 hours after discontinuation of dosing at steady state. The final model accommodated the change in turnover rate with time, by stepping the BTK resynthesis rate constant approximated on Days 1 and 2 to a lower fixed value on Day 3 and on the days thereafter. The data from this study led to the discovery that treatment of healthy volunteers with Formula (II) causes a change in the BTK resynthesis rate over time, from a t_{1/2} of 20 hours to a t_{1/2} of 119 hours, leading to slower declines in BTK target occupancy following attainment of steady state (FIG. 3). Early manual fits of 15 mg date showed that steady state data cannot be adequately fit with initial model parameters and vice versa, due to the change in BTK resynthesis rate (FIG. 4A and FIG. 4B).

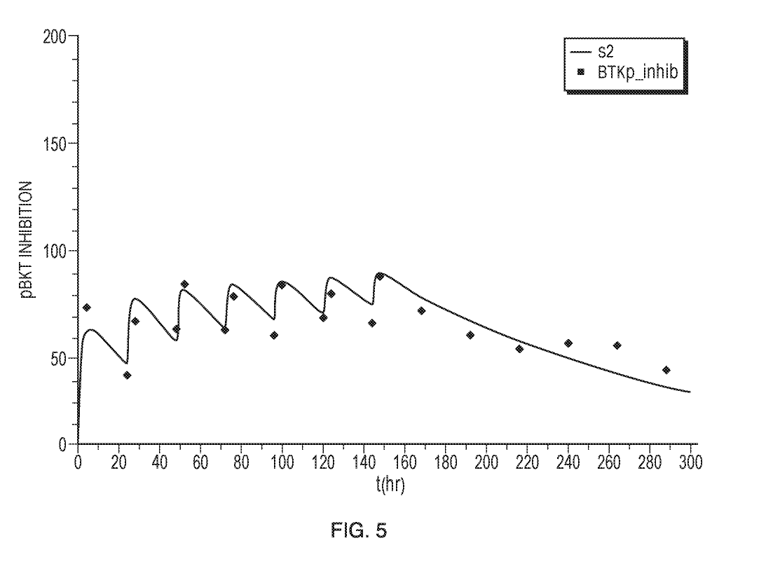

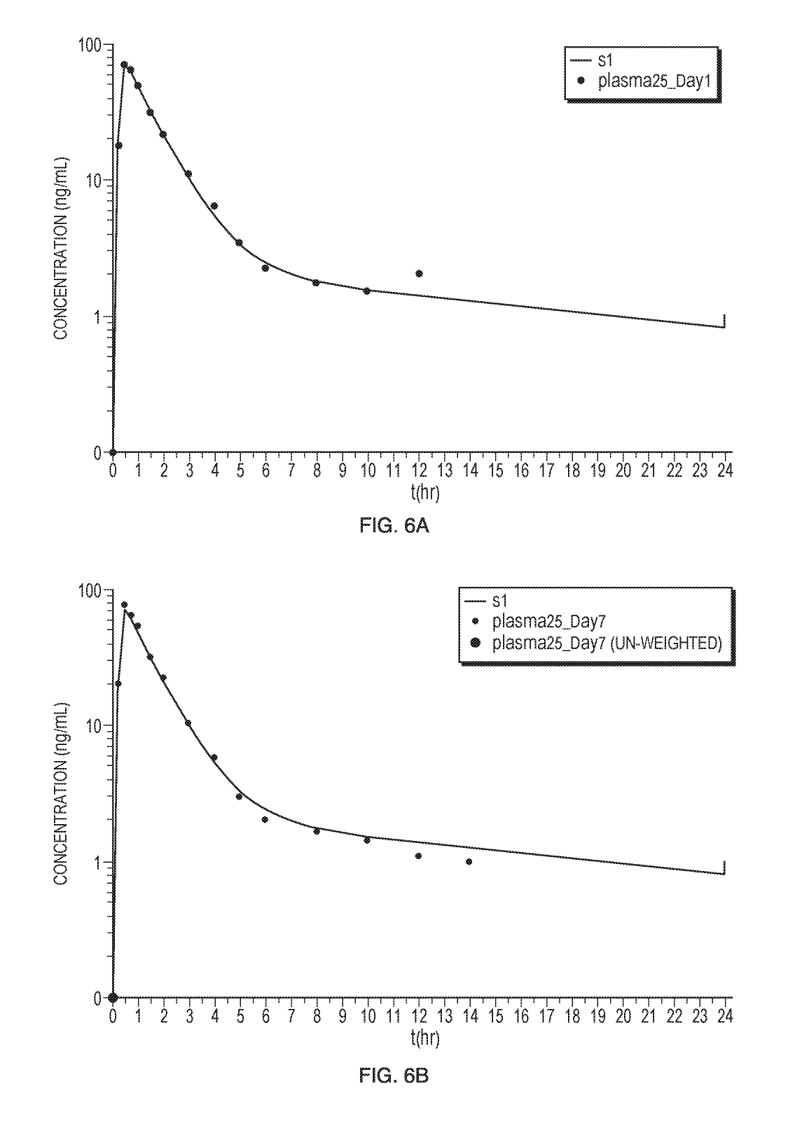

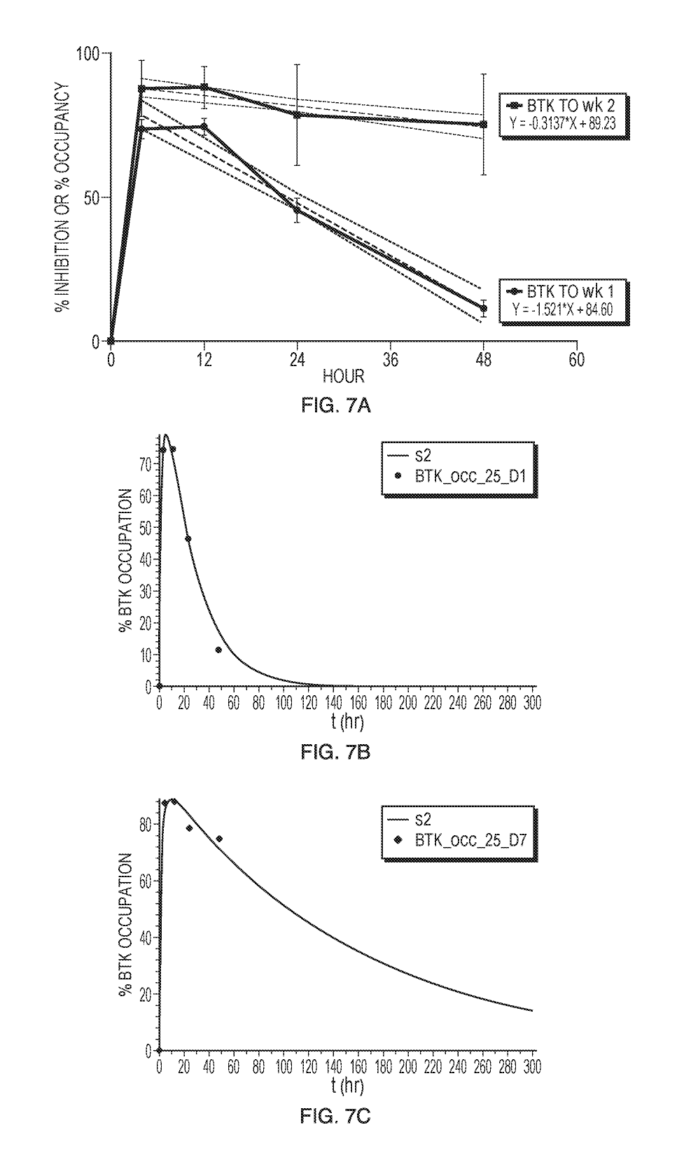

The model has potential to fit phospho-BTK data from healthy volunteers treated with 15 mg QD Formula (II) for 7 days, as illustrated in FIG. 5. Peripheral blood B cells were sampled at the indicated times and stimulated ex vivo with anti-IgM antibody to activate BCR signaling through BTK. The phosphorylation status of BTK was evaluated by phospho-flow cytometry and expressed as a percentage of the control (pre-study) sample.

Whereas there is a change in the pharmacodynamic markers of BTK inhibition and in the t1/2 of BTK target occupancy over time with Formula (II) dosing, the pharmacokinetic plasma concentration versus time profiles in plasma do not change with repeated dosing. This is congruent with pharmacokinetic half lives of Formula (II) that are much shorter than the dose interval. FIG. 6A and FIG. 6B show that the 15 mg PK model accurately predicted the pharmacokinetic profiles for a 25 mg oral dose of Formula (II) administered on Day 1 and again on Day 7 to 40 healthy volunteers, showing that the model can predict drug exposure at different doses and that exposure does not change from day to day. In contrast, the rapid BTK resynthesis rate observed after the initial 25 mg dose was lower when the second dose was given a week later (FIG. 7A, FIG. 7B, and FIG. 7C). PD parameters from the first dose in the 15 mg trial fit the occupancy data for the first 25 mg dose. Surprisingly, the PD parameters from the 7th consecutive 15 mg dose were required to fit the second 25 mg dose that was given one week after the first. These surprising data, and the increase in occupancy in the first several doses of the 15 mg study, indicated a long lived effect of a single dose of Formula II on BTK resynthesis, which manifests as an accrual of BTK occupancy in multiple dose regimens.

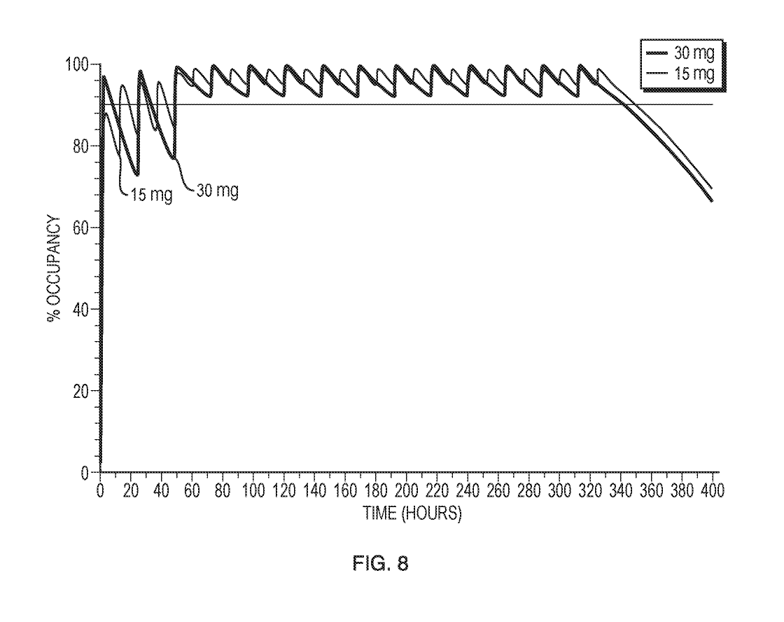

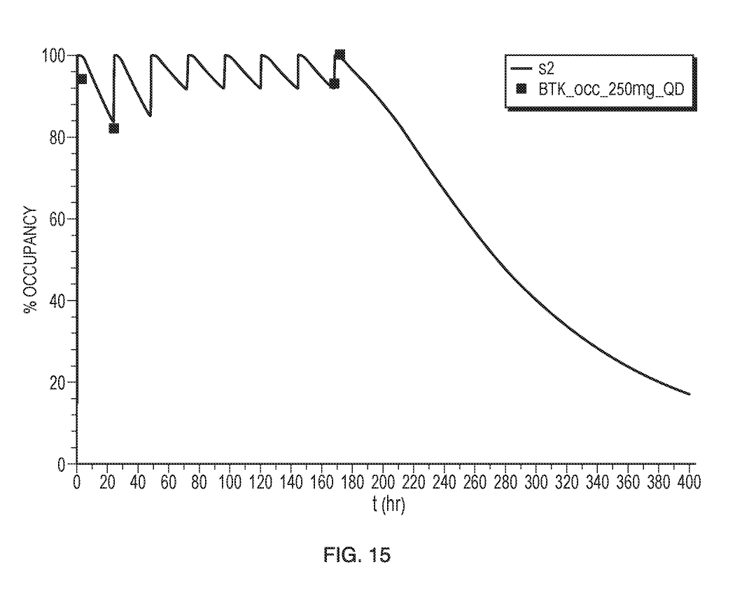

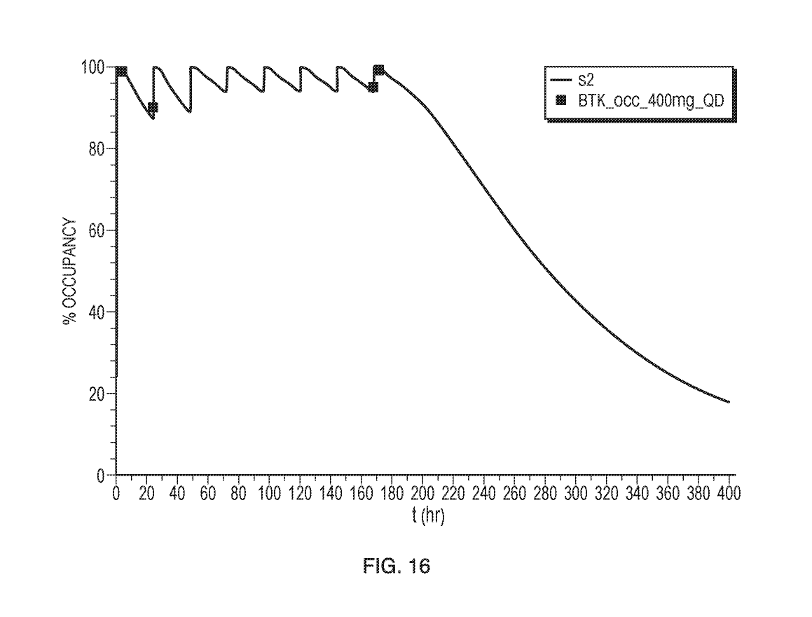

Among healthy volunteer studies with Formula (II), this phenomenon has been observed repeatedly, with a trend to observe slower BTK resynthesis rates in the compartment comprised of normal peripheral blood B cells when a higher degree of BTK target occupancy is attained with initial dosing. This discovery led to development of the PK/PD model which can accurately predict the level of BTK target occupancy across dose groups and provides a method to predict the effects of the BTK resynthesis rate on the biological efficacy of specific dosing regimens, as illustrated in FIG. 8 to FIG. 19.

The PD model was used to simulate the performance of a 15 mg BID dosing regimen of Formula (II) in comparison to a 30 mg QD dosing regimen. The results, which are shown in FIG. 8, illustrate the superior occupancy obtained by using the low dose of 15 mg in a BID dosing regimen. Surprisingly, a steady state occupancy of approximately 95% or greater is possible using a low dose of Formula (II) because of relatively low resynthesis rate of BTK in healthy volunteers.

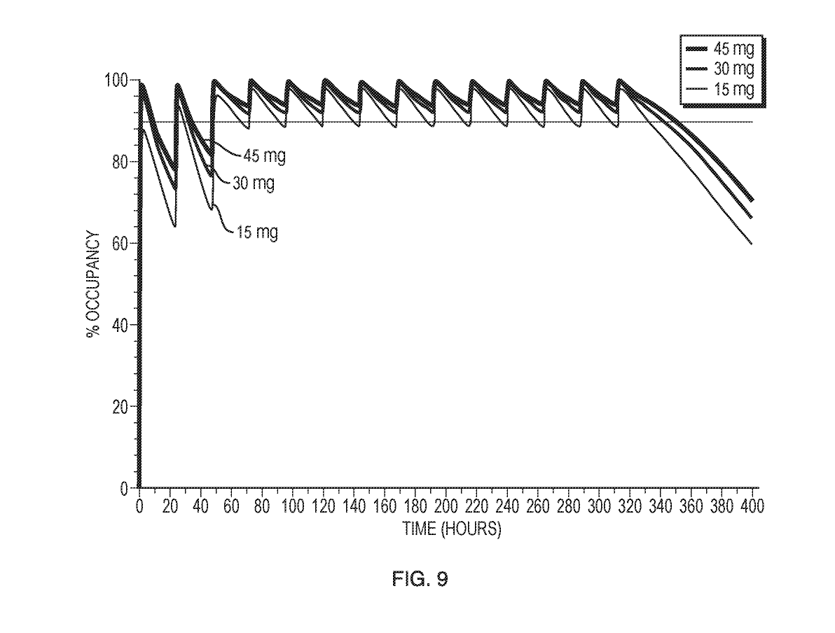

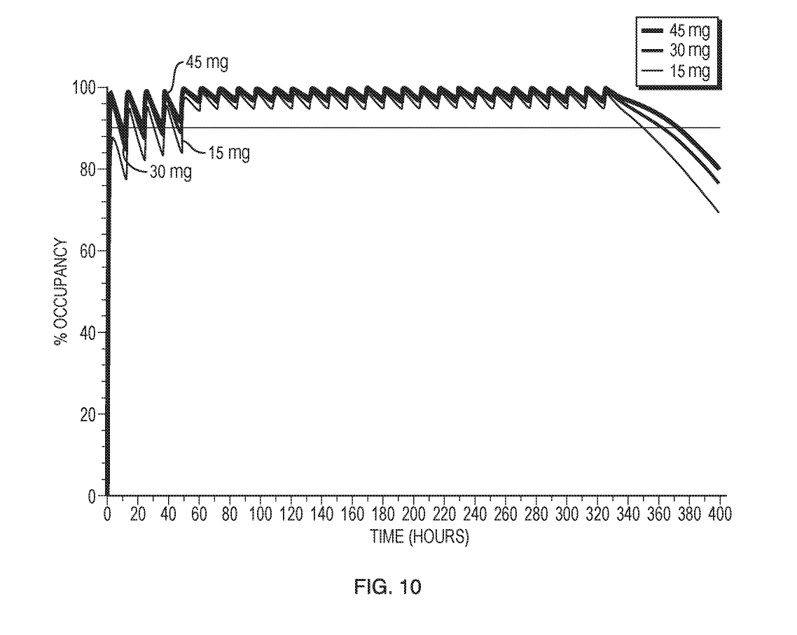

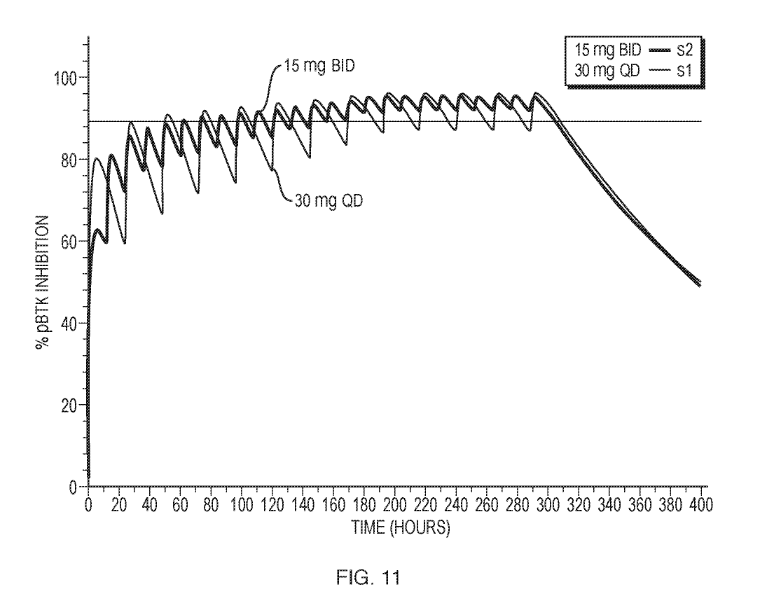

The PD model was used to simulate the performance of 15, 30, and 45 mg QD dosing regimens of Formula (II), as illustrated in FIG. 9 and to simulate the performance of 15, 30, and 45 mg BID dosing regimens of Formula (II), as illustrated in FIG. 10. The inhibition of BTK, as measured by pBTK levels following ex vivo stimulation of the BCR in normal peripheral blood B lymphocytes, was simulated for dose regimens of 15 mg BID in comparison with 30 mg QD in healthy volunteers (FIG. 11).

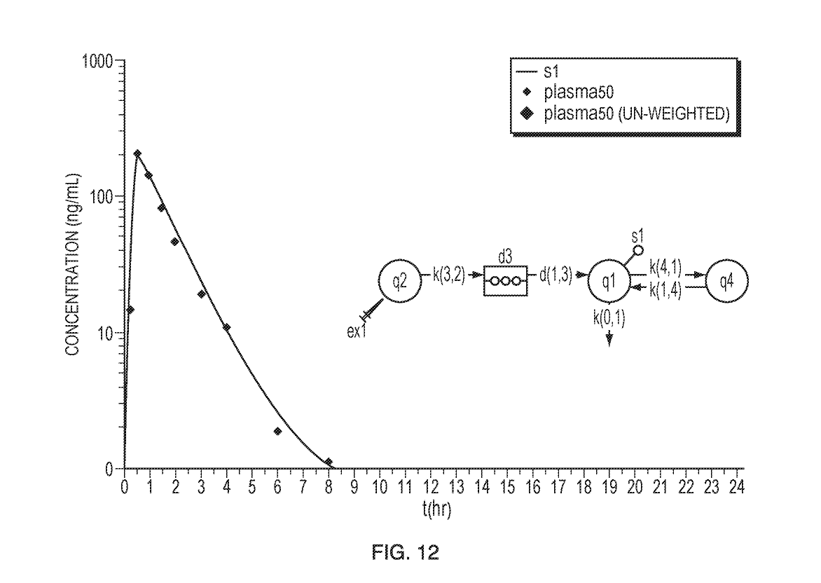

In the 15 mg and 25 mg healthy volunteer studies Formula (II) PK was well behaved with little to no accumulation or change in PK after repeat dosing. Model PK parameter estimates based on 15 mg PK data were essentially unchanged when the model was fit to 25 mg PK data (FIG. 7A, FIG. 7B, and FIG. 7C). However, the PK model based upon these lower doses did not scale well to 50 mg PK data obtained in healthy volunteers in a dose escalation study unless the k(4,1) rate constant was decreased (FIG. 12).

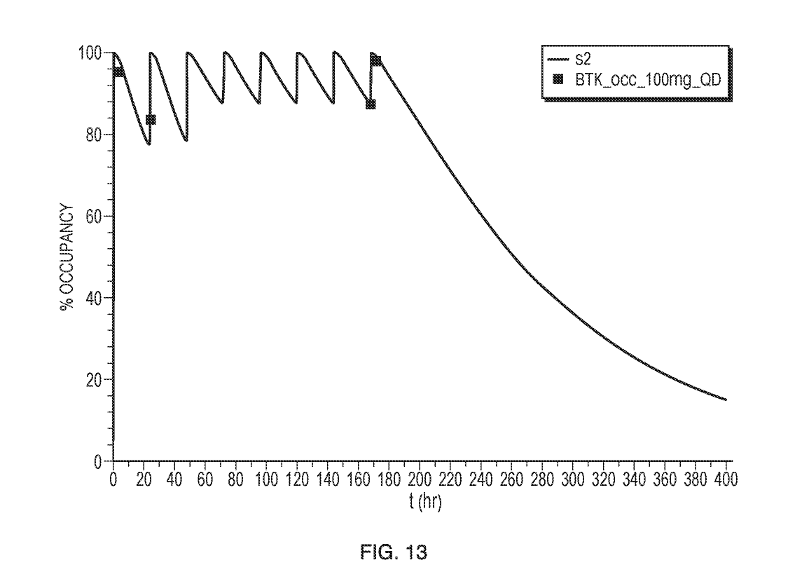

BTK resynthesis rate varies by disease and disease burden, and differs from BTK in the normal tissues of healthy volunteers. As such, in FIG. 13 to FIG. 16, the PK/PD model was used to estimate steady state BTK occupancy for higher Formula (II) doses, i.e., oncology doses, using the adjustment to k(4,1) derived from fitting the higher dose PK data. Mean patient data are overlaid on the simulated BTK target occupancy profiles showing good steady state model approximations to mean occupancy data obtained at doses of 100 mg QD, 100 mg BID, 250 mg QD and 400 mg QD. The data are limited by the sparse two point mean estimates of occupancy at steady state obtained in the clinical studies, as opposed to the multiday washout data obtained at lower doses in healthy volunteers. Nonetheless, the estimates provide additional support for the performance of the PK/PD model.

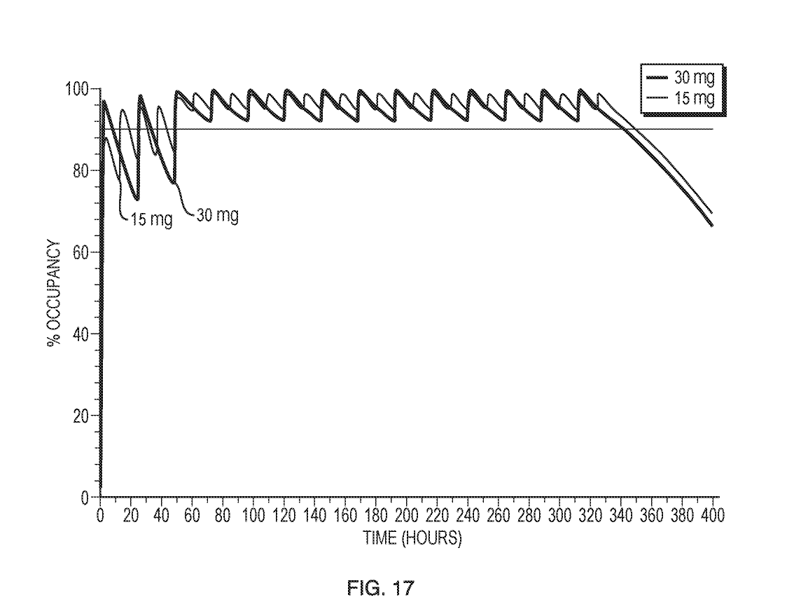

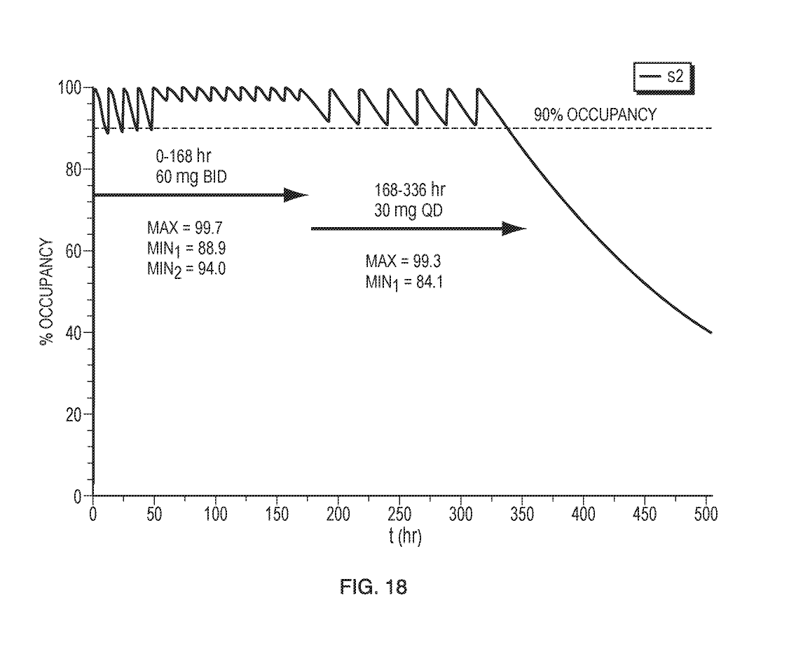

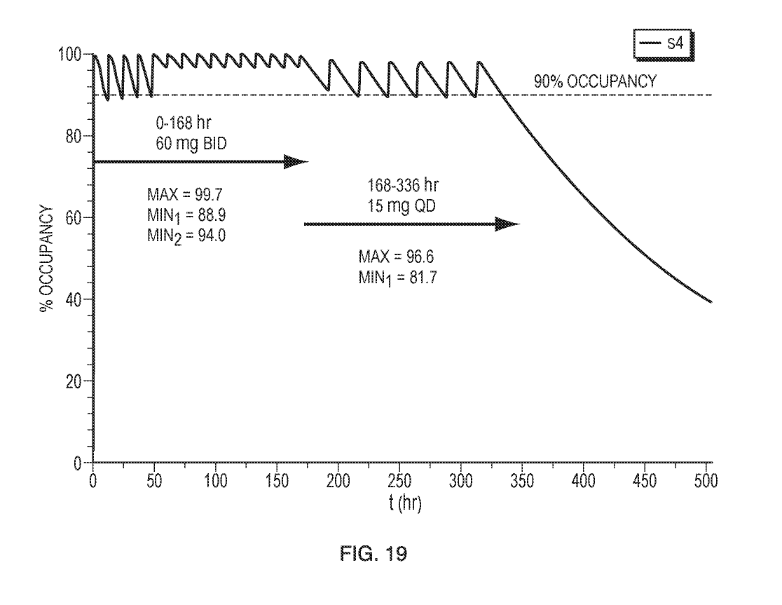

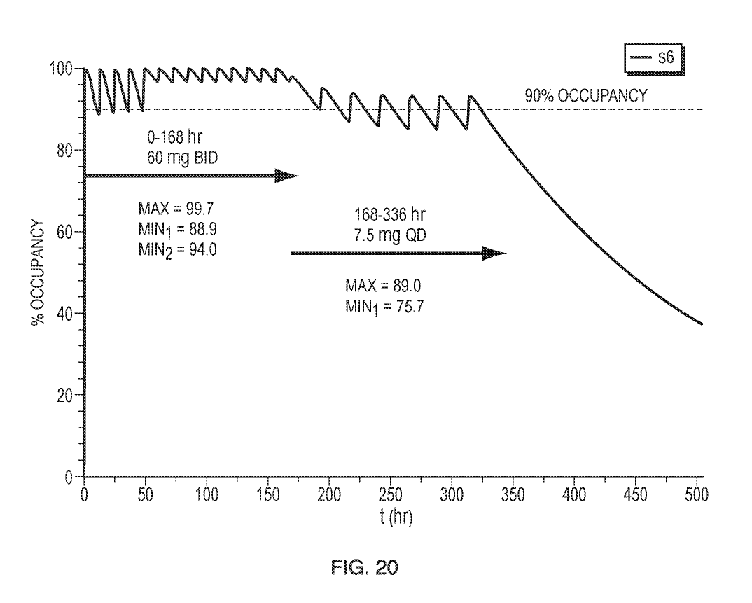

FIG. 17 to FIG. 20 show results of simulations to predict BTK target occupancy over two weeks of dosing Formula (II) with different dosing strategies. The PK/PD model was applied to predict BTK occupancy for different dose regimens over 2 weeks of dosing (FIGS. 17-20). FIG. 17 compares a 30 mg QD to a 15 mg BID dose regimen. FIG. 18 shows a 60 mg BID loading dose followed by a 30 mg QD maintenance dose after one week. FIG. 19 shows 60 mg BID loading dose followed by a 15 mg QD maintenance dose after one week and FIG. 20 shows a 60 mg BID loading dose followed by a 7.5 mg QD maintenance dose after one week. These dosing regimens are examples of strategies which could be used to control BTK mediated disease states in which the levels of BTK resynthesis change during acute and chronic forms of the disease, with disease control, or during the course of disease amelioration.

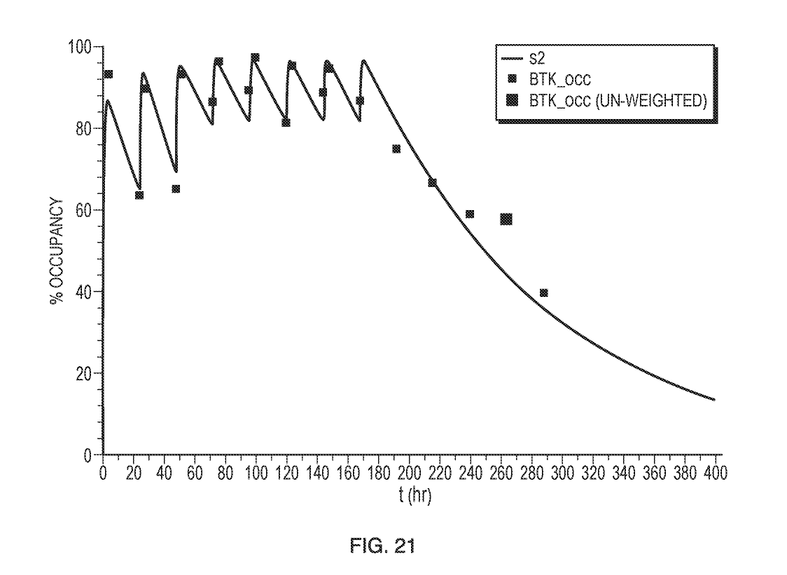

FIG. 21 illustrates the model fit to the 7-day BTK target occupancy results from healthy human volunteers after oral dosing with 15 mg QD Formula (II), demonstrating that at steady state, peak occupancy >90% is achieved with this low dose after the resynthesis rate declines.

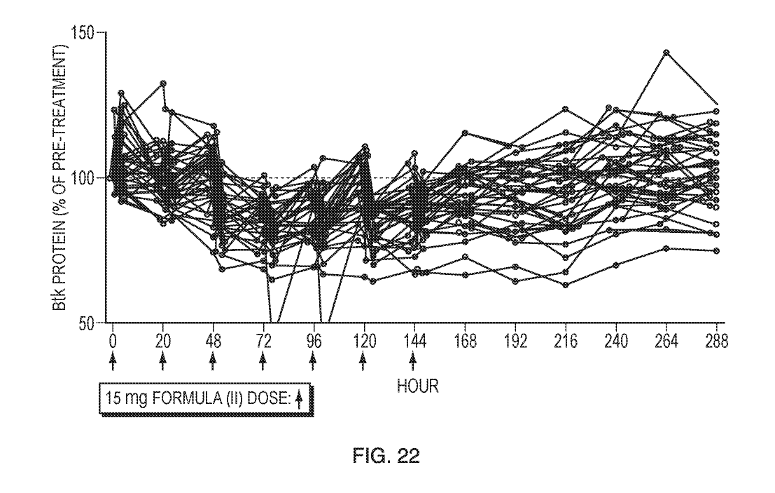

FIG. 22 illustrates the surprising discovery that initially during treatment of healthy human volunteers with Formula (II), the mean intracellular protein levels of BTK increased, as measured by flow cytometry after the initial dose. Remarkably, a decrease was observed in intracellular protein levels of BTK at the same critical time (between Day 2 and Day 3) as the modeled BTK resynthesis rate was found to decrease. The effects of Formula (II) on BTK resynthesis rates in healthy human volunteers is an important novel finding that was confirmed by the two orthogonal methods (i.e., flow cytometry and BTK target occupancy ELISA combined with PK modeling). A similar trend in the BTK resynthesis rate was discovered as a dose responsive (rather than time response) phenomenon after dosing healthy human volunteers across a range of QD and BID doses for a single day, as illustrated in FIG. 23.

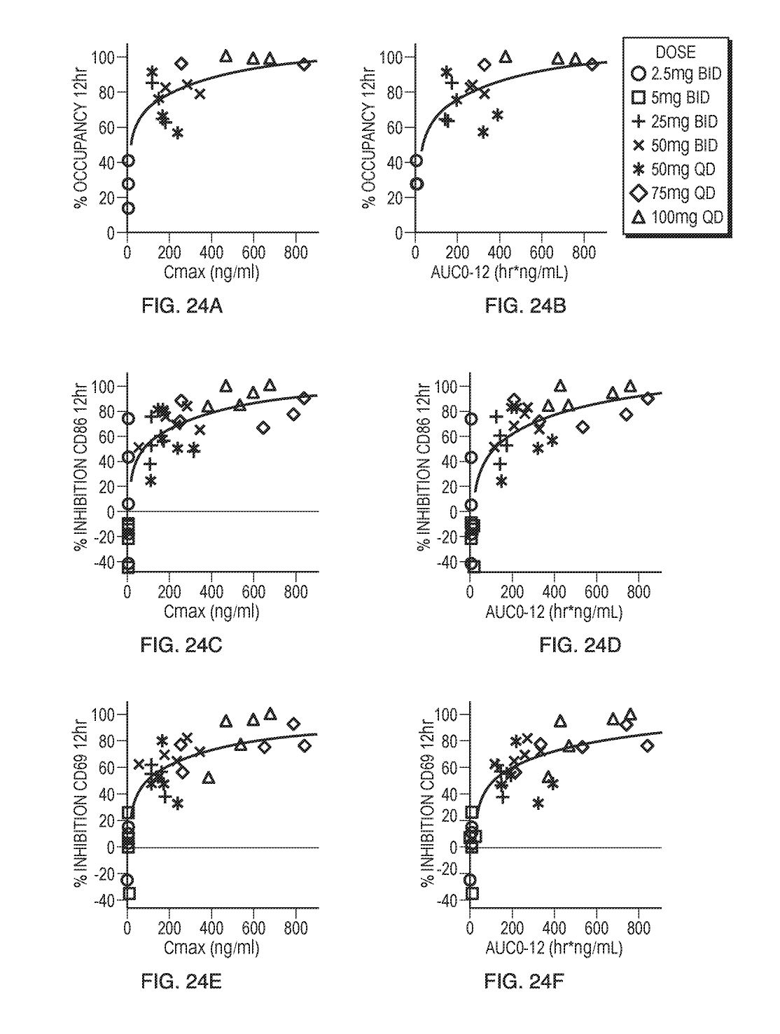

As illustrated in FIG. 24A, FIG. 24B, FIG. 24C, FIG. 24D, FIG. 24E, and FIG. 24F, the link between exposure and response can be described by Emax curves that estimate pharmacodynamic effects at given plasma concentrations of the active agent. Pharmacodynamic data from healthy volunteers administered a single dose of Formula (II) at 50, 75 or 100 mg, or two doses of Formula (II) at 2.5, 5, 25 or 50 mg are presented. The data are from samples obtained 12 hours into the wash-out period, representing an integrated BTK resynthesis rate and the degree of functional return among the subjects in each dose cohort. These results did not predict the ability of repeated low doses of Formula (II) to substantively increase the BTK target occupancy and provide deeper inhibition of downstream PD markers of BCR signaling, as was observed with 7 days of dosing at 15 mg QD.

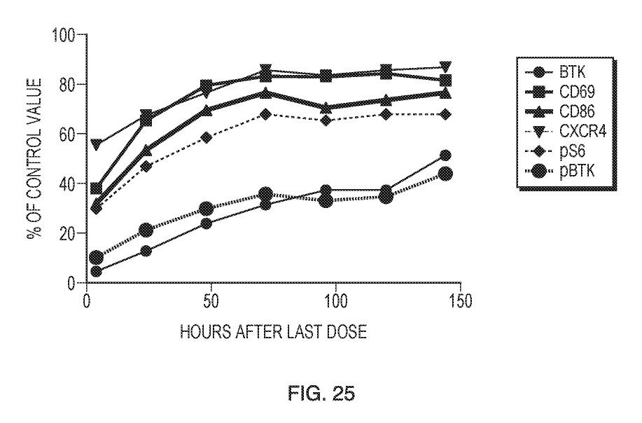

The PK/PD model was used to simulate pBTK inhibition for Formula (II) using a 15 mg BID and 30 mg QD dosing regimens (FIG. 11). Inhibition of pBTK was 92% or greater for the BID regimen. Resynthesis rates from the end of the treatment interval for BTK, and the return of function for BTK and downstream markers of BCR stimulation such as CD69 up-regulation, CD86 up-regulation, and CXCR4 down-regulation, are shown in FIG. 25. The difference between the percentage BTK occupancy/inhibition and the return of function for the selected downstream PD markers is likely due to compensatory signaling pathways activated by ex vivo BCR stimulation in normal B cells.

The adjustments to BTK resynthesis rate in the PD model were required due to a feedback loop in the BTK pathway, wherein the partial inhibition of BTK stimulates an increased rate of BTK resynthesis and stronger inhibition leads to a reduced rate of BTK resynthesis. The accrual of BTK target occupancy to meet the resynthesis demands in the tissue compartment of therapeutic interest, and/or in the tissue compartment that has the most rapid BTK resynthesis rate, is therefore essential to overcome compensatory mechanisms. Although the signaling pathway through NFkB was reported to induce mRNA expression, the discovery that treatment with low doses of Formula (II) leads to the modulation of BTK in humans is a novel finding that has not been previously reported. This combined with the development of a PK/PD modeling tool that effectively describes the exposure-response relationship for a covalent BTK inhibitor, including long-lasting actions on the BTK and the BCR pathway long after the plasma Formula (II) levels have diminished, provides a unique and novel method for identifying effective low-dose regimens for the treatment of autoimmune disorders, allergic and atopic diseases, inflammation, and other chronic maladies that may respond to selective BTK inhibition.

In summary, the results of the healthy volunteer studies are as follows. A PK/PD model was developed based on low doses in healthy volunteers and models were adjusted to fit data for higher doses based on PK data from oncology patients. The PK of Formula (II) was well behaved, with little to no accumulation or change in PK after repeat dosing. Model PK parameter estimates based on 15 mg PK data scaled reasonably well to 25 mg PK, above that an adjustment of the rate constant between the central compartment and the peripheral compartment was needed to simulate PK for the higher doses used in oncology. In the PD model, changes in the rate of BTK resynthesis with time were observed after a single dose of Formula (II). This may reflect change in parameters such as the migration of lymphocytes between compartments (i.e., from peripheral compartments with faster BTK resynthesis rates to the central compartment), as well as an increased rate of BTK protein synthesis. The total BTK per cell in the central compartment was increased after doses that resulted in incomplete BTK target occupancy and was decreased after doses that resulted in full BTK target occupancy, consistent with a feedback loop involving BTK or a downstream member of the BTK signaling pathway in the control of BTK resynthesis rates. The modeled rate of BTK resynthesis (t_{1/2}=119 h) appears to be fit the data from healthy volunteers after 7 days dosing with 15 mg, and after a 25 mg BID dose of Formula (II). Early doses of 15 mg QD, or a single 25 mg QD dose, had much faster BTK resynthesis rates (t_{1/2}=20 h).

In healthy volunteers, BID dosing increased the trough occupancy to >90% after 2 weeks of repeat 15 mg dosing. Similar results were obtained with pBTK. A visual check of modeled data indicated an approximate steady state in BTK occupancy was attained by about 8-9 days. However, the time to attain an apparent steady state of high BTK occupancy was reduced with the higher dose levels. With the loading dose/maintenance dose regimen, high mean BTK occupancy was rapidly achieved and the >90% mean BTK occupancy at C_{trough} was subsequently achieved with lower BID doses of Formula (II).

### Example 2. A Rat Study Comparing QD Bolus Administration by Oral Gavage and Low Dose Continuous PO Administration of Formula (II) in Chow: Pharmacokinetic Profiles and BTK Target Occupancy

To evaluate the effects of continuous low dose administration versus oral gavage administration of Formula (II), six male rats per group were treated with oral gavage administration of vehicle or Formula (II) at a dose of 30 mg/kg/day for 14 days. Six male rats per group were treated with dietary Formula (II) in chow at concentrations of 100 and 500 ppm; a control group (no vehicle, no chow) was also included in the study. All animals were evaluated for pharmacokinetics on Day 14, during the night cycle to accommodate the dietary groups. The spleens were collected at necroscopy and splenocytes were harvested and cryopreserved for BTK target occupancy analysis. The presence of inactivated BTK in normal splenocytes was measured using a BTK active-site specific probe in an ELISA assay. Plasma concentrations of Formula (II) were measured using liquid chromatography/mass spectroscopy; pharmacokinetic parameters were estimated using WinNonlin (Pharsight, Cetara).

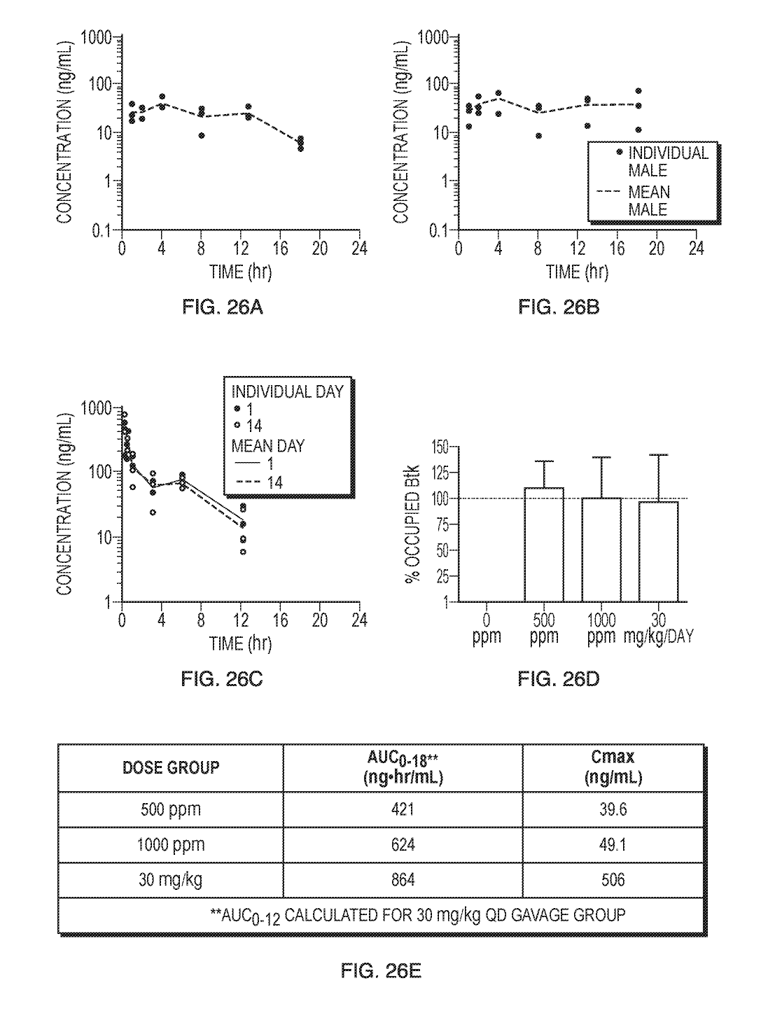

In the group treated by oral gavage, there was rapid absorption of Formula (II), and the Cmax was relatively high (506 ng/mL) when compared with the AUC0-12 value (864 ng·h/mL). In contrast, the rats treated with the dietary formulations had relatively flat concentration versus time profiles and prolonged exposures over the feeding cycle, with Cmax values ≦ 1/10^{th} of the AUC0-18 values, as illustrated in FIG. 26A, FIG. 26B, FIG. 26C, FIG. 26D, and FIG. 26E. In these unstimulated rats there was clear evidence of activity of Formula (II) in the tissue compartment of interest, as remarkably the splenocytes from the dietary groups had similar BTK target occupancy as the splenocytes from the 30 mg/kg/day gavage group. This strongly supports that low level, prolonged administration of Formula (II) with an extended release PO formulation or a controlled release PO formulation that delivers small or pulsatile doses, could achieve a therapeutic degree of target occupancy in diseased tissues, provided the dose is sufficient to address the rate of BTK synthesis therein.

### Example 3. Return of BCR Function after Formula (II) Treatment to Inhibit BTK in a Mouse in Vivo Model

B cell stimulation through the B cell receptor (BCR) with antibodies (e.g., anti-IgM) results in the activation of BTK and subsequent cellular changes, including the upregulation of various cell surface receptors. Measuring the inhibition of BCR induced protein phosphorylation downstream of Btk, and the inhibition of surface receptor upregulation provides a way to assess the Btk inhibitor activity. In the first study, the BTK target occupancy in splenocytes was measured over time, providing evidence for BTK resynthesis in the tissue compartment of interest.

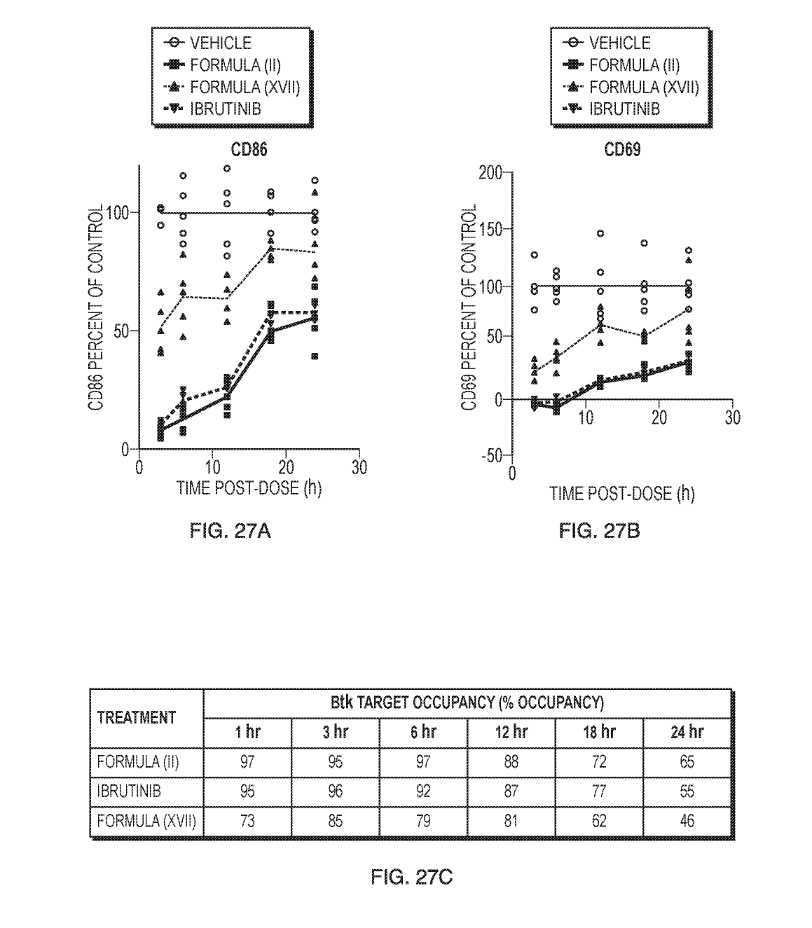

Cohorts comprising 25 mice each were given a single oral dose of 25 mg/kg of ibrutinib, CC-292 (Formula (XVII)), or Formula (II). Mice were sacrificed at 3, 6, 12, 18, and 24 hours after treatment with the BTK inhibitors (FIG. 27A, FIG. 27B, and FIG. 27C). Spleens were harvested and splenocyte preparations were made for flow cytometry analysis of B cell functions. Fresh or cryopreserved splenocytes were cultured with a-IgM to stimulate BCR, and the up-regulation of CD69, an early functional marker of B cell activation, was analyzed by flow cytometry.

As shown in FIG. 27A, FIG. 27B, and FIG. 27C, Formula (II) and ibrutinib showed near complete inhibition of ex vivo anti-IgM induced responses in B cells 3 hours post-dose, with partial inhibition of CD86 and CD69 expression persisting for 24 hours. In contrast, incomplete inhibition was observed for Formula (XVII) (CC-292) at 3 hours, consistent with incomplete BTK target occupancy by Formula (XVII). Over the entire time course, the CD86% inhibition for Formula (II), ibrutinib, and Formula (XVII) (CC-292) were 67.9%, 62.9%, and 28.1% respectively; and for CD69, 84.3%, 83.4%, and 44.8% respectively.

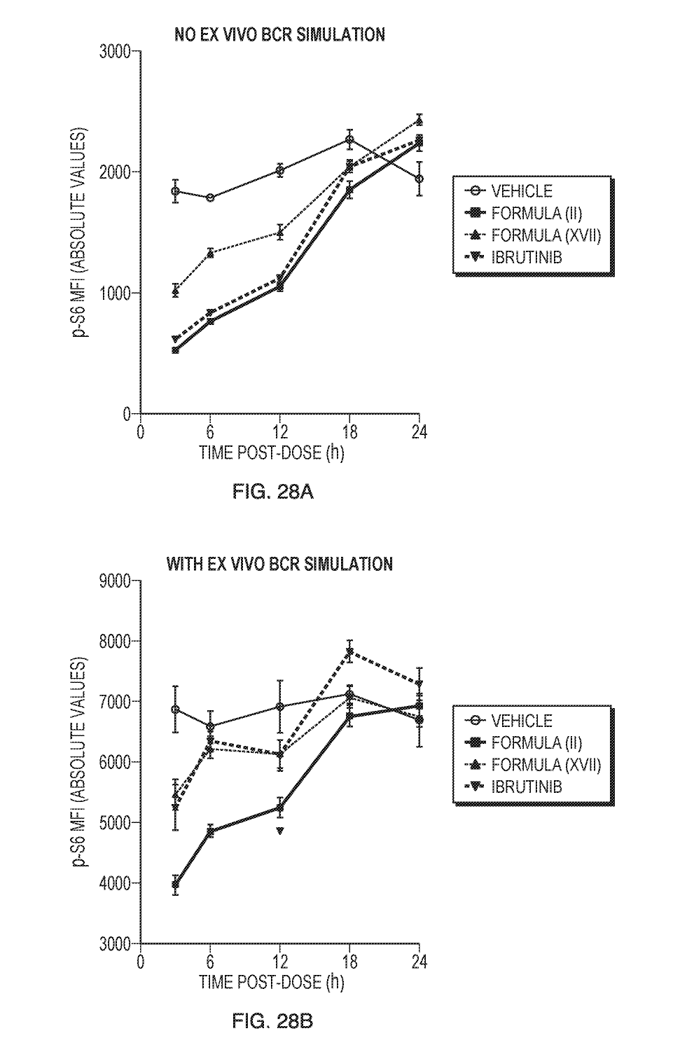

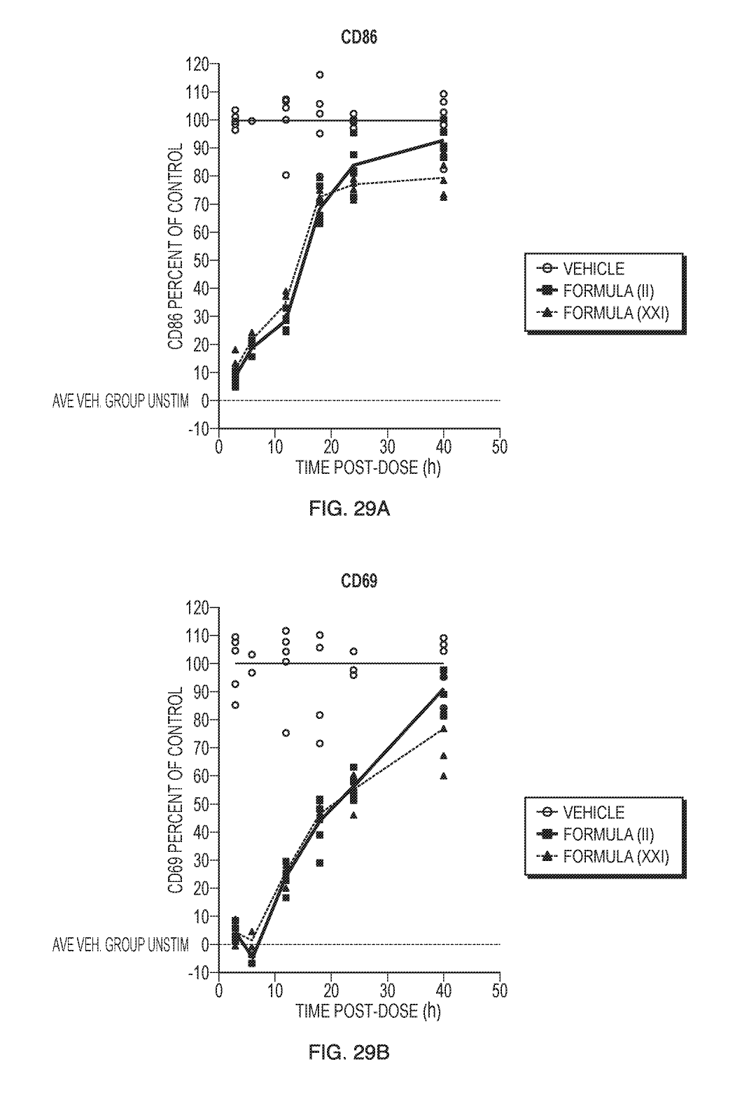

A downstream marker of BTK activity, S6 phosphorylation, was also monitored in this study. The three BTK inhibitors inhibited both basal state and anti-IgM induced S6 phosphorylation and showed the same rank-order of activity (Formula (II)>ibrutinib>Formula (XVII)), as illustrated in FIG. 28A and FIG. 28B. In a second study, the effects of another inhibitor of BTK that covalently inactivates the kinase, Formula (XXI), were compared with Formula (II) to evaluate the return of BCR function and B cell activation, as illustrated in FIG. 29A and FIG. 29B.

Evaluation of the BTK target occupancy in splenocytes from mice demonstrates that BTK resynthesis rates among the groups treated with covalent inhibitors of BTK were slower than the BTK resynthesis among the group treated with Formula (XVII) (CC-292). This suggests an additional component of BTK target resynthesis in the tissue compartment (spleen) comprising of (1) the BTK protein liberated by reversal of the binding of Formula (XVII) and (2) the increased resynthesis rate of BTK within splenocytes in which BTK signal transduction was only partially inhibited. Comparison with the BTK resynthesis analysis in human subjects treated with Formula (II) shows that splenocytes from treated mice mice have a faster resynthesis rate following a single oral dose than was observed in human B cells.

### Example 4. Evaluation of BTK Target Occupancy in Peripheral Blood and Nodal Lymphoma Lesions of Companion Dogs with Spontaneously Occurring Lymphoma Following Treatment with Formula (II)

Dogs were administered Formula (II) orally once daily and evaluations occurred every 7 days for the first 4 weeks of the study, then every 14 days thereafter. PBMCs and lymph node fine needle aspirates (lymphoma patients only) were obtained for PD analysis on day 0 at time 0 hr and 3 hr, and again at pre-dose (Cmin) on day 7 of drug administration. In each cohort, PK analysis was performed in 2 dogs on day 14 of the study consisting of blood sampling at 0, 0.5, 1, 2, 3, 4, 6, 8, 12, and 24 hr following drug administration.

Peripheral blood mononuclear cells (PBMCs) were isolated from blood and the CD21+ B-cells were enriched by positive magnetic selection. For fine needle aspiration (FNA) samples, erythrocytes were lysed with ammonium chloride lysis buffer. Purified peripheral B-cells and FNA samples were snap-frozen for measurement of BTK target occupancy. BTK target occupancy was measured using an ELISA with anti-BTK as a capture antibody and a biotinylated active site probe for BTK, followed by streptavidin-HRP as a detection reagent. The ex vivo incubation with Formula (II) provided a baseline signal for each sample. BTK occupancy in the B cell lysates from peripheral B-cells and lymph node FNA preparations was calculated as a percentage of the control after correction for baseline chemoluminescence. All samples were run in quadruplicate.

Full BTK occupancy (defined as ≧90% BTK occupied) occurred in Peripheral B-Cells in all cohorts 3 hr after administration. Full BTK occupancy was also observed in the day 7 pre-dose peripheral blood sample at steady state (at 24 hr [QD cohorts] or 12 hr [BID cohorts] after dose administration). The 5 mg/kg QD cohort was excluded from analysis due to sample quality, and no FNA samples were taken from the 2.5 mg/kg QD cohort. Full BTK occupancy (>90%) was observed in the lymph node FNAs for all cohorts at 3 hr after administration. In all measured cohorts, mean BTK occupancy was lower in lymph node FNAs at pre-dose on treatment day 7, either 24 hr (QD cohorts) or 12 hr (BID cohorts) after dose administration, compared to in the peripheral blood sample.

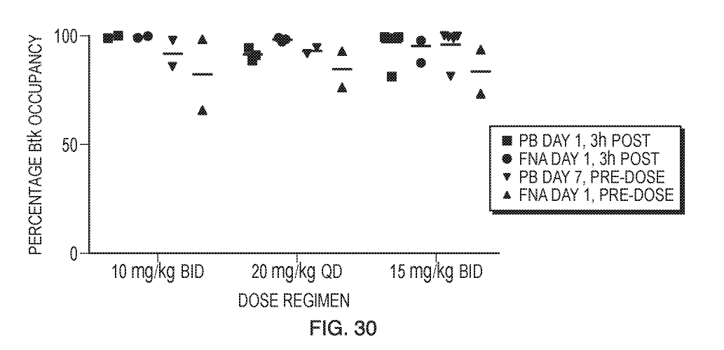

FIG. 30 shows that after administration of Formula (II), matched samples from peripheral blood mononuclear cells and FNAs from tumor bearing lymph nodes showed full BTK occupancy in the blood and lymph nodes at 3 hr post-dose. However in the compartment of tumor-bearing lymph nodes, the target occupancy was lower than that observed in the peripheral blood at the pre-dose Day 7 timepoint. This is consistent with the higher rate of BTK resynthesis in the proliferative tumor compartment, compared to the normal B lymphocytes that were sampled in the central (peripheral blood) compartment. Although high occupancy was observed in tumor bearing lymph nodes (82-88%) the faster resynthesis rate accounts for the shorter half-life when compared with peripheral blood.

### Example 5. Dose-Response of BTK Target Occupancy, and Effect on Cellular Total BTK Protein Expression after Formula (II) Treatment in Relapsed and Refractory CLL Patients

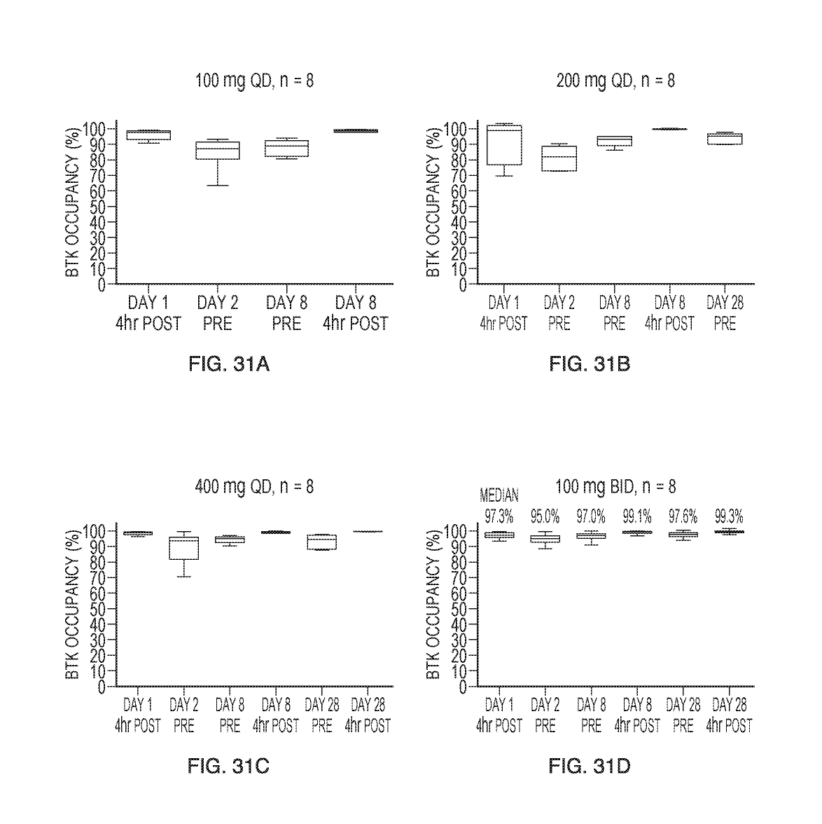

Prior dosing of Formula (II) was performed using much higher dosages, from 100 to 400 mg QD or 100 to 200 mg BID, based on the establishment and maintenance of a high degree of BTK target occupancy during the dose escalation phase of a trial in subjects with relapsed/refractory CLL. FIG. 26 shows the BTK target occupancy results from selected dose groups over the first 28 days of treatment. Evaluation of circulating tumor cells within the peripheral blood compartment demonstrated that total daily doses above 200 mg, delivered with once-daily or twice-daily dosing schedules, resulted in full (>90%) BTK target occupancy throughout the dosing interval in CLL patients. However, higher percentage BTK occupancy was observed in subjects dosed with 100 mg BID, compared with any QD dosing schedule. With treatment regimes employing daily doses up to 400 mg, the evaluation of BTK occupancy demonstrates that a swift conversion to fully occupied BTK occurred during the absorption and early elimination phases of the pharmacokinetic concentration-time profile. In all treated subjects, samples taken at 3-4 hours post dose from the peripheral blood compartment showed full BTK occupancy, which was maintained despite decreased in plasma concentrations of Formula (II). Notably, the resynthesis of BTK following 100 mg QD dosing with Formula (II) was more rapid than after 100 mg BID dosing, as illustrated in FIG. 31.

The rapidly occurring Tmax at ˜1 hour following oral administration of Formula (II), and the sharp peak in plasma concentrations were sufficient to saturate BTK sites while the covalent interaction at the kinase target site resulted in prolonged target occupancy in this patient population. Penetration of the Formula (II) into tissue compartments such as bone marrow, spleen and tumor affected lymph nodes was demonstrated by the advent of or increase in CLL lymphocytosis within the peripheral blood compartment, which was readily evident in both the hematology samples taken during the first week of dosing and in the PBMC samples used for evaluation of BTK target occupancy. In this indication, the rapid resynthesis of BTK in the tumor cells in the CLL cell compartment is observed as a steeper rate of decline in the percentage of BTK sites occupied by Formula (II), compared with what has been observed in normal peripheral blood cells of healthy volunteers (compare linear slope estimates of FIG. 25 with FIG. 34).

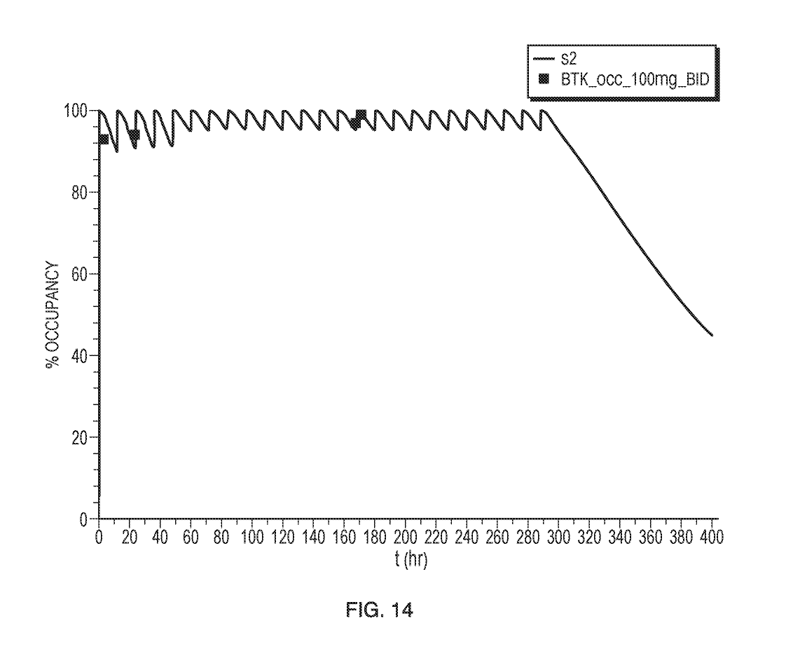

FIG. 14 demonstrates a good fit of the Formula (II) repeat dose BTK occupancy data using a modification of the PK/PD model described above, with a slower k(4,1) rate than was used at the lower doses. This rate reflects penetration to peripheral compartments and may involve a saturable component of target mediated elimination.

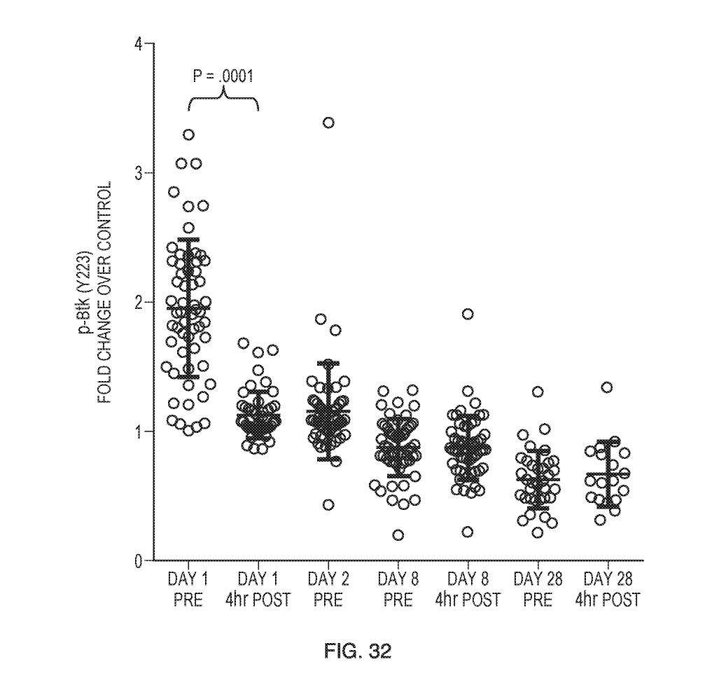

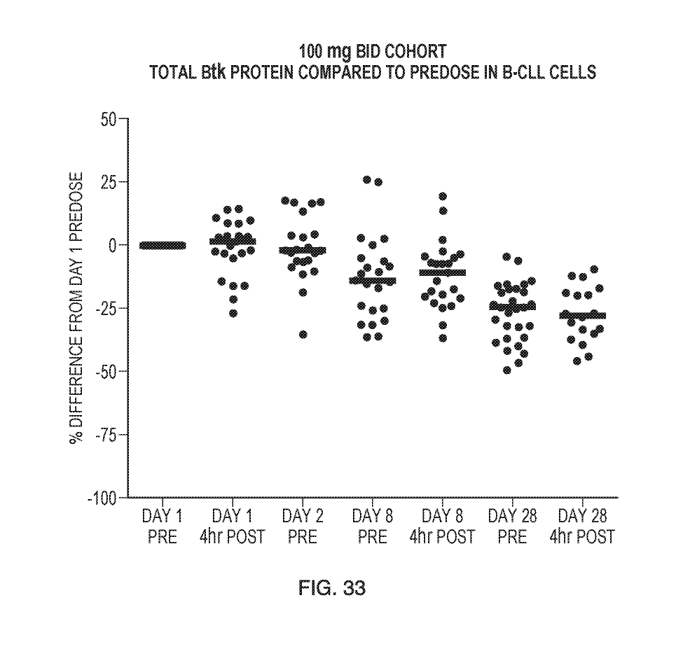

In CLL patients, BTK target occupancy leads to decreased BTK activation following ex vivo BCR stimulation, as illustrated in FIG. 32. Surprisingly, we also noted that treatment with Formula (II) caused a reduction in the cellular BTK content in the CLL tumor cells sampled after the attainment of full BTK target occupancy. This finding demonstrates that in B-CLL, the BTK the resynthesis rate can be altered by treatment as was discovered with Formula (II) in healthy volunteers (i.e., normal B cells). The higher rates of BTK resynthesis in the relevant tissue compartments of cancer patients and patients with autoimmune or inflammatory disorders can be addressed by adjusting the dosing strategy to the compartment of interest.

### Example 6. BTK Inhibitory Effects on Solid Tumor Microenvironment in an Ovarian Cancer Model

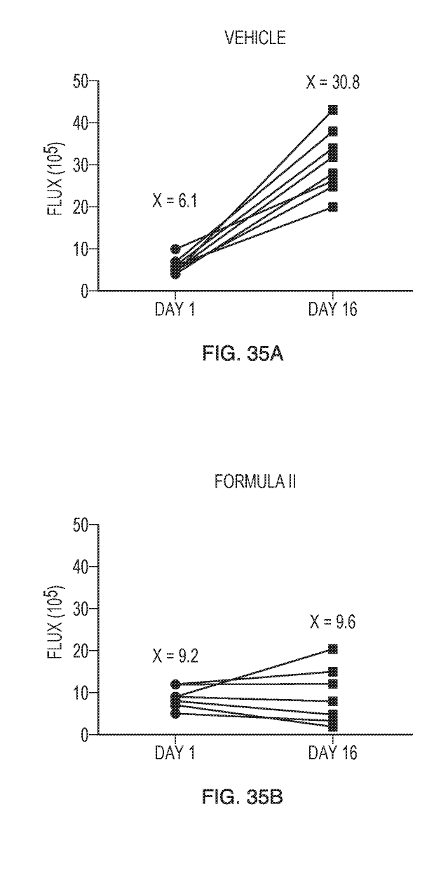

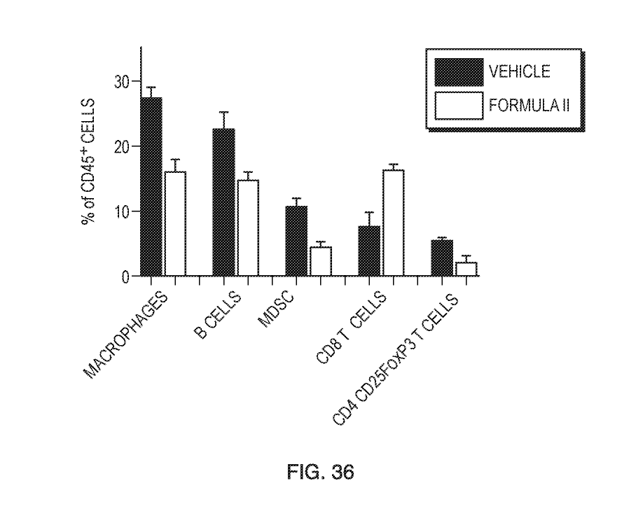

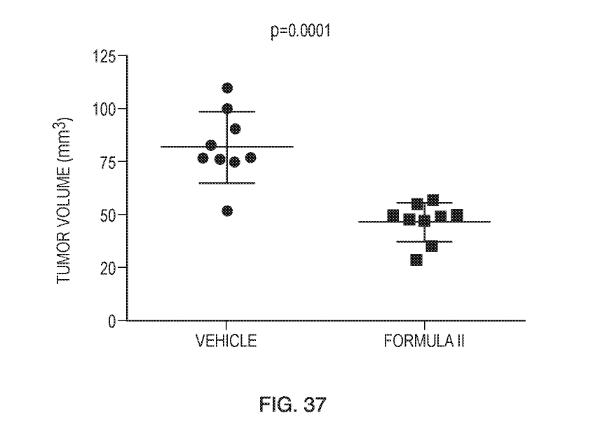

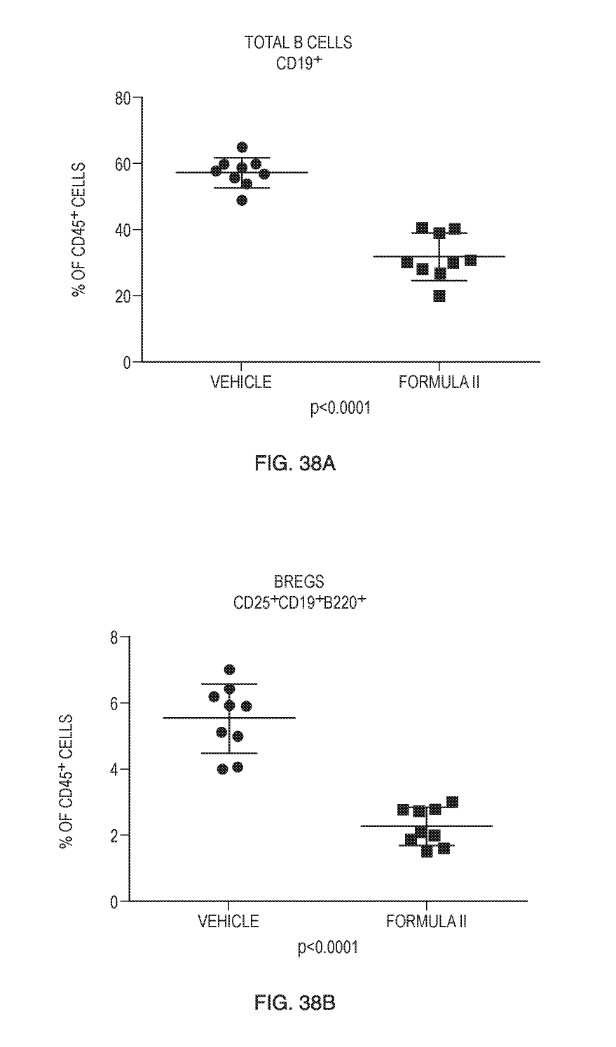

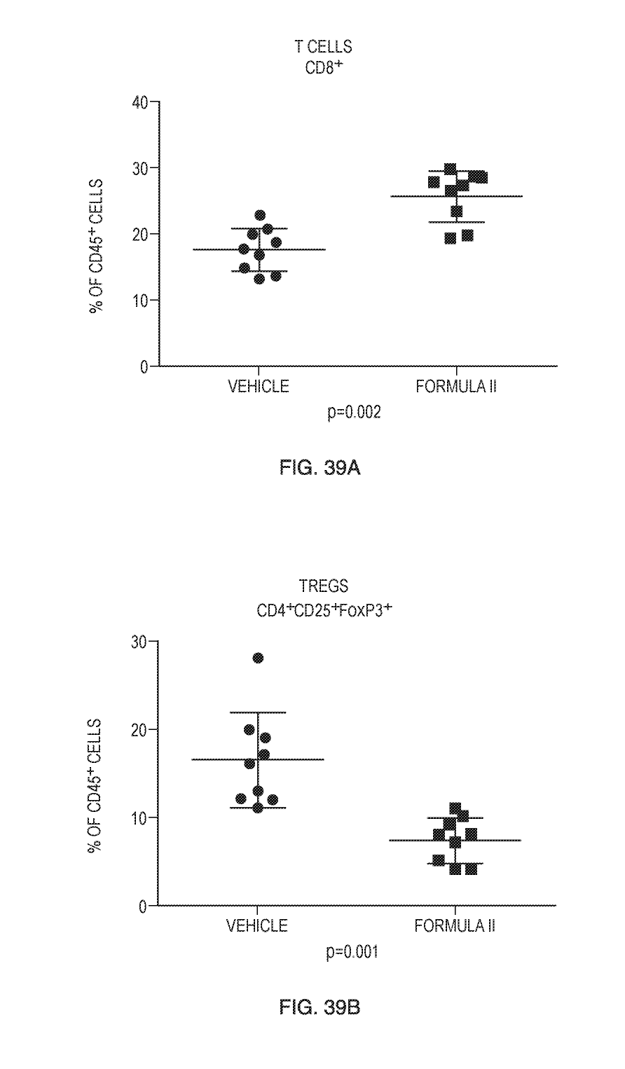

The ID8 syngeneic orthotropic ovarian cancer murine model was used to investigate the therapeutic efficacy of the BTK inhibitor of Formula (II) through treatment of the solid tumor microenvironment. Human ovarian cancer models, including the ID8 syngeneic orthotropic ovarian cancer model and other animal models, are described in Fong and Kakar, J. Ovarian Res. 2009, 2, 12; Greenaway, et al., Gynecol. Oncol. 2008, 108, 385-94; Urzua, et al., Tumour Biol. 2005, 26, 236-44; Janat-Amsbury, et al., Anticancer Res. 2006, 26, 3223-28; Janat-Amsbury, et al., Anticancer Res. 2006, 26, 2785-89. Animals were treated with vehicle or Formula (II), 15 mg/kg/BID given orally. The results of the study are shown in FIG. 35A, FIG. 35B, FIG. 36, FIG. 37, FIG. 38A, FIG. 38B, FIG. 39A, and FIG. 39B.

FIG. 35A and FIG. 35B demonstrate that the BTK inhibitor of Formula (II) impairs ID8 ovarian cancer growth in the ID8 syngeneic murine model. FIG. 36 shows that tumor response to treatment with the BTK inhibitor of Formula (II) correlates with a significant reduction in immunosuppressive tumor-associated lymphocytes in tumor-bearing mice. FIG. 37 shows treatment with the BTK inhibitor of Formula (II) impairs ID8 ovarian cancer growth (through reduction in tumor volume) in the syngeneic murine model. FIG. 38 shows that the tumor response induced by treatment with the BTK inhibitor of Formula (II) correlates with a significant reduction in immunosuppressive B cells in tumor-bearing mice. FIG. 39 shows that the tumor response induced by treatment with the BTK inhibitor of Formula (II) correlates with a significant reduction in immunosuppressive tumor associated Tregs and an increase in CD8+ T cells.

The results shown in FIG. 35 to FIG. 39 illustrate the surprising efficacy of the BTK inhibitor of Formula (II) in modulating tumor microenvironment in a model predictive of efficacy as a treatment for ovarian cancer in humans.

### Example 7. Effects of Sub-Chronic Low Dose Administration of Formula (II) on Development of T-Cell Dependent Antibody Responses (TDAR) in Male and Female Rats

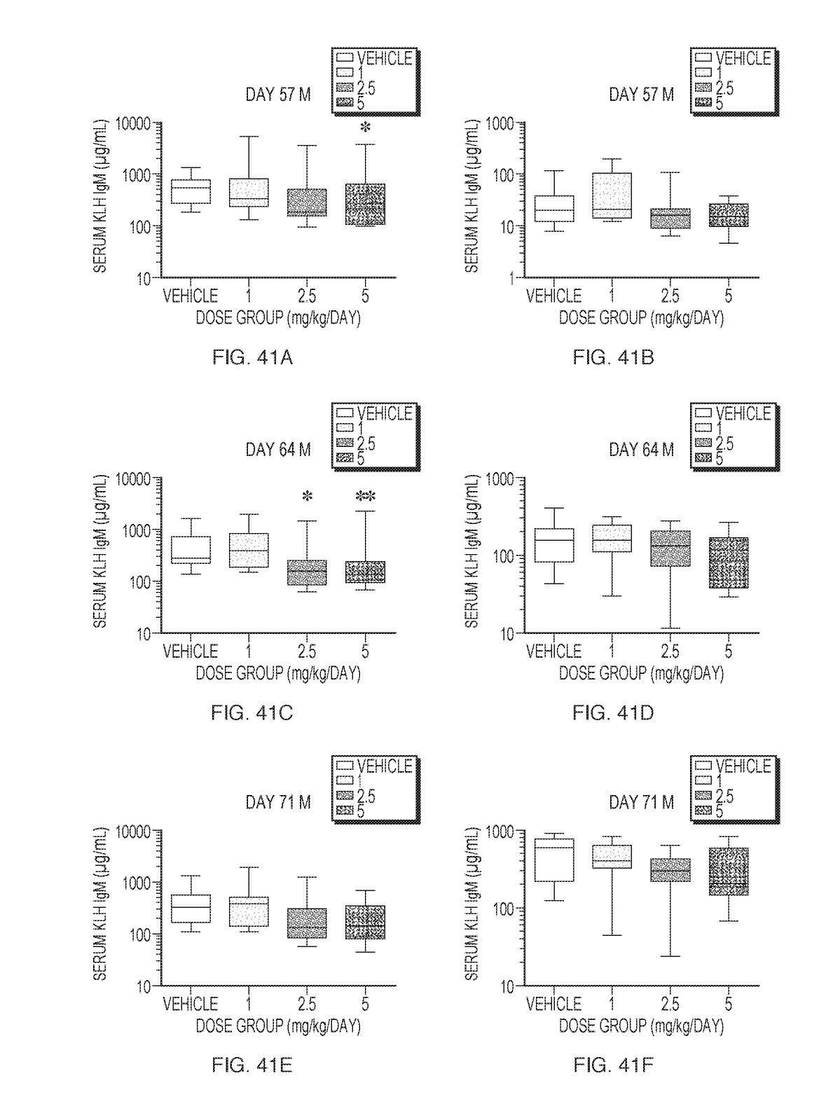

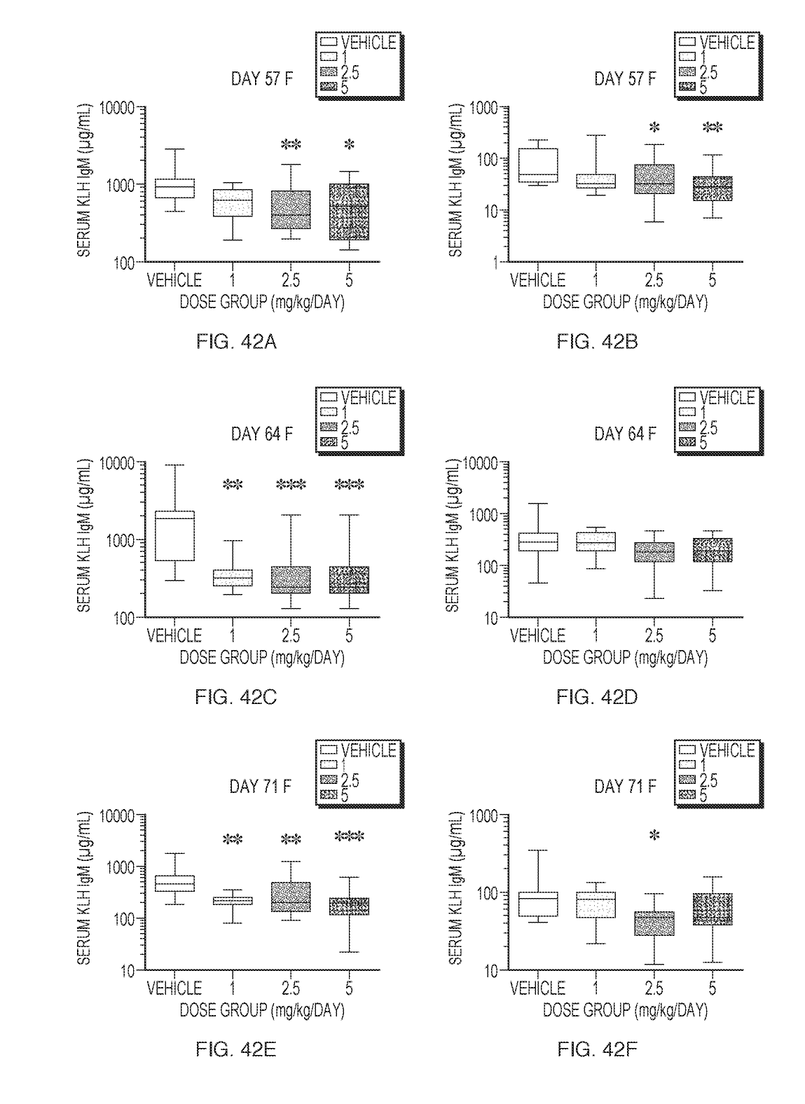

Rats were treated with oral gavage administration of vehicle or Formula (II) at doses of 1, 2.5 and 5 mg/kg/day for 91 days. Sixteen rats per gender were evaluated for development of T cell dependent antibody response (TDAR), with primary immunization on Day 50 with keyhole limpet hemocyanin (KLH) by subcutaneous injection. Dosing continued and on Days 57, 64, and 71, blood samples were collected via the sublingual vein into tubes without anticoagulant and allowed to clot at room temperature for at least 30 minutes, then the serum was divided into 4 approximately equal aliquots and stored frozen at approximately −60 to −90° C. Anti-KLH IgM and IgG antibody levels were determined using an enzyme-linked immunosorbent assay (ELISA). FIG. 41A, FIG. 41B, FIG. 41C, FIG. 41D, FIG. 41E, FIG. 41F, FIG. 42A, FIG. 42B, FIG. 42C, FIG. 42D, FIG. 42E, and FIG. 42F illustrate the trends in anti-KLH antibody responses among male and female rats.

Surprisingly, even these low doses had significant pharmacological effects on the magnitude of IgM and IgG production in response to a foreign antigen (Kruskal-Wallis test performed using non-transformed data for each post-immunization timepoint; GraphPad Prism). The dose range tested in this rat model is equivalent to human doses of 10, 24, and 48 mg QD. This result is consistent with discovery presented in Example 1, which shows that low daily doses of Formula (II) can accrue BTK target occupancy over time and reach a steady state of BTK inhibition in the tissue compartment of interest (in this case, the lymph nodes) to exert desired physiological changes. Specific reductions in the TDAR response, without adverse immunological suppression or a loss of host resistance, support the use of a selective covalent BTK inhibitor in chronic autoimmune and allergic disease indications.

### Example 8. Effects of Formula (II) on Osteoclastic Bone Lesions in a Rat Model of Tumor Bone Metastasis

Rats were inoculated with MDA-MB-231 tumor cells on Day 1, baseline radiographs were taken on Day 2, and treatment with Formula (II) began on Day 8. Radiographs were taken on Days 10, 17, 24, 31, 38, and 41. Anti-osteolytic activities were determined from differences in bone density and visual lysis scores between the drug-treated and vehicle-treated groups on each observation day, and also from changes in bone density and visual lysis scores in tumor-inoculated left tibias at baseline (Day 2) compared to Days 10, 17, 24,31, 38, and 41.

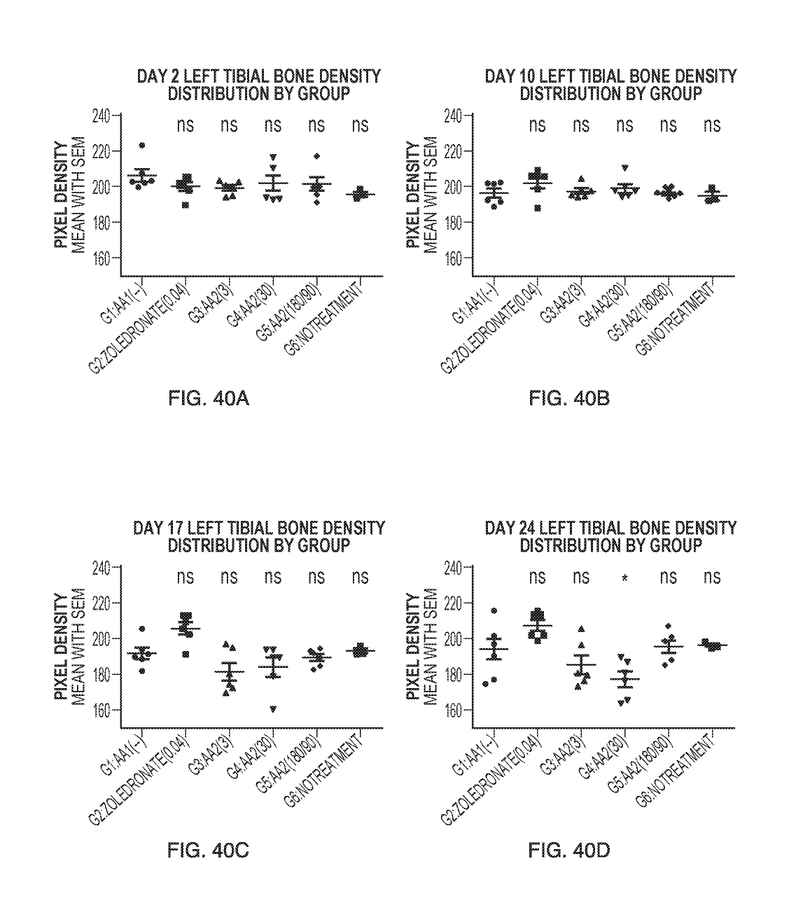

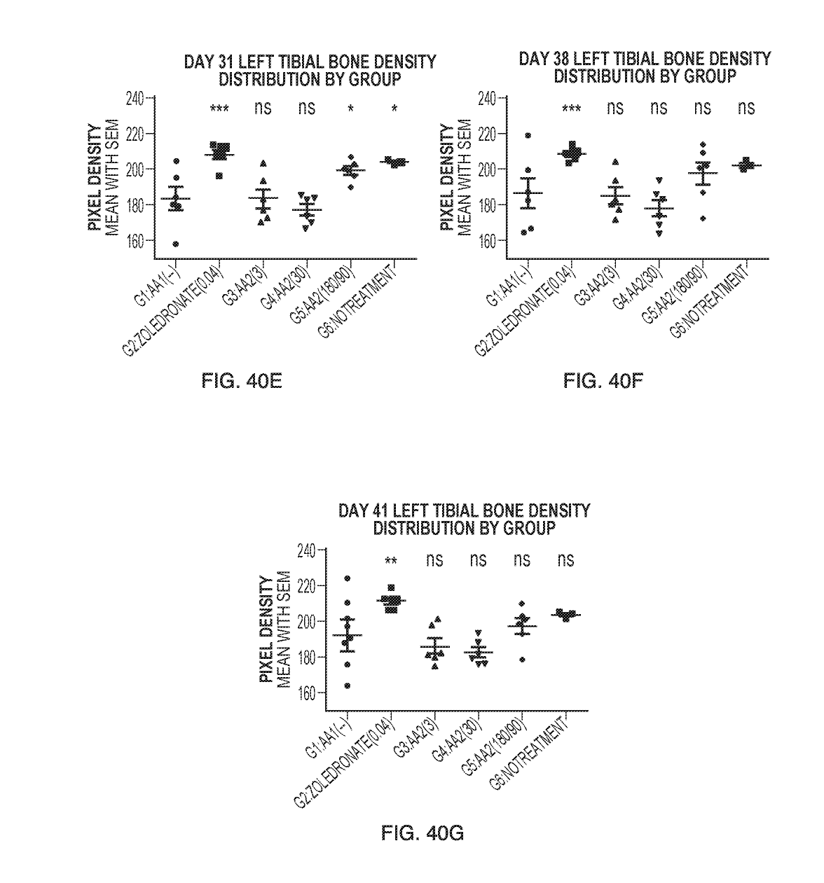

Activity of Formula (II) was determined based on quantitative assessment of bone density changes as illustrated in FIG. 40A, FIG. 40B, FIG. 40C, FIG. 40D, FIG. 40E, FIG. 40F, and FIG. 40G, and on semi-quantitative visual scoring of osteolytic lesions on digital images of tibial radiographs. Radiographs were blinded for both analyses. The nave group (Group 6, n=3), which was not inoculated and untreated, had a baseline mean left tibial density of 195.7±1.7, which increased statistically significantly to 203.6±1.7 on Day 41. Right tibias in this group exhibited comparable changes. Visual osteolysis was not detected in any rat in the nave group. The 4% increase in mean bone density from baseline to study end presumably reflected normal growth in these rats.

The vehicle control group had a baseline mean density of 206.2±1.7, which decreased statistically significantly by 11% to a nadir of 183.4±1.7 by Day 31, but was slightly higher at 192.0±1.7 on Day 41 and corresponded to a non-significant 7% decrease versus baseline (day 2). From Days 31 to 41, the mean visual lysis scores for the control group were each 2.3, reflecting an average of moderate to severe bone lysis. Osteolytic lesions were visible in 6/6 rats in the control group from Days 24 to 41. While radiograph data were evaluated for all observation days, overall outcomes in this model are normally based on observed changes for the last measurements. However, bone density and visual lysis outcomes for the control group discussed above suggested that Day 31 data were more robust, compared with data for Day 41. Due to concerns about apparent tumor rejection by Day 41, treatment outcomes in Groups 2-5 were considered for both Days 31 and 41.

The zoledronate group (Group 2) produced expected anti-osteolytic activity in this model. Mean left tibial densities increased 4-6% from 200.0±1.6 on Day 2 to 207.8±1.6 and 211.2±1.6 on Days 31 and 41, respectively, and were significantly greater compared to control Group 1 on these days. Mean visual lysis scores on Days 31 and 41 were each 0.7, reflecting an average of no to minimal lysis.

Rats treated with 3 and 30 mg/kg Formula (II) (Groups 3 and 4, respectively) produced bone density and visual lysis results that were similar to those for vehicle controls. There were no significant differences in left tibial bone density for either 3 or 30 mg/kg group compared to vehicle controls on any observation day, excluding Day 24 for the 30 mg/kg Formula (II) group, which resulted in significantly decreased density versus Group 1. On Days 31 and 41, the mean visual lysis scores for the 3 and 30 mg/kg groups ranged from 2.2 to 3.0, reflecting an average of moderate to severe bone lysis. In Group 3, one animal had no visible osteolytic lesions, but all other rats in Groups 3 and 4 had visual scores that increased in severity.

Rats treated with 180 QD/90 BID mg/kg Formula (II) (Group 5) exhibited little change in bone density and visual lysis from Days 2-41. Mean left tibial densities decreased by 1-2% from 201.4±0.6 on Day 2 to 199.1±0.6 and 197.2±0.6 on Days 31 and 41, respectively. Left intratibial densities for the Group 5 were significantly greater compared to control Group 1 on Day 31, but not Day 41. On Days 31 and 41, the mean visual lysis scores for Group 5 were 1.2 and 1.5, respectively, reflecting an average of minimal to moderate osteolysis. Two rats in this group never developed visible left tibial lesions, and three rats had no visible lesions on Days 31 and 41.

Results from this study are novel in two regards. First, they demonstrate the activity of Formula (II) on osteoclastic processes in vivo, in a relevant tissue compartment at tolerable doses in the nu/nu rat, showing activity on BTK downstream of RANKL and other myeloid effectors. Second, the dosing strategy in Group 5, where efficacy was observed, delivered larger bolus QD doses during the first three dosing days, converting to a BID dosing strategy for the next four weeks on study. The decrease in osteolytic bone lesions observed in this group was impressive when compared to appropriate control rats on Days 2-41. It should be noted that the zoledronate reference regimen administered in this model corresponded to a human equivalent dosage estimated to be approximately four-fold higher than the clinical dosage, was delivered by intravenous injection, and increased bone density in Group 5 beyond the physiological levels observed in the tibias of the untreated nave rats. The therapeutically relevant decreases in bone loss that were observed in this model with Formula (II) treatment show that BTK inhibition may provide benefits to patients with osteolytic bone diseases such as bone metastases, osteoarthritis, and osteoporosis.

### Example 9. BTK Expression in Different Cell Types and Tissues

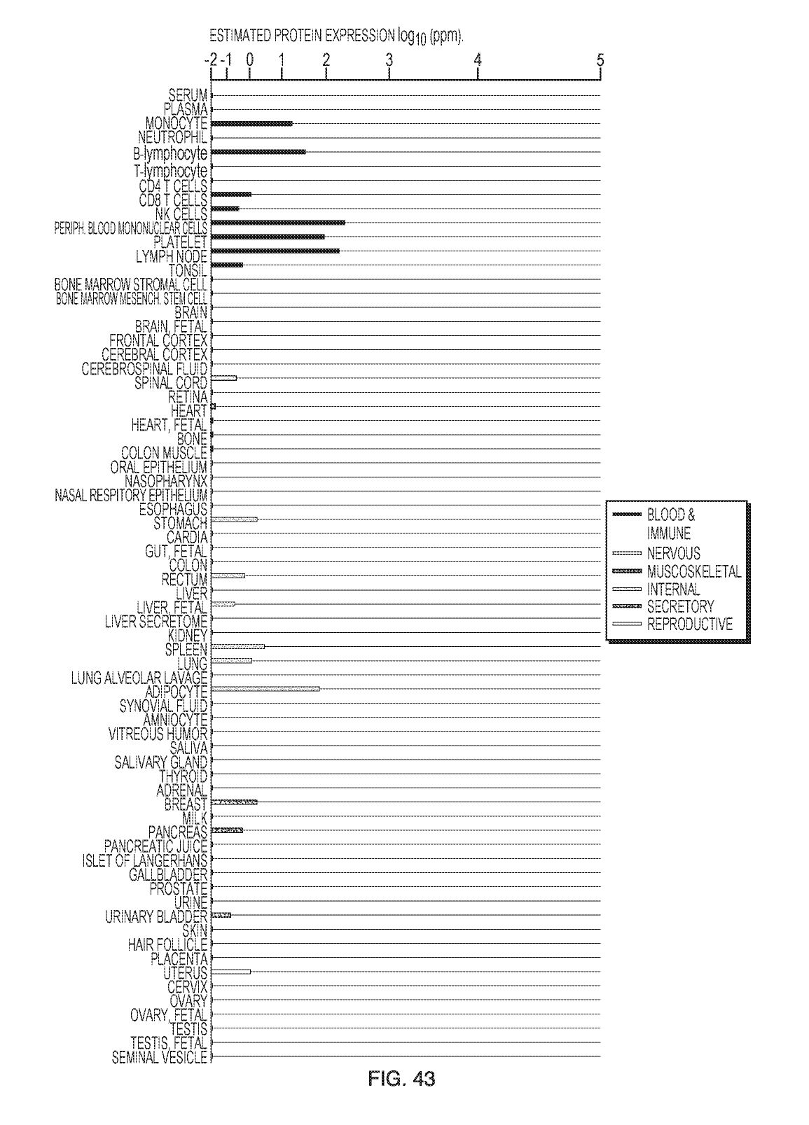

The differential protein expression of BTK across various cell types and tissues suggest different rates of BTK synthesis (see FIG. 43). In mice, B cells from the bone marrow were shown to have greater BTK protein expression compared to B cells from the spleen (Nisitani, et al., Proc. Nat'l. Acad. Sci. USA 2000, 97, 2737-2742). Bone marrow is the tissue compartment where B cell development and proliferation occurs, suggesting high BTK levels may be important for efficient B cell development. BTK protein expression also increases after stimulation through the B cell receptor (Nisitani, et al., PNAS. 2000, 97, 2737-2742). Lymph nodes are sites where B cells can be stimulated by antigen binding, leading to B cell activation and increased BTK synthesis. Other sites of high BTK syntheses are the tonsils, where immunohistochemical staining of BTK is considered high (see The Human Protein Atlas, entry for BTK and Tonsil, available at: proteinatlas.org/ENSG00000010671-BTK/tissue/tonsil).

### Example 10. Effects of Three Antiinflammatory Candidates Dosed PO and QD in a 14-Day Mouse Established Type II Collagen-Induced Arthritis Model

A study was designed to determine the efficacy of candidate anti-inflammatory agents Formula (II), ibrutinib, or Formula (XXI) in inhibiting inflammation, pannus formation, cartilage destruction, and bone resorption associated with established type II collagen-induced arthritis (CIA) in DBA/1 mice. Formula (II), ibrutinib, Formula (XXI), and the BTK inhibitor (R)-4-(8-amino-3-(4-(but-2-ynoyl)morpholin-3-yl)imidazo[1,5-a]pyrazin-1-yl)-N-(pyridin-2-yl)benzamide were preformulated in vehicle (0.4% HPMC, 0.1% Tween 80, with simethicone for antifoaming) for oral (PO) dosing at 10 mL/kg. Prior to dosing, the candidate anti-inflammatory agents were reconstituted either by vortexing or stirring. The candidate anti-inflammatory agents were administered to the DBA/1 mice daily (QD) by the oral (PO) route.

Immunization with heterologous type II collagen (CII) induces arthritis in mice of the DBA/1 strain, which is genetically susceptible to this disease. To develop an experimental model of autoimmunity more adequate for the study of human rheumatoid arthritis (RA), male DBA/1 mice were injected intradermally (ID) with bovine type II collagen to induce arthritis as reported by Trentham et al (Trentham D E, Townes A S, Kang A H Autoimmunity to type II collagen: an experimental model of arthritis. J. Exper. Med. 1977, 146:857-868) and Bendele A (Bendele A, Animal models of rheumatoid arthritis. J. Musculoskelet. Neuronal Interact. 2001, 1(4):377-85). Male DBA/1 mice (n=114) that were 6-7 weeks old and weighed approximately 18-27 grams (mean 22 g) at enrollment (arthritis day 1) were obtained from Taconic Farms, Inc. The male DBA/1 mice were at least 6 weeks old at time of first immunization and were housed 3-5/cage in shoe-box polycarbonate cages with wire tops, wood chip bedding, and suspended food and water bottles. These animals were identified by a distinct number of ink marks at the base of the tail delineating animal number. After enrollment, all cages were labeled with protocol number, group number, and animal numbers.

The male DBA/1 (n=10/group for arthritis) were anaesthetized with Isoflurane, shaved at the base of the tail, and injected intradermally with 150 μL of Freund's Complete Adjuvant (Difco, Detroit, Mich.) containing bovine type II collagen (BBP, batch #7) (1 mg/ml) at the base of the tail on day 0 and again on day 21. On study days 25-31, onset of arthritis occurred and mice were randomized into treatment groups. Randomization was done after swelling was obviously established in at least one paw (score of 1) and attempts were made to ensure approximately equal mean scores of 0.5-1 across groups at the time of enrollment. Once arthritis was established, the male DBA/1 mice were dosed PO, QD on arthritis days 1-14 with vehicle (0.4% HPMC, 0.1% Tween 80, with simethicone for antifoaming), Formula (II) (1, 5, or 25 mg/kg), ibrutinib (1, 5, or 25 mg/kg), Formula (XXI) (1 or 5 mg/kg), (R)-4-(8-amino-3-(4-(but-2-ynoyl)morpholin-3-yl)imidazo[1,5-a]pyrazin-1-yl)-N-(pyridin-2-yl)benzamide (5 mg/kg), or the reference compound dexamethasone (Dex, 0.2 mg/kg). The male DBA/1 mice were terminated on arthritis day 14 (3 hours post-dose).

Efficacy evaluation of the administered anti-inflammatory agents was based on animal body weights, clinical arthritis scores, arthritis scores expressed as area under the curve (AUC), and histopathology on fore paws, hind paws, and knees. Histopathology results were expressed as 4 paws, knees only, or 6 joints (knees included). Day 13 was used as the endpoint for analysis of body weight change since the male DBA/1 mice were fasted overnight prior to necropsy on day 14. All animals survived to study termination.

Treatment with Formula (II), ibrutinib, Formula (XXI), and (R)-4-(8-amino-3-(4-(but-2-ynoyl)morpholin-3-yl)imidazo[1,5-a]pyrazin-1-yl)-N-(pyridin-2-yl)benzamide showed significant beneficial effect in the established CIA model as determined by evaluation of disease-induced body weight loss, clinical arthritis scores, and histopathology of the joints. Disease-induced body weight loss (measured as percent change from baseline) was significantly inhibited toward normal for the male DBA/1 mice given 1 mg/kg Formula (II) (*p<0.05 on days 7-11), 5 mg/kg Formula (II) (*d5-14), 25 mg/kg Formula (II) (*d3-14), 1 mg/kg ibrutinib (*d7-9), 5 mg/kg ibrutinib (*d5-14), 25 mg/kg ibrutinib (*d3-14), 1 mg/kg Formula (XXI) (*d3-14), 5 mg/kg Formula (XXI) (*d3-14), or 5 mg/kg (R)-4-(8-amino-3-(4-(but-2-ynoyl)morpholin-3-yl)imidazo[1,5-a]pyrazin-1-yl)-N-(pyridin-2-yl)benzamide (*d5-14) as compared to vehicle controls.

Total body weight loss from enrollment to study termination (d1-14) was significantly inhibited for the male DBA/1 mice given Formula (II) (5 or 25 mg/kg), ibrutinib (5 or 25 mg/kg), or Formula (XXI) (1 or 5 mg/kg).

Arthritis scores measured daily were significantly reduced toward normal for the male DBA/1 mice given 5 mg/kg Formula (II) (*d3-14), 25 mg/kg Formula (II) (*d2-14), 5 mg/kg ibrutinib (*d3-14), 25 mg/kg ibrutinib (*d2-14), 1 mg/kg Formula (XXI) (*d8-14), 5 mg/kg Formula (XXI) (*d2-14), or 5 mg/kg (R)-4-(8-amino-3-(4-(but-2-ynoyl)morpholin-3-yl)imidazo[1,5-a]pyrazin-1-yl)-N-(pyridin-2-yl)benzamide (*d3-14) as compared to vehicle controls. When considering only those paws showing clinical signs of arthritis at enrollment (therapeutic paws), daily scores were significantly reduced toward normal for mice given 5 mg/kg Formula (II) (*d5-14), 25 mg/kg Formula (II) (*d2-14), 25 mg/kg ibrutinib (*d3-14), or 5 mg/kg Formula (XXI) (*d5-14) as compared to vehicle controls. When considering only those paws showing no clinical signs of arthritis at enrollment (prophylactic paws), daily scores were significantly reduced toward normal for mice given 1 mg/kg Formula (II) (*d2, 7-14), 5 mg/kg Formula (II) (*d2-14), 25 mg/kg Formula (II) (*d2-14), 1 mg/kg ibrutinib (*d2-7), 5 mg/kg ibrutinib (*d2-14), 25 mg/kg ibrutinib (*d2-14), 1 mg/kg Formula (XXI) (*d2-14), 5 mg/kg Formula (XXI) (*d2-14), or 5 mg/kg (R)-4-(8-amino-3-(4-(but-2-ynoyl)morpholin-3-yl)imidazo[1,5-a]pyrazin-1-yl)-N-(pyridin-2-yl)benzamide (*d2-14) as compared to vehicle controls.

Clinical arthritis scores AUC were significantly reduced toward normal for the male DBA/1 mice given 5 mg/kg Formula (II) (82% reduction, 99% when corrected for initial enrollment score), 25 mg/kg Formula (II) (95%, 115%), 5 mg/kg ibrutinib (73%, 89%), 25 mg/kg ibrutinib (93%, 112%), 1 mg/kg Formula (XXI) (65%, 79%), 5 mg/kg Formula (XXI) (86%, 104%), or 5 mg/kg (R)-4-(8-amino-3-(4-(but-2-ynoyl)morpholin-3-yl)imidazo[1,5-a]pyrazin-1-yl)-N-(pyridin-2-yl)benzamide (74%, 90%) as compared to vehicle controls. When considering therapeutic paws only, clinical arthritis scores AUC were significantly reduced toward normal for mice given 5 mg/kg Formula (II) (64% reduction, 110% when corrected for initial enrollment score), 25 mg/kg Formula (II) (89%, 152%), 25 mg/kg ibrutinib (82%, 141%), or 5 mg/kg Formula (XXI) (66%, 113%) as compared to vehicle controls. When considering prophylactic paws only, clinical arthritis scores AUC were significantly reduced toward normal for mice given 5 mg/kg Formula (II) (94% reduction), 25 mg/kg Formula (II) (100%), 5 mg/kg ibrutinib (96%), 25 mg/kg ibrutinib (100%), 1 mg/kg Formula (XXI) (99%), 5 mg/kg Formula (XXI) (100%), or 5 mg/kg (R)-4-(8-amino-3-(4-(but-2-ynoyl)morpholin-3-yl)imidazo[1,5-a]pyrazin-1-yl)-N-(pyridin-2-yl)benzamide (95%) as compared to vehicle controls.

Evaluation of histopathology confirmed the clinical findings; all six-joint histopathology parameters were significantly reduced toward normal for mice given 5 mg/kg Formula (II) (86% reduction of summed scores), 25 mg/kg Formula (II) (93%), 5 mg/kg ibrutinib (79%), 25 mg/kg ibrutinib (94%), 1 mg/kg Formula (XXI) (73%), 5 mg/kg Formula (XXI) (85%), or 5 mg/kg (R)-4-(8-amino-3-(4-(but-2-ynoyl)morpholin-3-yl)imidazo[1,5-a]pyrazin-1-yl)-N-(pyridin-2-yl)benzamide (75%) as compared to vehicle controls. Knee histopathology parameters were significantly improved for mice given 1 mg/kg Formula (II) as compared to 1 mg/kg ibrutinib. Spleen weights were not significantly affected by treatment with Formula (II), ibrutinib, Formula (XXI), or (R)-4-(8-amino-3-(4-(but-2-ynoyl)morpholin-3-yl)imidazo[1,5-a]pyrazin-1-yl)-N-(pyridin-2-yl)benzamide as compared to vehicle controls. Splenocyte preparations were used to evaluate BTK occupancy at this timepoint, which occurred at 3 hours after the final oral dose administration; similar BTK occupancy was observed in the mice treated with Formula (II), ibrutinib and Formula (XXI).

Results of treatment with Dex were as expected, in that Dex treatment significantly ameliorated arthritis.

Vehicle-treated control mice had body weight loss (measured as percent change from baseline) that began at the onset of arthritis and peaked at 10.86% on study day 13 (day 13 was used as the endpoint for analysis of body weight change since mice were fasted overnight prior to necropsy on day 14).

Disease-induced body weight loss was significantly inhibited toward normal for mice given 1 mg/kg Formula (II) (*p<0.05 on days 7-11), 5 mg/kg Formula (II) (*d5-13), 25 mg/kg Formula (II) (*d3-13), 1 mg/kg ibrutinib (*d7-9), 5 mg/kg ibrutinib (*d5-13), 25 mg/kg ibrutinib (*d3-13), 1 mg/kg Formula (XXI) (*d3-13), 5 mg/kg Formula (XXI) (*d3-13), or 5 mg/kg (R)-4-(8-amino-3-(4-(but-2-ynoyl)morpholin-3-yl)imidazo[1,5-a]pyrazin-1-yl)-N-(pyridin-2-yl)benzamide (*d5-13) as compared to vehicle controls. Total body weight loss from enrollment to study termination (d1-13) was significantly inhibited for mice given Formula (II) (5 or 25 mg/kg), ibrutinib (5 or 25 mg/kg), or Formula (XXI) (1 or 5 mg/kg).

The experimental design is shown in Table 1.

TABLE 1
Experimental design.

| | | | | | Dose | Dose | Dose |
|---|---|---|---|---|---|---|---|
| | | | | | Level | Conc. | Conc. |
| | | | | Regi- | mg/ | mg/ | mg/ |
| Group | n | Compound | Route | men | kg | ml | ml |
| 1 | 4 | Naive | N/A | N/A | N/A | N/A | N/A |
| 2 | 10 | VehicleControl | PO | QD | N/A | 10 | N/A |
| 3 | 10 | Dex | PO | QD | 0.2 | 10 | 0.02 |
| 4 | 10 | Formula (II) | PO | QD | 1 | 10 | 0.1 |
| 5 | 10 | Formula (II) | PO | QD | 5 | 10 | 0.5 |
| 6 | 10 | Formula (II) | PO | QD | 25 | 10 | 2.5 |
| 7 | 10 | ibrutinib | PO | QD | 1 | 10 | 0.1 |
| 8 | 10 | ibrutinib | PO | QD | 5 | 10 | 0.5 |
| 9 | 10 | ibrutinib | PO | QD | 25 | 10 | 2.5 |
| 10 | 10 | Formula (XXI) | PO | QD | 1 | 10 | 0.1 |
| 11 | 10 | Formula (XXI) | PO | QD | 5 | 10 | 0.5 |
| 12 | 10 | (R)-4-(8-amino- | PO | QD | 5 | 10 | 0.5 |
| | | 3-(4-(but-2- | | | | | |
| | | ynoyl)morpholin- | | | | | |
| | | 3-yl)imidazo[1,5- | | | | | |
| | | a]pyrazin-1-yl)- | | | | | |
| | | N-(pyridin-2- | | | | | |
| | | yl)benzamide | | | | | |

The results are summarized in Table 2.

TABLE 2
Experimental results.

| | | Clinical Data | | | | | |
|---|---|---|---|---|---|---|---|
| Experimental Results | | | | AUC (All | | | |
| | Treatment | Change in | Paw Score | Paws) - | | | |
| | (Groups 2-12: | Body | AUC | Corrected for | Histopathology Summed Score | | |
| Group | PO, QD) | Weight (g) | (All Paws) | Initial Score | Six-Joints | Paws | Knees |
| 1. | Naive | †0.32(SE0.42) | †0.00(0.00) | †0.00(0.00) | †0.00(0.00) | †0.00(0.00) | †0.00(0.00) |
| 2 | Vehicle Control | −2.40(0.68) | 41.30(2.52) | 34.15(2.15) | 14.61(0.75) | 15.84(0.99) | 12.15(1.23) |
| 3 | Dex (0.2 mg/kg) | −2.22(0.54) | *1.30(0.37) | *−5.85(0.73) | *0.66(0.21) | *0.09(0.09) | *1.80(0.66) |
| 4 | Formula (II) | −0.36(0.77) | 22.14(4.11) | 14.99(3.62) | 6.93(1.22) | 7.95(1.54) | 4.90(1.32) |
| | (1 mg/kg) | | | | | | |
| 5 | Formula (II) | *1.23(0.52) | *7.58(2.63) | *0.43(2.76) | *1.98(0.69) | *2.23(0.72) | *1.48(0.94) |
| | (5 mg/kg) | | | | | | |
| 6 | Formula (II) | *1.01(0.66) | *1.91(0.96) | *−5.24(1.36) | *1.04(0.36) | *0.94(0.40) | *1.25(0.43) |
| | (25 mg/kg) | | | | | | |
| 7 | ibrutinib | −1.28(0.69) | 28.29(5.18) | 20.81(4.47) | 10.25(1.36) | 10.30(1.84) | ‡10.15(1.16) |
| | (1 mg/kg) | | | | | | |
| 8 | ibrutinib | *0.45(0.65) | *11.15(3.37) | *3.68(2.18) | *3.11(0.95) | *3.63(1.07) | *2.08(0.99) |
| | (5 mg/kg) | | | | | | |
| 9 | ibrutinib | *0.83(0.62) | *3.06(1.08) | *−4.09(1.58) | *0.83(0.30) | *0.91(0.48) | *0.65(0.29) |
| | (25 mg/kg) | | | | | | |
| 10 | Formula (XXI) | *0.33(0.82) | *14.43(4.11) | 7.28(3.26) | *3.91(1.13) | *4.14(1.27) | *3.45(1.22) |
| | (1 mg/kg) | | | | | | |
| 11 | Formula (XXI) | *0.89(0.71) | *5.83(1.53) | *−1.33(1.48) | *2.13(0.66) | *1.46(0.47) | *3.48(1.17) |
| | (5 mg/kg) | | | | | | |
| 12 | (R)-4-(8-amino-3- | −0.04(0.92) | *10.58(2.95) | *3.43(2.22) | *3.71(0.87) | 4.24(1.07) | *2.65(0.79) |
| | (4-(but-2- | | | | | | |
| | ynoyl)morpholin- | | | | | | |
| | 3-yl)imidazo[1,5- | | | | | | |
| | a]pyrazin-1-yl)-N- | | | | | | |
| | (pyridin-2- | | | | | | |
| | yl)benzamide | | | | | | |
| | (5 mg/kg) | | | | | | |

*p < 0.05 ANOVA to Vehicle Control
†p < 0.05 t-test to Vehicle Control
‡p < 0.05 ANOVA to Formula (II) (1 mg/kg)

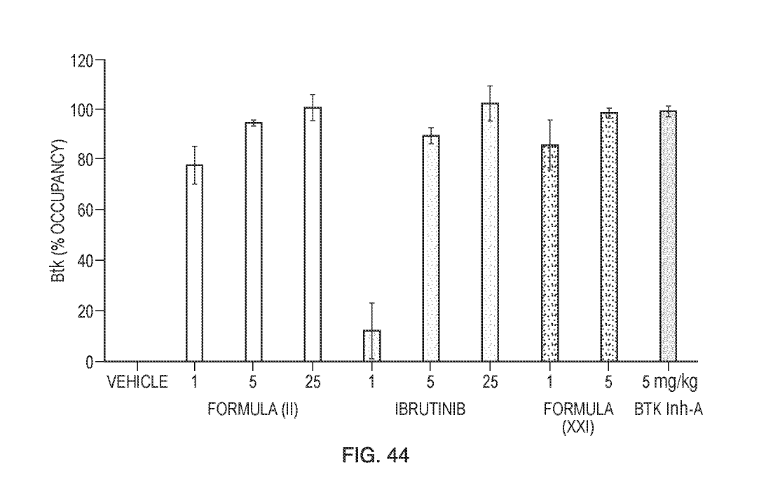

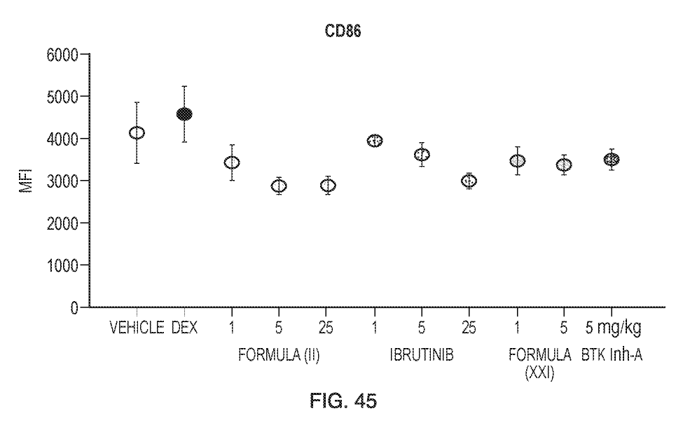

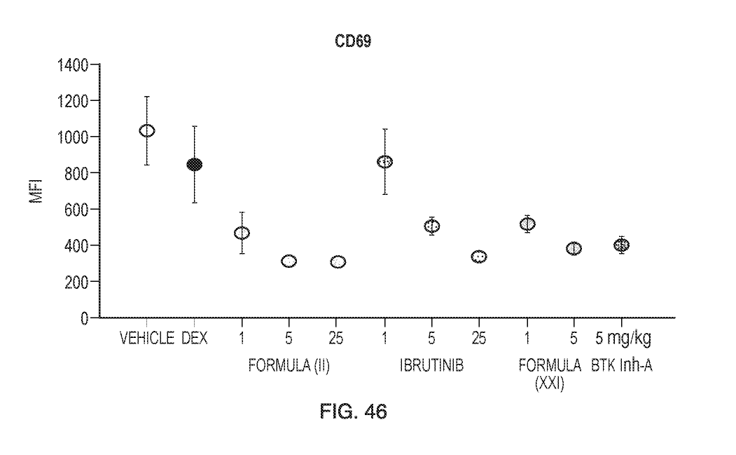

Similar BTK occupancy was observed in the mice treated with Formula (II), ibrutinib, Formula (XXI), and (R)-4-(8-amino-3-(4-(but-2-ynoyl)morpholin-3-yl)imidazo[1,5-a]pyrazin-1-yl)-N-(pyridin-2-yl)benzamide (“BTK Inh-A”), as illustrated in FIG. 44. Inhibition of the BCR signaling through BTK was evaluated by ex vivo stimulation of splenocytes using IgM and evaluation of CD86 and CD69 as functional responses of BCR stimulation. FIG. 45 and FIG. 46 show that in mice treated with Formula (II), ibrutinib, Formula (XXI) and (R)-4-(8-amino-3-(4-(but-2-ynoyl)morpholin-3-yl)imidazo[1,5-a]pyrazin-1-yl)-N-(pyridin-2-yl)benzamide (“BTK Inh-A”) had dose-responsive decreases in BCR-mediated signaling, whereas in mice treated with dexamethasone, no effect on BCR signaling was observed. The similarity in levels of functional inhibition of BCR with Formula (II) and Formula (XXI), along with similar BTK occupancy, reflected their similar mode of action on the BTK active site kinase.

### Example 11. Effects of Formula (II) when Dosed PO, QD or BID in 14-Day Mouse Established Type II Collagen Arthritis

A study was designed to determine the efficacy of a covalently acting BTK inhibitor, Formula (II) in inhibiting acute inflammation, pannus formation, cartilage destruction, and bone resorption associated with established type II collagen-induced arthritis (CIA) in DBA/1 mice. Immunization with heterologous type II collagen (CII) induces arthritis in mice of the DBA/1 strain, which is genetically susceptible to this disease. To develop an experimental model of autoimmunity more adequate for the study of human rheumatoid arthritis (RA), male DBA/1 mice were injected intradermally (ID) with bovine type II collagen to induce arthritis as reported by Trentham et al (Trentham D E, Townes A S, Kang A H Autoimmunity to type II collagen: an experimental model of arthritis. J. Exper. Med. 1977, 146:857-868) and Bendele A (Bendele A, Animal models of rheumatoid arthritis. J. Musculoskelet. Neuronal Interact. 2001, 1(4):377-85).

Following acclimatization, 6-7 week old male DBA/1 mice (n=10/group for arthritis) were immunized at the base of the tail, with 150 μL of Freund's Complete Adjuvant (Difco, Detroit, Mich.) containing 1 mg/mL bovine type II collagen by intradermal injection. Immunizations were performed day 0 and again on day 21. On study days 25-26, on day 8 following onset of arthritis, the mice were randomized into treatment groups. Day 8 generally represents peak acute disease state (score of 4/affected joint) in this model; treatment groups were balanced with approximately equal mean scores of 3.5 at the time of enrollment. Dosing began on arthritis day 8 by oral gavage. Groups were treated with vehicle (0.4% HPMC, 0.2% Tween 80), Formula (II) at 25 mg/kg QD, Formula (II) at 12.5 mg/kg BID, or the reference compound dexamethasone (Dex) at 0.2 mg/kg QD. These animals were fasted overnight on arthritis day 20 (13 days after enrollment) prior to necropsy on arthritis day 21. Clinical scores were given for each of the paws (right front, left front, right rear, left rear) on arthritis days 1-21.

Efficacy evaluation was based on animal body weights, clinical arthritis scores, arthritis scores expressed as area under the curve (AUC), and histopathology on fore paws, hind paws, and knees. Histopathology results were expressed as 4 paws, knees only, or 6 joints (knees included). Day 19 was used as the endpoint for analysis of body weight change since the male DBA/1 mice were fasted overnight prior to necropsy on day 21. All animals survived to study termination. Formula (II) was well tolerated under the conditions of this study with no adverse effects resulting from treatment.

Treatment with the BTK inhibitor (25 mg/kg QD or 12.5 mg/kg BID) resulted in significant improvements in the established CIA model as determined by evaluation of disease-induced body weight loss, clinical arthritis scores, and histopathology of the joints. Results of QD and BID treatment were mostly similar, with the exception of body weights, which were significantly increased for mice given 25 mg/kg QD as compared to treatment with 12.5 mg/kg BID, and spleen weights, which were significantly reduced toward normal for mice given 12.5 mg/kg BID.

Arthritis scores measured daily were significantly reduced toward normal for mice treated QD with 25 mg/kg Formula (II) (*p<0.05 on days 11-21) or BID with 12.5 mg/kg Formula (II) (*p<0.05 on days 9-21) as compared to vehicle controls. Initial reductions in arthritis scores from Formula (II) treatment were stronger with BID treatment; however, the prolonged treatment effect was similar for the two dosing regimens.

Area under the curve (AUC) for clinical scores was calculated from dosing initiation (d8) through study termination (d21). Clinical arthritis score AUCs were significantly reduced toward normal for mice given 25 mg/kg QD Formula (II) (56% reduction) or 12.5 mg/kg BID Formula (II) (59% reduction) as compared to vehicle controls. When corrected for initial enrollment scores (d8), arthritis scores AUCs were also significantly reduced for mice treated with Formula (II) at 25 mg/kg QD, 12.5 mg/kg BID, or with Dex as compared to vehicle controls.

Necropsies were conducted on fasted mice and terminal serum, fore paws, hind paws, and knees were collected into 10% neutral buffered formalin (NBF) for microscopy. Spleens were harvested and weighed.

Absolute spleen weights for vehicle control mice were significantly increased as compared to nave controls. Absolute spleen weights were significantly reduced for mice given Dex or 12.5 mg/kg BID Formula (II), as compared to vehicle controls, by 156% and 64%, respectively. Absolute spleen weights for mice given 12.5 mg/kg BID Formula (II) were also significantly reduced as compared to treatment with 25 mg/kg QD treatment group.

All six joint histopathology parameters were significantly reduced toward normal for mice given Formula (II) 25 mg/kg QD (56% reduction of summed scores) and at 12.5 mg/kg BID (53%), or the Dex group (57%), as compared to vehicle controls. Six-joint mean periosteal bone widths were significantly reduced toward normal for all groups as compared to vehicle controls.

Male DBA/1 mice treated with 25 mg/kg QD Formula (II) had significantly reduced knee inflammation (53% reduction), pannus formation (82%), cartilage damage (28%), bone resorption (82%), and summed knee scores (51%) as compared to vehicle controls. Mice treated with the 12.5 mg/kg BID regimen had significantly reduced knee inflammation (49%), pannus formation (80%), bone resorption (78%), and summed knee scores (45%). Male DBA/1 mice treated with Dex had significantly reduced knee inflammation (57%), pannus formation (86%), cartilage damage (39%), bone resorption (88%), and summed knee scores (57%).

Results of the study show similar efficacy between the split dose (12.5 mg/kg BID) and the QD dose of 25 mg/kg Formula (II), when administered to DBA/1 mice following the onset of acute inflammation in a CIA model. Formula (II) was active at the sites of established disease, including joints of the paws and knees, along with ameliorating clinical signs and improving weight gain over the course of treatment.

The BID dosing regimen had a more pronounced effect on spleen enlargement, compared with the QD dosing regimen for the BTK inhibitor. Otherwise, clinical and histopathological evaluations of disease in collagen induced arthritis in DBA/1 mice showed improvement following treatment with Formula (II) when delivered on a QD or BID schedule with a total daily dose of 25 mg/kg. A similar degree of improvement was observed with Formula (II) and Dex, the positive control in this acute model of active rheumatoid arthritis.

The results are summarized in Table 3.

TABLE 3
Experimental results.

| | | Clinical Data | | | | | |
|---|---|---|---|---|---|---|---|
| | | | | AUC (All | | | |
| | | Change in | Paw Score | Paws)- | | | |
| Experimental Results | | Body Weight (g) | AUC | Corrected for | Histopathology Summed Score | | |
| Group | Treatment | Days 1-19 | (All Paws) | Initial Score | Six-Joints | Paws | Knees |
| 1 | Naïve | 0.80(SE 0.36) | ‡0.00(0.00) | 0.00(0.00) | ‡0.00(0.00) | ‡0.00(0.00) | ‡0.00(0.00) |
| 2 | Vehicle Control | 1.15(0.42) | 54.18(3.43) | 2.83(1.05) | 15.23(0.76) | 16.78(1.09) | 12.15(0.53) |
| | PO, QD | | | | | | |
| 3 | Dex (0.2 mg/kg) | −0.17(0.28) | *16.61(2.05) | *−29.54(2.39) | *6.54(0.91) | *7.20(1.00) | *5.23(0.89) |
| | PO, QD | | | | | | |
| 4 | Formula (II) | 2.37(0.28) | *23.63(3.37) | *−24.48(3.85) | *6.74(0.80) | *7.11(0.98) | *6.00(0.76) |
| | (25 mg/kg) | | | | | | |
| | PO, QD | | | | | | |
| 5 | Formula (II) | †0.64(0.69) | *22.45(2.68) | *−23.05(2.58) | *7.23(0.77) | *7.49(1.03) | *6.73(0.60) |
| | (12.5 mg/kg) | | | | | | |
| | PO, BID | | | | | | |

*p < 0.05 ANOVA to Vehicle Control
†p < 0.05 t-test to Formula (II) (25 mg/kg) PO, QD
‡p < 0.05 t-test to Vehicle Control

### Example 12. Efficacy of Formula (II) on Inflammation in the Kidney in the MRL/MpJFASlpr Mouse Model of Systemic Lupus Erythematosus

A study designed to determine the effects of candidate anti-inflammatory agent Formula (II) in oral (PO), daily (QD) administration in the treatment of systemic lupus erythematosus (SLE) in MRL/MpJFASlpr mice. Formula (II) was preformulated in vehicle (0.4% HPMC, 0.1% Tween 80, with simethicone for antifoaming). The female MRL/MpJFASlpr mice were dosed with vehicle or Formula (II) (1, 5, or 25 mg/kg) daily (QD) by the oral (PO) route dosing, or they were dosed QD by the intraperitoneal (IP) route with the reference compound cyclophosphamide (15 mg/kg). Prior to dosing, the candidate anti-inflammatory agent was reconstituted either by vortexing or stirring. The candidate anti-inflammatory agent was administered to the MRL/MpJFASlpr mice for two weeks.

Female MRL/MpJFASlpr mice (n=50) that were 8-9 weeks old and weighed approximately 28-41 grams (mean 34 g) at enrollment (mouse age approx. 12 weeks) were obtained from The Jackson Laboratory, Bar Harbor, Me. (stock number 000485). These mice were housed 3-5/cage in shoe-box polycarbonate cages with wire tops, wood chip bedding, and suspended food and water bottles. No concurrent medications were given. The experimental design is shown in Table 4.

TABLE 4
Experimental results.

| | | | | | Dose | Dose | Dose |
|---|---|---|---|---|---|---|---|
| | | | | | Level | Vol. | Conc. |
| | | | | Regi- | mg/ | ml/ | mg/ |
| Group | n | Compound | Route | men | kg | kg | ml |
| 1 | 10 | Vehicle Disease | PO | QD | N/A | 10 | N/A |
| | | Control | | | | | |
| 2 | 10 | Formula (II) | PO | QD | 1 | 10 | 0.1 |
| 3 | 10 | Formula (II) | PO | QD | 5 | 10 | 0.5 |
| 4 | 10 | Formula (II) | PO | QD | 25 | 10 | 2.5 |
| 5 | 10 | Cyclophos- | IP | QD | 15 | 10 | 1.5 |
| | | phamide | | | | | |

When they were 12 weeks old (study day 0), the female MRL/MpJFASlpr mice (n=10/group) were randomized by body weight into 5 groups as shown above. Treatment was initiated after randomization and continued for 12 weeks (mouse age 12 weeks to 23 weeks).

Starting on study week one, and then every week thereafter urine from each animal was tested for proteinuria using the Clinitech Multistick test strip (Bayer). The animals were also observed daily for significant clinical signs, moribundity and mortality. At approximately 12-14 weeks of age, onset of disease occurred. Efficacy evaluation was based on animal body weights, proteinuria analysis, lymphadenopathy scores, skin lesion scores, organ weights, anti-dsDNA titers, clinical chemistry parameters, and histopathologic evaluation of the kidneys. Three (3) of 10 Vehicle Disease Control and 1 of 10 Formula (II) (5 mg/kg) mice died or were sacrificed moribund prior to study termination. All other mice survived to the scheduled termination.

Scoring of lymphadenopathy (cervical, brachial, and inguinal) and skin lesions for all animals was recorded starting at study week 4 once lesions were apparent in 50% of animals in the vehicle-treated group. Any animals showing signs of morbundity were terminated immediately. At necropsy (3 h post-dose, mouse age 23 weeks), all remaining animals were anesthetized with Isoflurane and bled via cardiac puncture for serum and plasma (Li Heparin). The animals were bled to exsanguinate then euthanized by cervical dislocation. Spleens, kidneys (paired), and lymph nodes were collected and weighed. Kidneys were collected into 10% neutral buffered formalin (NBF). Spleens were placed in RPMI 1% FBS media for splenocyte isolation. Serum samples for potential clinical chemistry analysis were stored frozen at −80° C. until shipment to Antech Diagnostics. Plasma samples were stored frozen at −80° C. for mouse antidsDNA IgG analysis by ELISA.

Clinical chemistry parameters were measured for 3 mice that were killed interim (moribund): Vehicle Disease Control animals #1 and #6, and Formula (II) (5 mg/kg) animal #4. Blood samples were drawn via cardiac puncture from Isoflurane anesthetized animals into serum separator microtainer tubes for clinical chemistries. Samples were shipped to Antech Diagnostics for analysis.

As shown in the following table, treatment with Formula (II) showed significant and dose responsive beneficial effect in the treatment of lupus nephritis in MRL/MpJ-FASlpr mice as determined by evaluation of proteinuria scores, kidney weights, mesenteric lymph node weights, and histopathology scores including significantly reductions in glomerulus diameter, percent of glomeruli with crescents, protein cast score, vasculitis and summed histopathology scores compared to Vehicle Disease Controls. Body weight measurements, lymphadenopathy scores, skin lesion scores, spleen weights, cumulative lymph node weights, and mouse anti-dsDNA IgG levels in plasma for mice treated with Formula (II) did not differ significantly from Vehicle Disease Controls. Results of treatment with cyclophosphamide were as expected, in that treatment resulted in significant beneficial effects on clinical and histopathologic parameters.

The results are shown in Table 5.

TABLE 5
Experimental results.

| | | Clinical Data | | | | | | |
|---|---|---|---|---|---|---|---|---|
| | | | | Change in | | Histopathology Data | | |
| | | | Kidney (paired) | Body Weight (g) | Percent | Glomerulus | Interstitial | Summed |
| Group | Treatment | | Weight (g) | wk 1-12 | Survival | Diameter | Inflammation | Score |
| 1 | Vehicle Disease | All Animals | 0.564 (0.04) | — | 70% | 106.35 (6.57) | 3.70 (0.40) | 15.23 (1.93) |
| | Control | Term Animals | 0.630 (0.03) | 4.99 (2.47) | | 94.89 (3.62) | 3.14 (0.40) | 12.04 (1.53) |
| | PO, QD | | | | | | | |
| 2 | Formula (II) | All Animals | 0.576 (0.03) | — | 100% | *87.78 (3.82) | 2.45 0.28) | 7.70 (1.07) |
| | (1 mg/kg) | Term Animals | 0.576 (0.03) | 8.10 (0.66) | | 87.78 (3.82) | 2.45 0.28) | 7.70 (1.07) |
| | PO, QD | | | | | | | |
| 3 | Formula (II) | All Animals | 0.544 (0.02) | — | 90% | *80.53 (5.64) | 1.98 (0.29) | *5.60 (0.98) |
| | (5 mg/kg) | Term Animals | *0.531 (0.02) | 7.01 (0.67) | | *80.17 (6.29) | 1.97 (0.33) | *5.50 (1.08) |
| | PO, QD | | | | | | | |
| 4 | Formula (II) | All Animals | 0.511 (0.01) | — | 100% | *74.78 (1.62) | *1.53 (0.15) | *3.88 (0.34) |
| | (25 mg/kg) | Term Animals | *0.511 (0.01) | 6.90 (0.56) | | *74.78 (1.62) | *1.53 (0.15) | *3.88 (0.34) |
| | PO, QD | | | | | | | |
| 5 | Cyclophosphamide | All Animals | *0.432 (0.01) | — | 100% | *67.73 (2.10) | *0.95 (0.19) | *2.18 (0.57) |
| | (15 mg/kg) | Term Animals | *0.432 (0.01) | 3.30 (0.32) | | *67.73 (2.10) | *0.95 (0.19) | *2.18 (0.57) |
| | IP, QD | | | | | | | |

*p < 0.05 ANOVA or Kruskal Wallis test to Vehicle Disease Control
(SE) = Standard errors displayed in parenthesis

All groups had body weights (measured as percent change from baseline) that increased over the course of the study. When considering all animals (including those that died interim), mice treated with cyclophosphamide had significantly reduced body weight gain at mouse age 14-16 and 19 weeks compared to Vehicle Disease Controls. When considering only those animals that survived to study termination (term animals), mice treated with cyclophosphamide had significantly reduced body weight gain at mouse age 14-17 and 19-20 weeks compared to Vehicle Disease Controls. From week 1 to week 5, Vehicle Disease Controls had mean body weight gain of 3.39 g for all animals and 3.85 g for term animals. Over that same period, mice given cyclophosphamide had significantly reduced body weight gain of 1.03 g. Body weight change for mice treated with Formula (II) did not differ significantly from Vehicle Disease Controls.

Vehicle Disease Controls had mean urine protein scores that increased over the course of the study. When considering all animals, urine protein scores were significantly reduced at mouse age 14-15 and 21-22 in mice treated with 25 mg/kg Formula (II) and at mouse age 14-23 weeks in mice given cyclophosphamide compared to Vehicle Disease Controls. When considering term animals only, urine protein scores were significantly reduced at mouse age 22 in mice treated with 25 mg/kg Formula (II) and at mouse age 14-16 and 19-23 weeks in mice given cyclophosphamide compared to Vehicle Disease Controls. Vehicle Disease Controls had mean lymphadenopathy scores that increased over the course of the study. Lymphadenopathy scores were significantly reduced at mouse age 15-23 weeks for mice given cyclophosphamide compared to Vehicle Disease Controls, whether considering all animals or term animals only. Lymphadenopathy scores were not significantly affected for mice treated with Formula (II) compared to Vehicle Disease Controls. Vehicle Disease Controls had mean skin lesion scores that increased over the course of the study. Skin lesion scores were significantly reduced at mouse age 20-23 weeks for mice given cyclophosphamide compared to Vehicle Disease Controls, whether considering all animals or term animals only. Skin lesion scores were not significantly affected for mice treated with Formula (II) compared to Vehicle Disease Controls.

Vehicle Disease Control mice had a 70% survival rate at study termination. Mice treated with 5 mg/kg Formula (II) had a 90% survival rate at study termination. All other groups had 100% survival. When considering all animals, absolute kidney weights were significantly reduced in mice given cyclophosphamide (23% reduction) compared to Vehicle Disease Controls. When considering term animals only, absolute kidney weights were significantly reduced in mice treated with 5 mg/kg Formula (II) (16%), 25 mg/kg Formula (II) (19%), or cyclophosphamide (32%) compared to Vehicle Disease Controls.

Absolute spleen weights were significantly reduced for mice given cyclophosphamide (77% and 82% reduction, respectively, for all animals and term animals) compared to Vehicle Disease Controls. Spleen weights for mice treated with Formula (II) did not differ significantly from Vehicle Disease Controls.

Absolute cumulative lymph node weights were significantly reduced for mice given cyclophosphamide (87% and 90% reduction, respectively, for all animals and term animals) compared to Vehicle Disease Controls. Cumulative lymph node weights for mice treated with Formula (II) did not differ significantly from Vehicle Disease Controls. When considering all animals, absolute mesenteric lymph node weights were significantly reduced in mice given cyclophosphamide (76% reduction) compared to Vehicle Disease Controls. When considering term animals only, absolute mesenteric lymph node weights were significantly reduced in mice treated with 5 mg/kg Formula (II) (52%), 25 mg/kg Formula (II) (49%), or cyclophosphamide (82%) compared to Vehicle Disease Controls. Vehicle Disease Controls had mouse anti-dsDNA IgG levels in plasma of 3,078.59 kU/ml. AntidsDNA IgG levels were significantly reduced in mice treated with cyclophosphamide (76% reduction) compared to Vehicle Disease Controls. Anti-dsDNA IgG levels were not significantly affected in mice treated with Formula (II) compared to Vehicle Disease Controls.

Evaluation of serum clinical chemistries revealed that mice given 1 mg/kg Formula (II) had significantly increased albumin (ALB), glucose (GLU), and sodium/potassium ratio (Na/K) and significantly reduced blood urea nitrogen (BUN), BUN/creatinine, phosphorus (P), potassium (K), cholesterol (CHOL), and amylase (AMY) compared to Vehicle Disease Controls when comparing all animals. When comparing term animals only, mice given 1 mg/kg Formula (II) had significantly reduced BUN and BUN/creatinine compared to Vehicle Disease Controls. When comparing all animals, mice treated with 5 mg/kg Formula (II) had significantly increased ALB, GLU, calcium (CA), and Na/K and significantly reduced BUN, BUN/creatinine, K, CHOL, and AMY compared to Vehicle Disease Controls. When comparing term animals only, mice treated with 5 mg/kg Formula (II) had significantly increased ALB, albumin/globulin ratio (A/G), GLU and Na/K and significantly reduced BUN, BUN/creatinine, and K compared to Vehicle Disease Controls. When comparing all animals, mice treated with 25 mg/kg Formula (II) had significantly increased ALB, GLU, calcium (CA), and Na/K and significantly reduced BUN, BUN/creatinine, K, CHOL, and AMY compared to Vehicle Disease Controls. When comparing term animals only, mice treated with 25 mg/kg Formula (II) had significantly increased ALB and CA and significantly reduced BUN and BUN/creatinine compared to Vehicle Disease Controls. Mice treated with cyclophosphamide had significantly increased ALB, A/G, alkaline phosphatase (ALK), GLU, and Na/K and significantly reduced globulin (GLOB), BUN, BUN/creatinine, K, and AMY compared to Vehicle Disease Controls, whether considering all animals or term animals only.

When considering all animals (including those that died interim), mice treated with 1 mg/kg Formula (II) had significantly reduced glomerulus diameter (43% reduction) and percent of glomeruli with crescents (97%) compared to Vehicle Disease Controls. Mice treated with 5 mg/kg Formula (II) had significantly reduced glomerulus diameter (60%), glomerulus score (58%), percent of glomeruli with crescents (99%), protein cast score (92%), vasculitis (52%), and summed histopathology scores (63%) compared to Vehicle Disease Controls. Mice treated with 25 mg/kg Formula (II) had significantly reduced glomerulus diameter (73%), glomerulus score (76%), glomerulus crescent score (100%), percent of glomeruli with crescents (100%), protein cast score (98%), interstitial inflammation (59%), vasculitis (52%), and summed histopathology scores (75%) compared to Vehicle Disease Controls. Mice treated with cyclophosphamide had significantly reduced glomerulus diameter (89%), glomerulus score (88%), glomerulus crescent score (100%), percent of glomeruli with crescents (100%), protein cast score (90%), interstitial inflammation (74%), vasculitis (82%), and summed histopathology scores (86%) compared to Vehicle Disease Controls.

When considering term animals only, mice treated with 1 mg/kg Formula (II) had significantly reduced percent of glomeruli with crescents (90% reduction) compared to Vehicle Disease Controls. Mice treated with 5 mg/kg Formula (II) had significantly reduced glomerulus diameter (46%), percent of glomeruli with crescents (98%), protein cast score (90%), and summed histopathology scores (54%) compared to Vehicle Disease Controls. Mice treated with 25 mg/kg Formula (II) had significantly reduced glomerulus diameters (63%), glomerulus score (72%), glomerulus crescent score (100%), percent of glomeruli with crescents (100%), protein cast score (97%), interstitial inflammation (51%), vasculitis (47%), and summed histopathology scores (68%) compared to Vehicle Disease Controls. Mice treated with cyclophosphamide had significantly reduced glomerulus diameter (85%), glomerulus score (86%), glomerulus crescent score (100%), percent of glomeruli with crescents (100%), protein cast score (86%), interstitial inflammation (70%), vasculitis (80%), and summed histopathology scores (82%) compared to Vehicle Disease Controls.

### Example 13. A Controlled-Release Formulation of a BTK Inhibitor

A dry-granulate of Formula (II) containing 10-50% Formula (II), 10-33% microcrystalline cellulose, 1-10% modified starch, 0.1-10% crospovidone, 0.05-1% magnesium stearate, and 0-1% fumed silicon dioxide may be prepared.

Portions of uncoated granules are coated on a fluidized bed using top spray to a 2% weight gain with poly(methacrylic acid-co-ethyl acrylate) 1:1, poly(methacylic acid-co-methyl methacrylate) 1:1, or poly(methacylic acid-co-methyl methacrylate) 1:2. Equal portions of the coated granules and uncoated granules are blended and filled into hard gelatin capsules to target a 100 mg total Formula (II) dose. 25 mg doses are targeted to be delivered in a pulsatile fashion in the stomach, duodenum, jejunum, and colon.

### Example 14. An Extended-Release Formulation of a BTK Inhibitor

A dry-granulate of Formula (II) containing 40-60% Formula (II), 15-30% microcrystalline cellulose, 1-10% modified starch, 0.1-10% crospovidone, 0.05-1% magnesium stearate, and 0-1% fumed silicon dioxide is blended with extragranular 4000 cP hypromellose substitution type 2208 (10-25% total weight) is prepared and is then lubricated with 0.05-1% magnesium stearate. The blend is then compressed using a rotary tablet press equipped with 10 mm round bi-convex dies.

### Example 15. Clinical Observation of Effects of BTK Inhibition on Dermatoses

Clinical studies have shown that targeting the BCR signaling pathway by inhibiting BTK produces significant clinical benefit in patients with various types of leukemias and lymphomas. Formula (II) achieves significant oral bioavailability and potency, and has favorable preclinical characteristics, as described above. The purpose of this study is to evaluate the safety and efficacy of the second generation BTK inhibitor of Formula (II) in treating subjects with chronic lymphocytic leukemia (CLL) and small lymphocytic lymphoma (SLL).

The primary objectives of the clinical study are as follows: (1) establish the safety and the MTD of orally administered Formula (II) in subjects with CLL/SLL; (2) determine pharmacokinetics (PK) of orally administered Formula (II) and identification of its major metabolite(s); and (3) measure pharmacodynamic (PD) parameters including drug occupancy of BTK, the target enzyme, and effect on biologic markers of B cell function. The secondary objective of the clinical study is to evaluate tumor responses in patients treated with Formula (II). In addition, effects of Formula (II) on other patient characteristics, such as dermatoses, were evaluated for particular patients enrolled in the study.

This study is a multicenter, open-label, nonrandomized, sequential group, dose escalation study. The following dose cohorts are evaluated:

Cohort 1: 100 mg/day for 28 days (=1 cycle)

Cohort 2: 175 mg/day for 28 days (=1 cycle)

Cohort 3: 250 mg/day for 28 days (=1 cycle)

Cohort 4: 400 mg/day for 28 days (=1 cycle)

Cohort 5: 450 mg/day for 28 days (=1 cycle)

Cohort 6: To be determined amount in mg/day for 28 days (=1 cycle)

Each cohort will be enrolled sequentially with 6 subjects per cohort. If ≦1 dose-limiting toxicity (DLT) is observed in the cohort during Cycle 1, escalation to the next cohort will proceed. Subjects may be enrolled in the next cohort if 4 of the 6 subjects enrolled in the cohort completed Cycle 1 without experiencing a DLT, while the remaining 2 subjects are completing evaluation. If ≧2 DLTs are observed during Cycle 1, dosing at that dose and higher will be suspended and the MTD will be established as the previous cohort. The MTD is defined as the largest daily dose for which fewer than 33% of the subjects experience a DLT during Cycle 1. Dose escalation will end when either the MTD is achieved or at 3 dose levels above full BTK occupancy, whichever occurs first. Full BTK occupancy is defined as Formula (II) active-site occupancy of >80% (average of all subjects in cohort) at 24 hours postdose. Should escalation to Cohort 6 be necessary, the dose will be determined based on the aggregate data from Cohorts 1 to 5, which includes safety, efficacy, and PK/PD results. The dose for Cohort 6 will not exceed 900 mg/day.

Treatment with Formula (II) may be continued for >28 days until disease progression or an unacceptable drug-related toxicity occurs. Subjects with disease progression will be removed from the study. All subjects who discontinue study drug will have a safety follow-up visit 30 (±7) days after the last dose of study drug unless they have started another cancer therapy within that timeframe. Radiologic tumor assessment will be done at screening and at the end of Cycle 2, Cycle 4, and Cycle 12 and at investigator discretion. Confirmation of complete response (CR) will require bone marrow analysis and radiologic tumor assessment. For subjects who remain on study for >11 months, a mandatory bone marrow aspirate and biopsy is required in Cycle 12 concurrent with the radiologic tumor assessment.

All subjects will have standard hematology, chemistry, and urinalysis safety panels done at screening. This study also includes pancreatic function assessment (serum amylase and serum lipase) due to the pancreatic findings in the 28-day GLP rat toxicity study. Once dosing commences, all subjects will be evaluated for safety once weekly for the first 4 weeks, every other week for Cycle 2, and monthly thereafter. Blood samples will be collected during the first week of treatment for PK/PD assessments. ECGs will be done at screening, and on Day 1-2, 8, 15, 22, 28 of Cycle 1, Day 15 and 28 of Cycle 2, and monthly thereafter through Cycle 6. ECGs are done in triplicate for screening only. Thereafter, single ECG tests are done unless a repeat ECG testing is required.

Dose-limiting toxicity is defined as any of the following events (if not related to disease progression): (1) any Grade≧3 non-hematologic toxicity (except alopecia) persisting despite receipt of a single course of standard outpatient symptomatic therapy (e.g., Grade 3 diarrhea that responds to a single, therapeutic dose of Imodium® would not be considered a DLT); (2) grade≧3 prolongation of the corrected QT interval (QTc), as determined by a central ECG laboratory overread; (3) grade 4 neutropenia (absolute neutrophil count [ANC]<500/μL) lasting >7 days after discontinuation of therapy without growth factors or lasting >5 days after discontinuation of therapy while on growth factors (i.e., Grade 4 neutropenia not lasting as long as specified will not be considered a DLT), (4) grade 4 thrombocytopenia (platelet count <20,000/μL) lasting >7 days after discontinuation of therapy or requiring transfusion (i.e., Grade 4 thrombocytopenia not lasting as long as specified will not be considered a DLT), and (5) dosing delay due to toxicity for >7 consecutive days.

The schedule of assessments is as follows, with all days stated in the following meaning the given day or +/−2 days from the given day. A physical examination, including vital signs and weight, are performed at screening, during cycle 1 at 1, 8, 15, 22, and 28 days, during cycle 2 at 15 and 28 days, during cycles 3 to 24 at 28 days, and at follow up (after the last dose). The screening physical examination includes, at a minimum, the general appearance of the subject, height (screening only) and weight, and examination of the skin, eyes, ears, nose, throat, lungs, heart, abdomen, extremities, musculoskeletal system, lymphatic system, and nervous system. Symptom-directed physical exams are done thereafter. Vital signs (blood pressure, pulse, respiratory rate, and temperature) are assessed after the subject has rested in the sitting position. Eastern Cooperative Oncology Group (ECOG) status is assessed at screening, during cycle 1 at 1, 8, 15, 22, and 28 days, during cycle 2 at 15 and 28 days, during cycles 3 to 24 at 28 days, and at follow up, using the published ECOG performance status indications described in M. M. Oken, et al., Am. J. Clin. Oncol. 1982, 5, 649-655. ECG testing is performed at screening, during cycle 1 at 1, 2, 8, 15, 22, and 28 days, during cycle 2 at 15 and 28 days, during cycles 3 to 24 at 28 days, and at follow up. The 12-lead ECG test will be done in triplicate (≧1 minute apart) at screening. The calculated QTc average of the 3 ECGs must be <480 ms for eligibility. On cycle 1, day 1 and cycle 1, day 8, single ECGs are done predose and at 1, 2, 4, and 6 hours postdose. The single ECG on Cycle 1 Day 2 is done predose. On cycle 1, day 15, day 22, and day 28, a single ECG is done 2 hours post-dose. Starting with cycle 2, a single ECG is done per visit. Subjects should be in supine position and resting for at least 10 minutes before study-related ECGs. Two consecutive machine-read QTc>500 ms or >60 ms above baseline require central ECG review. Hematology, including complete blood count with differential and platelet and reticulocyte counts, is assesed at screening, during cycle 1 at 1, 8, 15, 22, and 28 days, during cycle 2 at 15 and 28 days, during cycles 3 to 24 at 28 days, and at follow up. Serum chemistry is assesed at screening, during cycle 1 at 1, 8, 15, 22, and 28 days, during cycle 2 at 15 and 28 days, during cycles 3 to 24 at 28 days, and at follow up. Serum chemistry includes albumin, alkaline phosphatase, ALT, AST, bicarbonate, blood urea nitrogen (BUN), calcium, chloride, creatinine, glucose, lactate dehydrogenase (LDH), magnesium, phosphate, potassium, sodium, total bilirubin, total protein, and uric acid. Cell counts and serum immunoglobulin are performed at screening, at cycle 2, day 28, and at every 6 months thereafter until last dose and include T/B/NK/monocyte cell counts (CD3, CD4, CD8, CD14, CD19, CD19, CD16/56, and others as needed) and serum immunoglobulin (IgG, IgM, IgA, and total immunoglobulin). Bone marrow aspirates are performed at cycle 12. Pharmacodynamics samples are drawn during cycle 1 at 1, 2, and 8 days, and at follow up. On days 1 and 8, pharmacodynamic samples are drawn pre-dose and 4 hours (±10 minutes) post-dose, and on day 2, pharmacodynamic samples are drawn pre-dose. Pharmacokinetics samples are drawn during cycle 1 at 1, 2, 8, 15, 22, and 28 days. Pharmacokinetic samples for Cycle 1 Day 1 are drawn pre-dose and at 0.5, 1, 2, 4, 6 and 24 hours (before dose on Day 2) post-dose. Samples for Cycle 1 Day 8 are drawn pre-dose and at 0.5, 1, 2, 4, and 6 hours post-dose. On Cycle 1 Day 15, 22, and 28, a PK sample is drawn pre-dose and the second PK sample must be drawn before (up to 10 minutes before) the ECG acquisition, which is 2 hours postdose. Pretreatment radiologic tumor assessments are performed within 30 days before the first dose. A computed tomography (CT) scan (with contrast unless contraindicated) is required of the chest, abdomen, and pelvis. In addition, a positron emission tomography (PET) or PET/CT must be performed for subjects with SLL. Radiologic tumor assessments are mandatory at the end of Cycle 2 (−7 days), Cycle 4 (−7 days), and Cycle 12 (−7 days). Otherwise, radiologic tumor assessments are done at investigator discretion. A CT (with contrast unless contraindicated) scan of the chest, abdomen, and pelvis is required for subjects with CLL. In addition, a PET/CT is required in subjects with SLL. Bone marrow and radiologic assessments are both required for confirmation of a complete response (CR). Clinical assessments of tumor response should be done at the end of Cycle 6 and every 3 months thereafter. Molecular markers are measured at screening, and include interphase cytogenetics, stimulated karyotype, IgHV mutational status, Zap-70 methylation, and beta-2 microglobulin levels. Urinalysis is performed at screening, and includes pH, ketones, specific gravity, bilirubin, protein, blood, and glucose. Other assessments, including informed consent, eligibility, medical history, and pregnancy test are done at the time of screening.

The study scheme is a sequential cohort escalation. Each cohort consists of six subjects. The sample size of the study is 24 to 36 subjects, depending on dose escalation into subsequent cohorts. Cohort 1 (N=6) consists of Formula (II), 100 mg QD for 28 days. Cohort 2 (N=6) consists of Formula (II), 175 mg QD for 28 days. Cohort 3 (N=6) consists of Formula (II), 250 mg QD for 28 days. Cohort 4 (N=6) consists of Formula (II), 350 mg QD for 28 days. Cohort 5 (N=6) consists of Formula (II), 450 mg QD for 28 days. Cohort 6 (N=6) consists of Formula (II), at a dose to be determined QD for 28 days. The dose level for Cohort 6 will be determined based on the safety and efficacy of Cohorts 1 to 5, and will not exceed 900 mg/day. Escalation will end with either the MTD cohort or three levels above full BTK occupancy, whichever is observed first. An additional arm of the study will explore 100 mg BID dosing. Treatment with oral Formula (II) may be continued for greater than 28 days until disease progression or an unacceptable drug-related toxicity occurs.

The inclusion criteria for the study are as follows: (1) men and women ≧18 years of age with a confirmed diagnosis of CLL/SLL, which has relapsed after, or been refractory to, ≧2 previous treatments for CLL/SLL; however, subjects with 17p deletion are eligible if they have relapsed after, or been refractory to, 1 prior treatment for CLL/SLL; (2) body weight ≧60 kg, (3) ECOG performance status of ≦2; (4) agreement to use contraception during the study and for 30 days after the last dose of study drug if sexually active and able to bear children; (5) willing and able to participate in all required evaluations and procedures in this study protocol including swallowing capsules without difficulty; or (6) ability to understand the purpose and risks of the study and provide signed and dated informed consent and authorization to use protected health information (in accordance with national and local subject privacy regulations).

The dosage form and strength of Formula (II) used in the clinical study is a hard gelatin capsules prepared using standard pharmaceutical grade excipients (microcrystalline cellulose) and containing 25 mg of Formula (II) each. The color of the capsules is Swedish orange. The route of administration is oral (per os, or PO). The dose regimen is once daily or twice daily, as defined by the cohort, on an empty stomach (defined as no food 2 hours before and 30 minutes after dosing).

Other details of the study are described in WO 2015/110923, the disclosure of which is incorporated by reference herein.

Subject 1 in the foregoing clinical study is a male CLL patient in his 60's with a history of psoriatic skin disease. Subject 1 had psoriasis since the age of 25 years, without involvement of joints, and with diffuse plaques on his trunk and extremities. His psoriasis was severe, with ˜30% body surface area (BSA) affected. He was treated over time with several systemic therapies including etanercept, adalimumab, and a 2010 clinical study of an experimental agent. He initially responded to etanerceptbut continued to have flares, and was under the care of a dermatologist prior to enrolling in the clinical study. His treatment history included topical medications, and recently included Taclonex (calcipotriene/betamethasone diproprionate), without much improvement in skin lesions.

When treatment with Formula (II) was initiated, Subject 1's skin symptoms started to improve rapidly. There was some improvement during the first month of therapy (noticed by the subject); a consulting dermatologist confirmed that the lesions became less bothersome/inflamed prior to receding. During the first 6 months of treatment, Subject 1's psoriasis had mostly resolved. By Cycle 10 (10 months on Formula (II)), his skin was clear. The results suggest a Psoriasis Area and Severity Index of 90% (PASI90) had been achieved by the subject at the 10 month visit. The only remainder was some scaling in the same areas that were once affected by psoriasis lesions. The patient's history of 30+ years with psoriatic skin disease, with multiple courses of systemic therapy including two TNF inhibitors and inadequate treatment with active topicals, suggests that the resolution of his longstanding disease was not due to a placebo effect, nor to improvement in his CLL.

Changes in Subject 1's serum cytokine and chemokine levels during the first 4 weeks of dosing demonstrate systemic decreases in inflammatory cytokines associated with psoriasis. Notably, interleukin-6 (IL-6), MIP-1α, MCP-1β, TNFA, TARC, CXCLS, CD40L, TRAIL, EGF, and CXCL1 were decreased on Day 28 of treatment, relative to the subject's baseline levels.

Subject 2 was enrolled in a clinical study similar to that described above, and also experienced resolution of moderate-to-severe plaque psoriasis (a “florid” case) while on treatment with Formula (II). The subject's disease history included extensive body surface area involvement, mainly on the back and thighs, for most of his adult life. The subject's disease cleared quickly after initiation of Formula (II) treatment.

The clinical resolution of psoriasis using Formula (II) is surprising because of the selectivity of Formula (II) for BTK and its lack of off-target effects on the T cells normally thought to be of most importance in treatment of psoriasis. The BTK inhibitor of Formula (II) may instead achieve its results through a novel mechanism by inhibition of BTK in other cells, including infiltrating neutrophils, macrophages, mast cells and dendritic cells. Some tyrosine kinase inhibitors used for the treatment of malignancies, such as imatinib, may cause worsening of psoriasis. Dasatinib is a multi-kinase inhibitor that reportedly inhibits BTK, but dasatinib has not been reported to improve psoriasis. Ponatinib causes an extreme degree of skin drying and induces an almost psoriasis-like disease in subjects without a history of skin disease. Formula (II) thus demonstrates a surprising and unexpected result in the successful resolution of moderate-to-severe psoriasis.

### Example 16. Topical Formulations of a BTK Inhibitor

A topical suspension containing 1-10% Formula (II) in an emolient base consisting of an oil phase and an aqueous phase may be formulated as follows. Formula (II) can be introduced into either phase by means of an overhead high shear rotor-stator homogenizer. The oil phase may be heated to 60° C. to decrease the viscosity in order to ensure even distribution of Formula (II). The cream is then emulsified by homogenization while slowly introducing premixed aqueous phase, with or without vacuum to prevent aeration. Alternatively the two phases may be milled using a colloid mill or high pressure piston gap homogenizer. If the input Formula (II) solid is not run through a colloid mill to reduce particle size, it should have a particle size distribution with a D90 less than 100 μm and a D10 greater than 5 μm before introduction to the base.

TABLE 6
Water-Oil (WO) and Oil-Water (OW) Topical Formulations

| | Formulation WO-1 | Formulation OW-1 |
|---|---|---|
| | w/w % | w/w % |
| Oil Phase | | |
| Fractionated lanolin | 2-5% | 25-40% |
| Mineral oil | 20-30% | 5-15% |
| Petrolatum | 20-30% | — |
| Beeswax | 5-15% | 5-15% |
| Sorbitan sesquioleate | 0-2% | — |
| Propyl paraben | 0-0.2% | 0-0.2% |
| Formula (II) | 1-10% | 1-10% |
| Aqueous Phase | | |
| Purified water | 30-50% | 40-60% |
| Sodium borate | 0-2% | — |
| Polyethylene glycol | — | 1-10% |
| (1500-30000 Da | | |
| molecular weight) | | |
| Methyl paraben | 0.05-0.5% | 0.05-0.5% |

Formula (II) may also be formulated as a low dose ointment with a 0.05-1% w/w drug loading. A polyethylene glycol based ointment may be made by softening at 60° C. 25-40% w/w polyethylene glycol 4000 and combining with 50-70% polyethylene glycol 400. To this liquid base, Formula (II) powder is added at 0-1% w/w and dissolved by low shear mixing, with or without vacuum. To make a more liquid base, 25-40% w/w polyethylene glycol 4000 is combined with 50-70% polyethylene glycol 400, 0-10% stearyl alcohol and 6-25% water with 0-0.2% methyparaben.

### Example 17. Nonclinical Models of Psoriasis in Mice

Induction of psoriasis-like lesions is achieved in the BK5.Stat3C transgenic mouse model, in which the constitutive activation of STAT3 in basal keratinocytes predisposes for development of spontaneous chronic psoriaform lesions at the base of the tail, and the induced development of psoriaform lesions after tape-stripping, wounding, or application of 12-O-tetradecanoylphorbol-13-acetate (TPA). In this model, adult BK5.Stat3C are tape-stripped on the shaved dorsal skin to induce the development of psoriaform lesions.

Five BK5.Stat3C transgenic mice per group are treated with vehicle or Formula (II) by oral gavage at doses of 1, 5 and 25 mg/kg daily for 5 days. A positive control treatment is also administered. The development of psoriatic lesions in the tape-stripped region is evaluated with clinical and histological scoring. Briefly, acanthosis, vascularity, expression of Ki67, and infiltrating CD3⁺ T cells will be scored microscopically. Additional markers of inflammation include the characterization of specialized immune cell subsets, activated mast cells, neutrophils, and extracellular traps in the psoriatic plaques by immunohistochemistry or by flow cytometry analysis of dermal and epidermal immune infiltrates. Acute lesions are evaluated on treatment Day 5. The resolution of spontaneous tail-base lesional skin is evaluated after 1, 2, and 4 weeks of dose administration in adult mice. Gene expression analysis by targeted arrays (i.e., inflammation signature) or by RNAseq in psoriatic skin of BK5.Stat3C transgenic mice in the vehicle, positive control, and Formula (II) treated mice are used to evaluate changes in the expression of cytokines, chemokines, and other mechanistic markers of inflammation. BTK target saturation associated with each dose level of Formula (II) are measured in splenocyte preparations as well as in preparations of dermal infiltrates isolated from psoriatic and non-lesional skin. In addition, this model may be conducted using a topical formulation of Formula (II) applied directly to the tape-stripped skin for evaluation of efficacy in the early stages of psoriatic lesion development, and to the psoriaform skin associated with the chronic tail-base lesions that occur spontaneously in BK5.Stat3C transgenic mice.

Another mouse model of psoriasis involves the transplantation of a human xenograft from a psoriasis patient into the dorsal skin of a SCID mouse (also known as a Hu-SCID psoriasis model) to allow for mechanistic evaluations of experimental therapeutics. The Hu-SCID psoriasis model may require activated human T cells to be infused into the mouse to induce or maintain the autoimmune response within the plaque, however due to presence of NK cells in SCID mice, the survival of xenografted skin is tenuous. The use of the AGR129 mouse (deficient in IFN-α, IFN-β and RAG-2) allows the induction of full-fledged psoriatic phenotype from pre-lesional skin, based solely on the resident cells within the xenograft, in 6-8 weeks without the additional stimulus of donor T cells. Treatment of AGR129 mice xenografted with pre-lesional skin from psoriasis patients with Formula (II) may be used during the induction phase, or, following establishment of psoriaform lesions in the xenograft, as a therapeutic model.

To evaluate the dose-response and efficacy of Formula (II), doses of 1, 5, or 25 mg/kg administered daily by oral gavage are compared with vehicle control and with an active immunosuppressive agent (e.g., an anti-TNF-α antibody). The clinical features of the plaque are scored weekly during the in-life phase, using a standardized scale for lesion severity. Histopathological features of the xenograft are evaluated microscopically, including acanthosis, development of rete ridges, dermal and epidermal lymphoid aggregates, inflammatory infiltrates, epidermal markers of keratinocyte growth and differentiation (e.g., Ki67, K16, K17, pyknosis, and S100), and markers for infiltrating myeloid cells including mast cells, myeloid and plasmacytoid dendritic cells, macrophages, and neutrophils. Gene expression analysis of xenografts at pre-treatment and post-treatment timepoints may be used to evaluate the activation status of infiltrating cells, pro-inflammatory mediators, and expression of antimicrobial peptides. In addition, this model may be conducted using a topical formulation of Formula (II) applied directly to the xenograft during the disease induction phase or following development of the psoriaform lesion.

### Example 18. Nonclinical Models of Scleroderma in Mice

Induction of scleroderma (or systemic sclerosis, SSc) can be evaluated in mouse models including a genetic model (i.e., the tight-skin mouse, TSK/+) and a bleomycin-induced dermal fibrosis model. To evaluate the effects of Formula (II) in a mouse model of scleroderma, female C3H/HeJ mice aged 6-8 weeks are injected subcutaneously with bleomycin solution on selected dorsal locations, every other day for 4 weeks. During the induction period, vehicle or Formula (II) is administered by oral gavage at dosages of 1, 5, or 25 mg/kg daily. The mice are euthanized and dorsal skin is removed for fixation with 10% neutral buffered formalin and paraffin embedding for histological evaluation of fibrosis by histochemistry and dermal thickness by quantitative image analysis. Myelofibroblasts are identified by immunohistochemistry staining of a-smooth muscle actin. Dermal infiltrates, immune complex formation, and proliferation index are measured by methods known to those skilled in the art. Ten mice per treatment group are included for statistical evaluation of the histopathology scores. Peripheral blood cytokine profiles are evaluated using a multiplex bead-based ligand-binding assay. In a subset of samples, FFPE sections are submitted to quantitative mRNA analysis by in situ hybridization to evaluate fibrogenic cytokine profiles (IL-6, TGF-β) within the lesional skin.

### Example 19. Nonclinical Models of Atopic Dermatitis in Mice and Dogs

The skin phenotype and pruritis associated with atopic dermatitis (AD) can be modeled using specific strains of mice, with or without disruption of the cutis, exposure to specific pathogens, and induction with antigenic stimuli such as extract of Dermatophagoides farina (Df) or trinitrochlorobenzene. Under non-SPF (specific pathogen free) conditions, the NC/Nga mouse is prone to dermatitis, and with tape-stripping and/or epidermal stimulus will develop an AD phenotype even in cleaner facilities. Clinical and biochemical signs include an inflammatory skin phenotype of erythema, edema, hyperplasia, hemorrhage, crusting, and excoriation/erosion, with increased expression of IL-5, IL-13, TARC, C-TACK, and eotaxin, dermal accumulation of lymphocytes and mast cells, epidermal migration of neutrophils, neurite outgrowth, hyperplasia, acanthosis, deepening rete ridges, and dermal inflammatory infiltrates rich in lymphoctyes and eosinophils.

These phenotypic changes in mice can be monitored clinically and histopathologically. Experimental agents may be tested in the NC/Nga mice to evaluate the aggregate scores from the histopathological features of disease. Additional quantitative data from piezometric analysis of motion as relates to itching and/or in-cage motion may be obtained. Increases in numbers of circulating Th2 and CLA⁺ T cells, serum levels of IL-5, IL-13, TARC and eotaxin, specific receptors such as PAR-2, IL-31R, FcεR1 in the dermis and/or epidermis, and skin eosinophils and mast cells are observed concurrently with worsening disease phenotype.

For example, NC/Nga mice are tape-stripped and induced with Df antigen, and dosing with Formula (II) is initiated upon the onset of a moderate disease score (e.g., 3 out of 4 on the clinical assessment scale). Treatment of groups consisting of 5-10 mice administered vehicle control, using Formula (II) at doses from 5 mg/mL to 50 mg/mL or a positive control agent, is initiated on a rolling basis, as the aging rats develop sufficiently high clinical scores. The amelioration of pruritis is a strong clinical indicator of efficacy in this mouse model, whereas additional mechanistic information can be derived from histological and molecular analysis of the affected skin. Expression of neuroinflammatory mediators including IL-31, PAR-2, and substance P, and the effects on pruroceptors, neurite outgrowth, and dermal infiltrates may be evaluated using immunohistochemistry, in situ hybridization, or gene expression arrays.

Dogs with pruritic eczema are typically treated with sedatives and/or topical corticosteroids, or therapies such as oral glucocorticoids for acute flares and glucocorticoids, cyclosporine, and antibiotics or antifungal agents for chronic atopic dermatitis. Antihistamines in combination with oral glucocorticoids may improve the control of pruritis, however antihistamines alone are insufficient to control atopic disease despite the presence of IgE and activation of dermal mast cells in affected skin.

In companion dogs with acute flares of AD or with chronic AD, Formula (II) treatment is administered daily at doses of 2.5 to 30 mg/kg by oral capsule. Capsules are administered daily and the clinical features of disease are evaluated once weekly after baseline assessment of the percentage of skin involvement, presence and grade of disease at paws, ears and axilliary folds, and the owner's subjective scoring of itching behavior (i.e., a Visual Analogue Scale, VAS). As an option, dogs may be fitted with psiesometry devices on the front or hind paws to quantitatively evaluate the percentage time engaged in scratching. Histological examination of affected skin is performed at baseline and weeks 2, 4, and 12. All quantitative and qualitative measures of disease activity from dogs treated with Formula (II) are evaluated at baseline, weeks 2, 4 and 12; at the owner's option, Formula (II) treatments may be extended up to 26 weeks in responding animals. Clinical evaluations include the owner's VAS, estimated percentage of body surface area affected, severity grading of skin lesions, presence and count of exorciations, involvement of paws and ears, and composite scores. Biological markers, including serum chemistry, serum IgE levels, serum cytokines and chemokines, circulating myeloid and lymphoid cell subsets, circulating CLA+ lymphocytes, and histopathological scoring of biopsies from affected versus non-lesional skin sites, are evaluated to determine extent of biological response. Dogs will be treated for up to 12 weeks, and then followed for another 4 weeks after cessation of therapy. If efficacy is observed in more than one dose level, then additional studies may be merited to select a dose for further evaluation of Formula (II) in dogs. Dogs may be treated orally with capsule, tablet, or gavage dosing of a liquid formulation of Formula (II). In addition, Formula (II) may be formulated as described in Example 16 and applied topically to affected areas of skin for the treatment of acute AD, and/or combined with a short course of an antimicrobial or antifungal therapy for the treatment of chronic AD in companion dogs.

### Example 20. Nonclinical Models of Cutaneous Lupus Erythematosus in Mice

The skin phenotypes associated with systemic lupus erythematosus and discoid lupus can be modeled using specific strains of mice. In lupus patients, sensitivity to minor skin damage and UV irradiation leads to erythema and skin manifestations of disease, due to dysregulated innate and adaptive immune reactivity in combination with epidermal barrier disruption. Generation of antimicrobial peptides, cytokine, TLR or UV-mediated activation of keratinocytes, and the activation of dermal plasmacytoid dendritic cells may also result in increased systemic autoimmune reactivity and disease flares. The NZB×NZW (NZBW) F1 mouse is a preclinical model used for investigating development and treatment of systemic and cutaneous lupus erythematosus. Female 11 week old NZBW F1 mice are aged until 15-20 weeks, when proteinuria and/or serum blood urea nitrogen indicates the onset of disease. Mice are then shaved twice weekly on their dorsal skin and exposed to 500 mJ/cm² of UVB light in photoirradiation cages, every other day, for 8 weeks. Subsequently, treatment with Formula (II) is initiated by administration of 0, 1, 5, or 25 mg/kg Formula (II) by oral gavage once daily for the duration of the experiment. Alternatively, cutaneous flare prevention can be evaluated by initiating treatment with Formula (II) by oral gavage once daily during the photoinduction period. Skin is scored twice weekly for cutaneous manifestations of disease. At the end of the treatment period, the mice are euthanized and the dorsal skin is shaved and removed for histopathological evaluation, in situ or gel zymography, gene expression analysis, flow cytometry analysis of resident lymphocytes, and the characteristic deposition of immune complexes in the dermal/epidermal junction. Immuno-fluorescence techniques may be used in frozen-embedded or formalin-fixed paraffin-embedded skin samples from control and treated NZBW mice. Treatment effects on subcutaneous edema, inflammatory cell infiltrates, and parakeratosis may be evaluated semi-quantitatively, or with the aid of an image analysis system. At the time of sacrifice, evaluation of the plasma for BUN/creatinine, cytokine/chemokine, immunoglobulins, and specific autoantibodies including ssDNA, dsDNA and other autoimmune markers may be performed. Renal histopathology may also be evaluated to generate systemic disease scores.

Tape stripping of the MLR/lpr mouse also produces cutaneous lesions, with correlated systemic effects such as increased proteinuria. Thus, the cutaneous manifestations of systemic disease may be induced in this strain of mouse by epithelial barrier disruption. Similar studies with Formula (II) may be conducted to evaluate the effects of treatment during the induction interval or after clinical induction. The clinical, histopathological, and biochemical (serum) manifestations of disease in these lupus-prone mice may be scored as described above.

1-85. (canceled)

86. A method of treating a BTK mediated disease in a human comprising the steps of:
(a) administering a Bruton's tyrosine kinase (BTK) inhibitor at a first dose for a first period sufficient to provide a target BTK occupancy in a tissue compartment; and
(b) administering the BTK inhibitor at a second dose for a second period, wherein the second dose is less than the first dose and is sufficient to provide the target BTK occupancy in the tissue compartment.

87. The method of claim 86, wherein the BTK inhibitor is selected from the group consisting of: 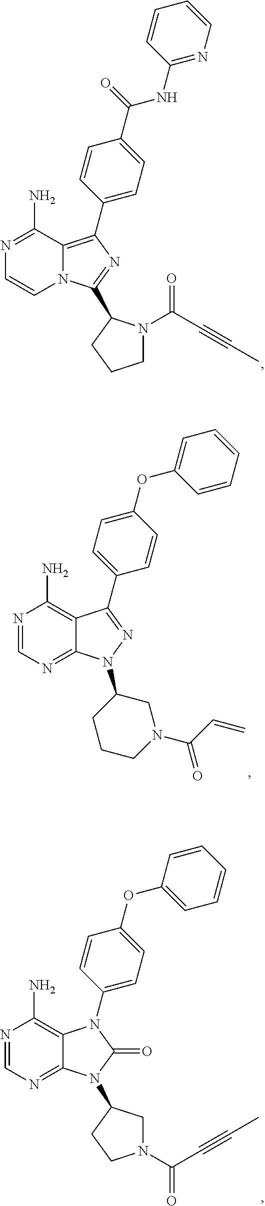 [SMILES: CC#CC(=O)N1CCC[C@H]1c1nc(-c2ccc(C(=O)Nc3ccccn3)cc2)c2c(N)nccn12 ; C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1] 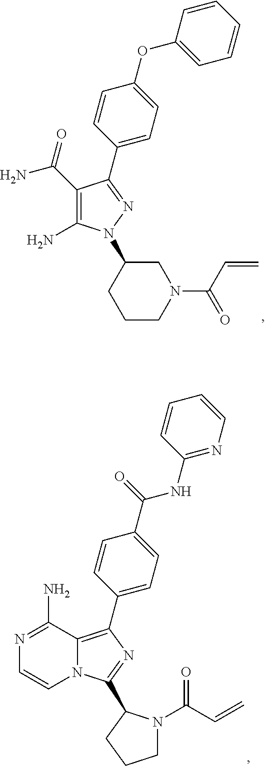 [SMILES: C=CC(=O)N1CCC[C@@H](n2nc(-c3ccc(Oc4ccccc4)cc3)c(C(N)=O)c2N)C1 ; C=CC(=O)N1CCC[C@H]1c1nc(-c2ccc(C(=O)Nc3ccccn3)cc2)c2c(N)nccn12] 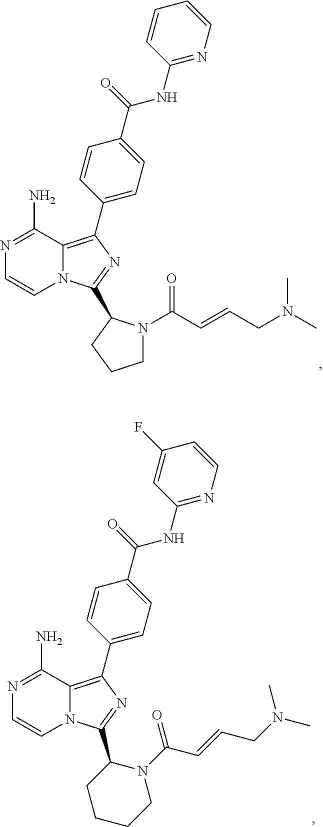 [SMILES: CN(C)C/C=C/C(=O)N1CCC[C@H]1c1nc(-c2ccc(C(=O)Nc3ccccn3)cc2)c2c(N)nccn12 ; CN(C)C/C=C/C(=O)N1CCCC[C@H]1c1nc(-c2ccc(C(=O)Nc3cc(F)ccn3)cc2)c2c(N)nccn12] 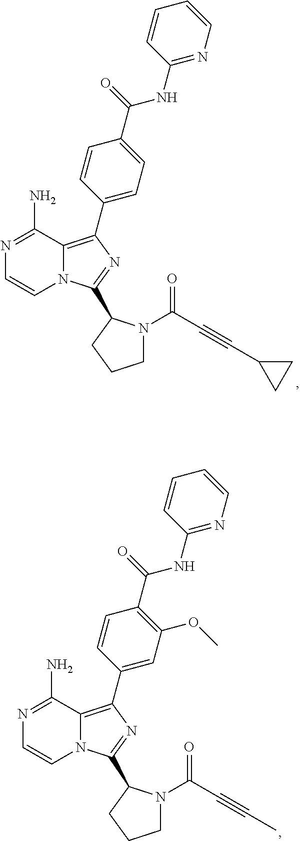 [SMILES: Nc1nccn2c([C@@H]3CCCN3C(=O)C#CC3CC3)nc(-c3ccc(C(=O)Nc4ccccn4)cc3)c12 ; CC#CC(=O)N1CCC[C@H]1c1nc(-c2ccc(C(=O)Nc3ccccn3)c(OC)c2)c2c(N)nccn12]
or a pharmaceutically-acceptable salt, cocrystal, hydrate, solvate, or prodrug thereof.

88. The method of claim 87, wherein the target BTK occupancy is selected from the group consisting of greater than 85%, greater than 90%, greater than 91%, greater than 92%, greater than 93%, greater than 94%, greater than 95%, greater than 96%, greater than 97%, greater than 98%, and greater than 99%.

89. The method of claim 87, wherein the first dose of the BTK inhibitor is administered once daily, twice daily, or three times daily.

90. The method of claim 87, wherein the second dose of the BTK inhibitor is administered once daily, twice daily, or three times daily.

91. The method of claim 87, wherein the first dose of the BTK inhibitor is selected from the group consisting of 5 mg, 10 mg, 15 mg, 20 mg, and 25 mg.

92. The method of claim 87, wherein the second dose of the BTK inhibitor is selected from the group consisting of 5 mg, 10 mg, 15 mg, 20 mg, and 25 mg.

93. The method of claim 87, wherein the first period is selected from the group consisting of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, and 21 days.

94. The method of claim 87, wherein the second period is selected from the group consisting of 2 weeks, 1 month, 2 months, 3 months, 6 months, 9 months, 1 year, 2 years, 3 years, 4 years, 5 years, 6 years, 7 years, 8 years, 9 years, and 10 years.

95. The method of claim 87, wherein the first dose of the BTK inhibitor is administered orally or topically.

96. The method of claim 87, wherein the second dose of the BTK inhibitor is administered orally or topically.

97. The method of claim 87, wherein the BTK mediated disease is a cancer selected from the group consisting of a cancer selected from the group consisting of non-Hodgkin's lymphoma, acute myeloid leukemia, chronic lymphocytic leukemia, small lymphocytic lymphoma, diffuse large B cell lymphoma, mantle cell lymphoma, MALT lymphoma, Waldenstrom's macroglobulinemia, follicular lymphoma, B cell acute lymphoblastic leukemia, Burkitt's leukemia, juvenile myelomonocytic leukemia, prolymphocytic leukemia, mast cell leukemia, hairy cell leukemia, Hodgkin's disease, multiple myeloma, thymus cancer, brain cancer, glioma, lung cancer, squamous cell cancer, skin cancer, melanoma, eye cancer, retinoblastoma, intraocular melanoma, oral cavity cancer, oropharyngeal cancer, bladder cancer, gastric cancer, stomach cancer, pancreatic cancer, breast cancer, cervical cancer, head cancer, neck cancer, renal cancer, kidney cancer, liver cancer, ovarian cancer, prostate cancer, colorectal cancer, bone cancer, esophageal cancer, testicular cancer, gynecological cancer, thyroid cancer, central nervous system cancer, cancer related to acquired immune deficiency syndrome, cervical carcinoma, nasopharyngeal carcinoma, Kaposi's sarcoma and, primary effusion lymphoma, adenocystic carcinoma, hepatocellular carcinoma, T-cell leukemia, and mastocytosis.

98. A method of treating a disorder in a human comprising the step of administering a dose of a compound selected from the group consisting of: 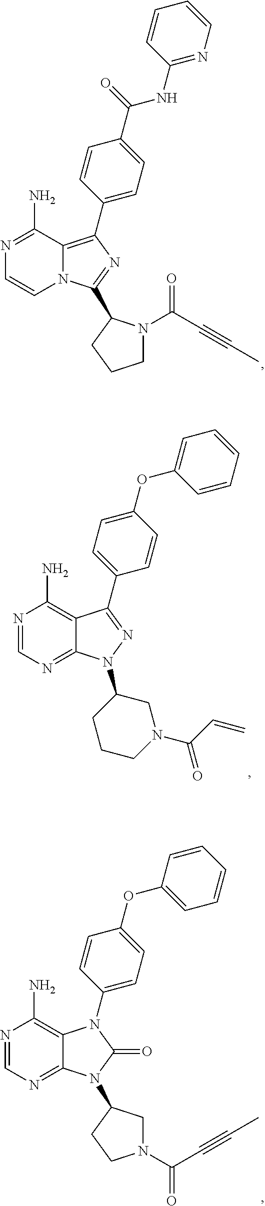 [SMILES: CC#CC(=O)N1CCC[C@H]1c1nc(-c2ccc(C(=O)Nc3ccccn3)cc2)c2c(N)nccn12 ; C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1] 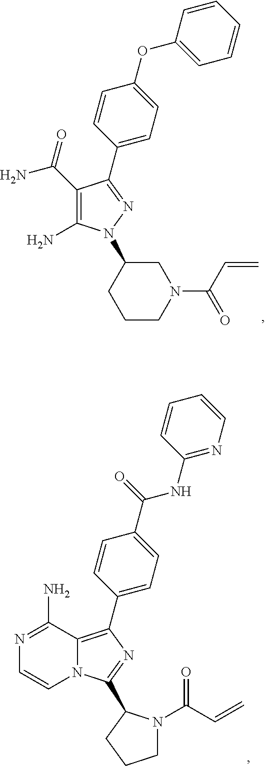 [SMILES: C=CC(=O)N1CCC[C@@H](n2nc(-c3ccc(Oc4ccccc4)cc3)c(C(N)=O)c2N)C1 ; C=CC(=O)N1CCC[C@H]1c1nc(-c2ccc(C(=O)Nc3ccccn3)cc2)c2c(N)nccn12] 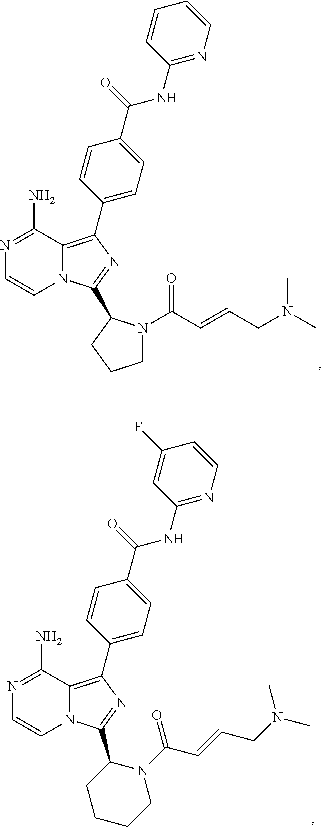 [SMILES: CN(C)C/C=C/C(=O)N1CCC[C@H]1c1nc(-c2ccc(C(=O)Nc3ccccn3)cc2)c2c(N)nccn12 ; CN(C)C/C=C/C(=O)N1CCCC[C@H]1c1nc(-c2ccc(C(=O)Nc3cc(F)ccn3)cc2)c2c(N)nccn12] 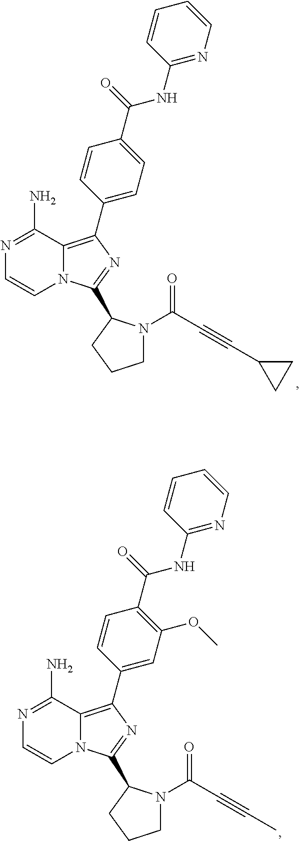 [SMILES: Nc1nccn2c([C@@H]3CCCN3C(=O)C#CC3CC3)nc(-c3ccc(C(=O)Nc4ccccn4)cc3)c12 ; CC#CC(=O)N1CCC[C@H]1c1nc(-c2ccc(C(=O)Nc3ccccn3)c(OC)c2)c2c(N)nccn12]
or a pharmaceutically-acceptable salt, cocrystal, hydrate, solvate, or prodrug thereof, wherein the dose is selected from the group consisting of 5 mg, 10 mg, 15 mg, 20 mg, and 25 mg, wherein the dose is administered once daily, twice daily, or three times daily, and wherein the dose is administered by a route of administration selected from the group consisting of oral administration, topical administration, and combinations thereof.

99. The method of claim 98, wherein the disorder is a cancer, and wherein the cancer is selected from the group consisting of non-Hodgkin's lymphoma, acute myeloid leukemia, chronic lymphocytic leukemia, small lymphocytic lymphoma, diffuse large B cell lymphoma, mantle cell lymphoma, Waldenstrom's macroglobulinemia, follicular lymphoma, B cell acute lymphoblastic leukemia, Burkitt's leukemia, juvenile myelomonocytic leukemia, mast cell leukemia, hairy cell leukemia, Hodgkin's disease, multiple myeloma, thymus cancer, brain cancer, glioma, lung cancer, squamous cell cancer, skin cancer, melanoma, eye cancer, retinoblastoma, intraocular melanoma, oral cavity cancer, oropharyngeal cancer, adenocystic carcinoma, bladder cancer, gastric cancer, stomach cancer, pancreatic cancer, breast cancer, cervical cancer, head cancer, neck cancer, renal cancer, kidney cancer, liver cancer, ovarian cancer, prostate cancer, colorectal cancer, bone cancer, esophageal cancer, testicular cancer, gynecological cancer, thyroid cancer, central nervous system cancer, cancer related to acquired immune deficiency syndrome, cervical carcinoma, nasopharyngeal carcinoma, Kaposi's sarcoma and, primary effusion lymphoma, hepatocellular carcinoma, T-cell leukemia, and mastocytosis.

100. A method of treating a BTK mediated disease in a human comprising the step of:
(a) administering a BTK inhibitor in a dosage form that provides controlled release of the active pharmaceutical agent over time, wherein the release is sufficient to provide a target BTK occupancy in a tissue compartment over the course of sub-chronic or chronic administration.

101. The method of claim 100, wherein the BTK inhibitor is selected from the group consisting of: 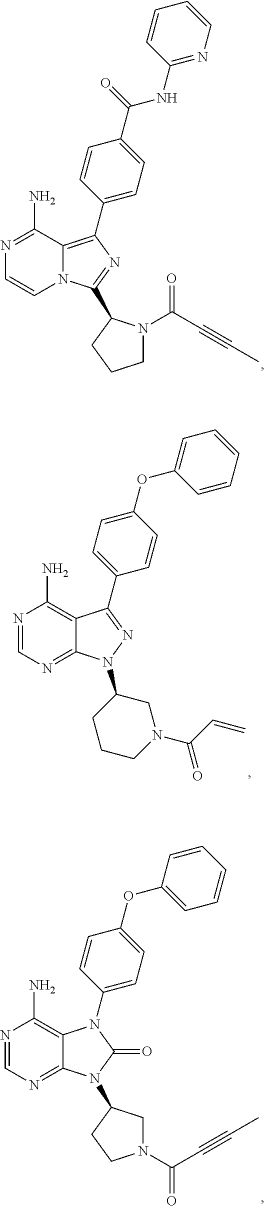 [SMILES: CC#CC(=O)N1CCC[C@H]1c1nc(-c2ccc(C(=O)Nc3ccccn3)cc2)c2c(N)nccn12 ; C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1] 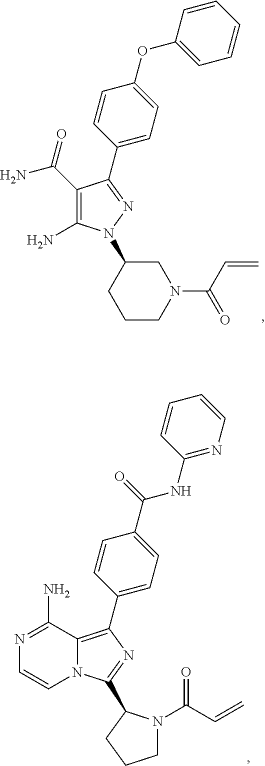 [SMILES: C=CC(=O)N1CCC[C@@H](n2nc(-c3ccc(Oc4ccccc4)cc3)c(C(N)=O)c2N)C1 ; C=CC(=O)N1CCC[C@H]1c1nc(-c2ccc(C(=O)Nc3ccccn3)cc2)c2c(N)nccn12] 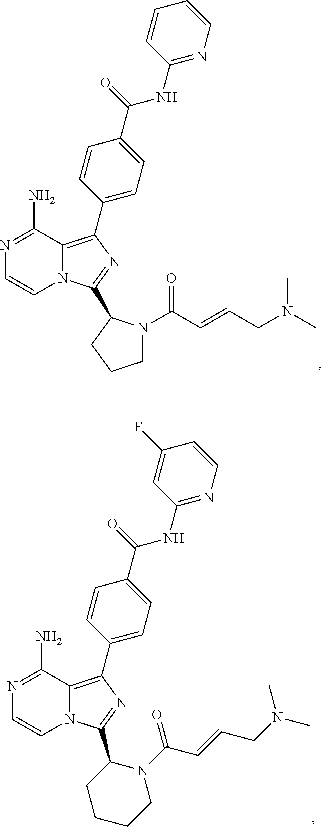 [SMILES: CN(C)C/C=C/C(=O)N1CCC[C@H]1c1nc(-c2ccc(C(=O)Nc3ccccn3)cc2)c2c(N)nccn12 ; CN(C)C/C=C/C(=O)N1CCCC[C@H]1c1nc(-c2ccc(C(=O)Nc3cc(F)ccn3)cc2)c2c(N)nccn12] 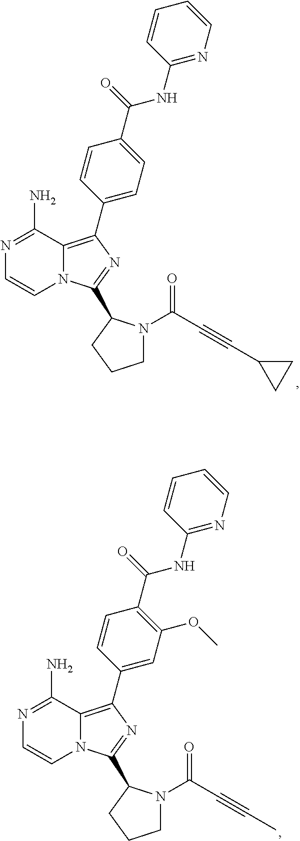 [SMILES: Nc1nccn2c([C@@H]3CCCN3C(=O)C#CC3CC3)nc(-c3ccc(C(=O)Nc4ccccn4)cc3)c12 ; CC#CC(=O)N1CCC[C@H]1c1nc(-c2ccc(C(=O)Nc3ccccn3)c(OC)c2)c2c(N)nccn12]
or a pharmaceutically-acceptable salt, cocrystal, hydrate, solvate, or prodrug thereof.

102. The method of claim 101, wherein the BTK mediated disease is a cancer selected from the group consisting of a cancer selected from the group consisting of non-Hodgkin's lymphoma, acute myeloid leukemia, chronic lymphocytic leukemia, small lymphocytic lymphoma, diffuse large B cell lymphoma, mantle cell lymphoma, Waldenstrom's macroglobulinemia, follicular lymphoma, B cell acute lymphoblastic leukemia, Burkitt's leukemia, juvenile myelomonocytic leukemia, mast cell leukemia, hairy cell leukemia, Hodgkin's disease, multiple myeloma, thymus cancer, brain cancer, glioma, lung cancer, squamous cell cancer, skin cancer, melanoma, eye cancer, retinoblastoma, intraocular melanoma, oral cavity cancer, oropharyngeal cancer, adenocystic carcinoma, bladder cancer, gastric cancer, stomach cancer, pancreatic cancer, breast cancer, cervical cancer, head cancer, neck cancer, renal cancer, kidney cancer, liver cancer, ovarian cancer, prostate cancer, colorectal cancer, bone cancer, esophageal cancer, testicular cancer, gynecological cancer, thyroid cancer, central nervous system cancer, cancer related to acquired immune deficiency syndrome, cervical carcinoma, nasopharyngeal carcinoma, Kaposi's sarcoma and, primary effusion lymphoma, hepatocellular carcinoma, T-cell leukemia, and mastocytosis.

103. The method of claim 101, wherein the target BTK occupancy is selected from the group consisting of greater than 85%, greater than 90%, greater than 91%, greater than 92%, greater than 93%, greater than 94%, greater than 95%, greater than 96%, greater than 97%, greater than 98%, and greater than 99%.

104. The pharmaceutical composition of claim 101, wherein the dose is selected from the group consisting of 5 mg, 10 mg, 15 mg, 20 mg, and 25 mg.

105. A method of treating a dermatosis comprising the step of administering a therapeutically-effective amount of a BTK inhibitor selected from the group consisting of: 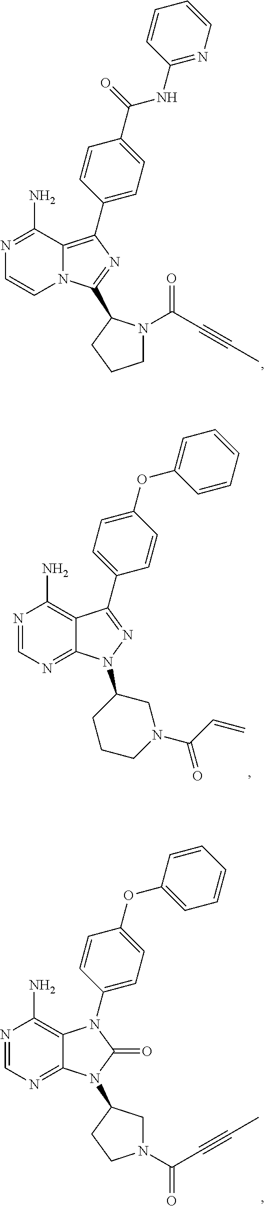 [SMILES: CC#CC(=O)N1CCC[C@H]1c1nc(-c2ccc(C(=O)Nc3ccccn3)cc2)c2c(N)nccn12 ; C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1] 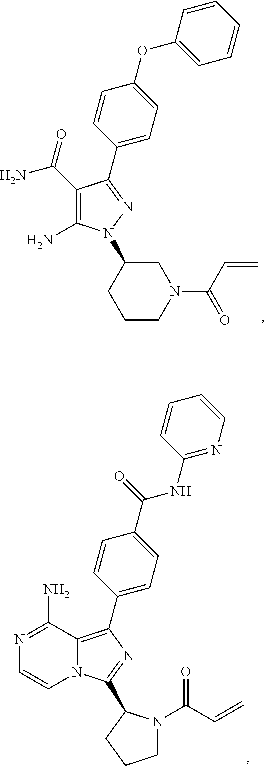 [SMILES: C=CC(=O)N1CCC[C@@H](n2nc(-c3ccc(Oc4ccccc4)cc3)c(C(N)=O)c2N)C1 ; C=CC(=O)N1CCC[C@H]1c1nc(-c2ccc(C(=O)Nc3ccccn3)cc2)c2c(N)nccn12] 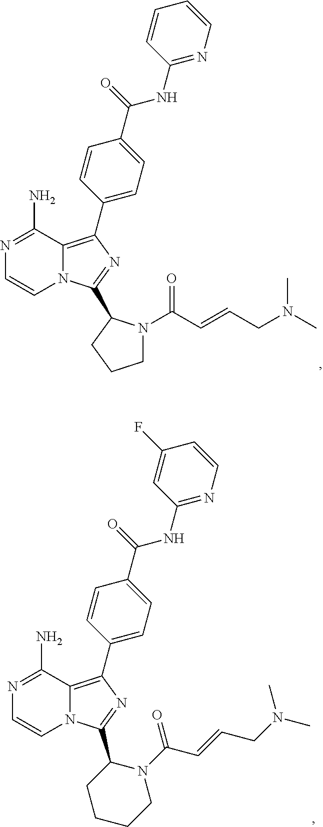 [SMILES: CN(C)C/C=C/C(=O)N1CCC[C@H]1c1nc(-c2ccc(C(=O)Nc3ccccn3)cc2)c2c(N)nccn12 ; CN(C)C/C=C/C(=O)N1CCCC[C@H]1c1nc(-c2ccc(C(=O)Nc3cc(F)ccn3)cc2)c2c(N)nccn12] 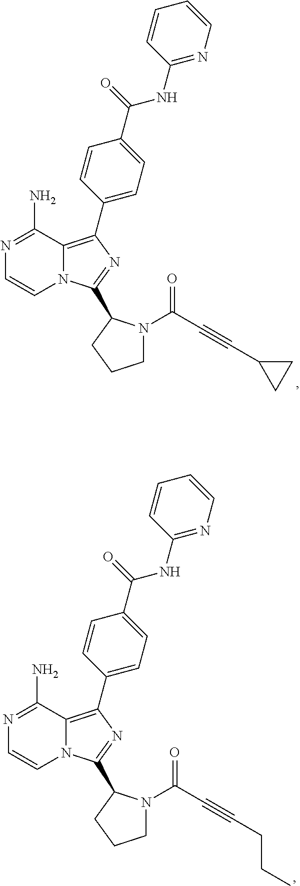 [SMILES: Nc1nccn2c([C@@H]3CCCN3C(=O)C#CC3CC3)nc(-c3ccc(C(=O)Nc4ccccn4)cc3)c12 ; CCCC#CC(=O)N1CCC[C@H]1c1nc(-c2ccc(C(=O)Nc3ccccn3)cc2)c2c(N)nccn12]
or a pharmaceutically-acceptable salt, cocrystal, hydrate, solvate, or prodrug thereof.

106. The method of claim 105, wherein the dermatosis is selected from the group consisting of psoriasis vulgaris, guttate psoriasis, erythrodermic psoriasis, psoriatic nails, annular pustular psoriasis, pustular psoriasis, inverse psoriasis, psoriatic arthritis, keratoderma blennorrhagicum, parapsoriasis, erythema nodosum, palmoplantar hidradentitis, atopic dermatitis, atopic eczema, seborrheic eczema, seborrheic dermatitis, dyshidrosis, rosacea, cutaneous lupus erythematosus, acute cutaneous lupus erythematosus, subacute cutaneous lupus erythematosus, discoid lupus erythematosus, lupus erythromatosus tumidus, lupus nephritis, lupus erythematosus panniculitis, erythema multiforme, verruca, verrucous lupus erythematosus, vitiligo, alopecia areata, purigo nodularis, lichen planus, purigo pigmentosum, pemphigus vulgaris, bullous pemphigoid, pemphigus erythematosus, pemphigus nodularis, erythrodermic sarcoidosis, granulomatous dermatisis, scleroderma, systemic sclerosis, cutaneous manifestations of systemic sclerosis, diffuse cutaneous mastocytosis, erythrodermic mastocytosis, granuloma annulare, chondrodermatitis nodularis, contact dermatitis, drug eruptions, linear IgA bullous dermatosis, eosinophilic dermatitis, keratosis pilaris, lymphomatoid papulosis, pityriasis lichenoides et varioliformis acuta (PLEVA), lichenoides chronica (PLC), febrile ulceronecrotic Mucha-Habermann disease (FUMHD), chronic urticaria, rheumatoid neutrophilic dermatitis, cutaneous manifestations of graft-versus-host disease, cryoglobulinemic purpura, and purpura hyperglobulinemica.

107. The method of claim 105, wherein the dermatosis is selected from the group consisting of psoriasis, scleroderma, atopic dermatitis, and cutaneous lupus erythematosus.

108. The method of claim 105, wherein the therapeutically effective dose of the BTK inhibitor is selected from the group consisting of 5 mg, 10 mg, 15 mg, 20 mg, 25 mg, 30 mg, 45 mg, 60 mg, 75 mg, 90 mg, and 100 mg, wherein the therapeutically effective dose is administered at an interval selected from the group consisting of once daily, twice daily, three times daily, and four times daily, and wherein the therapeutically effective dose is administered by a route of administration selected from the group consisting of oral administration, topical administration, and combinations thereof.
